SEQUENCE LISTING (44 sequences)

SEQ ID NO: 1 - protein, 424 aa, Arabidopsis thaliana
MAVFKKKKTSLTSLFLGCYKAKNVSKYEGGEKAVMKIRTCPAFKRLSLSDISDPSSPMSVMDDLSHSFTSQKLRLFTLSELRVITHNFSRSNMLGEGGFGPVYKGFIDDKVKPGIEAQPVAVKALDLHGHQGHREWLAEILFLGQLSNKHLVKLIGFCCEEEQRVLVYEYMPRGSLENQLFRRNSLAMAWGIRMKIALGAAKGLAFLHEAEKPVIYRDFKTSNILLDSDYNAKLSDFGLAKDGPEGEHTHVTTRVMGTQGYAAPEYIMTGHLTTMNDVYSFGVVLLELITGKRSMDNTRTRREQSLVEWARPMLRDQRKLERIIDPRLANQHKTEAAQVAASLAYKCLSQHPKYRPTMCEVVKVLESIQEVDIRKHDGNNNKEGKKFVDINKFRHHRKGKRRVNIAYSDSLVYKESKAKQNDGI

SEQ ID NO: 2 - RNA, 1706 nt, Arabidopsis thaliana
  source 1..1706: mol_type = mRNA
gtacgaataagacacaacaactactataaattagaggactttgaagacaagtaggttaactagaacatccttaatttctaaacctacgcactctacaaaagattcatcaaaaggagtaaaagactaactttctccattttttccccaaataacacaaaagaagaagttgaaaatggcagtgttcaagaagaagaaaacatcattgacatctctgttcttggggtgttacaaggcaaagaacgcaagcaaatacgaaggaggggagaaggctgtaatgaaaataagaacttgtccagcgttcaagaggctgtctttatcggacataagcgatccaagctcgcccatgtcggttatggatgatctctctcactccttcacatcgcagaagcttcgtttgttcacgttgtctgagctgagagtgattacgcataatttctctagaagtaacatgttgggggaaggagggtttgggccggtttacaaagggtttattgatgataaggttaaaccgggtatcgaagcccaaccggttgctgtaaaggctcttgatcttcatggccatcaaggccatagagaatggctggcggagatattatttttgggacaactaagtaacaaacatcttgtgaagctgatagggttttgttgtgaagaagagcaaagagttcttgtttatgagtacatgcctcgaggtagcttggagaatcagctctttcgaagaaatagcttagcaatggcttggggaataaggatgaagatagctctcggagccgccaaaggtttagcttttctccatgaagcagagaaacctgtcatctacagagactttaaaacctctaacatattgttagactccgactataacgccaaattatctgacttcggtctagccaaagatggacctgaaggcgaacatacgcatgtaactacccgagtgatgggaacacaaggttatgctgcgcccgaatatatcatgactggtcatttgacgacgatgaatgatgtatacagttttggagtagttttgttagagctgataaccggaaaacggtcaatggacaatacccgaacacggagggaacaaagcctagtggaatgggcgcggcctatgttaagggaccaaagaaagttagagaggataattgacccgaggctcgcgaaccagcacaaaacagaagcggctcaggtagctgcttccctagcatacaagtgtctaagccaacatcctaagtatagacctacaatgtgtgaggtggttaaggtcttagagtctatccaagaagttgatattaggaagcatgatggcaataacaacaaagaaggaaagaagtttgttgatataaataagtttaggcatcaccgtaaaggcaaaagacgtgtgaacatcgcttattcagattctctcgtttacaaggaatccaaggcaaagcaaaacgatggcatttgaaatacatcattttctcatatgaattattaagcattcatattctatgaacgcaattcttatcttaagttatgaccattcttccaaaaatttgaaggaaagaaaggtgatgttataagtatcctagttctctggtattttagattaatttcctttacataattcaaaaatactttgtacaaaagagtcatgtacaagaaatataaaataaatgtttgtaaatcatatatatatacaccagtttgttgggcggacaacgttg

SEQ ID NO: 3 - protein, 382 aa, Zea mays
MGKPAESSSTSWSALFGFGCFSSSHADRSGDGPGKVASAGSRPPAPPAPLLSPDDLSLSLAGSDVLAFTVEELRVATRDFSMSNFVGEGGFGPVYKGRVDERVRPGLRQPQAVAVKLLDLEGSQGHKEWLAEVIFLGQLRHPHLVKLIGYCYQDEHRLLVYEFMARGSLEKHLFKRHSASLPWSTRMRIAIGAAKGLAFLHDAAKPVIYRDFKTSNILLDSDYTAKLSDFGLAKAGPGEDETHVSTRVMGTQGYAAPEYIMTGHLTTKSDVYSFGVVLLELLTGRKALDKNRPPREQSLVEWARPCLRDARRLERVMDRRLPRPTTPTRAAQKAAGIAHQCLSVSPKSRPQMSHVVQALESLLALDDAAVEPFVYTAPPESR

SEQ ID NO: 4 - RNA, 1836 nt, Zea mays
  source 1..1836: mol_type = mRNA
cactcctcccccctggaggattaagcgtaatcaacccaccccacacagccaccgtatatgtatatataccccgatgcgtccagccaaagccgctggtgggaggccggccgggtaaaggtagtaaaccgtcgcagcgcgcggagtccctccatctccatccggcgaccgaccaaccgacctagactagacgcggccggccgggcatggcgaggccggggtggggctcgctgttcggctgcttcagcagcagcagcagctcccgcggggacggtaagaagaagcagggcaagggcaagggaaagggcgccgggaataagatgaagaagaagcagaagagcaagcagcagcagaaggtagcggcggggagcggcggcggtagccggccgcggtcgctgcagagccggatgtcgttcacggagctgagcctcagcgggatggtgtcgcccgaggacctgtcgctctcgctcgtcggctccaacctccacgtcttcaccatcgccgagctgcgcgccgtcacccgcgacttctccatgacccacttcatcggcgagggcggcttcggccccgtctacaggggctacgtcgacgacaagaccaagcgggggctcgccgcgcagcccgtcgccgtcaagctgctcgacctcgagggcggccaaggacacaccgaatggctgacggaggtcttcttcctgggacaactgaggcaccctcacctcgtgaagctgatcggctactgctacgaggacgagcacaggctgctcgtctacgagttcatgaccaggggcagcctggagaagcatctcttcaaaaagtacgcggcgtcgttgccttggtccacacggctgaagatcgccattggcgctgccaaagggttggccttcctccatgaagccgagaagccggtcatctaccgggacttcaagacctccaacatcctgctggactcggattacaaggcgaagctgtcggatttcggtctagccaaagacgggccggaagacgacgagacgcacgtgtcgacccgggtgatggggacccaaggatacgccgcgccggagtacatcatgacgggccacttgacggcgaagagcgacgtgtacggcttcggcgtggtgctgctggagctgctgtcggggcggaaggcggtggacaagagccgccccccgcgggagcagaacctggtggagtgggcgcggccgtacctgacggacgcgcgccgcctggaccgcgtcatggaccccagcctggtgggccagtactccagccgggccgcgcacaaggccgccgccgtggcgcaccagtgcgtcgccctcaaccccaagtcccgcccacacatgtccgccgtcgtcgaagcgctcgagccgctcctcgcgctcgacgactgcctcgtcgggcccttcgtctacatcgcaccgccggagacgacgaccgccaacggggacgaagcggcgccggtgagtagtaataaggaaggatccggcagacgaggaggccggaggaggtcgtcaggcgaaggcgcggccgtcgtgcggccagaggagtgatgttccctcttccgtccgtgctatgacgacgatgataatatgggtggatttactagcttccacgtcttgtacgtgaccggactgttgttgcctgtcgcgtgcatgcagcttggctgagatatatctatcgatcgagcagctatacgatcggtggaagttaacgtcttcgtttgtgtgattgaaagcatccagctggttgcgtgttttagagagaactctatagattgaatttgttgcatatacagtaatctaaccaaacttctctatttttatagatctaaaacgacttgta

SEQ ID NO: 5 - protein, 451 aa, Glycine max
MGSSFSSCYEGESVSPSPKPTKVVATKGGSSSNRVSITDLSFPGSTLSEDLSVSLVGSNLHVFSLSELKIITQSFSSSNFLGEGGFGPVHKGFIDDKLRPGLEAQPVAVKLLDLDGSQGHKEWLTEVVFLGQLRHPHLVKLIGYCCEEEHRLLVYEYLPRGSLENQLFRRYTASLPWSTRMKIAAGAAKGLAFLHEAKKPVIYRDFKASNILLDSDYNAKLSDFGLAKDGPEGDDTHVSTRVMGTQGYAAPEYIMTGHLTAMSDVYSFGVVLLELLTGRRSVDKGRPQREQNLVEWARPALNDSRKLGRIMDPRLEGQYSEVGARKAAALAYQCLSHRPRSRPLMSTVVNVLEPLQDFDDVPIGPFVYTVPAEQHNEVAKESETPKERKRENDHHHHNRHHHHHRHNGHRHHPLKSPKTPMSSDQSQNDEHRNGRRSGSNSPDTSNASEAQ

SEQ ID NO: 6 - RNA, 1371 nt, Glycine max
  source 1..1371: mol_type = mRNA
atgggatcaagcttctctagttgttatgagggtgaatctgtgtctccttctccaaagccaaccaaagtggtggctacgaagggtggttcttcttccaacagggtctctataacggacttgagttttcctggctcaacgctttcggaggatctctccgtttctctagtggggtctaacctgcatgtgttttcactcgctgagctcaagatcataacgcagggtttctcttcgagtaactttctcggagaaggaggtttcggacccgtgcataagggcttcattgatgacaagcttaggcctggtctcgaggctcaaccggttgccgtcaagcttttggacttggacggttctcagggccataaagagtggttgactgaagttgtgtttctgggtcaactaaggcatccacatctcgtgaagctgattggatattgctgtgaagaagaacacaggcttctggtatatgagtatttaccacgaggcagcttagagaatcaactatttagaagatatacggcatcgttaccatggtcaacaagaatgaaaattgcagctggagctgcaaagggtctagcctttctacatgaagcaaagaaaccagtcatctatagagattttaaagcttcaaacatcttgttagactcggattataatgcaaaactttccgattttggcttggccaaagatggtcctgaaggagatgacacacacgtttcaactagagtcatgggcacacaaggctatgcggccccagaatacatcatgacaggacacttgacagcaatgagtgatgtgtacagttttggagtcgtgctcttggagcttctaacaggacgaaggtcagtggacaaagggcgcccacaaagagaacagaacctcgtggaatgggccaggtctgctttgaatgattcccggaaacttagcagaataatggatccaaggcttgagggtcagtattctgaggttggggcaagaaaagcagctgcattggcttaccaatgccttagccacaggccaaggagtagacccttgatgagcactgtggttaatgttctcgagccacttcaggactttgatgatgttccaattggaccctttgtttacactgttccagctgaacaacaacaatacaatgaagtcgcaaaagagagtgaaacacctaaggagaggaagagggaaaatggtcatcatcataaccgacgtcatcatcatcatcgccacaatggtcataggcatcatccacttaaatctcctaagacaccaatgccatcagatcagtcacagaatgatgaacaccgaaatggtagaaaaagtgggtctaattcacctgatacattcaatgcatcagaagctcaacgaagcatgggttag

SEQ ID NO: 7 - protein, 424 aa, Brassica napus
MAVTKNKKTSLTSLFLGCYRTKNASRYEVEERGNAMKIRTCPVIKRLSLSDISDPSSPMSVMDDLSNSFTSQKLRMFTLSELRVITHNFSRSNMLGEGGFGPVYKGFIDDKVKPGLEAQPVAVKALDLHGHQGHREWLAEIIFLGQLSNKHLVKLIGFCCEEEQRVLVYEYMPRGSLENQLFRRNSVAMAWGIRMKIALGAAKGLAFLHEAEKPVIYRDFKTSNILLDCNYNAKLSDFGLAKDGPEGEHTHVTTRVMGTQGYAAPEYIMTGHLTTMNDVYSFGVVLLELITGKRSVDTTRARREQSLVEWARPMLRDQRKLDRIIDPRLECQYKTEAAQAAAALAFKCLSQNPKYRPTMSDVVKVLESIQEVETKERNSNNKETKKFVDKRIIRHHRKGQKRVNIAYSDSLLYKESRAKQNNGV

SEQ ID NO: 8 - RNA, 1584 nt, Brassica napus
  source 1..1584: mol_type = mRNA
taggttaactaaaacacccttaatttctaaacctacgcaatctacaaaagatacatcaaaaagagtaaaacactatctatcttccattttctccaaaaagttgaagaagaagaagaagaagaagaagaagaagaaaatggcagtaaccaagaacaagaagacatcattgacatctctgtttttaggatgttacaggacaaagaacgcgagtagatacgaagtagaggagaggggcaacgcaatgaaaataagaacttgtccggtgatcaagaggctgtcgttatcggacataagtgatccaagctcgcccatgtcggtcatggatgacctctcaaactccttcacatctcaaaagcttcgtatgttcaccttgtctgagctgagagttattacgcataatttctcaagaagtaacatgctgggagaaggagggtttgggccagtttacaaagggttcattgatgacaaggttaaaccgggtttagaagcccaaccggttgctgttaaggcacttgatctccatggtcaccaaggccatagagaatggctggcggagataatctttttgggacagctaagtaacaaacatctcgtgaagctgataggattttgttgtgaagaagagcaaagagtacttgtttatgagtacatgcctcgaggtagcttggagaatcagctctttagacgaaacagtgtagcaatggcatggggaataaggatgaagatagcccttggagccgctaaaggcttagcttttctccatgaagcagagaaacctgtcatctaccgagacttcaaaacctccaacatattgttagactgcaactataacgcaaaattatctgatttcggcctagccaaagatggacctgaaggagaacatacacatgtaacaactcgagtgatgggaacgcaaggttatgctgcacccgaatatatcatgactggtcacttgacgacaatgaatgacgtctacagttttggagtcgttctgttggaactgataacagggaaacggtcagtggacactacccgggcacgaagggaacaaagcctcgtggaatgggcgcgtccaatgttgagggaccaaagaaagctagatcggataatcgatccaaggctcgaatgccagtacaaaactgaggcagctcaggcagcagctgccttagccttcaagtgtctaagccaaaatcccaagtatagaccaacaatgtctgacgtagtaaaggttttggagtctatccaagaagttgagacaaaggagcgtaatagtaacaacaaagaaacaaagaagtttgttgataaaagaatcattagacatcaccgtaaaggccaaaaaagagtgaacatcgcttattcagattctctcctttacaaggaatccagggcaaagcagaacaatggggtttgaaatacatcagttttcatattaattattaagcattgatatttctatatatgcacttcttatatttagttctaagagccttcttccataaattttgggatggaaaggtggtgttatttaatgttaaaaaaaaaaggtggtattatttctcctagttatctggggtttcagattag

SEQ ID NO: 9 - protein, 422 aa, Gossypium hirsutum
MTVKKKITWKSLMPSCYKSKDTSDSGENRLKLKPCQFQRLSLSDVSDPSSPICVNGLSTSLFGSNLYVFTLAELRLITHNFARCNLLGEGGFGPVYKGFVDDKLRPGLKAQPVAVKTLDLDGLQGHREWLAEIIFLGQLRHPHLVKLIGYCYEDENRLLVYEYMPRGSLENQLFRRYSATLPWSTRMKIALGAAKGLAFLHEADKPVIYRDFKSSNILLDSDYNCKLSDFGLAKDGPEGGETHVTTRVMGTQGYAAPEYIMTGHLTTRSDVYSFGVVLLELLTGRRSMDNTRPSREQSLVEWARPLLRDPKRLDRVIDPRLEGQYSNKGAQKVAALAYKCLSHYPKPRPTMGDVVIVLESLQGFEDEFVGPFVYVVPNETENSNEFLAKKGENEHDSPLRGWRNRIKLPPSSVANAESPCSI

SEQ ID NO: 10 - RNA, 1626 nt, Gossypium hirsutum
  source 1..1626: mol_type = mRNA
gtcttctcacgcccaaagaaaagtgaaaatgtgtttgaaaaattagcaaattattttaaaaagaaaatgaagagggcattgtatacatgaagcatataattacttttttttccatctaaattttaaatacattcggatttgagaagtggtaggtaagtgaatgaattactataaaaatctctaaagccttcacaaaaatccatagaccctggcagaaattttccattttctttgtattttcttgcatattgcaaaagcaaacaattcattcaaacatgacagtgaagaagattacatggaaatctctcatgttaagctgttacaatagcaaggacatcatctctccatctgattcaggggagaaggattcgaaaccatgtcaattccaaagactatcaatgtccgacgtaagtgatcgcagctcgccactctctgtcgacgatctctcgacctctctactcggctcaaatcttcatgtgtttacctttgcggagctaagactaataacccataatttcgcacgctgtaatctgctcggtgaaggagggttcggaccggtttacaaaggtttcattgatgacaagcttaggcctggattgaaggctcagccggttgctgtcaaggcactggacttgtatggcttgcagggtcatagggagtggctggctgaaataatctttcttggtcaactacggcatcctcatctggttaagttgattggatattgttatgaagaagataacagagttttagtgtatgaatatatgccaagaggaagcttagaaaatcaactctttagaaggtattcagctcccttaccatggtctgctaggatgaaaattgccttggcagcagcaaagggcctggctttcctacatgaagctgataaaccagtaatatttcgggactttaaatcttcaaacatcttgttagactctgattacaattgcaaactatcggatttcgggttagcaaaggatggtccagaaggcgaacagactcatgtaacgacccgagttatgggcacacaaggatatgcagctccagagtatatcatgactggtcatttgacaacaatgagtgatgtgtacagctttggagtggttctcttagagttactaacagggaaacgatctgtggacaatagccggcccggtcgagagcagagccttgtggaatgggcaaggccattattaagggaccctaagaagcttgataagctaatagatgctagacttgagagacaattttccaacaagggggctcaaaaggtggctgcgttggcatacaaatgcttgagccaccaaccaaagccaaggcctagcatgggcgatgtggtcaagatcctggactctgttcaggggtttgaggatgaatttgttggaccttttgtttatgtggtgccaaatgaaaccgatggtgacaaggagttgtttacgaccaaggattgcggtgtagaaaaacacgaactacatcgttgtggttggcgaaatgatcaaattgcctctttctttggtagctaatgctgaatctccatgtggcataagcatttgagaattgcagatatatgtggtcgaatttatgtggtgcgaaatgaaaaacaaaggtagaaatttcgactggaaa

SEQ ID NO: 11 - protein, 406 aa, Saccharum sp.
MTSPKRAAGREGCGCWAAVARGLRGACFRPAAPADGDGGGSAKGSHVHDAAAETRYLNASNRELGDHFQTKHDGENGVDASIEKRTPKLLQFTFQELKSATLNFRPDSILGEGGFGYVFKGWIEPNSTAPAKPGTGVTVAVKSLKPDALQGHREWVAEVDFLGQLHHKHLVKLIGYCIEDDQRLLVYEFMARGSLENHLFRRALPLPWSNRMKIALGAAKGLAFLHGGPKPVIYRDFKTSNVLLDAEYNAKLSDFGLAKAGPQGDKTHVSTRVVGTYGYAAPEYVMTGHLTSKSDVYSFGVVLLEMLTGRRSMDKKRPTGEQNLVAWARPYLNDRRRLYQLIDPRLGLNYSVKGVQKVAQICHYCLTRDSKSRPSMDEVVKQLTPLQDLNDMASASPRPRSTQRGE

SEQ ID NO: 12 - RNA, 1070 nt, Saccharum sp.
  source 1..1070: mol_type = mRNA
ctactcaccgcgctgggttgagcgaggccttggcgatgcagatgccatgtcatttagatcttgtaatggtgtgagttgcttgacaacttcatccatggatggacgagacttactatcacgggtaaggcaatagtggcatatctgagcaaccttttgcacccctttgacagagtagttcagccccaagcgaggatctatgagctgatagagccttcgccgatcattcaggtaaggcctcgcccatgcaaccaggttctgctcccctgtaggtcgcttcttgtccattgatcttctcccagtcaacatctcgagtaacacaactccgaagctatagacatcactcttagatgtcaagtgccctgtcattacatactctggtgcagcataaccgtaagtgccaacaacccgagtagatacatgagttttatcaccctgaggaccagcttttgctaaaccgaaatcagatagttttgcgttgtattccgcatcaagaagaacattcgatgtcttaaaatccctgtaaataacaggtttgggacctccatggaggaaggctagtcctttcgcggcacctagagctatcttcattctgttggaccaaggtaggggaagggcccttctgaaaagatggttttcaagacttccccgtgccatgaattcatatacaagtaacctttgatcatcctcaatacaatatccaatcagcttaacaaggtgtttatgatgcaactgtcccagaaagtcaacttctgccacccattctctatgaccttgaagcgcatctggcttcaaacttttgactgccacagtaacccctgtgccaggttttgcaggagcagtgctgtttggctcaatccaccccttgaagacataaccaaacccaccttccccaagaatactgtctggcctgaagttaagggtggcagatttcaactcttgaaaggtaaactgaagtagcttgggtgttctcttttcaattgacgcatcaacaccattttcaccatcatgttttgtctgaaaatgatcaccaagctcccgattgctagcattcaggtatcttgtctctgctg

SEQ ID NO: 13 - protein, 389 aa, Oryza sativa
MGKKTTTSSSPSWSSLFGLGCFTSSHSDGGSAAAKNPGTPLPARPSSCNSNDGVAAAVMPSPEDLSQSLAGSGVEAFTVEELRRATRDFSVSNFVGEGGFGPVYKGYVDERLKPGVRAQAVAVKLLDLEGSQGHKEWLAEVIFLGQLRHHHLVKLIGYCYEDEHRLLVYEFMARGSLEKHLFKKYSASLPWSTRLKIAIGAARGLAFLHEAAKPVIYRDFKTSNILLNSDYEAKLSDFGLAKDGPQEDETHVSTRVMGTQGYAAPEYIMTGHLTTKSDVYSYGVVLLELLTGRKAVDKKRPPREQNLVEWARPCLHDSRRLNRVIDKSLNGQYSTRAVQKAAAIAYQCLSVSPKSRPRMSAVVEALEPLLAMDDGIVEPFVYMAPPESK

SEQ ID NO: 14 - RNA, 1468 nt, Oryza sativa
  source 1..1468: mol_type = mRNA
caccccacccacaaattaaacacaccaccctcaatatatatcatcatcatcatcccccaattcccaacaccgcgacgtgcttcctataaattaactccccccgatcctcctcccaaccattccacaccttggcaatcatggggaagaagacgacgacgtcgtcgtcgccgtcgtggagctcgctgttcgggctcggctgcttcacctcctcccacagcgacggcggctcggcggcggccaagaacccggggacgccgctgccggcgcggccgtcgtcgtgcaacagcaacgacggggtggcggcggcggtgatgccgtcgccggaggacctgtcgcagtcgctggcggggagcggggtggaggcgttcacggtggaggagctgaggagggcgacgcgggacttctcggtgagcaacttcgtgggcgagggggggttcgggccggtgtacaaggggtacgtcgacgagcggctcaagcccggcgtgcgcgcgcaggccgtcgccgtcaagctgctcgacctcgagggctcccagggccacaaggaatggctggctgaagtcatatttcttgggcaattgagacatcatcacctcgtcaagctgatcggatactgctacgaggatgagcacagacttctcgtctacgagttcatggctaggggcagcttggagaagcacctcttcaaaaagtactctgcgtcgctgccgtggtcaacacgcttgaagatcgccattggagcggccagaggtttggccttcctccatgaagctgccaagcccgtcatctatcgcgacttcaagacctccaacatcctactcaactcggactacgaagcaaaactttcggatttcggactggccaaagatgggcctcaagaagacgagacacatgtctccactagagtgatgggcacacagggctatgccgcaccagaatacatcatgactggtcatctaacgacaaagagcgatgtgtatagctatggtgtggtgctgctggagctactaaccggacggaaagccgtggataagaagagaccgccgcgagagcaaaacctagtggaatgggctcggccatgcctacatgactctcgccggctcaaccgtgtcatcgacaaaagcttgaacgggcagtactcgactcgagccgtacaaaaggctgcggccatagcgtatcagtgcttgagtgttagccctaagtctcgaccccggatgtcagcggttgtggaggccctggaaccacttctggccatggatgatggcattgtagagccatttgtgtacatggcaccacccgagagtaaataaatgaatcaataatttgtgtggagcatatgtacaaacatgccattctccaatccttgcaatcaatttaaaatatagaaaattctccatttgcatttagaaatttcagcatatccctgtatgcctctgagtttttcttaagagtaaattatattggtgataca

SEQ ID NO: 15 - protein, 412 aa, Solanum tuberosum
MKITWESLVPSCCISENPKPKPKQSKLVTKQSSFHRISVSDFSGSEDLSISLAGSNLHIFSVQELKVITQNFSSSNFLGEGGFGPVHKGFIDDKLRPGLKAQPVAVKLLDLDGTQGHREWLTEVIFLGQLRHPHLVKLIGYCCEEEHRLLAYEYMPRGSLENQLFRRYSVSLPWSTRMKIALGAAKGLAFLHEAEKPVIYRDFKASNILLDSDYNAKLSDFGLAKDGPEGDDTHVSTRVMGTQGYAAPEYLMTGHLTAASDVYSFGVVLLELLTGRRSVDKSRPNREQNLADWARPQLKDARKLGRIIDPRLEGLYSEEGVQKAALVAYQCLSHRPKARPDMSTVVKTLETLKDYKDISTMTFVYIAPVDQHKQSPQKELVNKRNTNSHHHTKSPHKRLTPNSPLHNDFHRA

SEQ ID NO: 16 - RNA, 1568 nt, Solanum tuberosum
  source 1..1568: mol_type = mRNA
gagcagttgggtacgacatatttgaatattattattaccctctattcctttcttgctcatcaatcatttttctatatatataaacacacaattctcttcaaggcaattaagagaatttattcattttatttcaattaaagctagcgatgaagattacatgggaatctctagtacctagctgttgtatatccgaaaatccaaagccaaagccaaagcaatcgaaattagtaacaaaacaaagttcatttcatcggatttcagtttcagatttcagtggttcagaagatttatcaatatcattagctggttcaaatcttcatattttcagtgttcaagaattgaaagtgattactcagaatttttcgtcgagtaattttctaggggagggaggttttggaccagttcataaaggttttattgatgataaacttagacctggtctaaaagctcaacctgttgctgtaaagcttttagatttagatggcactcaaggtcatcgagaatggctgacagaagtgatatttttggggcaattgagacatccacatctggtgaagttgattggatattgttgtgaagaggaacacaggctgttggcctatgaatatatgcccagaggcagcttggagaatcaactttttcgaagatattctgtatcacttccatggtcaacgcggatgaaaatagcacttggagctgcaaaaggcttagctttcctccatgaagctgaaaaaccagtcatatatcgtgatttcaaggcttcaaacatcttgttagactccgattacaatgctaaactgtcagattttggacttgcaaaagatgggccagaaggagatgacacacacgtctccacccgagtcatgggaacgcaaggctacgctgctcctgaatacctcatgaccggtcatttgacagctgcaagtgatgtatacagtttcggagtagtattactggagcttctaactgggagaagatcggtagataaaagtcgtccaaacagagaacaaaatttagcagattgggcaagacctcaattgaaagatgcgcgtaaactaggtagaataattgatccaagattagaaggtttatattcagaagaaggggttcaaaaagcagcattagtagcttaccaatgcttaagccataggccaaaagctagaccagatatgagtacagttgtcaaaacacttgaaacattgaaggattataaggatatttcaaccatgacatttgtgtatatagctccagttgatcaacacaaacaaagtcctcaaaaggaattagtcaacaaaagaaatactaattctcatcatcatacaaaaagtccacataagagattaactccaaattcaccattgcacaatgatttccatagagcttaaacaattgtatacatccacatttttttaattttttttacctcataatactcaaaattctttattttttcattgagtgtatttgctagatatcagtgttagtgaagacaaggcagatatcaactttgtatatgttatatttccttttggagtttttaaagtaatatatacataccgtttaaataa

SEQ ID NO: 17 - protein, 434 aa, Triticum aestivum
MARSAWRSLFGCFSSHAEAAAAAGKGEKMSKASKKKKSKKKVAAAGKAPRPSRSLHGRMSFSDLSMSGGMVSPEDLSLSLAGSDLHVFTIAELRAVTRDFSMTNFIGEGGFGPVYKGYVDEKVKPGLRAQPVAVKLLDLEGNQGHNEWLTEVIFLGQLRHPHLVKLIGYCYEDEHRLLVYEFMTRGSLEKLLFKKYAASLQWSTRLKIALGAAKGLAFLHEAENPVIYRDFKTSNILLDSDYKAKLSDFGLAKDGPEEDETHVSTRVMGTQGYAAPEYIMTGHLTAKSDVYGFGVVLLELLTGRKSVDKSRPPREQNLVDWARPYLNDSRRLDRVMDPNLAGQYAGGAVQKAAALAYKCVSLNPKSRPHMSAVVSALEPLLALEEDSLAGPFVYVAPAENGNSGGGREGRHRRRAGRRKSGDAAESVAVVVERE

SEQ ID NO: 18 - RNA, 1815 nt, Triticum aestivum
  source 1..1815: mol_type = mRNA
gcagcaggtagagcagagcgacgcgcacacgcggaaggcacgcacggatggcgaggtcggcgtggaggtccctcttcggctgcttcagctcgcacgccgaggccgcggccgccgccggcaagggcgagaagatgagcaaggcctccaagaagaagaagagcaagaagaaggtggcggcggcggggaaggcgcctcggccgagccggtcgctgcacggccggatgtccttctcggacctcagcatgagcggcggcatggtgtcgccggaggacctgtcgctgtctctggcggggtcggacctgcacgtcttcaccatcgccgagctgcgcgccgtcaccagggacttctccatgaccaacttcatcggcgagggcggcttcggccccgtctacaagggctacgtcgacgagaaggtcaagccggggctccgcgcgcagcccgtcgccgtcaagctgctcgacctcgagggcaaccagggccacaacgagtggctgaccgaagtcatattcctggggcagttgaggcatcctcacctggtcaaactgatcggttactgctacgaggacgagcacaggctcctcgtgtacgagttcatgaccaggggcagcctagagaagcttctcttcaaaaaatacgcggcgtcgttgcaatggtcaacacggctgaagatcgccctcggcgccgccaaagggttggcgttcctccatgaggccgagaacccggtcatctaccgggacttcaagacctccaacatcctgctagactcggattacaaagcgaagctatcggatttcgggctagccaaagatggaccagaggaagacgagacgcacgtctccacccgggtcatgggcacgcagggctacgccgcaccggagtacatcatgacagggcatctgacggccaagagcgacgtgtacggtttcggcgtggtgctgctggagctgctcaccgggcgcaagtcggtggacaagagccggccgccgcgggagcagaacctggtggactgggcgcgcccctacctcaacgactcccggcgcctggaccgcgtcatggaccccaacctcgcggggcagtatgccggcggggccgtgcagaaggccgcggccctggcgtacaagtgcgtcagcctcaaccccaagtcccgcccgcacatgtccgccgtcgtcagcgcgctcgagccgctcctcgcgctcgaggaggatagcctcgccgggcccttcgtgtacgttgcgccggctgagaacgggaacagcggcggcggcagggaggggcgccacaggaggcgagcgggccggaggaagtccggagacgcggccgagtcggtggccgttgttgttgagcgcgagtgatggtcttgtgtaagcgtgtttccgaacttggacgggtcgaactcgaacagtggggaggagcgcgggcgcattcagattcggatatttgtggaacaacaaggctgcctatgatacgagaagaggggaggcttgacggattggagtccgtaaatagagcaatgtcatgaaagaaaaatggaccatctctctcatactactcctcctactttgcattggcatgcgcttcagtttcttagtgttttaattcagttccttcagagtttgtggatacatgtttccccaggactagtagtagggcagaaaaaagtacacggatttgcaccgagtataaattaggcaaggtagtgtttctgtatatgtacgatcacggtctagttctcaaattcgctgtcatattggtagacatcggactgaacttgtggtcatgaaagtaatcataggcaagcgaagatcttgtgtgttt

SEQ ID NO: 19 - protein, 438 aa, Triticum aestivum
MARSAWRSLFGCFSSHAEAAAAAAAAGKGEKKGKASKKKKSKKKVAAAGKAPRPSRSLHGRMSFSDLSMSGGMVSPEDLSLSLAGSDLHIFTIAELRAVTRDFSMTNFIGEGGFGPVYKGYVDEKVKPGLRAQPVAVKLLDLEGNQGHNEWLTEVIFLGQLRHPHLVKLIGYCYEDEHRLLVYEFMTRGSLEKLLFKKYAASLQWSTRLKIALGAAKGLAFLHEAENPVIYRDFKTSNILLDSDYKAKLSDFGLAKDGPEEDETHVSTRVMGTQGYAAPEYIMTGHLTAKSDVYGFGVVLLELLTGRKSVDKSRPPREQNLVDWARPYLNDSRRLDRVMDPNLAGQYAGGAAQRAAALAYKCVSLNPKSRPHMSAVVSALEPLLALEEDSLAGPFVYVAPPDNGSSGGGSREGRHRRRAGRRESGDVAESVAVVVERE

SEQ ID NO: 20 - RNA, 1788 nt, Triticum aestivum
  source 1..1788: mol_type = mRNA
gcacacgcggaaggcacgcacggatggcgaggtcggcgtggaggtccctgttcgggtgcttcagctcgcacgccgaggccgcggccgccgcagccgccgccggcaagggcgagaaaaagggcaaggcctccaagaagaagaagagcaagaagaaggtggcggcggcgggcaaggcgccgaggccgagccggtcgctgcacggccggatgtccttctcggacctcagcatgagcggcggcatggtgtcgccggaggacctgtcgctgtcgctggcggggtccgacctccacatcttcaccatcgccgagctgcgcgccgtcaccagggacttctccatgaccaacttcatcggcgagggcgggttcggccccgtctacaagggctacgtcgacgagaaggtcaagcccggcctccgcgcgcagcccgtcgccgtcaagctgctcgacctcgaggggaaccaggggcacaacgagtggctgactgaagtcatattcctggggcagttgaggcatcctcacctggtcaaactgattggttattgctacgaggatgagcacaggctcctcgtgtacgagttcatgaccaggggcagcctggagaaacttctcttcaaaaaatacgcggcgtcgttgcaatggtcaacacggctgaagatcgccctgggcgccgccaaagggttggcgttcctccatgaggccgagaacccggtcatctaccgggacttcaagacctccaacatcctgctagactcggattacaaagcgaagctgtcggatttcgggctagccaaagatggaccggaagaagacgagacgcacgtctccacccgggtcatgggcacgcagggctacgccgcgccggagtacattatgacagggcatctgacggccaagagcgacgtgtacggcttcggcgtggtgctgctggagctgctgacggggcgcaagtcggtggacaagagccggccgccgcgggagcagaacctggtggactgggcgcgcccctacctcaacgactcccggcgcctggaccgcgtcatggacccaaacctcgccgggcagtacgccggcggggccgcgcagagggccgccgcactggcctacaagtgcgtcagcctcaatcccaagtcccgcccgcacatgtccgccgtcgtcagcgcgctcgagccgctcctcgcgctcgaagaggatagcctcgccgggccgttcgtctacgttgcgccgcctgacaacggcagcagcggcggtggcagcagggaggggcgccacaggaggcgagcgggccggagggagtccggagacgtggccgagtcggtggccgttgttgttgagcgcgaatgatggtctcagcttgacggagcgtgtttctgaacttggaccggtccaacgaacagtggagaggagcgcgggcgcattcagcttcgaatatttgtggaacaacaagggttgcctatgatacgagaagaggagaggcttgacggactggagtccgtaaatagagcaatttcatgaaagaaaaaatggactatctctctcgtgctactcctcctactttgcattggcatgcacttcagtttgttagtgttttctgagtttacggacaagtactagtagcagggaaaaaagtacacggacttgcaccgagtataaattaggcaaggtagtgtttctatatatgtacctcacggtctagttctcaaatttgcactcatctctactcctaatgtctcagttagtagaatagtccatggtttttttccgtctgattaatttttatcgggtttatttt

SEQ ID NO: 21 - protein, 440 aa, Triticum aestivum
MARSAWRSLFGCFSSHAEAAAAAAAAGKGERKPPGGKKKKKSKKKVAAAGKAPRPSRSLHGRMSFSDLSMSGGMVSPEDLSLSLAGSDLHVFTIAELRAVTRDFSMTNFIGEGGFGPVYKGYVDEKVKPGLRAQPVAVKLLDLEGNQGHNEWLTEVIFLGQLRHPHLVKLIGYCYEDEHRLLVYEFMTRGSLEKLLFRKYAASLQWSTRLKIALGAAKGLAFLHEAQNPVIYRDFKTSNILLDSDYKAKLSDFGLAKDGPEEDETHVSTRVMGTQGYAAPEYIMTGHLTAKSDVYGFGVVLLELLTGRKSVDKSRPPREQNLVDWARPYLNDSRRLDRVMDPNLAGQYAGGAAQRAAALAYKCVSLNPKSRPHMSAVVSALEPLLALEEDSLAGPFVYVAPPDNGSSGGGGSREGRHRRRAGRRKSGDVAESVAVVVERE

SEQ ID NO: 22 - RNA, 1948 nt, Triticum aestivum
  source 1..1948: mol_type = mRNA
cagagcgacgcgcacacggggaaggcacgcacgcacgcgtacggaatcgcccgcgcgcgccgatggcgaggtcggcgtggaggtccctcttcgggtgcttcagctcgcacgccgaggccgcggccgccgcagccgccgccggcaagggcgagaggaagccccccggcggcaagaagaagaagaagagcaagaagaaggtggcggcggcggggaaggcgccgaggccgagccggtcgctgcacggccggatgtccttctcggacctcagcatgagcggcggcatggtgtcgccggaggacctgtcgctgtcgctggcggggtccgacctgcacgtcttcaccatcgccgagctgcgcgccgtcaccagggacttctccatgaccaacttcatcggcgagggcggcttcggccccgtctacaagggctacgtcgacgagaaggtcaagcccgggctccgcgcgcagcccgtcgccgtcaagctgctcgacctcgaggggaaccagggccacaacgagtggctgactgaagtcatattcctggggcagttgaggcatcctcacctggtcaaactgatcggttactgctacgaggatgagcacaggctcctcgtgtacgagttcatgaccaggggcagcctggagaagcttctcttcagaaaatacgcggcgtcgttgcaatggtcaacacggctgaagatcgccctgggtgccgccaaagggttggcgttcctccatgaggcccagaacccggtcatctaccgggacttcaagacctccaacatcttgctcgactcggattacaaagcgaagctatcggatttcgggctagccaaagatgggcccgaagaggacgagacgcacgtctccacccgggtcatgggcacgcagggctacgccgcacccgagtacatcatgacagggcatctgacggcaaagagcgacgtgtacggcttcggcgtggtgctgctggagctgctgacggggcgcaagtcggtggacaagagccggccgccgcgggagcagaacctggtggactgggcccggccctacctcaacgactcccggcgcctggaccgcgtcatggaccccaacctcgccgggcagtacgccggcggggccgcgcagagggccgccgcgctggcctacaagtgcgtcagcctcaaccccaagtcccgcccgcacatgtccgccgtcgtcagtgcgctcgagccgctcctcgcgctcgaagaggatagcctcgccggcccgttcgtgtatgttgcgccgcctgacaatggcagcagcggcggcggcgggagcagggaggggcgccacaggaggcgagcgggccggaggaagtccggagacgtggccgagtcggtggccgttgttgttgagcgtgaatgatggtctcagcttgacggagcgtgttttctgaacttggacgcgtcgaacagtggagaggagcgcgagcgcattcagattcgaatatttgtggaacaacaagggttgcctatgatacgagaagaggggaggcttgacggattggagtccataaatagagcaatttcatgaaagaaaaatggacaatctctctcgtactactcctcctactttgcattggcatgcacttcagtttcttagtgttttaattcagttcatttagagtttgtacatacatgtttccccaggactaagtagtagggcaaaaaaagtacacggatttgtaccgagtataaattaggcaaggtctgtctaggtctaagtcgactgagacttcgcgaagtctaagttgctgacgtcatcaaattttcttttccttttgagagtgtcatccatgtttgtacgcagcctattgtttgtgcattctcacggctgttacttcttcctgtgttgggctgggcccgcctctctctctgtgtacccttttcttcttttgtattttgtattattaggctggacttgtctca

SEQ ID NO: 23 - protein, 49 aa, synthetic construct
  VARIANT 7: note = D or A
  VARIANT 10: note = E, Q, or G
  VARIANT 11: note = G or E
  VARIANT 12: note = D, E, or G
  VARIANT 13: note = E, H, D, or K
  VARIANT 17: note = S or T
  VARIANT 21: note = M or V
  VARIANT 24: note = Q or Y
  VARIANT 32: note = I, V, or L
  VARIANT 39: note = T, A, or S
  VARIANT 40: note = M, K, R, or A
  VARIANT 41: note = S or N
  VARIANT 46: note = F or Y
SDFGLKXGPXXXXTHVXTRVXGTXGYAAPEYXMTGHLTXXXDVYSXGVV

SEQ ID NO: 24 - protein, 50 aa, synthetic construct
  VARIANT 4: note = R or K
  VARIANT 7: note = L or V
  VARIANT 8: note = A or T
  VARIANT 10: note = V or I
  VARIANT 11: note = I, V, L, or D
  VARIANT 17: note = R, S, or H
  VARIANT 18: note = H or N
  VARIANT 19: note = P, K, or H
  VARIANT 27: note = Y or F
  VARIANT 29: note = C, Y, or I
  VARIANT 30: note = E or Q
  VARIANT 31..32: note = E or D
  VARIANT 33: note = H, Q, or N
  VARIANT 35: note = L or V
  VARIANT 37: note = V or A
  VARIANT 40: note = Y or F
  VARIANT 41: note = M or L
  VARIANT 42: note = P or A
  VARIANT 48: note = N or K
  VARIANT 49: note = Q or H
QGHXEWXXEXXFLGQLXXXHLVKLIGXCXXXXXRXLXYEXXXRGSLEXXL

SEQ ID NO: 25 - protein, 50 aa, synthetic construct
  VARIANT 1: note = S, T, or A
  VARIANT 2: note = A, V, or L
  VARIANT 3: note = S, A, T, or P
  VARIANT 4: note = L or M
  VARIANT 5: note = P or A
  VARIANT 7: note = S or G
  VARIANT 8: note = T, I, or N
  VARIANT 10: note = M or L
  VARIANT 11: note = K or R
  VARIANT 14: note = L, I, or A
  VARIANT 18: note = K or R
  VARIANT 25: note = E, G, or D
  VARIANT 26: note = A or G
  VARIANT 27: note = E, A, P, K, or D
  VARIANT 37: note = T, A, or S
  VARIANT 40: note = I or V
  VARIANT 43: note = D or N
  VARIANT 44: note = S, C, or A
  VARIANT 45: note = D, N, or E
  VARIANT 47: note = N, T, or E
  VARIANT 48: note = A or C
XXXXXWXXRXXIAXGAAXGLAFLHXXXKPVIYRDFKXSNXLLXXXYXXKL

SEQ ID NO: 26 - protein, 1344 aa, Arabidopsis sp.
MALGDLMVSRFSQSSVSLVSNHRYDEDCVSSHDDGDSRRKDSEAKSSSSYGNGTTEGAATATSMAYLPQTIVLCELRHDASEASAPLGTSEIVLVPKWRLKERMKTGCVALVLCLNITVDPPDVIKISPCARIEAWIDPFSMAPPKALETIGKNLSTQYERWQPRARYKVQLDPTVDEVRKLCLTCRKYAKTERVLFHYNGHGVPKPTANGEIWVFNKSYTQYIPLPISELDSWLKTPSIYVFDCSAARMILNAFAELHDWGSSGSSGSSRDCILLAACDVHETLPQSVEFPADVFTSCLTTPIKMALKWFCRRSLLKEIIDESLIDRIPGRQNDRKTLLGELNWIFTAVTDTIAWNVLPHELFQRLFRQDLLVASLFRNFLLAERIMRSANCNPISHPMLPPTHQHHMWDAWDMAAEICLSQLPQLVLDPSTEFQPSPFFTEQLTAFEVWLDHGSEHKKPPEQLPIVLQVLLSQCHRFRALVLLGRFLDMGSWAVDLALSVGIFPYVLKLLQTTTNELRQILVFIWTKILALDKSCQIDLVKDGGHTYFIRFLDSSGAFPEQRAMAAFVLAVIVDGHRRGQEACLEANLIGVCLGHLEASRPSDPQPEPLFLQWLCLCLGKLWEDFMEAQIMGREANAFEKLAPLLSEPQPEVRAAAVFALGTLLDIGFDSNKSVVEDEFDDDEKIRAEDAIIKSLLDVVSDGSPLVRAEVAVALARFAFGHKQHLKLAAASYWKPQSDSLLTSLPSIAKFHDPGSATIVSLHMSPLTRASTDSQPVARESRISSSPLGSSGLMQGSPLSDDSSLHSDSGMMHDSVSNGAVHQPRLLDNAVYSQCVRAMFALAKDPSPRIASLGRRVLSIIGIEQVVAKPSKPTGRPGEAATTSHTPLAGLARSSSWFDMHAGNLPLSFRTPPVSPPRTNYLSGLRRVCSLEFRPHLLGSPDSGLADPLLGASGSERSLLPLSTIYGWSCGHFSKPLLGGADASQEIAAKREEKEKFALEHIAKCQHSSISKLNNNPIANWDTRFETGTKTALLHPFSPIVVAADENERIRVWNYEEATLLNGFDNHDFPDKGISKLCLINELDDSLLLVASCDGSVRIWKNYATKGKQKLVTGFSSIQGHKPGARDLNAVVDWQQQSGYLYASGETSTVTLWDLEKEQLVRSVPSESECGVTALSASQVHGGQLAAGFADGSLRLYDVRSPEPLVCATRPHQKVERVVGLSFQPGLDPAKVVSASQAGDIQFLDLRTTRDTYLTIDAHRGSLTALAVHRHAPIIASGSAKQLIKVFSLQGEQLGIIRYYPSFMAQKIGSVSCLTFHPYQVLLAAGAADSFVSIYTHDNSQAR

SEQ ID NO: 27 - protein, 1344 aa, Arabidopsis sp.
MALGDLMVSRFSQSSVSLVSNHRYDEDCVSSHDDGDSRRKDSEAKSSSSYGNGTTEGAATATSMAYLPQTIVLCELRHDASEASAPLGTSEIVLVPKWRLKERMKTGCVALVLCLNITVDPPDVIKISPCARIEAWIDPFSMAPPKALETIGKNLSTQYERWQPRARYKVQLDPTVDEVRKLCLTCRKYAKTERVLFHYNGHGVPKPTANGEIWVFNKSYTQYIPLPISELDSWLKTPSIYVFDCSAARMILNAFAELHDWGSSGSSGSSRDCILLAACDVHETLPQSVEFPADVFTSCLTTPIKMALKWFCRRSLLKEIIDESLIDRIPGRQNDRKTLLGELNWIFTAVTDTIAWNVLPHELFQRLFRQDLLVASLFRNFLLAERIMRSANCNPISHPMLPPTHQHHMWDAWDMAAEICLSQLPQLVLDPSTEFQPSPFFTEQLTAFEVWLDHGSEHKKPPEQLPIVLQVLLSQCHRFRALVLLGRFLDMGSWAVDLALSVGIFPYVLKLLQTTTNELRQILVFIWTKILALDKSCQIDLVKDGGHTYFIRFLDSSGAFPEQRAMAAFVLAVIVDGHRRGQEACLEANLIGVCLGHLEASRPSDPQPEPLFLQWLCLCLGKLWEDFMEAQIMGREANAFEKLAPLLSEPQPEVRAAAVFALGTLLDIGFDSNKSVVEDEFDDDEKIRAEDAIIKSLLDVVSDGSPLVRAEVAVALARFAFGHKQHLKLAAASYWKPQSSSLLTSLPSIAKFHDPGSATIVSLHMSPLTRASTDSQPVARESRISSSPLGSSGLMQGSPLSDDSSLHSDSGMMHDSVSNGAVHQPRLLDNAVYSQCVRAMFALAKDPSPRIASLGRRVLSIIGIEQVVAKPSKPTGRPGEAATTSHTPLAGLARSSSWFDMHAGNLPLSFRTPPVSPPRTNYLSGLRRVCSLEFRPHLLGSPDSGLADPLLGASGSERSLLPLSTIYGWSCGHFSKPLLGGADASQEIAAKREEKEKFALEHIAKCQHSSISKLNNNPIANWDTRFETGTKTALLHPFSPIVVAADENERIRVWNYEEATLLNGFDNHDFPDKGISKLCLINELDDSLLLVASCDGSVRIWKNYATKGKQKLVTGFSSIQGHKPGARDLNAVVDWQQQSGYLYASGETSTVTLWDLEKEQLVRSVPSESECGVTALSASQVHGGQLAAGFADGSLRLYDVRSPEPLVCATRPHQKVERVVGLSFQPGLDPAKVVSASQAGDIQFLDLRTTRDTYLTIDAHRGSLTALAVHRHAPIIASGSAKQLIKVFSLQGEQLGIIRYYPSFMAQKIGSVSCLTFHPYQVLLAAGAADSFVSIYTHDNSQAR

SEQ ID NO: 28 - protein, 5045 aa, Arabidopsis thaliana
TTTGATCTGAAGAGGGTAAAACTAAAAACGGAAAGGAGAAAAAGAATGAAAGAGAAGTGGAAAGAAAACGCAAATGCATTGAAGACTTTCCCTTTGTCTCTCTCTTTGACAAAATCCCAATTTTCGCATCCTCACTTTCTCCTCTGCCTTTTTTTTTTCTCTTCTGCCGCCACCACCACCACCACTGTTAATTCGCCGCCGCTTCCACATCAATCGCCGTCGTAGCCAATCATCATCGTTCTTCATCCTCCGCTTCTCCTGATTCTTAGGGTTTCCGCTTCCGCTTCTCTGTCGAGGGTTTTTCGAGCTGGATTCTCTGCTCCTGCGCCGCTGGTGCAGCGGCTTTGTTGATTGATCTGGATTTCTCCGTCTTCCTTTCTATTTTATTTTCTGTATCTCGCACAATCCAAACAAAAAAGCTAGGGCTGAGGATTTGATTTGCGTGATTTCTGGGGGTGTGTTAATTCGGGGGTGTGTTCTCCGATTTGGATGGCATTAGGAGACTTAATGGTGTCTCGGTTCTCGCAATCTTCTGTTTCTTTGGTCTCCAATCACCGTTACGACGAAGACTGTGTCTCTAGTCACGATGACGGTGATTCCCGGAGGAAGGATTCTGAGGCTAAGAGCAGTAGTAGCTACGGGAATGGTACAACGGAGGGAGCCGCCACCGCCACCAGCATGGCTTACTTGCCTCAGACTATTGTGCTCTGCGAGCTTAGACACGACGCATCCGAGGCTTCTGCTCCCTTGGGGACTTCTGAGATCGTATTGGTCCCCAAATGGCGGCTTAAAGAACGAATGAAAACAGGATGTGTAGCTTTAGTTCTGTGTTTAAACATTACTGTTGATCCGCCGGATGTTATAAAGATATCTCCTTGTGCTAGAATCGAGGCATGGATAGATCCATTTTCTATGGCACCGCCTAAAGCTCTCGAGACAATTGGAAAAAATTTGAGCACTCAGTATGAGAGATGGCAACCCAGGGCCCGCTATAAGGTTCAGCTTGATCCGACAGTAGATGAGGTGAGGAAGCTGTGCTTGACTTGTCGGAAATATGCAAAGACCGAGAGAGTTCTATTCCATTATAATGGGCATGGTGTGCCAAAACCTACAGCTAATGGTGAAATTTGGGTATTTAACAAGAGTTATACACAGTACATTCCCTTGCCAATCAGTGAACTTGATTCCTGGTTGAAGACACCCTCCATCTATGTTTTCGACTGCTCTGCTGCTAGGATGATTCTGAATGCCTTTGCTGAGCTTCATGATTGGGGTTCTTCTGGTTCCTCTGGATCCTCAAGGGACTGCATTCTACTTGCTGCTTGTGATGTACATGAGACACTTCCTCAGAGCGTTGAGTTTCCAGCTGATGTTTTTACGTCTTGCCTGACAACGCCCATTAAAATGGCGTTGAAATGGTTCTGCAGGCGTTCGCTTCTGAAAGAAATTATCGATGAATCACTTATCGACAGGATTCCAGGCCGGCAAAACGATCGTAAGACATTGTTAGGGGAGTTGAACTGGATTTTCACAGCAGTGACGGATACAATTGCTTGGAATGTGCTTCCTCATGAACTTTTCCAGAGATTATTCAGACAGGACTTGTTGGTCGCTAGCCTTTTCCGGAATTTCTTACTTGCTGAGAGAATAATGCGGTCCGCAAACTGTAATCCAATATCTCACCCTATGCTGCCTCCTACGCATCAACATCATATGTGGGATGCATGGGACATGGCTGCTGAAATCTGTCTTTCTCAGCTTCCCCAACTTGTTCTGGACCCAAGCACAGAGTTCCAGCCAAGTCCGTTTTTTACTGAGCAACTGACAGCTTTTGAGGTGTGGCTTGATCATGGATCTGAGCATAAGAAGCCACCGGAGCAGTTACCTATTGTTCTTCAGGTGTTACTTAGCCAGTGCCATCGGTTTCGTGCTCTTGTACTTCTTGGAAGATTTCTTGATATGGGTTCATGGGCTGTGGATCTGGCCTTGTCTGTTGGAATATTCCCATATGTGCTGAAGCTTCTGCAAACAACAACGAATGAGCTAAGACAGATCCTGGTTTTCATATGGACAAAAATTCTTGCACTTGACAAGTCATGTCAAATTGATCTTGTGAAGGATGGGGGGCATACATACTTCATACGATTTCTAGATAGCTCGGGTGCATTTCCAGAACAACGAGCTATGGCTGCTTTTGTTCTGGCTGTCATTGTGGATGGACATCGACGAGGCCAGGAAGCATGTCTTGAAGCTAATTTAATTGGTGTTTGTCTCGGGCACCTTGAAGCATCCAGACCAAGTGATCCACAACCAGAACCATTGTTTCTACAATGGCTTTGTCTTTGTCTTGGAAAGTTATGGGAAGATTTTATGGAGGCCCAAATAATGGGCAGGGAGGCAAATGCTTTTGAAAAGTTGGCACCTCTGCTTTCCGAGCCCCAACCTGAGGTAAGGGCTGCTGCAGTTTTTGCCCTGGGTACCTTGCTTGATATTGGGTTTGACTCTAATAAAAGTGTGGTGGAGGATGAATTTGATGATGATGAAAAGATTAGAGCCGAAGACGCTATCATTAAAAGTCTCTTAGATGTAGTTTCAGATGGGAGTCCACTTGTCCGAGCAGAGGTTGCCGTAGCTCTTGCACGTTTTGCCTTTGGCCACAAACAGCACCTTAAGTTAGCCGCAGCTTCATATTGGAAGCCTCAGTCAAGTTCTTTGCTTACTTCTCTACCTTCAATAGCTAAATTCCATGATCCTGGGAGTGCAACAATTGTTTCTTTACACATGAGTCCTCTGACTAGAGCTAGCACGGATAGCCAACCAGTGGCTCGTGAGTCTAGGATCTCAAGCAGCCCTCTTGGCTCCTCTGGGCTGATGCAAGGATCTCCATTATCTGATGATTCTTCGTTACATTCTGATTCTGGAATGATGCATGACAGTGTCAGCAATGGAGCGGTCCATCAGCCAAGACTGTTGGATAATGCTGTTTATTCGCAGTGCGTCCGAGCTATGTTTGCATTAGCTAAAGATCCATCTCCACGTATTGCAAGTCTCGGACGGCGTGTGCTTTCTATTATTGGAATCGAACAGGTTGTTGCGAAACCCTCGAAGCCCACTGGCCGACCAGGGGAAGCTGCAACGACATCTCACACTCCACTTGCTGGCCTAGCTCGTTCATCCTCATGGTTTGATATGCATGCAGGTAATCTGCCTTTAAGTTTTAGGACTCCCCCGGTCAGCCCCCCTAGAACAAACTACCTGAGTGGATTGAGGAGAGTTTGTTCATTAGAGTTCAGGCCTCATCTATTGGGTTCACCCGACTCAGGATTGGCTGATCCGCTTTTAGGCGCCAGTGGATCTGAACGGAGTTTGCTTCCACTATCAACTATCTACGGCTGGAGTTGTGGGCACTTTTCTAAGCCACTTCTTGGTGGTGCGGATGCTAGTCAAGAGATTGCAGCCAAAAGAGAAGAGAAAGAAAAATTCGCACTTGAGCATATTGCAAAATGCCAGCACTCTTCTATTAGCAAGCTCAACAATAATCCTATTGCCAACTGGGATACAAGGTTTGAAACGGGAACAAAGACAGCCCTCCTTCACCCATTCTCTCCTATTGTAGTTGCTGCAGACGAGAATGAACGGATCAGAGTGTGGAACTATGAGGAAGCAACTCTTCTCAATGGCTTTGACAATCATGATTTTCCTGACAAAGGAATTTCAAAGCTCTGCCTCATCAATGAACTTGACGACTCTCTGCTACTTGTTGCATCATGCGATGGGTCGGTCCGGATATGGAAAAACTATGCAACTAAGGGTAAACAAAAGCTTGTTACTGGGTTTTCTTCAATCCAGGGTCACAAGCCCGGTGCCCGTGACTTGAACGCTGTCGTGGACTGGCAACAACAGTCCGGTTACCTGTATGCTTCTGGGGAGACGTCAACAGTCACACTTTGGGACCTGGAGAAAGAACAGCTTGTCAGATCTGTTCCCTCTGAATCAGAATGTGGAGTTACAGCACTTTCTGCTTCTCAAGTGCACGGAGGCCAACTCGCTGCTGGTTTTGCTGATGGATCTTTGAGACTCTATGATGTTCGGTCACCTGAACCGCTTGTCTGCGCGACTCGGCCTCATCAGAAAGTTGAAAGGGTGGTTGGCCTCAGTTTTCAACCTGGACTTGACCCCGCAAAGGTGGTGAGTGCATCACAGGCGGGTGACATACAGTTTCTTGACCTTAGAACAACAAGGGACACATACCTGACGATTGATGCACACAGGGGTTCACTCACGGCCTTAGCTGTTCACAGACACGCTCCAATAATCGCGAGTGGATCTGCAAAACAGCTCATTAAAGTATTCAGTCTTCAAGGGGAACAACTAGGGATAATCCGCTACTACCCATCCTTCATGGCTCAAAAGATTGGATCAGTGAGTTGCCTCACATTTCATCCGTACCAGGTTCTGCTAGCAGCTGGAGCTGCTGACTCATTTGTCTCCATATACACCCACGACAACTCGCAAGCAAGATGAGTCACAACCGTAAGTGAATATGTTGCATTTAGCATTGCTCTTGTGATAAAAAGAAAAGCATAAATCAGTGCAGGTCGGGTCGGTTTTGGGTCGGGGTAAAGTGGGTGTGGTTTATAGTATTCGTTAGCCTCCGTCCAAGATAAAACAAGATCTAAGTTAACACTAAAGAAGGAGGCTTCACCAAATAAGTTATATCTCTACCTATGCAACAACGCACGTAAATGGTGTCTGTAAATAGTTTCCCTGTTTTGCTTTTCATTTTCTCATCTCTTGGGTGTGTGATTTCCTTTTTATTAATCTTTTAAGTCATTCTGTTTCCTAATTTGTTTCATGTGTAAAAAAAATGGTAAAAGTAATGGTGGAAGAAAATTTGCATTTTCGTGACGATGCTTTGAAATGTTGTCTGTTGTTTGTTCAAGTTGGGATCCGTTGGAAATAGGCTTGCAATTTTTCATGCTACGCATCTCTATGATTGGTCAAATACCAGTTTATGTCAGAATCCAAGTAATTATCGAAAAATC

SEQ ID NO: 29 - protein, 1355 aa, Zea mays
MALGDLMASRLVHSSSSRGLSSSPTPPLPNQHHHQNHNHTGDGLLAASGPEPRNGLEADEVDKSAPVPYLPQLVVLCEQRHEPEGIDEAAAAAAGPSTSGLVSKWRPKDRMKTGCVALVLCLNISVDPPDVIKISPCARMECWIDPFSMAPPKALESIGKTLHSQYERWQPKARYKLQLDPTVEEVKKLCNTCRKYARSERVLFHYNGHGVPKPTSNGEIWVFNKSYTQYIPLPITDLDSWLKTPSIYVFDCSASGIIVKAFLERLDWSSSSSASSRKDCILLAACEAHQTLPQSAEFPADVFTACLTTPIKMALHWFCKRSLLRGSLDLSLIDQIPGRQNDRKTLLGELNWIFTAITDTIAWNVLPHDLFQRLFRQDLLVASLFRNFLLAERIMRSANCSPVSYPLLPPTHQHHMWDAWDMAAEICLSKLPQLIADPNAEFQPSPFFTEQLTAFEVWLDHGSEDKKPPEQLPIVLQVLLSQSHRFRALVLLGRFLDMGPWAVDLALSVGIFPYVLKLLQTSAMELRQILVFIWTKILSLDKSCQVDLVKDGGHTYFVRFLDSLDAYPEQRAMAAFVLAVIVDGHRTGQEACMNAGLIDVCLRHLQPENPHDSQTEPLLLQWLCLCLGKLWEDFPDVQLLTLQTNTPEIVLCLLSEPQPEVRAAAVFALGTLLDTTSSNGADDDSDYDEKVKAEINVVRSLLQISSDASPLVRSEVAIALTRFALGHNKHLKSVAAEYWKPQSNSLLKSLPSLANINNPNNVYSPNNILQGSIGPVLRVGSDSSAAGRDGRMSTSSPIATSSIMHGSPQSDDSSQLSDSGLLLRENASNGGPGYTRSRPVDNVIYSQFISTMCSFAKDPYPRIATIGRRALSLIGVEQVVMKNTRFNSGGAHRGETSASNFGMARSSSWLDMNSGNNLIKFRTPPVSPPQHDFLPGLRRVCSMEFGQHPISSPDGLAVPLLSSAAATSNAELSILPQSTIYNWSCGHFSRPLLIGSDDNEEANARREERERLALDYIAKCQRSSACKMTNQIASWDTRFELGTKTALLLPFSPIVVAADENEQIRVWNYDDALPVNSYQNHNLSDRGLSKLLLINELDESLLLAASSDGNVRVWKNFTQKGGQKLVTAFSSVQGHRTAGRSIVIDWQQQSGYLYASGDMSSILLWDLDKEQQLSTIQSSGDSAISSLSASQVCSGHFAAGFAAGSVRIYDIRSPDRVVYTARPHAPRTEKVVGIGFQPGFDPYKIVSASQAGDIQFLDVRRSAEPYLTIEAHRGSLTALAVHRHAPVVASGSAKQMIKVFSLEGEQLTIIRYQPSFMGQRIGTVNCLSFHPYKSLLSAGAGDNALVSIYAEENYK

SEQ ID NO: 30 - DNA, 5012 bp, Zea mays
  source 1..5012: mol_type = genomic DNA
gggcaggcaatttccatttccaacctttatactataaattttattttgttttccctcaatctctctcctcggctgctcgcctggcgtcgcgtcgcctgcttcctcgttcccgcatcgagacgccgcttcccgtcccttccccctcctctctctctcctcccggccaaccaaaccctaaccctaaacccctcccccaccaacctcggcccagtctccccacccccactgacccggctccccacctccgactcgccacgctggcccgattcgatcgatgtacgtgagcgagctcccgacctcatgatggttttggctgcggggtgaaaagaggcacagcaagcgtcgatggcattgggagatctcatggcgtccaggctcgtccactcctcgtcctccaggggactgtcgtcctcgccgacgccgccgttgcccaatcaacaccaccaccagaaccacaaccataccggggatggcctcctggctgccagcgggccggagcccaggaacgggctggaggccgacgaggtggataagtcggcgcccgtgccgtacctgccccagctggtcgtgctgtgcgagcagcgccacgagcctgaggggatcgatgaggctgcggccgcagccgcagggccctccaccagtggcctcgtctccaagtggcgccccaaggatcggatgaagacaggatgtgttgcacttgtattatgtttaaacataagtgtggatccacctgatgtaattaaaatttccccttgtgcaagaatggagtgctggatagatcctttttctatggcacctcctaaagcccttgagagtattggaaaaacgttgcattcacagtatgagcgctggcaacctaaggctcgatacaagcttcagctggacccaacagtggaggaagttaagaagctttgcaatacttgccgcaaatatgccagatcagagagagttcttttccattataatggccatggcgtaccaaagcctacatctaatggtgagatttgggtgtttaacaagagttacacacagtatattccacttccaattactgatcttgattcatggctgaagacaccttcaatttacgtttttgattgctcagcgtctggaattattgtgaaagcttttctagagcgcctagattggagttctagctcctctgcatcttcacggaaggactgcattcttcttgctgcctgtgaggcacatcaaactcttcctcagagtgcagaatttcctgccgatgtgtttacagcttgcctcacaacacccatcaaaatggctttgcactggttttgtaagcgatcattactccgtggttctctggatctttctcttattgaccaaattcctggaaggcaaaatgaccgtaaaactcttcttggggagctcaattggatttttactgctataacagatacaattgcatggaatgttcttcctcatgatctgttccaaaggcttttcaggcaagatcttcttgttgctagcctctttcgtaacttcttgcttgctgagagaatcatgcgatcagcaaattgttctccagtttcttatccgttgttacctccaacacatcaacaccatatgtgggatgcatgggacatggctgctgagatttgcctttccaagcttcctcagttaattgctgatccaaatgcagagtttcagccaagtccgttttttacggaacaactgacggctttcgaagtgtggcttgatcatggctccgaggacaagaaacctcctgaacagttacccatagttcttcaggtcctgcttagtcaatcacacagatttagagcacttgttctgctcggaagatttcttgacatggggccgtgggcagttgatttggccctgtctgtcggaatatttccttatgtactcaaactgcttcaaacaagtgcaatggagttgcgtcaaattcttgtgtttatatggacaaaaattctctctcttgataagtcgtgccaggttgatttggttaaagatggaggacacacatattttgttaggtttcttgacagtttggatgcttacccagaacagcgtgcaatggctgcttttgttttagcagtcattgtggatgggcataggacaggccaagaggcttgtatgaatgcagggcttatagatgtttgcttgagacacctgcaacctgagaatcctcatgattcacagacagaacctttacttctacagtggctttgtttatgccttgggaaactctgggaagatttccctgatgttcagctgcttactttacaaacaaatacgccagaaattgtactatgcttattgtcggagcctcaacctgaggtcagagctgctgctgtttttgcacttgggactctcctggatactacatcatcaaatggcgctgatgatgattctgactatgatgaaaaggtgaaagctgaaataaatgtagttcgaagccttctgcagatatcttcagatgccagtccccttgttcgatctgaggttgctatagcgcttactcgctttgcattgggtcataacaaacatctcaaatctgttgcggctgagtattggaaacctcaatccaattcattgctgaagtcactgccatcattggcaaatataaataacccaaacaatgtttacagtcccaacaatattctacaaggcagcattggtcctgtgctgagggttggcagtgatagcagtgctgctggtcgtgatggaagaatgtctacaagtagcccaattgcaacaagtagcatcatgcatggctctccccagtcagacgattcttcccaactctctgattctggcttattactgagagagaatgctagtaatggtggtcccggctacactaggtccaggcctgttgataatgtcatttattctcaatttatatcaactatgtgttcttttgctaaagatccttacccaagaattgcaactattggtcggagagcactgtcccttataggtgttgaacaagtagtcatgaaaaatactagatttaatagtggaggtgcacatcggggagagacatctgcttcaaactttggaatggcacgctcttcatcctggcttgatatgaattctggaaacaacttgataaaatttaggacccctcctgttagccctcctcagcatgattttcttccaggattacgccgagtgtgttctatggagtttggacaacatcctataagttcacctgatggcttagctgttccccttttaagctccgctgcagctaccagtaatgctgaactaagtatactccctcaatcaacaatttataactggagttgtggtcacttctctaggcctcttttaattggctctgatgataacgaagaggcaaatgctagaagggaagagagagaacgacttgcactagattatatcgctaagtgccagcgctcctcagcttgcaagatgacaaaccaaattgctagttgggatacgaggtttgagttgggaacaaaaacagctttattgttgccattttctccgatcgttgttgcagctgatgaaaatgagcaaataagagtgtggaactacgacgatgcgctgccagtgaactcttatcaaaaccataatttgtcagacagagggctatccaaacttttgctgattaatgagcttgatgagagcttgcttttagctgcctcaagtgatggaaatgtacgtgtatggaaaaattttactcaaaagggaggacaaaaacttgtaacggctttctcatccgttcagggacatcgaactgctggtcgcagtattgtgattgactggcagcagcaatctggttatctttatgcatctggtgacatgtcttccatcttgctatgggatcttgacaaggaacaacaactcagcactattcagtcatctggtgatagtgccatttcttcactgtctgcatctcaggtctgctctggacattttgctgctggttttgctgctggttctgtcaggatatatgacatacgctctcctgatagggttgtctatacggccaggccacacgcaccaagaacagaaaaggttgtaggcatagggtttcaacctgggtttgatccttacaagattgtaagcgcatctcaagctggagacattcagtttcttgatgttagaaggtcagcagaaccctacctgactattgaggcacacaggggttcacttacagcattggcggttcaccggcatgccccagttgttgctagtggctcagccaagcagatgattaaagtgtttagtcttgaaggagagcagttgacaataattcgctaccagccatcttttatgggccaaagaataggcactgtgaactgcctttcttttcacccatacaaatcactcctatctgctggtgctggtgataatgctcttgtctctatctacgccgaggaaaattacaagtaaggtgaggcctacatctcatgacaatgcagccacaaggcaaccggcaggcctggtgttttttggccaagtgatgctcccccgcagcatggtccagctacaagatccaagatggtgtgaagaggctcccggtgcgtattgcctgttgttggacgacgtgtgcaatttgtaagatagagccaagattcgttagccgcctccttgcagaaggtttgtggaacccctaccctgatcatgtcattgaacttggaggctccccccaaataattaatcatacctaggttaaccaaaatgtaaatagacaccgcgtttcgggttgtcggtggtcagcattcctccttttgaagatgcttgtgatgatatgagaaatcaggtttggtaggggctgctgcctgggcaagtggggtttgtgcatatgggcccaggtagtatggttgcacatgtctgctcacagatgctgttgaatggaaggaatgttgtctagtcgcttgcattgtaaccattggagattgtccatttgtatgtatggatcttgtctgtacatctgtaatccgtgcgatgatgtgttttatggaacagtccaaggcttcaggtcttgtgga

SEQ ID NO: 31 - protein, 1401 aa, Glycine max
MALGDLMASRFSQSTVLVVPTSHNHLDDSTTASSSSSAAAAVAALNNNSSSNDDADFAHRRDSEAAIAIISSGNYAGNAATSMAYLPHTVVLCELRHDAFEAAVPAGPSDSGLVSKWRPKDRMKTGCVALVLCLNISVDPPDVIKISPCARMECWIDPFSMAPQKALESIGKTLSSQYERWQPKARYKCQLDPTVDEVKKLCTTCRKYAKSERVLFHYNGHGVPKPTANGEIWVFNKSYTQYIPLPINELDSWLKTPSIYVFDCSAAGMIVNSFIELHEWSASNSSVSQRDCILLAACEAHETLPQSAEFPADVFTSCLTTPIKMALRWFCTRSLLRESLDYSLIDKIPGRPNDRKTLLGELNWIFTAVTDTIAWNVLPHDLFQRLFRQDLLVASLFRNFLLAERIMRSANCSPVSHPMLPPTHQHHMWDAWDMAAELCLSQLPSLVEDPNAEFQPSTFFTEQLTAFEVWLDHGSEHKKPPEQLPIVLQVLLSQCHRFRALVLLGRFLDMGPWAVDLALSVGIFPYVLKLLQTTTPELRQILVFIWTKILALDKSCQVDLVKDGGHIYFIKFLDSMEAYPEQRAMAAFVLAVIVDGHRRGQEACIEAGLIHVCLKHLQSSCPNDSQTEPLFLQWLCLCLGKLWEDFSEAQTIGLQEDATTIFAPLLSEPQPEVRASAVFALGTLLDVGFDSCRSVGGDEECDDDDKFRAEVSIVKSMLDVASDGSPLVRAEVAVALARFAFGHNKHLKSIAAAYWKPQANSLINSLPSLTNIKGSVGGYAKQNQHMPHGSIVSPQIGPIRVGNDNSPVVRDGRVSSSSPLAGSGIMHGSPLSDDSSHHSDSGILNDGFSNGVANHTGPKPFDNALYSQCVLAMCTLAKDPSPRIANLGRRVLSIIGIEQVVAKPLKSSGVRTAESTASPLARSSSWFDMNGGHLPLTFRTPPVSPPRPSYITRMRRVCSLEFRPHLMDSPDSGLADPLLGSGGASGTSDRSFLPQSTIYSWSCGHFSKPLLTAADDSEEVSARREEREKFALEHIAKCQHSAVSRLTNPIAKWDIKGTQTALLQPFSPIVIAADENERIRIWNHEEATLLNSFDNHDFPDKGISKLCLVNELDESLLLAASCTSLYCLIFFILFSNYCQNINVRESFDAADGNIRIWKDYTLRGKQKLVTAFSSIHGHKPGVRNLNAVVDWQQQCGYLYASGEISSIMLWDVDKEQLVNSKSSSSDCSVSALAASQVHGGQFTAGFIDGSVRLYDVRTPDMLVCGLRPHTQRVEKVVGIGFQPGLDQGKIVSASQAGDIQFLDIRNHSSAYLTIEAHRGSLTALAVHRHAPIIASGSAKQLIKVFSLEGDQLGTIRYYPTLMAQKIGSVSCLNFHPYQVLLAAGAADACVCIYADDNTQAR

SEQ ID NO: 32 - DNA, 5041 bp, Glycine max
  source 1..5041: mol_type = genomic DNA
taatttttctcgtttggctttacaaactcgctatttcgtatcgtatcactcgtttcggttttcccttcaaggagagacagtgcctgccgccactgttctatgcgcccccttcatcatcatcatcatcaccatccccatccctaccatcatcaacgccaacaccataaccatcaccatcgccatcatcatcaccttcaccttcaccttaatcttcatcttcatatccctcacactgcgaaaacctagggttccttctttcctcaaaacctttcctattattattccatgtacgatcgccactgaggagggtttccgatttggatggcattgggagatttgatggcctctcgcttttcccaatccacagttctcgtcgttcccacctctcacaaccacctcgacgattccaccaccgcctcctcttcctcctctgctgccgccgccgtcgccgctcttaacaacaacagcagcagcaacgacgacgccgatttcgcgcaccgccgcgactctgaagccgccatcgccatcattagcagcggcaactacgccgggaatgccgccaccagcatggcctacctgccccacaccgtcgtcctgtgcgagctccgccacgacgccttcgaggccgccgtccccgccggcccctccgacagcggcctcgtctccaagtggcgacctaaggacagaatgaagacaggatgtgtagctctagtattatgtttaaacattagtgtcgatccaccagatgtaataaagatatccccttgtgccagaatggagtgctggatagatcctttttctatggcaccgcaaaaggcattggagtcaattggaaaaactttgagcagtcagtatgaaagatggcaaccaaaggcccgctataaatgtcaacttgatccgacagtggatgaggtgaagaagctttgtactacttgtcgtaaatatgcaaagtcggaaagagttttattccattacaatgggcatggtgtgccaaaaccaactgctaatggtgaaatttgggtcttcaataagagttatacacagtatattcccttacctatcaatgaacttgattcctggctgaagaccccatcaatttatgtttttgattgctctgcggctgggatgattgtgaattccttcattgagcttcatgagtggagtgcttccaactcctctgtgtcccaaagggattgcattctgcttgcagcatgtgaagcacatgagactttgcctcagagtgcagaattcccagctgatgtatttacatcttgccttacaacacctataaagatggcattgcgatggttttgtactcgttcattacttcgtgaatcacttgattattcacttatagacaagatccctggccgtccaaatgaccgaaagacacttctgggtgaattgaattggatctttacagccgtaactgatacgattgcctggaatgttcttcctcatgatctttttcagagactgttcagacaggatttgcttgttgcaagtctgtttcgaaattttctacttgctgagcgaatcatgcgatctgcaaattgttctcctgtctctcacccaatgttacctccgacccatcagcatcatatgtgggatgcatgggacatggctgctgagctctgcctctctcaacttccgtctttagttgaggatcctaatgctgaatttcagcctagtacatttttcactgagcagttgacagcatttgaggtatggcttgaccatggttctgaacataagaaaccaccagaacagttgcctatagttcttcaggttttacttagtcaatgccatcggtttcgggctttagtactccttggaaggttccttgatatggggccatgggcagtagatctggcattatctgttggaatatttccatatgttctaaagctgttacaaacaacaacacccgaactacgccagatccttgtattcatatggacaaagattcttgcacttgacaagtcatgtcaggttgatctagtgaaggatggcggtcatatctattttattaagtttcttgatagcatggaagcatatccagagcaacgtgcaatggctgcttttgttttggctgtgattgtggatggtcatagacgaggccaagaagcttgtattgaagctggtttaattcatgtctgcttaaagcaccttcagagttcatgtcctaatgattcacaaactgaaccccttttccttcagtggctttgcctgtgtctggggaaattgtgggaagacttttcagaggcacaaacaatcggtttgcaggaagatgccactactatatttgctcctctactgtctgaaccccagccagaggttagggcatctgctgtttttgcactaggcacccttcttgatgtggggtttgattcatgtagaagtgttggtggagatgaagaatgtgatgatgatgataagtttagggctgaagttagtatagttaagagtatgttagatgttgcttcagatgggagtccattggtgagggcagaggtagcagtggctctagcacgatttgcttttggccacaacaagcacctgaaatctattgctgctgcatattggaagcctcaagctaattctttgattaattccttaccttcgttgactaatataaaaggttcggttggtggatatgctaaacagaaccaacacatgcctcatggaagtattgtttctcctcaaattggtcctatcagggttggaaatgacaactctccagtggttcgagatggccgcgtctcttctagcagtcctctcgctggttcaggaatcatgcatgggtccccattgtctgatgattcatctcatcattctgattcaggaattctgaatgatggtttcagcaatggagtggctaaccacactggaccaaagccctttgacaatgcattgtattcacaatgtgtacttgctatgtgtactttggcaaaggatccatctccacgcattgcaaatcttggtcgccgggtactgtccataataggtattgaacaagtggttgcaaaaccattgaagtctagtggtgttcggactgctgaatctacagcttctccactggctcgttcatcttcttggtttgacatgaatggaggacacttgcctctgaccttcagaactcctccagtcagtcctcctcgcccaagttatataactcgaatgcgtcgagtttgttcattggagtttaggccccacctgatggattctccagactcaggattagctgatccactcttaggttctggtggagcttctgggacttcagatcgaagctttcttccacaatcaactatatatagttggagttgtgggcacttttccaaaccacttttaactgctgcagatgatagcgaagaggtatcagccagaagagaagagagggaaaaatttgcattggagcacatagcaaaatgccagcactctgctgttagcaggctaacaaatccaattgctaaatgggacataaagggtacacaaacagcattgctgcaacctttctctcctatagtgatagctgctgatgagaatgaacgtatcaggatatggaaccatgaggaggcaacactactgaacagttttgataatcatgattttcctgacaaaggaatttctaagctctgccttgtaaatgagcttgatgagagcttgcttcttgctgcttcatgtacttctctctactgtttaatatttttcattttattctcaaattattgtcagaatattaatgttagggaatcttttgatgcagctgatggaaacattcggatttggaaagattatactctgaggggtaaacaaaaacttgtcactgcattctcttcaattcatggtcataaacctggtgtgaggaatctgaatgcagttgttgattggcaacaacaatgtggttatctgtatgcatcgggtgagatatcatctattatgctatgggatgttgataaagagcagcttgtcaattctaaatcttcatcatcagattgcagtgtctcagcattggctgcatctcaagttcatgggggacaatttacagctggtttcatagatggttctgtccgactttatgatgtcagaacacctgatatgctcgtttgtggattaaggcctcatacacaaagagtagaaaaggttgtggggattggctttcagcctggattagaccaaggaaagattgttagcgcttctcaggctggggatatccaatttcttgatataagaaatcatagcagtgcctatcttactattgaagctcataggggttcactcacagctttagcagtacatagacatgccccaattattgccagtggttcagcgaaacaactcattaaggtctttagcctggagggtgatcaactaggcaccattcgatactatcctaccctaatggcccagaaaattggttctgtaagctgcctcaatttccatccataccaagtattacttgctgctggtgctgcagatgcatgtgtttgtatttatgctgatgacaacactcaagctagatgaatccgcattttttccaatgatttcatcggttgtctccagcttagaagagtgcatcaaacatctctcaggattaccatgcatattttcttgtagttggataatcccgtgatgatgctttaatccctctccgctgtgaatacatctgaaaatattggtaggggtacattctgctgatagtgtatgcaagatattcattagcctccatgcttagagggttgcactcgacaggaagaaagtcatgcttattgattggaggcttcggcaaataatgtcaattcgtaaatgtgtaaatatcattttctccataattatttttcgttcctgttttgcgtgctattcccatccttccttgatttgtaaaatttttagtcagaatacataagctagtttgctgacaaatatgggcgactggctcctgaaaatgagccatgtcatgattgtagtttctgttgaatcgggggacccttgtttttaaccaacatgtaattaaattcaagtttgtccaagtgtgcaa

SEQ ID NO: 33 - protein, 1341 aa, Brassica napus
MALGDLMVSRFSQSSVSLASSHRYDDDCVSSSQGDSSRRKDSDATSSIYGNGTAERASATSMAYLPQTIVLRELRHDASEASALLGTSEGIVLAPKWRLKERMKTGCVALVLCLNITVDPPDVIKISPCARIEAWIDPFSMAPPKALETIGQNLSTQYERWQPRARYKVQLDPTVDEVRKLCLTCRKYAKTERVLFHYNGHGVPKPTANGEIWVFNKSYTQYIPLPISDLDSWLKTPSIYVFDCSAARMILNAFAELHDWGSSGSSGSTRDCILLAACDVHETLPQSVEFPADVFTACLTTPIKMALKWFCRRSLLKEIIDESLIDRIPGRQNDRKTLLGELNWIFTAVTDTIAWNVLPHELFQRLFRQDLLVASLFRNFLLAERIMRSANCNPISHPMLPPTHQHHMWDAWDMAAEICLSHLPQLVLDPNAEFQPSPFFTEQLTAFEVWLDHGSEHKKPPEQLPVVLQVLLSQCHRFRALVLLGRFLDMGSWAVDLALSVGIFPYVLKLLQTTTNELRQILVFIWTKILALDKSCQIDLVKDGGHTYFIRFLDSSGAFPEQRAMAAFVLAVIVDGHRRGQEACLEANLIGVCLGHLEASIPSDPQPEPLFLQWLCLCLGKLWEDFMEAQIMGREANAFQKLAPLVSEPQPEVRAAAVFALGTLVDIGFDSSKSVVDDEFDDDEKIRAEEAIIKSLLDVVSDGSPLVRAEVAVALARFAFGHKQHLKSAASSYCKPQSSSLLTSLPSIAKLHDAGNAKLVSLHMSPLTRASTDSQPVAREAKISSSPLGSAGLMNGSPLSDDSSLHSDSGIMLDIVSNGAGHHQRLLDNAVYSQCIRAMFALARDPSPRIASLGRRVLSIIGIEQVVAKPSKPTGRLGEAATTSNTPLTGLARSSSWFDMHAGNLPLSFRTPPVSPPRTNYLLRRVCSLEFKPHLLGSPDSGLADPPLGVSGSERSLLPLSTIYSWSCGHFSKPLLGGTDASQEIVTKREEKEKFALEHIAKCQHSSISKLSNNPIANWDTRFETGTKTALLHPFSPIVVAADENERIRVWNYEEATLLNGFDNHDFPDKGISKLCLINELDDSFLLVASCDGSVRIWKNYATKGKQKLVTGFSSIQGHKPSARDLNAVVDWQQQSGYLYASGEVSTVALWDLEKEQLLRSIPSESECGVTALSASQVHGGQLAAGFADGSLRLYDIRSPEPLVCATRPHQKVERVVGLSFQPGLDLAKVVSASQAGDIQFLDLRTTKDTYLTIDAHRGSLTALAVHRHAPIIASGSAKQLIKVFSLQGEQLGIIRYYPSFMAQKIGSVSCLAFHPYQVLLAAGAADSFVSVYTHDNSQAR

SEQ ID NO: 34 - DNA, 4521 bp, Brassica napus
  source 1..4521: mol_type = genomic DNA
cgaacctcgccccaatcgtgacaataactctagggccgagcgattcgattttgagggatttggggtgtttgattcttctctggtgtgttctccgatttggatggcattgggagacttaatggtatctcggttctcgcaatcctcagtttctctggcttccagtcaccgctacgacgacgactgtgtctcctccagtcaaggtgattcctcgcggaggaaagattccgatgctacgagtagtatctacggaaatggcacggcggagagagcctccgctaccagcatggcgtacttgcctcagactatcgtcctccgggagcttaggcacgacgcgtccgaggcgtctgcgcttttggggacttccgaggggattgtgttggctcctaaatggcggcttaaagagcgaatgaaaacaggatgtgtagctctagttctgtgtttaaacattactgttgatccgccggatgttataaagatatctccttgtgcaagaatcgaggcttggatagacccattttccatggcgccgccaaaagctctcgagactattggacaaaacttgagcactcagtatgaaagatggcaaccaagggctcgatataaggttcagttagatccgacggtagacgaagtcaggaagctgtgcttgacttgtcggaaatatgcaaagaccgagagggttctattccattataatgggcatggtgtgccaaagcctacagctaatggggaaatttgggtattcaacaagagttatacgcagtacattccattgccaatcagtgatcttgattcctggttgaagacaccgtccatctatgtttttgactgctctgctgctaggatgattcttaatgcgtttgctgagcttcatgattggggttcgtctggttcctctggatccacaagggattgtattctacttgctgcttgtgacgtacatgagacacttcctcagagcgttgaatttccagctgatgtttttactgcttgcctcacgacacccattaaaatggcgttaaaatggttctgcaggcgttctcttctaaaagaaataatcgatgaatcacttattgacagaattcccggccgccaaaatgaccgtaagacattgttaggggagttgaattggatcttcacagcagtgacggatacaattgcatggaatgtgcttcctcatgaacttttccagagattattcagacaggatttgttggtcgctagccttttccgaaatttcttacttgctgagagaataatgcggtccgcaaattgtaatccaatatctcaccctatgctacctcctacgcatcaacatcatatgtgggatgcatgggatatggctgctgaaatctgcctttcgcaccttccccaacttgttctggacccaaacgcagagttccagccaagtccattttttactgagcaattaacagcttttgaggtgtggcttgatcatggatctgagcataagaagccaccggagcaattgcctgttgttcttcaggtgttacttagccaatgccatcggtttcgtgctcttgtacttcttggaagatttcttgatatgggttcatgggccgtggatctggctttgtctgttggaatattcccatatgtgctgaagcttctgcaaacaacaacaaatgagctaagacagatccttgttttcatatggacaaaaattctggcacttgataagtcatgtcaaattgaccttgtgaaggatggggggcatacatatttcataagatttctagatagctcgggtgcatttccagaacaacgagctatggctgcttttgttctggctgtcatagtggacggacaccgacgaggccaggaagcatgtcttgaagcaaatttgattggtgtttgtctggggcaccttgaagcatcgataccaagtgatccacagccagaaccattgtttttacaatggctttgtctttgtcttggaaagttgtgggaagattttatggaggcccaaataatgggcagggaggcaaatgcttttcaaaaattggcacctctggtttccgagccccaacctgaggtaagggctgctgctgtttttgccctgggtaccttagttgatattgggtttgactctagtaaaagtgttgtggacgatgaatttgatgatgatgaaaagattagagcagaagaagctatcattaaaagtctcttagatgttgtctcagatgggagtccacttgtccgggcagaggttgctgtagctcttgcacgttttgccttcggccacaaacagcacctaaagtcagccgcatcttcatattgcaagcctcagtcaagttctttgcttacttcgctcccttcaatagctaaactccatgatgctgggaatgcaaaacttgtttctttacacatgagtcctctgactagagctagcactgatagccaaccagtggctcgtgaggccaagatctcaagcagccctcttggctcggctgggctgatgaatggatctccattatctgatgattcttcgttacattctgattctggaatcatgcttgacattgtcagcaatggagctggccatcaccaaagactattggacaatgctgtttattcgcagtgcattcgagctatgtttgccttagctagagacccatctccacgtattgcaagtctcgggcggcgtgtgctttctatcattggaatcgaacaagttgttgcgaaaccctcgaagcccactggccgactaggggaagccgcaacgacatctaacactcctcttactggcctagctcgttcatcctcatggtttgatatgcatgcaggtaatctgcccttaagttttaggactcctccggtcagccctcctagaacaaactatctactgaggagagtttgttcgctagagttcaaacctcatctgttgggttcaccagactcaggattggctgatccgcctttaggcgtcagtggatctgaaaggagtttgctcccattatcaactatctacagctggagctgtggccacttttctaaaccgcttcttggtgggacggatgctagtcaagagatagttaccaaaagagaagagaaagaaaaattcgcactcgagcatattgcaaaatgccagcactcctctattagcaagctcagcaataatcctattgccaactgggatacgaggtttgaaacgggaacaaagacagctctccttcaccctttctctcctattgtagttgctgcggatgagaatgaacggatcagagtctggaactatgaggaagcaactcttctcaatggctttgacaatcatgattttcctgacaaaggaatttcaaagctctgcctcatcaatgaacttgacgactcttttctacttgttgcatcatgtgatggctcggtccgaatatggaaaaactatgcaacgaagggtaaacaaaagcttgttacggggttttcttcaattcagggccacaagcccagtgcacgtgacttgaacgctgttgtggactggcaacaacagtctggctacctgtatgcttctggggaggtgtcaacagtcgcactttgggacctggagaaagaacagcttctcagatctattccctctgagtcagagtgcggagttacagcactttcggcttctcaagtgcacgggggtcaacttgctgctggtttcgctgatggatctttgagactctatgatattcggtcacctgaaccgcttgtctgtgcgactcggcctcatcagaaagttgaaagagtggttggactcagttttcagcctgggcttgatctcgcaaaggtggtgagtgcatcacaggccggtgacatacagttccttgacctcagaacaacaaaggacacatacctgacgattgatgcacacaggggttcactcacggccttagctgttcacagacacgctccaatcatcgcgagcggatctgcaaaacagctgattaaagtgtttagccttcagggagaacaactagggataatccgctactacccgtccttcatggcacagaagatcggttcagtgagctgcctcgcttttcatccgtaccaggttctgcttgcagctggagctgcggactcattcgtctctgtatacacccatgacaactcacaagcaaggtgagtcgcacaccgtaagtcaaaatgttgcatcgctcttgtgataaagaaaggtccagtcctggtcgggggtaaagtggtttatagttattcgttagcctccgttgaagtttagaaaggagatctaagttaacaactgatgaaggaggaggcttctccaaataagttctctatctcttcctatgccaactcaagtaaatggtatctgtaaatagtttcactgtttcgtaatttgttttttatttagcatgtttcattgggtgtgtgctttctttcctttctattaaaatcttaaaagtcttttccgtttcacaatttgtttcaaaatgttaaaaagtaattgtggaaaaaaaaaagtttgctttcgtgatattattagaaggattagtatattgaaaa

SEQ ID NO: 35 - protein, 1364 aa, Gossypium hirsutum
MALGDLMTSRFSQLSLAVSNHVMDGNGSNGDYLEDAASADMASQRRDFDTASTSSYANAVASSATVPTTMAYLPQTIVLCELRHAAFEASTPTGPSDSGLVSKWRPKDRMKTGCVALVLCLNISVDPPDVIKISPCARMECWIDPFSMAPQKALETIGKNLRDQYERWQPKARCKVELDPTVDEVKKLCNTCRRYAKSERVLFHYNGHGVPKPTANGEIWLFNKSYTQYIPLPISDLDSWLRTPSIYVFDCSAAGMIVNAFIELLDSGTSSYPGSARDCILLAACEAHETLPQSAEFPADVFTSCLTTPIKMALRWFCTRSLLHESLDCSLIDKIPGRQNDRKTLLGELNWIFTAVTDTIAWNVLPHELFRRLFRQDLLVASLFRNFLLAERIMRSANCSPVSYPMLPPTHQHHMWDAWDMAAEICLSQLPALVEDPNAEFQPSLFFTEQLTAFEVWLDHGSEHKKPPEQLPIVLQVLLSQCHRFRALVLLGRFLDMGPWAVDLALSVGIFPYVLKLLQTTTPELRQILVFIWTKILALDKSCQVDLVKDGGHVYFIRFLNSVEAYPEQRAMAAFVLAVIVDGHRRGQEACIEAGLIHVCLKHLHSSMQNDVQTEPLFLQWLCLCLGKLWEDFTEAQIIGLQADAPAICAPLLSEPQPEVRASAVFALATLLDIGFDSFRDGVGGDEECDDDEKTRAEIIVIIKSLLNIVSDGSPLVRAEVAVALARFAFGHKQHLKSIAAAYWKPQSNSLLSSLPSLANINGTGSGNIVSSQIGPLIRVGNDNSSVVRDGRVSTSSPLATAGVMHGSPLSDDSSQHSDSGILNDGISNGVIRHSRSKPLDNAMYSQCVPAMCTLAKDPSPRIATLGRRVLSIIGIEQVTKSVKSAGSSSARPGEPTTSSTTPSFAGLARSSSWFDMNGGHLPLTFRTPPVSPPRQNFLTGMRRVCSLEFRPHLMNSPDSGLADPLLGSSSGVSERSLLPQSTIYNFSCGHFSKPLLTASDDSEELLAIREERERFALERIAKCQHSSVSKLNNNSQIASWDTRFETGTRTALLQPFSPIVIAADESERIRVWNYEEATLLNGFDNHDFPEKGISKLCLLNELDESLLLVASGDGNIRIWKDYTVRGKQKLVTAFSSIQGHKPGVRSLSTVVDWQQQSGYLYASGEISSVMIWDLDKEQLINSIPSSSDCSVSALASSQVHAGQFAAGFVDGSVRLYDVRAPDMLVCATRPHTQQVERVVGIGFQPGLDQGKIVSASQAGDIQFLDIRNQRDAYLTIDVHRGSLTALAVHRHAPIIASGSAKQLIKVFSLEGEQLGVIRYQHTFMAQKIGSVSCLTFHPYQVLLAAGAADACVSIYADDNSQTR

SEQ ID NO: 36 - DNA, 4991 bp, Gossypium hirsutum
  source 1..4991: mol_type = genomic DNA
agaaggaaagagtacacaaaaattaaatatacagcaatgcacgcacaaaaactggaatgcatagacaaaatccttatttgagagagttgagttgaaaccccccttcctttctttctctctctctttccatttttcttttaatttatttttcttgtgccgccagtgttgtattcgccaccaccatcgtcgctgccgtaacctagggtttgttctagggtttgagatctcttttcgcttatcttttcttcgcacaactttccgagctggattctctctctttcctcaggcgaatctccatgcgccgctggttcagcggctttgttgattaaatcacttcctcttcttcctcctcctcctcctcctcctcttcttcttcttcttcttctctcgttctttcttgctttcattcctcgatcagtttcgagaagagaggctagggttttcttgttgtggaatttttctttgtttcttattaattaaattttcatccgagggctttgtttgtttcttgtttcattttgttgggggatttggatggcattgggcgatctgatgacgtcacggttttcgcaattgtcgttggcagtatccaatcatgtcatggacggcaacggcagcaatggcgactaccttgaggatgctgcttctgccgacatggcctctcaaaggagggattttgatactgcatccaccagtagttatgccaatgccgttgcatcctccgccacagtgcctacaactatggcttacttgcctcaaactattgtcttatgcgagcttcggcatgctgcgtttgaagcttccactcctactggaccctctgatagtggccttgtctccaaatggcgacccaaggatcgaatgaagacaggatgtgtagcactagttttatgtttaaatattagtgttgatccacctgatgtaataaagatatctccttgtgcaagaatggagtgttggattgatcctttttctatggcaccacaaaaagctctcgaaaccattgggaaaaatttgagagatcaatatgagaggtggcagcccaaggcacggtgcaaggttgagcttgatcctacagtggatgaagtgaagaaactttgtaatacatgtcgtagatatgccaagtcagagagagttctatttcattataatggacatggtgttccaaagccaactgcaaatggtgaaatctggcttttcaataagagttatacacagtatattcccttgcctatcagtgaccttgattcatggttaagaacaccttctatttatgtttttgattgctctgcggctggaatgattgttaatgccttcattgagcttcttgactctggcacttctagttaccctggatctgcacgagattgcattctgctggctgcatgtgaagcacatgagacacttcctcaaagtgctgaatttcctgctgatgtgtttacttcttgtcttactacacctatcaaaatggcattgagatggttttgcacacgttcgttgcttcatgagtctcttgactgttctcttatagataaaattcctggccgccaaaatgatcgtaaaacacttctaggggagttgaattggatttttactgcagtaacagacacaattgcttggaacgttctccctcatgagcttttccggagattgtttaggcaggatctactagttgccagtttgttcagaaattttttgcttgctgagaggattatgcgctctgcaaattgttctccagtttcttacccaatgttgccaccaactcatcagcaccatatgtgggatgcatgggacatggctgctgaaatttgcctttctcagcttccagcattggtcgaggatcctaatgcggagttccagccaagtctcttttttactgaacagctgacagcttttgaggtatggcttgaccatggatctgagcataaaaagccaccggagcaattgcctatcgttcttcaggttttacttagtcaatgccatagattccgtgcactggttcttcttggaagattccttgatatgggaccatgggctgtagatctggccctttctgttggaatattcccctatgtgctgaagctgttgcaaacaaccacaccagaactgcgtcaaatccttgtcttcatatggacaaaaattttggcccttgataagtcatgtcaggttgatctggtaaaagatggcggtcatgtttatttcattaggtttcttaatagcgtggaagcatatcctgaacagcgagcaatggctgcatttgttctagctgtcattgtggatgggcatagacggggccaggaagcctgtattgaggcagggttgatccacgtgtgcttgaagcaccttcacagttccatgcaaaatgatgtgcaaaccgaacccttgttccttcagtggctttgcttgtgtcttgggaagctctgggaggattttactgaggctcaaataataggtttgcaggcagatgcacctgcaatatgcgctcctcttctttctgagccgcagcctgaagttagggcttctgcagtttttgcattggctaccttgcttgacattgggtttgattcattcagagatggtgttggaggggatgaagaatgtgatgatgatgaaaagactagagccgagattattgttattattaaaagccttctgaatattgtttctgatggtagccctcttgtcagagcagaagtagctgtagctctagcaaggtttgcctttggacataagcagcatctcaagtcaattgctgctgcatattggaaacctcaatctaattccttgttgagttcattgccttcgctggctaatataaatggcacaggaagtgggaatatagtttcatcccagataggccctttaattcgtgtaggaaatgacaactcatctgtagttcgggatggaagggtttccaccagcagtcctcttgctactgctggagtcatgcatggatctcctttatctgatgattcatctcaacattctgattctgggattttgaatgatggtattagtaatggagtcattcgtcattcaaggtcaaaacctttggacaatgcaatgtattcacagtgtgtaccggctatgtgtactttagctaaggacccttctccacgcatagcaactctaggcaggcgggttctttcaattattgggattgaacaagtgacaaaatctgtgaagtctgctggtagcagcagtgctaggcctggtgagcctacaacttcctcaacgactcctagttttgctgggttagctcgttcttcttcatggtttgacatgaatggaggtcatctgcctttaacgttcagaactcctcctgttagcccccctcggcaaaattttttgacaggaatgcgtagagtttgctctttagaattcaggcctcatctgatgaattctccggactccggattggctgacccacttctagggtcttcttctggggtttctgagcgcagtttacttccacaatcaactatctataattttagttgtggccacttctcaaagccacttcttactgcatcagatgatagtgaagaactattggctataagagaagagcgggagagatttgcactggagcgtattgctaaatgtcagcactcctctgttagcaaacttaacaataatagtcaaattgctagttgggatacaagattcgagacaggtacaagaacagcattgctgcaacctttctctcctattgttatcgcggcagatgagagtgaacggatcagggtatggaattatgaggaagccacccttctcaatggttttgataatcatgattttccagagaagggaatttctaaactttgtcttttgaatgagcttgatgaaagcttgttgcttgttgcttcaggtgatggaaatatacggatttggaaagattatacagtcaggggcaaacagaaactcgttactgcattttcttctattcaaggtcacaaacctggtgtgcggagtttgagtactgttgtggactggcagcagcagtctggatatctttatgcatctggagagatatcatctgtcatgatctgggacctggataaggagcaacttattaattcaataccatcctcctcagattgcagtgtctcagcattggcttcttctcaagttcatgctggtcaatttgcagccggttttgtggatggttctgtcagactctatgacgtccgggcacctgacatgctggtttgtgcaactagaccacatacccagcaagttgaaagagttgtgggtattggctttcagcctggacttgatcaaggaaagattgttagtgcttcccaggcaggtgatattcagtttcttgatattagaaaccaaagagatgcttaccttacaatcgatgtgcacagaggctcacttacagctctagctgttcatagacatgccccgataattgccagtgggtcagcaaaacagctaatcaaagtcttcagtctggaaggcgagcaactaggcgtcattcggtaccaacataccttcatggcccagaagattggttctgtgagctgccttaccttccatccctaccaagtattgcttgctgctggtgctgcagatgcgtgtgtttctatctacgctgatgacaattctcagacaagatgaatataaattctttcccaatttcatgaattcgggaggtattgcacggtagaactggcatcggcacaaattgcacggagtcaggatcgaagataacctgtaaatgttgcctgccacatttaccatttcattgtgggtattcctggaaattggggggggggatctgctgattgtaatgctatatatatagtgggatatatattcattagcctccggtgtagatggtcttacacatgttcggaaaaaggaatcctatcctgcagatatttcatggatgggggggctttggcaaataatttgttcttataggtgcatgtaaatagttttcccattattatttcacccttttcagtgtgttcattgtt

SEQ ID NO: 37 - protein, 1353 aa, Solanum tuberosum
MALGDLMASRFSQSSAALDEFGNEDGERNNVRDLDTASSSYVGGGVADNAMTTTSMAYFPQTIVLCELRHDRFEDSVPSGPSDTGLVSKWRPRDRMKTGCVALVLCLNISVDPPDVIKISPCARMECWVDPFSMAPQKALETIGRTLNQQYERWQPRAKYKISLDPTVDEIKKLCTTCRKYAKSERVLFHYNGHGVPKPTANGEIWLFNKSYTQYIPLPISDLDSWLKTPSIYVFDCSAAGMIVNAFIELQDWTASGSSATSTRDSILLAACEAHETLPQSAEFPADVFTSCLTTPIKMALRWFCTRSLLHESLDYSLIDRIPGRQTDRKTLLGELNWIFTAVTDTIAWNVLPHDLFQRLFRQDLLVASLFRNFLLAERIMRSANCSPISYPMLPPTHQHHMWDAWDMAAEICLSQLPNLVEDPNAEFQPSPFFTEQLTAFEVWLDHGSKDKKPPEQLPIVLQVLLSQCHRFRALVLLGRFLDMGPWAVDLALSVGIFPYVLKLLQTTTPDLRQILVFIWTKILALDKSCQVDLVKDGGHTYFIRFLDSVEAYPEQRAMAAFVLAVIVDGHRRGQEACFEAALIHVCLKHLQGSTPNEAQTEPLFLQWLCLCLGKLWEDFTEAQVQGLQADAPAIFAPLLSEPQPEVRAAATFALGTLLDVGFDSARDGVGGDEDCDDEEKVRTEVSIIKSLLSVASDGSPLVRVEVAVALARFAFGHNKHLKSVAAAYWKPQANSLLTSLPSFAVKSSGSGYTTPTHSISHGSRVPSPIAPLLRVGGDSQSISRDGRVSTSSPLATPGVIHGSPLSDDSSQLSDPGILNDAVTNGVVNHTRSRPLDNALYSQCVLAMCALAKDPSPRIAGLGRRVLSIIGIEQVVAKSVKSTGESTTVPNTGYAGLARSSSWFDMNGGHLPLTFRTPPVSPPRPSYLTGMRRVCSLEFRPHLMHSQDSGLADPLLGSAGSSGPSERSFLPQPTIYNWSCGHFSKPLLTAADDSEEMVARREEKEKLALDLIAKCQHSSVSKLHNQIASWDTKFEIGTKTALLQPFSPIVIAADESERIRVWNYEEATLLNSFDNHSYPDKGISKLCLVNELDESLLLVASSDGNIRIWKDYTLRGRQRLVSAFSSIQGHRPGVRSVNAVVDWQQQSGYLFSSGEVSSIMAWDLDKEQLVNTIPTSSDCSISALSASQVHAGHFAAGFVDGCVKLFDIRMPELLVCASRPHTQRVERVVGIGFQPGLEPAKIVSASQAGDIQFLDMRNLKEAYLTIDAHRGSLTALAVHRHAPLIASGSAKQLIKVFNLEGEQLGTIRYLSTFMAQKIGSVRCLTFHPYQVLLAAGAADSCVSIYADEIAPTR

SEQ ID NO: 38 - DNA, 4850 bp, Solanum tuberosum
  source 1..4850: mol_type = genomic DNA
tacaaaacaacaactctctccttctcttctttttctcgttttgttgcttcaacagccgcactgtcctccttcttcttgttgttcttcttcatcaacaccatcttctagggtttcttttagggttttccagaaccttcttgaagctggattctccttccttccttctccccatctctcaccgtcgctggttcactggtttcattgattaatcctcaagaaacaacctttttcaatctccctttaccatcactactgaaaccctgatctttattgcaaattaccggataaaaacggtatctttagggtttttggtatctgggtcgatctgggttatgagtttcactccaaaatctgttcgattagggcaaggttaggaaaattttcagcttcaattattgcgatttgaatggcattgggggatttgatggcgtcgaggttttcacaatcatcagctgctttggatgagtttggaaatgaggatggggaaaggaataatgttagagatttggacactgctagtagtagctatgttggtggtggggttgctgataatgcgatgacgacaacaagtatggcttattttccgcagacaattgtgttgtgtgagcttaggcatgatcgttttgaggattctgttccttctggtccatctgacactgggttggtctctaaatggcggcctagggaccgaatgaaaacaggatgtgttgcccttgttttgtgtttaaacatcagcgttgatccacctgatgtaataaagatatctccttgtgctcgaatggagtgttgggttgatccattttcgatggcacctcaaaaagcacttgagacaattgggagaactttgaaccaacaatatgagaggtggcaacctagggctaaatataagatttccttggatcctactgttgatgaaatcaagaagctttgcacaacttgtcgaaagtatgctaagtcagagcgagttctattccattataatggacacggtgttcctaaacctacagccaatggtgaaatttggctatttaataagagttatacgcagtatattcctttgccaatcagcgacctggattcctggttgaagacaccatcgatctatgtatttgactgctctgctgctgggatgattgttaatgccttcatagagcttcaggactggactgcttctggttcttctgcaacttctacaagggatagtattctgcttgctgcttgtgaagcacatgagacccttccacagagtgctgagtttccagccgatgtgttcacttcctgcctcacaacacccatcaaaatggcattgagatggttttgcactcgttcgttgcttcatgagtcgcttgattactctctgattgatagaattcctggccggcaaactgatcggaagacacttcttggcgagctgaattggatttttactgcagttactgacacaattgcctggaatgttctaccacatgatctatttcagagactgttcaggcaagatctgttagttgctagtttgttcagaaacttcttacttgctgaaagaatcatgcgctctgcaaattgttcaccaatttcatacccgatgctaccacctactcatcagcaccatatgtgggatgcatgggacatggcagcagaaatctgtctttcacagcttccaaatttagttgaggatcccaatgcagaatttcagccaagtccatttttcacagagcagttgacagcttttgaagtatggcttgaccatggatcaaaggataaaaagcctccagagcagttgcccatagtgttacaggtgttacttagtcaatgccacaggtttcgtgcacttgtacttcttggaagatttctcgatatggggccttgggctgttgatctggccttgtctgttggaatatttccttatgttctaaaacttttgcaaaccaccactccagatcttagacaaattcttgtctttatctggacgaagatccttgcactcgataagtcttgtcaggttgaccttgtaaaggatggaggacatacatatttcattagatttcttgacagtgtggaggcttatccagagcaaagggcaatggctgcatttgttttagcagtcattgtggatggtcacagacggggtcaggaggcctgttttgaagcggctcttatacatgtctgcttgaagcatcttcagggatcgacgcctaatgaggcacaaactgaaccactttttcttcaatggctgtgcctttgtctgggaaaactgtgggaggatttcacagaggctcaagtacaaggtttgcaggcagatgcaccagcaatatttgcgcctctgctgtctgagccccaacctgaggtacgtgctgcagctacttttgctctagggactttacttgatgttggatttgactcggctagggatggtgttggaggagatgaagactgtgatgatgaggagaaggttaggactgaagtcagtataattaagagccttttaagtgttgcttctgatggaagccctttggttcgagtggaggttgctgtagctttggcacgttttgcttttgggcataacaagcatctgaagtcagtagccgctgcatattggaagcctcaggctaattccttgctaacttccttgccttctttcgcggtcaagagttctggtagtgggtatacaaccccgactcacagtatttcacatggaagtagagttccgtctcctattgctcctttattgagagtggggggtgacagtcagtccatatctcgggatgggagagtctcgaccagcagcccccttgcaacacccggcgttatacatggatcacctttatctgatgattcctctcaactttctgatcccggaatcttgaatgatgctgttaccaatggtgttgtaaatcatacaagatcaaggcctctagacaatgcactctattctcagtgtgtactggctatgtgtgccttggcaaaggatccgtcaccacgtattgcaggtcttggccggagagtcctttccattatcgggattgaacaggttgttgccaaatctgttaagtcaactggtgaatccacaacagttccaaacactggttatgctggattggctcgatcttcatcttggtttgatatgaacggaggacatttacctctgacttttagaactcctcctgttagtcccccacggcctagttacttgacaggaatgcgaagagtttgttctttagagttcaggccccacctaatgcactctcaagactcaggattagctgacccattgttggggtctgctggatcttctggaccttcagaacgcagttttcttccccaaccaactatctacaattggagttgtggccacttctcaaagccacttcttactgcagctgatgatagtgaagagatggttgctagaagggaagagaaggaaaagttggcccttgacctcattgcgaagtgccaacactcttctgtaagcaaactacacaatcaaattgctagttgggatactaagtttgagattggcaccaaaactgctttgctgcagcccttctcacctattgttattgctgcagatgagagtgaaagaatcagggtatggaattacgaggaggctacccttcttaatagctttgataaccacagttaccctgataaaggaatttcaaaactgtgtcttgtcaatgagctagatgagagtttgctccttgttgcttcaagtgatgggaacatccggatttggaaagactatactctaagaggccgacaaagactagtttctgcattttcatccattcaaggtcacagacctggtgtccgtagtgtgaacgctgttgtggattggcagcagcagtctggatatctgttttcatctggtgaagtttcgtcaatcatggcttgggatcttgacaaagaacaacttgttaatacaattccaacatcctcagattgcagcatctcagcattgtcagcttctcaagttcacgcaggacactttgctgctggctttgtggatggatgtgtcaaactatttgatattcgaatgcctgaactgcttgtttgtgcatcacggccgcacacccaaagagtagagagagtagtggggatcggctttcaacctggacttgaaccagcaaagatcgtcagtgcctctcaagctggtgatattcagttccttgatatgagaaatctcaaggaagcttacttaacaattgatgctcataggggatcgctaacagccctagctgttcatcgtcatgcaccccttatagcaagtggttcagctaaacagctaatcaaagtcttcaacctggaaggtgagcaattaggcaccataagatatttgtccaccttcatggctcagaagataggttcagtcagatgtcttaccttccatccctatcaagtgctgcttgctgctggagcagcagattcttgtgtttcaatctatgctgatgaaattgccccgacaagataattttctgaaacattcaagattgagaaaccaaaaaagattacagacataagttgaggtggtcagctgcaaaaatggactgttgacatctctgaaacatatcagctcactgaggtcctgtgctacttattcattgagcctctgcagcgacgagtgttgccaagttgggaaaaaattcaacatattggaggaggctttagcaaataatttgataaataggtggtaaaagaaggtgtaaatatctgttattcattgtttctagtttgtaattttctccgtgtgcttccttagttcagccccctctactttcttttctcccctggttcaatttgtacagctgaattcataatttatatatgttgttggcaaccagtgttctgtgttc

SEQ ID NO: 39 - protein, 1391 aa, Triticum aestivum
MALGDLMASRLVHSSSSSSSSSLPTPSLAAVNHQTDRVDGELPVANGPELRREDAGEEDEEGRAVALVPCLPQVVVLCEQRHEGFNEAAVAAAAGPSTSGLVSKWRPKDRMKTGCVALVLCLNISVDPPDVIKISPCARMECWIDPFSMPPPKALENIGKTLHSQYERWQPKARYKLQLDPTVEEVKKLCNTCRKYARSERVLFHYNGHGVPKPTANGEIWMFNKSYTQYIPLPITDLDSWLKTPSIYVFDCSAAGMIVKAFLERLDWSSSSSASSVKDCILLAACEAHQTLPQSAEYPADVFTACLTTPIKMALHWFCKRSLLSGSLDHSLIDQIPGRQNDRKTLLGELNWIFTAITDTIAWNVLPHELFQRLFRQDLLVASLFRNFLLAERIMRSANCSPITYPLLPPTHQHHMWDAWDMAAEICLSKLPHLIADPNAEFQPSPFFTEQLTAFEVWLDHGSEDKKPPEQLPIVLQVLLSQSHRFRALVLLGRFLDMGPWAVDLALSVGIFPYVLKLLQTSAMELRQILVFIWTKILSLDKSCQVDLVKDGGHAYFIRFLDSLDAYPEQRAMAAFVLAVIVDGHRRGQEACMSAGLIDVCLRHLQPENPHDAQTEPLLLQWLCLCLGKLWEDFPEAQLLGLQSNAPEIVTCLLSEPQPEVRASAVFALGNLLDIGSPSVNGGDDDSDDDEKARAEINVVRSLLQVTSDGSPLVRAEVSVALTRFALGHNKHLKSVAAEYWRPQTNSLLKSLPSLANINNPSNVYSPSNFLQGSSGLSSHIGPVLRVGSDTSATGRDGRISTSSPIATNSIMHGSPQSDDSSQHSDSGILLRDNASNGGLSYTRSRPIIDSGIYSLFISTMCSIAKDPYPRIANIGRRALSLVGVEQVVMRNTRFGSGGAGESSAPSSNIGMARSSSWFDMNSGISMAFRTPPVSPPQHDYLTGLRRVCSMEFRPHLLNSPDGLADPLLSSAAGPSTSELNILPQSTIYNWSCGHFSRPLLTGSDDNGEVSARREERERTALDCIAKCQRSSACKMTSQIASWDTKFELGTKSALLLPFSPIVVAADENEQIRVWNYDDALPVNTFENHKLSDRGLSKLLLINELDESLLLVGSSMPSLFMAFSCICTYVFLVTSFFHTVGDGNVRIWRNYTQKGGQKLVTAFSSVQGHRAAGRSVVIDWQQQSGYLYASGDMSSILVWDLDKEQLLNTIPSSADSGISALSASQVRSGQFAAGFIDASVRIFDVRTPDRLVYMARPHAPRTEKVVGIGFQPGFDPYKIVSASQAGDIQFLDVRRAAEPYLTIEAHRGSLTALAVHRHAPVIASGSAKQMIKVFSLEGEQLTIIRYQPSFMGQRIGSVNCLSFHPYKSLLAAGAGDNALVSIYAEDNYQVR

SEQ ID NO: 40 - DNA, 5274 bp, Triticum aestivum
  source 1..5274: mol_type = genomic DNA
gtttctctctctccctcttcctccccaccctctccgccgcatccccgtctcccccgaccctcacccccaccccgctgctcccgtccaaaccctaaccctaaaccccaccctgcgcctccatccaatccctgcctggcgtccacccgtccccggcggcacggattcggattcgccgcgccggaccgattcgatcgatgtaggcagcgagcatcgctgcccactccgtacacacgactcgcgcccccgcctcgagatggctgccgcggggtgacgaggaggaggggagagtagtagagggagcgcccaagcgtagatggcattgggagatctcatggcgtccaggctcgtgcactcgtcctcatcgtcgtcctcgtcctcgctgcccaccccctccttggcggcggtgaaccaccagacggaccgcgtggatggtgagctccctgtcgccaacgggccggagcttcggagggaggacgccggcgaggaggatgaggaggggagggccgtggcgctcgtgccgtgcctgccgcaggtggttgtgctgtgcgagcagcggcacgaggggttcaatgaggctgcggtggcggcggcggccgggccatcgacgagcgggctcgtctccaagtggcgccccaaggacaggatgaagactggatgtgttgcacttgtattatgtttaaacattagtgttgatccgccggatgtaattaaaatttccccttgtgcaagaatggagtgttggatagatccattttctatgccaccccccaaagcccttgaaaatattggaaaaacattacactcacagtatgagcgatggcagcctaaggctcgttacaagcttcagctggatccaacagtcgaagaagtcaagaagctttgtaatacttgccgcaaatatgccagatcagagagggttctttttcattacaatggtcatggtgtaccaaagcctacggctaatggagagatttggatgtttaacaagagttatacacagtatatccctcttcctattactgatctcgattcatggctgaaaacaccatcgatttatgtttttgactgctcagcagctggaatgattgtgaaagcttttctagagcgcctagactggagttcaagctcttctgcgtcttcagtgaaggattgcattctccttgctgcctgtgaggcacatcaaactcttcctcagagtgcagaatatcccgctgacgtgtttacagcttgcctgaccactcccataaaaatggcattgcactggttttgcaaacgatcattactcagtggttctctggatcactctcttattgaccaaattcctggaagacaaaatgaccgtaaaacacttctgggggagttgaactggattttcactgctattacagatactattgcatggaatgttcttcctcatgaattattccaaaggcttttcaggcaggatcttttggtcgccagtctctttcgcaacttcttacttgctgagagaatcatgcggtctgcaaactgctcaccaattacgtacccactattgccacccacacatcaacatcatatgtgggatgcatgggacatggctgcggagatctgcctttccaagcttcctcatttgattgctgatcctaatgcagagtttcagccgagcccatttttcacggaacaattgacagcattcgaagtgtggcttgatcatggttctgaggataagaaaccccctgaacagttacccattgttcttcaggtcctgcttagccaatcacacagatttagagcacttgttctgcttggaagatttcttgatatgggtccatgggcagttgatttggccttatctgttggtatcttcccttacgtgctcaaactgctccaaacaagtgcgatggagttgcgtcaaattcttgtgttcatatggacaaaaattctctctcttgataagtcatgccaggttgatttggtgaaagatggagggcatgcatattttatcaggtttcttgatagtttggatgcttacccggagcagcgtgcaatggctgctttcgttttagcagttattgtggatgggcataggaggggtcaagaggcttgcatgagtgcaggtcttatagatgtttgcctgagacatctgcaacctgagaatcctcatgatgcacagacagagcctttgcttttgcaatggctttgtttatgccttggcaaactctgggaagatttccctgaggctcagttacttggtctacaatcaaatgcacccgaaattgttacatgtttattgtctgagcctcaacctgaggtcagagcctctgctgtttttgcacttggaaatctgttggacattggatctccatcagtgaatggaggtgatgacgactctgatgatgatgaaaaggcgagagctgaaataaatgttgttcgaagccttctgcaagtcacttcagatggtagccctcttgttagagccgaggtttctgtagctcttactcgctttgcgctgggccacaacaaacatctcaaatctgttgctgcggagtactggagacctcaaaccaattcactgctgaagtcactaccatcattggctaatattaataacccaagcaatgtttacagtcccagtaactttttgcaaggcagcagtggcctttcttctcatattggtcctgtgttaagggttggtagtgataccagtgccacaggtcgtgatggaagaatttctacaagcagcccgattgcgacaaatagcattatgcatgggtctccccagtcagatgattcttctcagcactctgattcaggcatattgctgagagataatgcaagtaacggtggtctcagctacaccaggtcaaggcctattattgacagtggaatttattccctatttatttcgacgatgtgctctattgctaaagatccttaccctagaattgcaaatattggccggagagcactgtcccttgtaggggttgagcaagtggtcatgagaaatactagatttggcagtgggggtgcaggagagtcatctgctccttcatcaaacataggaatggcacgttcttcttcctggtttgacatgaattctggaatctcaatggcatttaggacccctcctgttagtccacctcagcatgattaccttacagggttgcgccgcgtgtgttctatggagttcagaccacatctcttgaactcacctgatggcttagctgatccgctcttaagctctgctgcaggccccagtaccagtgagcttaatatacttccccaatcaacaatttacaactggagttgtggccacttctctaggcctcttctaaccgggtctgatgataatggggaagtaagtgctagaagagaagagagggaacgaactgcactggattgcatcgctaagtgccagcgttcctcagcatgcaaaatgactagccaaattgctagctgggatacaaagtttgagttgggtacaaaatcagcattgctgttacctttttctccaatcgtcgttgcagctgatgaaaacgagcaaataagagtgtggaattatgacgatgctctaccagtgaatactttcgagaaccacaagttgtctgacagaggactatccaaacttttacttatcaatgagcttgatgaaagcttgcttttagttggctcaagtatgccttccctctttatggctttctcttgtatctgtacatatgtattccttgtgacatctttttttcatactgtaggtgatggaaatgtccgtatatggagaaactatactcaaaagggaggacagaaacttgtaactgctttctcatcagttcagggccatcgagctgcaggccgcagcgttgtgattgactggcaacagcaatctggttatctgtatgcatctggtgacatgtcttccatccttgtatgggatcttgacaaggagcaacttctcaacaccattccatcatctgctgatagtggcatttcagctctgtctgcatctcaggttagatctggtcaatttgctgccggttttattgacgcatccgtaaggatatttgatgttcgtacccctgacaggctagtttatatggcaagaccacatgccccaagaactgaaaaggtcgtcggtataggatttcagcctggatttgatccatacaagattgtaagcgcatctcaagctggagacattcagtttcttgatgttagaagggctgctgaaccctacctcactattgaagctcacaggggttcattaacagcattggctgttcaccggcatgccccagtcattgcaagtggctcagctaagcaaatgattaaagtgttcagtcttgaaggagaacagctaacgatcattcggtaccagccatcttttatgggccaaaggataggaagtgtaaactgcctttcgttccatccgtacaaatctctcctggctgctggagctggtgataatgctctggtctctatctatgctgaggacaattatcaagtaagatgaggtttacatcctgtggcatgcaatgacgacacaatcagtaggccatgattggccgactgctggatccgcagcaggttcagctacaagatacaagatgatacaaagcctggtcagtgacatggtggtgattgcagtgctgtggctttttggtccatattgcttgtgttgggcatctacatctctggtcggcgtgtgcaatttgtaagatacaaatcaagattcattagcctcccttgcagaaggttgtgaaagtgaaaggcagttgtggtccagtcagtgaattcggaggcttccccaataatcatacctaggttaactgaaatgtaaatagccgtccccatttggattgttgtcctgtcagtcagttttcttcctttcccaaagtgtaaaaaggtttgccgatgcttcatatcataagtcggggtctggcggggtctgcccgggcaagtggttgtttatatgggccaggaagggagaatgcatgcacatgtctcctcacagatgttcttcaatagatagatgctcctttccagctaatatagcattagaggtgagttgttactatggcttgctttaaccagtgttgtccaggtcattctttctaagcattggactatcatcatccagtgttgtcgtcatagcagggcttcattgtccatttgtacatatgaactgcatttgtacagtagttaattctcttaacataagttgtaatgcatgtttctccttgggtgtatctggcatattctgaatatttaagtgaaaaagaagcttgtctctgtgta

SEQ ID NO: 41 - protein, 1364 aa, Triticum aestivum
MALGDLMASRLVHSSSSSSSSSLPTPSLAAVNHQTDRVDGELPVANGPELRREDAGEEDEEGKAVALVPCLPQVVVLCEQRHEGFDEAVAAAAGPSTSGLVSKWRPKDRMKTGCVALVLCLNISVDPPDVIKISPCARMECWIDPFSMPPPKALETIGKTLHSQYERWQPKARYKLQLDPTVEEVKKLCNTCRKYARSERVLFHYNGHGVPKPTANGEIWVFNKSYTQYIPLPITDLDSWLKTPSIYVFDCSAAGMIVKAFLERLDWSSSSSASSVKDCILLAACEAHQTLPQSAEYPADVFTACLTTPIKMALHWFCKRSLLSGALDHSLIDQIPGRQNDRKTLLGELNWIFTAITDTIAWNVLPHELFQRLFRQDLLVASLFRNFLLAERIMRSANCSPITYPLLPPTHQHHMWDAWDMAAEICLSKLPHLIADPNAEFQPSPFFTEQLTAFEVWLDHGSEDKKPPEQLPIVLQVLLSQSHRFRALVLLGRFLDMGPWAVDLALSVGIFPYVLKLLQTSAMELRQILVFIWTKILSLDKSCQVDLVKDGGHAYFIRFLDSLDAYPEQRAMAAFVLAVIVDGHRRGQEACMSAGLIDVCLRHLQPENPHDAQTEPLLLQWLCLCLGKLWEDFPEAQLLGLQSNAPEIVTCLLSEPQPEVRASAVFALGNLLDIGSPSVNGGDDDSDDDEKVRAEINVVRSLLQVTSDGSPLVRAEVSVALTRFALGHNKHLKSVAAEYWRPQTNSLLKSLPSLANINNPSNVYSPSNFLQSSSGLSSHIGPVLRVGSDTSATGRDGRISTSSPIATNSIMHGSPQSDDSSQHSDSGILLRENASNGGLSYTRSRPIIDSGIYSLFISTMCSVAKDPYPRIANIGRRALSLVGVEQVVMRNTRFGSGGAGETSAPSSNIGMARSSSWFDMNSGISMAFRTPPVSPPQHDYLTGLRRVCSMEFRPHLLNSPDGLADPLLSSAAAPSTSELNILPQSTIYNWSCGHFSRPLLTGSDDNGEVSARREERERTALDCIAKCQRSSACKMTSQIASWDTKFELGTKSALLLPFSPIVVAADENEQIRVWNYDDALPVNTFENHKLSDRGLSKLLLINELDESLLLVGSSDGNVRIWRNYTQKGGQKLVTAFSSVQGHRAAGRSVVIDWQQQSGYLYASGDMSSILVWDLDKEQLLNTIPSSADSGISALSASQVRPGQFAAGFIDASVRIFDVRTPDRLVYMARPHAPRTEKVVGIGFQPGFDPYKIVSASQAGDIQFLDVRRAAEPYLTIEAHRGSLTALAVHRHAPVIASGSAKQMIKVFSLEGEQLTIIRYQPSFMGQRIGSVNCLSFHPYKSLLAAGAGDNALVSIYAEDNYQVR

SEQ ID NO: 42 - DNA, 5167 bp, Triticum aestivum
  source 1..5167: mol_type = genomic DNA
ctctccgccgcatccccgtctcccccgaccctcacccccaccccgctgctcccgtccaaaccctaaccctaaaccccacccgcgcctccatccaatccctgcctggcgcccacccgtccccggcggcccggattcggattcgccgcgccggaccgattcgatcgatgtaggcagcgagcatcgctgcccactccgtacacaagacacgcgcccccgcctcgagatggctgctgcggggtgacaaggaggaggggagagtagtagagggagcgcccaagcgtagatggcattgggagatctcatggcgtccaggctcgtgcactcgtcctcatcgtcgtcctcgtcctcgctgcccactccctccttggcggcggtgaaccaccagacggaccgcgtggatggtgagctccctgtcgcgaacgggccggagcttcggagggaggacgccggcgaggaggatgaggaggggaaggccgttgcgctcgtgccgtgcctgccgcaggtggttgtgctgtgcgagcagcggcacgaggggttcgatgaggccgtcgctgccgcggcggggccatcgaccagcggcctcgtctccaagtggcgccccaaggacaggatgaagactggatgtgttgcacttgtattatgtttaaacattagtgttgatccgccggatgtaattaaaatttccccttgtgcaagaatggagtgttggatagatccattttctatgccacctcccaaagcccttgaaactattggaaaaacattacactcacagtatgagcggtggcagcctaaggctcgttacaagcttcagctggatccaacagtcgaagaagtcaagaagctttgtaatacttgccgcaaatatgccagatcagagagagttctttttcattacaatggtcatggtgtaccaaagcctacagctaatggagagatttgggtgtttaacaagagttatacacaatatatccctcttcctattactgatctcgattcatggctgaaaacaccatcgatttatgtttttgactgctcagcagctggaatgattgtgaaagcttttctagagcgcctagactggagttcaagctcttctgcgtcctcagtgaaggattgcattctccttgctgcctgtgaggcacatcaaactcttcctcagagtgcagaatatcccgctgatgtgtttacagcttgcctgaccactcccataaaaatggcattgcactggttttgtaaacgatcattactcagtggtgctctggatcactctcttattgaccaaattcctggaaggcaaaatgaccgtaaaacacttctgggggagttgaactggattttcactgctattacagatactattgcatggaatgttcttcctcatgaattattccaaaggcttttcaggcaggatcttttggtcgccagtctctttcgcaacttcttacttgctgagagaatcatgcgatctgcaaactgttctccaattacatacccactattgccacccacacatcaacaccatatgtgggatgcatgggacatggctgcggagatctgcctttccaagcttcctcatttgattgctgatcctaatgcagagtttcagccgagcccatttttcacggagcaattgacagcatttgaagtttggcttgatcatggctctgaggataagaaaccacctgaacagttacccattgttcttcaggtcctgcttagccaatcacacagatttagagcacttgttctgcttggaagatttcttgatatggggccatgggcagttgatttggccttgtctgttggtatcttcccttacgtgctcaaactgctccaaacaagtgcgatggagttgcgtcaaattcttgtgttcatatggacaaaaattctctctcttgataagtcatgccaggttgatttggtgaaagatggagggcatgcatattttatcaggtttcttgatagtttggatgcttacccggagcagcgtgcaatggctgctttcgttttagcagttattgtggatgggcataggaggggtcaagaggcttgcatgagtgcaggtcttatagatgtttgcctgagacatctgcaacctgagaatcctcatgatgcacagacagagcctttgcttttgcaatggctttgtttatgccttggcaaactctgggaagatttccctgaggctcagttacttggtctacaatcaaatgcacccgaaattgttacatgtttattgtctgagcctcaacctgaggtcagagcctctgctgtttttgcacttggaaatctgttggacattggatctccatcagtgaatggaggtgatgacgactctgatgatgatgaaaaggtgagagctgaaataaatgttgttcgaagccttctgcaagtcacttcagatggtagccctcttgttagagctgaggtttctgtagctcttactcgctttgcgctgggccacaacaaacatctcaaatctgttgctgcggagtactggagacctcaaaccaattcactgctgaagtcactaccatcattggctaatattaataacccaagcaatgtttacagtcccagtaactttctgcaaagcagcagtggcctttcttcgcatattggtcctgtgttaagggttggtagtgataccagtgccacaggtcgtgatggaagaatttctacaagcagcccgattgcgacaaatagcatcatgcatgggtctccccagtcagatgattcttctcagcactctgattcaggcatattgcttagagagaatgcaagtaacggtggtctcagctacaccaggtcaaggcctattattgacagtggaatttattccctatttatttcgacgatgtgctctgttgctaaagatccttaccctagaattgcaaatattggccggagagcactgtcccttgtaggggtcgagcaagtggtcatgagaaatactagatttggcagtgggggtgcaggagagacatctgctccttcatcaaacataggaatggcacgttcttcttcctggttcgacatgaattctggaatctcaatggcatttaggactcctcctgttagtccacctcagcatgattaccttacagggttgcgccgcgtgtgttctatggagttcagaccacatctcttgaattcacctgatggcttagctgatccgctcttaagctctgctgcagcccccagtaccagtgagcttaatatacttcctcaatcaacaatatacaactggagttgtggccacttctctaggccacttctaaccggttctgatgataatggggaagtaagtgctagaagagaagagagagaacgaactgcactggattgcatcgctaagtgccagcgttcctcagcatgcaagatgactagccaaattgctagctgggatacaaagtttgagttgggtacaaaatcagcattgctgttacctttttctcctatcgtcgttgcagctgatgaaaacgagcaaataagagtgtggaattatgacgatgctctaccagtgaatacttttgaaaaccacaagttatctgacagaggactatccaaacttctacttatcaatgagcttgatgaaagcttgcttttagttggctcaagtgatggaaatgtccgtatatggagaaactatactcaaaagggaggacagaaacttgtaactgccttctcatcagttcagggccatcgagctgcaggccgcagcgttgtgatcgactggcaacagcaatctggttatctgtatgcatctggtgacatgtcttccatcctcgtatgggatcttgacaaggagcaacttctcaacaccattccatcatctgctgatagtggcatttcagctctgtctgcatctcaggttagacctggtcaatttgctgccggttttattgatgcatccgtaaggatatttgatgttcgtacccctgacaggctagtttacatggcaagaccacatgccccaagaactgaaaaggtcgtcggtataggatttcagcctggatttgatccatacaagattgtaagcgcatctcaagctggagacattcagtttcttgatgttagaagggctgctgaaccctacctcactattgaagctcacaggggttcgttaacagcattggctgttcaccggcatgctccagtcattgcaagtggctcagctaagcagatgattaaagtgttcagtcttgaaggagaacagctaacaatcattcgctaccagccatcttttatgggccaaaggataggaagtgtaaactgcctttcgttccatccgtacaaatctctcctggctgctggagctggtgataatgctctggtctctatctatgctgaggacaattatcaagtaagatgaggtctacatcctgtggcatgcaatgacgacacaatcagtaggccatatgattggctgactgctggatccgcagcaggttcagctacaagatacaagatgatacaaagcctggtcagtgacatggtggtgattgcagtgctgtggctttttggtccatattgcttgtgttgggcatctacatctctggtcggcgagtgcaatttgtaagatacaaatcaagattcattagcctcccttgcagaaggttgtgaaagtgaaaggcagttgtggtcctgtcagtgaatttggaggcttccccaataatcatacctaggttaactgaaatgtaaatagccgtccccatttggattgttgtcctgtcagtcagttttcttcctttcccaaagtgtaaaaaggtttgtcgatgcttcatcatatcataagtcgggtctggcggggtctgcccgggcaagtggttgtttatatgggccaggaagggagaatgcatgcacatgtctcctcacagatgttcttcaatagatagatgctcctttccagctaatatagcattagaggtgagttgttactatggcttgctttaaccagtgttgtccaggtcattctttctaagcattggactatcatcatccagtgttgtcgtcatagcagggcttcattgtccatttgtacatatgaactgcatttgtacagtagttaattctcttaagataagttgtaatgcatgtttgtccttgggtgtatctggcatattttgaataattaagtgaaaaagaagcttgtctctgtgta

SEQ ID NO: 43 - protein, 1390 aa, Triticum aestivum
MALGDLMASRLVHSSSSSSSSSLPTPSSAAVNHQTDRVDGELPVANGPELRREDAGEEDEEGRAVVLVPCLPQVVVLCEQRHEGFDEAAAAVAGPSTSGLVSKWRPKDRMKTGCVALVLCLNISVDPPDVIKISPCARMECWIDPFSMPPPKALESIGKTLHSQYERWQPKARYKLQLDPTVEEVKKLCNTCRKYARSERVLFHYNGHGVPKPTANGEIWVFNKSYTQYIPLPITDLDSWLKTPSIYVFDCSAAGMIVKAFLERLDWSSSSSASSVKDCILLAACEAHQTLPQSAEYPADVFTACLTTPIKMALHWFCKRSLLSGSLDHSLIDQIPGRQNDRKTLLGELNWIFTAITDTIAWNVLPHELFQRLFRQDLLVASLFRNFLLAERIMRSANCSPITYPLLPPTHQHHMWDAWDMAAEICLSKLPHLIADPNAEFQPSPFFTEQLTAFEVWLDHGSEDKKPPEQLPIVLQVLLSQSHRFRALVLLGRFLDMGPWAVDLALSVGIFPYVLKLLQTSAMELRQILVFIWTKILSLDKSCQVDLVKDGGHAYFIRFLDSLDAYPEQRAMAAFVLAVIVDGHRRGQEACMSAGLIDVCLRHLQPENPHDAQTEPLLLQWLCLCLGKLWEDFPEAQLLGLQSNAPEIVTCLLSEPQPEVRASAVFALGNLLDIGSPSVNGGDDDSDDDEKVRAEINVVRSLLQVTSDGSPLVRAEVSVALTRFALGHNKHLKSVAAEYWRPQTNSLLKSLPSLANINNPSNVYSPSNFLQGSSGLSSHIGPVLRVGSDTSAAGRDGRISTSSPIATNSIMHGSPQSDDSSQHSDSGILLRDNASNGGLSYTRSRPIIDSGIYSLFISTMCSIAKDPYPRIANIGRRALSLVGVEQVVMRNTRFGSGGAGETSAPSSNIGMARSSSWFDMNSGISMAFRTPPVSPPQHDYLTGLRRVCSMEFRPHLLNSPDGLADPLLSSAAAPSTSELNILPQSTIYNWSCGHFSRPLLTGSDDNGEVTARREERERTALDCIAKCQRSSACKMTSQIASWDTKFELGTKSALLLPFSPIVVAADENEQIRVWNYDDALPVNTFENHKLSDRGLSKLLLINELDESLLLVGSSTPCLFMAVSCICTYVLLVISIFHTVGDGNVRIWRNYAQKGGQKLVTAFSSVQGHRAAGRSVVIDWQQQSGYLYASGDMSSILVWDLDKEQLLNTIPSSADSGISALSASQVRPGQFAAGFIDASVRIFDVRTPDRLVYMARPHAPRTEKVVGIGFQPGFDPYKIVSASQAGDIQFLDVRRAAEPYLTIEAHRGSLTALAVHRHAPVIASGSAKQMIKVFSLEGEQLTIIRYQPSFMGQRIGSVNCLSFHPYKSLLAAGAGDNALVSIYAEDNYQVR

SEQ ID NO: 44 - DNA, 5246 bp, Triticum aestivum
  source 1..5246: mol_type = genomic DNA
ctccccaccctctccgccgcatccccgtctcccccgaccctcacccccaccccgctgctcccgtccaaaccctaaccctaaaccccacccgcgcctccatccaatccctgcctggcgtccacccgtccccggcggcccggattcggattcgccgcgccggaccgattcgatcgatgtaggcagcgagcatcgctgcccactccgtacacaggactcgcgcccccgcctcgagatggctgctgcggggtgacgaggaggaggggagagtagtagagggagcgcccaagcgtagatggcattgggagatctcatggcgtccaggctcgtgcactcgtcctcatcgtcgtcctcgtcctcgttgcccaccccctcctcggcggcggtgaaccaccagacggaccgcgtggatggtgagctccctgtcgcgaacgggccggagcttcggagggaggacgccggcgaggaggatgaggaggggagggccgtggtgctagtgccgtgcctgccgcaggtggttgtgctgtgcgagcagcggcacgaggggttcgatgaggccgccgctgccgtggccgggccatcgaccagcgggctcgtctccaagtggcgccccaaggacaggatgaagaccggatgtgttgcacttgtattatgtttaaacattagtgttgatccgccggatgtaattaaaatttcgccttgtgcaagaatggagtgttggatagatccattttctatgccacctcccaaagcccttgaaagtattggaaaaacattacactcacagtatgagcgatggcagcctaaggctcgttacaagcttcagctggatccaacagtcgaagaagtcaagaagctttgtaatacttgccgcaaatatgccagatcagagagggttctttttcattacaatggtcatggtgtaccaaagcctacagctaatggagagatttgggtgtttaacaagagttatacacagtatatccctcttcctattactgatctcgattcatggctgaaaacaccatcgatttatgtttttgactgctcagcagctggaatgatcgtgaaagcttttctagagcgtctagactggagttcaagctcttctgcgtcttcagtgaaggattgcattctccttgctgcctgcgaggcacatcaaactcttcctcagagtgcagaatatcctgctgatgtgtttacagcttgcctgaccactcccataaaaatggcattgcactggttttgcaaacgatcattactcagtggttctctggatcactctcttattgaccaaattcctggaagacaaaatgaccgtaaaacacttctgggggagttgaactggattttcactgctattacagatactattgcatggaatgttcttcctcatgaattattccaaaggcttttcaggcaggatcttttggtcgccagtctctttcgcaacttcttacttgctgagagaatcatgcggtctgcaaactgctctccaattacgtacccactattgccacccacacatcaacatcatatgtgggatgcatgggacatggctgcggagatctgcctttccaagcttcctcatttgattgctgatcctaatgcagagtttcagccgagcccatttttcacggaacaattgacagcatttgaagtgtggcttgatcatggctctgaggataagaaaccccctgaacagttacccattgttcttcaggtcctgcttagccaatcacacagatttagagcacttgttctgcttggaagatttcttgatatggggccatgggcagttgatttggccttatctgttggtatcttcccttacgtgctcaaactgctccaaacaagtgcgatggagttgcgtcaaattcttgtgttcatatggacaaaaattctctctcttgataagtcatgccaggttgatttggtgaaagatggagggcatgcatattttatcaggtttcttgatagtttggatgcttacccggagcagcgtgcaatggctgctttcgttttagcagttattgtggatgggcataggaggggtcaagaggcttgcatgagtgcaggtcttatagatgtttgcctgagacatctgcaacctgagaatcctcatgatgcacagacagagcctttgcttttgcaatggctttgtttatgccttggcaaactctgggaagatttccctgaggctcagttacttggtctacaatcaaatgcacccgaaattgttacatgtttattgtctgagcctcaacctgaggtcagagcctctgctgtttttgcacttggaaatctgttggacattggatctccatcagtgaatggaggtgatgacgactctgatgatgatgaaaaggtgagagctgaaataaatgttgttcgaagccttctgcaagtcacttcagatggtagccctcttgttagagctgaggtttctgtagctcttactcgctttgcgctgggccacaacaaacatctcaaatctgttgctgcggagtactggagacctcaaaccaattcactgctgaagtcactaccatcattggctaatattaataacccaagcaatgtttacagtcccagtaactttttgcaaggcagcagtggcctttcttctcatattggtcctgtgttaagggttggtagtgataccagtgccgcaggtcgtgatggaagaatttctacaagcagcccgattgcgacaaatagcattatgcatgggtctccccagtcagatgattcttctcagcactctgattcaggcatattgctgagagataatgcaagtaacggtggtctcagctacacaaggtcaaggcctattattgacagtggaatttattccctatttatttcgacgatgtgctctattgctaaagatccttaccctagaattgcaaatattggccggagagcactgtcccttgtaggggtcgagcaagtggtcatgagaaatactagatttggcagtgggggtgcaggagagacatctgctccttcatcaaacataggaatggcacgttcttcttcctggtttgacatgaattctggaatctcaatggcatttaggacccctcctgttagtccacctcagcatgattaccttacagggttgcgccgcgtgtgttctatggagttcagaccacatctcttgaactcacctgatggcttagctgatccgctcttaagctctgctgcagcccccagtaccagtgagcttaatatacttccccaatcaacaatttacaactggagttgtggccacttctctaggcctcttctaaccgggtctgatgataatggggaagtaactgctagaagagaagagagggaacgaactgcactggattgcatcgctaagtgccagcgttcctcagcatgcaagatgactagccaaattgctagctgggatacaaagtttgagttgggtacaaaatcagcattgctgttacctttttctccaatcgtcgttgcagctgatgaaaacgagcaaataagagtgtggaattatgacgatgctctaccagtgaatactttcgaaaaccacaagttatctgacagaggactatccaaacttttacttatcaatgagcttgatgaaagcttgcttttagttggctcaagtacgccttgcctctttatggctgtctcttgtatctgtacatatgtactccttgtgatatctatttttcatactgtaggtgatggaaatgtccgtatatggagaaactatgctcaaaagggaggacagaaacttgtaactgctttctcatcagttcagggccatcgagctgcaggccgcagcgttgtgattgactggcaacagcaatctggttatctgtatgcatctggtgacatgtcttccatccttgtatgggatcttgacaaggagcaacttctcaacaccattccatcatctgctgatagtggcatttcagctctgtctgcatctcaggttagacctggtcaatttgctgccggttttattgacgcatccgtaaggatatttgatgttcgcacccctgacaggctagtttatatggcaagaccacatgccccaagaactgaaaaggtcgtcggtatagggtttcagcctggatttgatccatacaagattgtaagcgcatctcaagctggagacattcagtttcttgatgttagaagggctgctgaaccctacctcactattgaagctcacaggggttcattaacagcactggctgttcaccggcatgccccagtcattgcaagtggctcagctaagcaaatgattaaagtgttcagtcttgaaggagaacagctaacgatcattcggtaccagccatcttttatgggccaaaggataggaagtgtaaactgcctttcgttccatccgtacaaatctctcctggctgctggagctggtgataatgctctggtctctatctatgctgaggacaattatcaagtaagatgaggtttacatcctgtggcatgcaatgacgacacaatcagtaggccatatgattggccgactgctggatccgcagcaggttcagctacaagatacaagatgatacaaagcctggtcagtgacatggtggtgattgcagtgctgtggctttttggtccatattgcttgtgttgggcatctacatctctggtcggcatgtgcaatttgtaagatacaaatcaagattcattagcctcccttgcagaaggttgtgaaagtgaaaggcagttgtggtccagtcagtgaatccggaggcttccccaataatcatacctaggttaactgaaatgtaaatagccgtccccatttggattgttgtcctgtcagtcagttttcttcctttcccaaagtgtaaaaaggtttgccgatgcttcatatcataagtcgggtctggcggggtctgcaagtggttgtttatatgggccaggaagggagaatgcatgcacatgtctcctcacagatgttcttcaatagatagatgctcctttccagctaatatagcatttagaggtgagttgttactatggcttgctttaaccagtgttgtccaggtcattctttgtaagcattggactatcatcatccagtgttgtcgtcatagcagggcttcattgtccatttgtacatatgaactgcatttgtacagtagttaattctcttaagataagttgtaatgcatgtttgtccttgggtgtatctggcatattctgaatatttaagtgaaaaagaagcttgtctctgtgta

(19) United States
(12) Patent Application Publication
(10) Pub. No.: US 2025/0215444 A1
(43) Pub. Date: Jul. 3, 2025

(54) PLANTS WITH IMPROVED PHOSPHORUS USE EFFICIENCY

(71) Applicants: BOARD OF TRUSTEES OF MICHIGAN STATE UNIVERSITY, East Lansing, MI (US); CARNEGIE INSTITUTION OF WASHINGTON, Washington, DC (US)

(72) Inventors: Hatem Rouached, East Lansing, MI (US); Seung Yon Rhee, Stanford, CA (US)

(21) Appl. No.: 18/850,364

(22) PCT Filed: Mar. 30, 2023
(86) PCT No.: PCT/US2023/065159

§ 371 Date: Sep. 24, 2024

Related U.S. Application Data
(60) Provisional application No. 63/362,155, filed on Mar. 30, 2022.

(51) Int. Cl. C12N 15/82; C12N 9/12

(52) U.S. Cl. CPC C12N 15/8261; C12N 9/1205; C12N 15/8227

(57) ABSTRACT

Plants having modified expression or activity of a protein kinase polypeptide are provided. Methods for modifying expression or activity of the protein kinase polypeptide include genome editing to modify the transcription regulatory region or sequence encoding the protein kinase polypeptide as well as transformation with a heterologous polynucleotide encoding the protein kinase polypeptide. Plants containing the modifications have improved phosphorus use efficiency, increased tolerance to phosphorus deficiency, and improved yield in phosphorus deficient conditions.

### CROSS REFERENCE TO RELATED APPLICATIONS

This application claims priority to provisional patent application U.S. Ser. No. 63/362,155, filed Mar. 30, 2022. The provisional patent application is herein incorporated by reference in its entirety, including without limitation, the specification, claims, and abstract, as well as any figures, tables, appendices, or drawings thereof.

### GRANT REFERENCE

This invention was made with government support under grant nos. IOS-1546838 and IOS-1026003 awarded by the National Science Foundation, and grant nos. DE-SC0018277, DE-SC0008769, and DE-SC0020366 awarded by the U.S. Department of Energy. The US government has certain rights in the invention.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Mar. 27, 2023, is named “P13702WO00_SequenceListing.xml” and is 139,874 bytes in size.

### TECHNICAL FIELD

The present disclosure generally relates to the field of biotechnology. More specifically, the present disclosure relates to improving phosphorus use efficiency of plants.

### BACKGROUND

Phosphorus is an essential nutrient for plant growth and development as a key component of energy sources (ATP, ADP), nucleic acids, and membranes (phospholipids). Plants acquire phosphorus from soil through their roots. Due to the inefficient rates of phosphorus uptake (approximately 10-20% of applied phosphorus), current strategies to produce food rely heavily on the intensive use of phosphorus-based fertilizers. Phosphate fertilization is a significant expense to farmers that has been exacerbated by phosphorus reserve shortages and supply chain disruption. To meet the needs of food production while limiting the use of phosphorus-based fertilizers, it is important to develop a better understanding of how plants regulate phosphorus homeostasis to generate crops with improved phosphorus use efficiency (PUE). Worldwide phosphorus reserves are becoming increasingly scarce, and a potential crisis looms for agriculture. Thus, reducing the need for phosphate fertilization would have salutary benefits both environmentally and for farm profitability.

### SUMMARY

Provided are plants, seeds, plant parts, and plant cells that have a genomic modification that increases expression or activity of a protein kinase polypeptide. The genomic modification can be a deletion, insertion, or substitution of nucleotides in a genomic sequence encoding a protein kinase having at least 80%, at least 90%, at least 95%, at least 98%, or at least 99% sequence identity to at least one of the amino acid sequences set forth in SEQ ID NOs: 1, 3, 5, 7, 9, 11, 13, 15, 17, 19, or 21, wherein the modification increases expression or activity of the protein kinase polypeptide, such that the plant has improved phosphorus use efficiency relative to that of a control plant not comprising the modification. In certain embodiments, the modification comprises a deletion, an insertion, or a substitution of one or more nucleotides in a transcription regulatory region of the genomic sequence. Also provided are plants which comprise a heterologous polynucleotide encoding a protein kinase having at least 80%, at least 90%, at least 95%, at least 98%, or at least 99% sequence identity to at least one of the amino acid sequences set forth in SEQ ID NOs: 1, 3, 5, 7, 9, 11, 13, 15, 17, 19, or 21.

Methods for increasing phosphorus use efficiency or tolerance to phosphorus deficiency in a plant are provided. In certain embodiments, the methods comprise introducing a modification in at least one genomic sequence encoding a protein kinase into at least one plant cell, wherein the protein kinase has at least 80%, at least 90%, at least 95%, at least 98%, or at least 99% sequence identity to at least one of the amino acid sequences set forth in SEQ ID NOs: 1, 3, 5, 7, 9, 11, 13, 15, 17, 19, or 21, and wherein the modification results in increased expression or activity of the protein kinase. In certain embodiments, the methods comprise introducing a heterologous polynucleotide encoding a protein kinase into at least one plant cell, wherein the protein kinase has at least 80%, at least 90%, at least 95%, at least 98%, or at least 99% sequence identity to at least one of the amino acid sequences set forth in SEQ ID NOs: 1, 3, 5, 7, 9, 11, 13, 15, 17, 19, or 21.

Methods for limiting use of phosphorus-based fertilizer in agricultural crop production are provided. The methods comprise planting a seedling, cutting, tuber, or seed of any of the plants disclosed herein and growing the seedling, cutting, tuber, or seed under conditions favorable for the growth and development of a plant resulting therefrom.

Methods of plant breeding are provided in which the plants disclosed herein are crossed with a second plant to produce progeny seed. The progeny seed produced may comprise the modification or heterologous polynucleotide and have improved phosphorus use efficiency.

Plants, and plant cells thereof, with increased phosphorus use efficiency or tolerance to phosphorus deficiency, wherein the plants comprise a polynucleotide encoding a Raptor polypeptide, and wherein the Raptor polypeptide comprises a serine to aspartic acid substitution at a position corresponding to position 740 of SEQ ID NO: 27 are provided. Also provided are methods of increasing phosphorus use efficiency or tolerance to phosphorus deficiency in a plant, the methods comprise expressing a polynucleotide encoding a Raptor polypeptide in at least one plant cell, wherein the Raptor polypeptide comprises a serine to aspartic acid substitution at a position corresponding to position 740 of SEQ ID NO: 27.

While multiple embodiments are disclosed, still other embodiments will become apparent to those skilled in the art from the following detailed description, which shows and describes illustrative embodiments. Accordingly, the figures and detailed description are to be regarded as illustrative in nature and not restrictive.

### BRIEF DESCRIPTION OF THE FIGURES

FIGS. 1A-1B show that early root growth inhibition by phosphorus (P) deficiency is not due to changes in iron (Fe) concentration. 1A: key Arabidopsis genes LPR1, PDR2, STOP1, and ALMT1 contribute to adjusting root growth during later stages of P deficiency (days of Pi deficiency stress), due to Fe overaccumulation in plant roots. 1B: 9 hour transfer of seedlings to P-deficient conditions Arabidopsis primary root growth is progressively inhibited, while Fe concentration (magenta line) in root remains unchanged. Therefore, the molecular mechanisms adjusting root growth to early Pi deficiency are Fe-independent. Δ Primary root length indicates the difference in root length under −P versus +P transfer treatments.

FIG. 2 shows predicted regulatory network of genes responsive to early stages of P deficiency. Mode of regulation between transcription factors (light gray circles) and target genes (dark gray circles) is indicated as activating and repressing links. Of the 10 annotated transcription factors, four (ERF37, ERF36, ERF6, and MYB49) target ARSK1.

FIGS. 3A-3F show that ARSK1 controls root growth in a P-dependent manner. 3A: Expression of the promoter-GUS fusion gene. The transgenic plants containing pARSK1::GUS gene grown on the surface of MS agar plates for 10 days and plants were stained with X-Gluc. 3B: Subcellular localization ARSK1 in tobacco leaves. 35Spro::RSK1-GFP was transiently expressed in N. benthamiana leaves via agroinfiltration. The infiltrated leaves were briefly co-stained with 0.2 mg/l DAPI for visualizing the nucleus and examined by confocal laser scanning microscopy. The scale bar indicates 50 μm. 3C: ARSK1 mRNA abundance relative to Ubiquitin 10 in the roots of Col-0 grown in the presence of +P for 7 days and transferred to +P or −P for 6 h. Data shown from 3 biological repeats. Error bars represent 95% confidence interval. Individual measurements were obtained from the analysis of roots collected from a pool of 4 plants. Student's t test, ***p<0.001. 3D: Relative mRNA abundance of the ARSK1 gene in roots of Col-0, arsk1-1, arsk1-2, ARSK1-OE1, and ARSK1-OE2. Data (means±95% confidence interval) from 3 independent plant cultures, each with a pool of 6 plants per genotype. Letters denote significant differences at p<0.05, one-way ANOVA with a Duncan post hoc test. 3E: Primary root length of Col-0, arsk1, lpr1, lpr2, and two independent lines overexpressing genomic ARSK1 (ARSK1-OE) after 3 hours of transfer to +P or −P treatments P<0.05. 3F: Change in primary root length of Col-0, arsk1, and ARSK1-OE lines after 5 days of transfer to −P. DAT: days of transfer. The letters represent statistically different means at p<0.05 (one-way ANOVA with a Duncan post hoc test).

FIGS. 4A-4I show regulation of ARSK1 expression via MYB49 and promoter methylation. 4A: Transactivation of ARSK1 promoter:GUS activity after coexpression with predicted transcription factor binding partners identified in our GRN analyses (FIG. 2), including MYB49 (AT5G54230). 4B: Y1H assay of MYB49, ERF37, ERF36, and ERF6 binding to the ARSK1 promoter. 4C: EMSA of MYB49 binding to an ARSK1 promoter probe. 4D: MYB49 mRNA abundance relative to Ubiquitin 10 in the roots of WT grown in the presence of +P for 7 days and transferred to +P or −P for 6 h. Data shown from 3 biological experiments. P<0.05. 4E: Correlation of MYB49 and ARSK1 expression across treatments, and developmental stages, as well as natural Arabidopsis accessions. 4F: Patterns of ARSK1 gene body (Gbm) and promoter (Prom-Meth) methylation in selected Arabidopsis accessions. The MYB49 binding motif is indicated with the site of methylation under −P indicated in red. 4G: ARSK1 expression associated with differential promoter methylation in various Arabidopsis accessions with ARSK1-A and ARSK1-C alleles, respectively. 4H: Methylation profile in the chromosome 2, 11196502 to 11196862 of Col in response to 9 h of P deficiency. 4I: Transactivation of native and mutated (C->A) ARSK1 promoter:GUS activity after coexpression with MYB49 transcription factor.

FIGS. 5A-5E show RAPTOR1B is a target of ARSK1. 5A: ARSK1 and RAPTOR1B interact in yeast as revealed by yeast-two hybrid (Y2H) assays. Yeast strains were plated on nonselective (NS) or on selective (S) medium. 5B: ARSK1 phosphorylates RAPTOR1B in vitro. ARSK1, RAPTOR1B, and LST8 were incubated with ATP-P32, separated by SDS-PAGE. Radioactivity was detected by autoradiography on a photographic film. The results indicate that ARSK1 is an active kinase that autophosphorylates (lane 1). In the presence of ARSK1, the RAPTOR1B phosphosignal (lanes 2 and 3, rectangle) is higher compared to the control (lane 4; no ARSK1). 5C and 5D: Changes in primary root length after 5 days of transfer to −P treatment for WT, ARSK1-RAPTOR1B pathway mutants, and transgenic lines expressing RAPTOR1B with a mutated S at positions 739, 740, or 741 in raptor1b (5C) or with a mutated S at position 740 in arsk1-1; raptor1b (5D). In response to P deficiency, root growth is affected by a point mutation of phosphosites in RAPTOR1B. 5E: Average primary root length of Col-0, raptor1b, RAPTOR1B S740D and RAPTOR1B S740A plants grown in +P for 5 days and transferred on the same condition (+P) for 5 additional days. Data shown are from three biological repeats, each experiment with 10 plants. Error bars represent 95% confidence interval.

FIGS. 6A-6C show that P availability influences TOR activity. 6A and 6B: The effect of combinatorial P and AZD-8055 (TOR inhibitor) treatments on primary root growth in Col-0 (6A) and arsk1-1 and raptor1b mutants (6B). 6C: Immunological detection of S6K phosphorylation status in wild-type Col-0, ARSK1-OE1, and RAPTOR1BS⁷⁴⁰D after 9 hours of transfer to +P or −P conditions. Apparent sizes of S6K-p, S6K: 52 kDa. Seedlings were used for this analysis. Coomassie is used as loading control in Western blot analysis.

FIG. 7 is a schematic model delineating a signaling pathway that integrates P availability cues to regulate root growth via TORC1. Under P sufficiency environments, MYB49 promotes ARSK1 expression. P deficiency induces methylation of ARSK1 promoter to repress its expression, likely by preventing accessibility to MYB49. In turn, ARSK1 protein may phosphorylate RAPTOR1B, and the phosphorylated RAPTOR1-B may promote TOR activity to maintain root growth. In the absence of ARSK1, RAPTOR1B is not phosphorylated, and TOR activity is altered, leading to the inhibition of root growth. These findings uncover a novel pathway which integrates phosphate availability and energy sensing TOR pathway.

### DETAILED DESCRIPTION

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

The term “cis-element” generally refers to transcriptional regulatory element that affects or modulates expression of an operably linked transcribable polynucleotide, where the transcribable polynucleotide is present in the same DNA sequence. A cis-element may function to bind transcription factors, which are trans-acting polypeptides that regulate transcription.

As used herein, the term “heterologous” refers to a polynucleotide that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

As used herein, the term “introducing” is intended to mean presenting to the plant a polynucleotide in such a manner that the polynucleotide gains access to the interior of a cell of the plant. The methods of the disclosure do not depend on a particular method for introducing a polynucleotide to a plant, only that the polynucleotide gains access to the interior of at least one cell of the plant.

As used herein, the term “operably linked” is intended to mean a functional linkage between two or more elements. For example, an operable linkage between a polynucleotide or gene of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame.

As used herein, the terms “orthologous” or “ortholog” are used to describe genes or proteins encoded by those genes that are from different species but which have the same function (e.g., encode enzymes that catalyze the same reactions). Orthologous genes will typically encode proteins with some degree of sequence identity (e.g., at least 40%, 50%, 60%, 70%, 80%, 90%, or 95% sequence identity, conservation of sequence motifs, and/or conservation of structural features).

“Plant” as used herein refers to whole plants, plant organs (e.g., leaves, stems, roots, etc.), seeds, plant cells, propagules, embryos, and progeny of the same. Plant cells can be differentiated or undifferentiated (e.g. callus, suspension culture cells, protoplasts, leaf cells, root cells, phloem cells and pollen).

The terms “polynucleotide” and “nucleic acid molecule” are used interchangeably herein. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide. Furthermore, it is understood by those of ordinary skill in the art that the nucleotide sequences disclosed herein also encompasses the complement of that exemplified nucleotide sequence.

“Promoter” generally refers to a nucleic acid fragment capable of controlling transcription of another nucleic acid fragment. A promoter generally includes a core promoter (also known as minimal promoter) sequence that includes a minimal regulatory region to initiate transcription, that is a transcription start site. Generally, a core promoter includes a TATA box and a GC rich region associated with a CAAT box or a CCAAT box. These elements act to bind RNA polymerase II to the promoter and assist the polymerase in locating the RNA initiation site. Some promoters may not have a TATA box or CAAT box or a CCAAT box, but instead may contain an initiator element for the transcription initiation site. A core promoter is a minimal sequence required to direct transcription initiation and generally may not include enhancers or other UTRs. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Core promoters are often modified to produce artificial, chimeric, or hybrid promoters, and can further be used in combination with other regulatory elements, such as cis-elements, 5′UTRs, enhancers, or introns, that are either heterologous to an active core promoter or combined with its own partial or complete regulatory elements.

A “transcription regulatory element” or a “regulatory element” generally refers to a transcriptional regulatory element involved in regulating the transcription of a nucleic acid molecule such as a gene or a target gene. The regulatory element is a nucleic acid and may include a promoter, an enhancer, an intron, a 5′-untranslated region (5′-UTR, also known as a leader sequence), or a 3′-UTR or a combination thereof. A regulatory element may act in “cis” or “trans”, and generally it acts in “cis”, i.e. it activates expression of genes located on the same nucleic acid molecule, e.g. a chromosome, where the regulatory element is located.

As used herein, the term “stable transformation” is intended that a polynucleotide introduced into a plant integrates into the genome of the plant and is capable of being inherited by progeny thereof. As used herein, the term “transient transformation” is intended that a polynucleotide introduced into a plant does not integrate into the genome of the plant.

As used herein, the terms “transgenic plant” and “transformed plant” are equivalent terms that refer to a “plant” as described above, wherein the plant comprises a heterologous polynucleotide that is introduced into a plant by, for example, any of the stable and transient transformation methods disclosed elsewhere herein or otherwise known in the art. Such transgenic plants and transformed plants also refer, for example, the plant into which the heterologous polynucleotide was first introduced and also any of its progeny plants that comprise the heterologous polynucleotide.

As defined herein, the “yield” of the plant refers to the quality and/or quantity of biomass produced by the plant. “Biomass” as used herein refers to any measured plant product. An increase in biomass production is any improvement in the yield of the measured plant product. Increasing plant yield has several commercial applications. An increase in yield can comprise any statistically significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase in yield.

Plants take up phosphorus in the form of inorganic phosphate (Pi) from the soil through the root system. Once inside the cell, Pi serves as a substrate donor for important biological molecules and plays key roles in a variety of processes, such as synthesis of nucleic acids, storage and transfer of energy (as ATP), regulation of cell metabolism and signaling (via protein phosphorylation), and maintenance of cell membrane integrity (as phospholipids). Although Pi is required for numerous biological processes, how cells integrate Pi-derived signals to control growth previously remained largely unknown.

Compositions and methods related to plants having improved phosphorus use efficiency are provided. Plants that have been modified using genomic editing techniques, transformation, or mutagenesis to have the increased phosphorus use efficiency are provided. Modifying expression or activity of a protein kinase polypeptide in a plant or modifying the transcription regulatory region of the protein kinase polypeptide results in a plant having improved phosphorus use efficiency relative to a comparable plant not comprising the modification. The modification can be introduced using genomic editing technology, transformation, or mutagenesis, such as described herein.

Provided are plants, plant cells, plant parts, and seeds which have had expression of a protein kinase polypeptide or polynucleotide sequence that encodes the protein kinase polypeptide increased and/or in which the activity of the protein kinase polypeptide is increased. An example of the protein kinase polypeptide is the Arabidopsis Root-Specific Kinase 1 (ARSK1) polypeptide shown in SEQ TD NO: 1, encoded by SEQ ID NO: 2. Amino acid and nucleotide sequences of ARSK1 and orthologs in crop species are summarized in Table 1.

TABLE 1

| | Amino acid | |
|---|---|---|
| | sequence | cDNA sequence |
| Arabidopsis | SEQ ID NO: 1 | SEQ ID NO: 2 |
| (Arabidopsis thaliana) | | |
| Maize | SEQ ID NO: 3 | SEQ ID NO: 4 |
| (Zea mays) | | |
| Soybean | SEQ ID NO: 5 | SEQ ID NO: 6 |
| (Glycine max) | | |
| Rapeseed | SEQ ID NO: 7 | SEQ ID NO: 8 |
| (Brassica napus) | | |
| Cotton | SEQ ID NO: 9 | SEQ ID NO: 10 |
| (Gossypium hirsutum) | | |
| Sugarcane | SEQ ID NO: 11 | SEQ ID NO: 12 |
| (Saccharum sp.) | | |
| Rice | SEQ ID NO: 13 | SEQ ID NO: 14 |
| (Oryza sativa) | | |
| Potato | SEQ ID NO: 15 | SEQ ID NO: 16 |
| (Solanum tuberosum) | | |
| Wheat | SEQ ID NOs: 17, | SEQ ID NOs: 18, |
| (Triticum aestivum) | 19, 21 | 20, 22 |

In certain embodiments, the modification results in an increase in expression or activity of the protein kinase polypeptide. The genome is modified to increase expression or activity of the protein kinase, such as by modifying a sequence in the transcription regulatory region to include, for example, an enhancer element, an enhancer binding element or to disrupt a promotor repressor element or a methylation site within the transcription regulatory region. In certain embodiments, the plant, plant cell, plant part, or seed include a heterologous polynucleotide encoding a protein kinase polypeptide described herein. Transformation methods for producing such plants, plant cells, plant parts or seeds are provided.

Provided are polynucleotides that have at least about or at least 40%, 45%, 50%, 51%, 52%, 53%, 54%, 55%, 56%, 57%, 58%, 59%, 60%, 61%, 62%, 63%, 64%, 65%, 66%, 67%, 68%, 69% 70%, 71%, 72%, 73%, 74%, 75%, 76%, 77%, 78%, 79%, 80%, 81%, 82%, 83%, 84%, 85%, 86% 87%, 88%, 89%, 90%, 91%, 92%, 93%, 94%, 95%, 96%, 97%, 98%, 99% or greater sequence identity to at least one of the nucleotide sequences set forth in SEQ TD NO: 2, 4, 6, 8, 10, 12, 14 or 16, using one of the alignment programs described herein using standard parameters, as well as nucleotide substitutions, deletions, insertions, fragments thereof, variants thereof, and combinations thereof.

Provided are polypeptides having at least about or at least 40%, 45%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity to at least one of the amino acid sequences set forth in SEQ ID NOs: 1, 3, 5, 7, 9, 11, 13, 15, 17, 19, or 21, as well as amino acid substitutions, deletions, insertions, fragments thereof, variants thereof, and combinations thereof.

Fragments and variants of the disclosed polynucleotides and proteins encoded thereby are also encompassed by the present disclosure. By “fragment” is intended a portion of the polynucleotide or a portion of the amino acid sequence and hence protein encoded thereby. Fragments of polynucleotides comprising coding sequences may encode protein fragments that retain biological activity of the full-length or native protein. Alternatively, fragments of a polynucleotide that are useful as hybridization probes generally do not encode proteins that retain biological activity or do not retain promoter activity. Thus, fragments of a nucleotide sequence may range from at least about 20 nucleotides, about 50 nucleotides, about 100 nucleotides, and up to the full-length polynucleotide of the disclosure. Polynucleotides that are fragments of a protein kinase polynucleotide comprise at least 16, 20, 50, 75, 100, 125, 150, 175, 200, 250, 300, 350, 400, 450, 500, 600, 700, 800, 900, 1000, 1500, 2000, 3000, 4000, 5000, 6000, 7000, 8000, or 9000 contiguous nucleotides, or up to the number of nucleotides present in a full-length protein kinase polynucleotide disclosed herein.

“Variants” is intended to mean substantially similar sequences. For polynucleotides, a variant comprises a polynucleotide having deletions (i.e. truncations) at the 5′ and/or 3′ end; deletion and/or addition of one or more nucleotides at one or more internal sites in the native polynucleotide; and/or substitution of one or more nucleotides at one or more sites in the polynucleotide. For polynucleotides, conservative variants include those sequences that, because of the degeneracy of the genetic code, encode the amino acid sequence of one of the protein kinase polypeptides of the disclosure. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a protein kinase polypeptide of the disclosure. Generally, variants of a particular polynucleotide of the disclosure will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. In certain embodiments, variants of a particular polynucleotide of the disclosure will have at least about 75%, 80%, 85%, 90%, 91%, 92%, 93%, 94%, 95%, 96%, 97%, 98%, 99% or more sequence identity to at least one nucleotide sequence selected from SEQ ID NOs: 2, 4, 6, 8, 10, 12, 14, 16, 18, 20, and 22, and optionally comprise a non-naturally occurring nucleotide sequence that differs from the nucleotide sequence set forth in SEQ ID NOs: 2, 4, 6, 8, 10, 12, 14, 16, 18, 20, or 22 by at least one nucleotide modification, wherein the at least one nucleotide modification comprises the substitution of at least one nucleotide, the addition of at least one nucleotide, or the deletion of at least one nucleotide. It is understood that the addition of at least one nucleotide can be the addition of one or more nucleotides within a nucleotide sequence of the present disclosure (e.g. SEQ ID NOs: 2, 4, 6, 8, 10, 12, 14, 16, 18, 20, and 22), the addition of one or more nucleotides to the 5′ end of a nucleotide sequence of the present disclosure, and/or the addition of one or more nucleotides to the 3′ end of a nucleotide sequence of the present disclosure.

Variants of a particular polynucleotide of the disclosure (i.e. the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to at least one polypeptide having the amino acid sequence selected from SEQ ID NOs: 1, 3, 5, 7, 9, 11, 13, 15, 17, 19, and 21 is disclosed. Percent sequence identity between any two polypeptides can be calculated using sequence alignment programs and parameters described elsewhere herein. Where any given pair of polynucleotides of the disclosure is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity. In certain embodiments, variants of a particular polypeptide will have at least about 60%, 65%, 70%, 75%, 80%, 85%, 90%, 91%, 92%, 93%, 94%, 95%, 96%, 97%, 98%, 99% or more sequence identity to at least one of the amino acid sequences set forth in SEQ ID NOs: 1, 3, 5, 7, 9, 11, 13, 15, 17, 19, or 21, and optionally comprises a non-naturally occurring amino acid sequence that differs from at least one amino acid sequence selected from SEQ ID NOs: 1, 3, 5, 7, 9, 11, 13, 15, 17, 19, or 21 by at least one amino acid modification, wherein the at least one amino acid modification comprises the substitution of at least one amino acid, the addition of at least one amino acid, or the deletion of at least one amino acid. It is understood that the addition of at least one amino acid can be the addition of one or more amino acids within an amino acid sequence of the present disclosure (e.g., SEQ ID NOs: 1, 3, 5, 7, 9, 11, 13, 15, 17, 19, or 21), the addition of one or more amino acids to the N-terminal end of an amino acid sequence of the present disclosure, and/or the addition of one or more amino acids to the C-terminal end of an amino acid sequence of the present disclosure.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Such variants may result from, for example, genetic polymorphism or from human manipulation. A variant of a protein of the disclosure may differ from that protein by as few as 1-15 amino acid residues, as few as 1-10, such as 6-10, as few as 5, as few as 4, 3, 2, or even 1 amino acid residue.

The proteins of the disclosure may be altered in various ways including amino acid substitutions, deletions, truncations, and insertions. Methods for such manipulations are generally known in the art. Methods for mutagenesis and polynucleotide alterations are well known in the art. See, for example, Kunkel (1985) Proc. Natl. Acad. Sci. USA 82:488-492; Kunkel et al. (1987) Methods in Enzymollette. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington. D.C.), herein incorporated by reference. Conservative substitutions, such as exchanging one amino acid with another having similar properties, may be optimal.

Thus, the genes and polynucleotides of the disclosure include both the naturally occurring sequences as well as mutant and other variant forms. Likewise, the proteins of the disclosure encompass naturally occurring proteins as well as variations and modified forms thereof. In some embodiments, the mutations that will be made in the DNA encoding the variant will not place the sequence out of reading frame. Optimally, the mutations will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by assays that are disclosed herein below.

Variant polynucleotides and proteins also encompass sequences and proteins derived from a mutagenic and recombinogenic procedure such as DNA shuffling. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

To determine the percent identity of two amino acid sequences or of two nucleic acids, the sequences are aligned for optimal comparison purposes. The percent identity between the two sequences is a function of the number of identical positions shared by the sequences (i.e. percent identity=number of identical positions/total number of positions (e.g. overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to the polynucleotides of the disclosure. BLAST protein searches can be performed with the XBLAST program, score=50, wordlength=3, to obtain amino acid sequences homologous to polypeptides of the disclosure. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g. XBLAST and NBLAST) can be used. BLAST, Gapped BLAST, and PSI-Blast, XBLAST and NBLAST are available on the World Wide Web at ncbi.nlm.nih.gov. Another non-limiting example of a mathematical algorithm utilized for the comparison of sequences is the algorithm of Myers and Miller (1988) CABIOS 4: 11-17. Such an algorithm is incorporated into the ALIGN program (version 2.0), which is part of the GCG sequence alignment software package. When utilizing the ALIGN program for comparing amino acid sequences, a PAM 120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences and using multiple alignment by means of the algorithm ClustalW (Nucleic Acid Research, 22(22):4673-4680, 1994) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website on the World Wide Web at ebi.ac.uk/Tools/clustalw/index).

The protein kinase polynucleotides of the disclosure can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire sequences set forth herein or to variants and fragments thereof are encompassed by the present disclosure. Such sequences include sequences that are orthologs of the disclosed sequences. Genes found in different species are considered orthologs when their nucleotide sequences and/or their encoded amino acid sequences share at least 60%, 70%, 75%, 80%, 85%, 90%, 91%, 92%, 93%, 94%, 95%, 96%, 97%, 98%, 99%, or greater sequence identity. Functions of orthologs are often highly conserved among species.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989)Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e. genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the polynucleotides of the disclosure. Methods for preparation of probes for hybridization and for construction of cDNA and genomic libraries are generally known in the art and are disclosed in Sambrook et al. (1989)Molecular Cloning: A Laboratory Manual (2d ed., Cold Spring Harbor Laboratory Press, Plainview, New York).

For example, an entire polynucleotide disclosed herein, or one or more portions thereof, may be used as a probe capable of specifically hybridizing to corresponding polynucleotide and messenger RNAs. To achieve specific hybridization under a variety of conditions, such probes include sequences that are unique among the sequence of the gene or cDNA of interest sequences and are optimally at least about 10 nucleotides in length, and most optimally at least about 20 nucleotides in length. Such probes may be used to amplify corresponding polynucleotides for the particular gene of interest from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989)Molecular Cloning: A Laboratory Manual (2d ed., Cold Spring Harbor Laboratory Press, Plainview, New York). An extensive guide to the hybridization of nucleic acids is found in Tijssen (1993) Laboratory Techniques in Biochemistry and Molecular Biology Hybridization with Nucleic Acid Probes, Part 1, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

The polynucleotide encoding a protein kinase polypeptide can be provided in a polynucleotide construct (e.g., an expression cassette) for expression in the plant. The polynucleotide construct can include in the 5′-3′ direction of transcription, a transcriptional and translational initiation region (i.e., a promoter), a protein kinase coding region, and a transcriptional and translational termination region (i.e. termination region) functional in a plant. The regulatory regions (i.e. promoters, transcriptional regulatory regions, and translational termination regions) and/or the protein kinase coding region may be native/analogous to the cell or to each other. Alternatively, the regulatory regions and/or the protein kinase coding region may be heterologous to the cell or to each other.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The polynucleotide constructs may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); poty virus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the polynucleotide construct, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g. transitions and transversions, may be involved.

A number of promoters can be used. The choice of heterologous promoter can depend on a number of factors such as, for example, the desired timing, localization, and pattern of expression as well as responsiveness to particular biotic or abiotic stimulus. The promoters can be selected based on the desired outcome. The nucleic acids can be combined with constitutive, tissue-preferred, or other promoters for expression in plants. Such constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, those described in U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced expression of the protein kinase polypeptide within a particular plant tissue. Such tissue-preferred promoters include, but are not limited to, leaf-preferred promoters, root-preferred promoters, seed-preferred promoters, and stem-preferred promoters. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6): 1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991)Mol. Gen. Genet. 227:229-237, and U.S. Pat. Nos. 5,814,618 and 5,789,156), herein incorporated by reference.

While it may be optimal to express the protein kinase polypeptide using heterologous promoters, the native promoter of the corresponding protein kinase gene may be used.

Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) (ell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

The polynucleotide construct can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004). J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990)Mol. Cell. Biol. 10:3343-3356; Zambretti et al. (1992) Proc. Natl. Acad Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference. The above list of selectable marker genes is not intended to be limiting. Any selectable marker gene can be used.

Provided are plants, plant cells, plant seeds, and plant nuclei that are modified by gene editing. Any methods known in the art for modifying DNA in the genome of a plant can be used to modify genomic nucleotide sequences in planta. Such methods include, but are not limited to, genome-editing (or gene-editing) techniques, such as, for example, methods involving targeted mutagenesis, homologous recombination, and mutation breeding. In certain embodiments, genome editing may be facilitated through the induction of a double-stranded break (DSB) or single-strand break, in a defined position in the genome near the desired alteration. Targeted mutagenesis or similar techniques are disclosed in U.S. Pat. Nos. 5,565,350; 5,731,181; 5,756,325; 5,760,012; 5,795,972, 5,871,984, and 8,106,259; all of which are herein incorporated in their entirety by reference. Methods for modification or replacement of genomic sequences comprising homologous recombination can involve inducing double breaks in DNA using zinc-finger nucleases (ZFN), TAL (transcription activator-like) effector nucleases (TALEN), Clustered Regularly Interspaced Short Palindromic Repeats/CRISPR-associated nuclease (CRISPR/Cas nuclease), or homing endonucleases that have been engineered endonucleases to make double-strand breaks at specific recognition sequences in the genome of a plant, other organism, or host cell. See, for example, Durai et al., (2005) Nucleic Acids Res 33:5978-90; Mani et al. (2005) Biochem Biophys Res Comm 335:447-57; U.S. Pat. Nos. 7,163,824, 7,001,768, and 6,453,242; Amould et al. (2006) J Mol Biol 355:443-58; Ashworth et al., (2006) Nature 441:656-9; Doyon et al. (2006) J Am Chem Soc 128:2477-84; Rosen et al., (2006) Nucleic Acids Res 34:4791-800; and Smith et al., (2006) Nucleic Acids Res 34:e149; U.S. Pat. App. Pub. No. 2009/0133152; and U.S. Pat. App. Pub. No. 2007/0117128; all of which are herein incorporated in their entirety by reference.

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010)PNAS 10.1073/pnas. 1013133107; Scholze & Boch (2010) Virulence 1:428-432; Christian et al. Genetics (2010) 186:757-761; Li el al. (2010) Nuc. Acids Res. (2010) doi:10.1093/nar/gkg704; and Miller et al. (2011) Nature Biotechnology 29:143-148; all of which are herein incorporated by reference.

The CRISPR/Cas nuclease system can also be used to make double-strand breaks at specific recognition sequences in the genome of a plant for gene modification or gene replacement through homologous recombination. The CRISPR/Cas nuclease is an RNA-guided DNA endonuclease system performing sequence-specific double-stranded breaks in a DNA segment homologous to the designed RNA. It is possible to design the specificity of the sequence (Cho S. W. et al., Nat. Biotechnol. 31:230-232, 2013; Cong L. et al., Science 339:819-823, 2013; Mali P. et al., Science 339:823-826, 2013; Feng Z. et al., Cell Research: 1-4, 2013).

In addition, a ZFN can be used to make double-strand breaks at specific recognition sequences in the genome of a plant for gene modification or gene replacement through homologous recombination. The Zinc Finger Nuclease (ZFN) is a fusion protein comprising the part of the FokI restriction endonuclease protein responsible for DNA cleavage and a zinc finger protein which recognizes specific, designed genomic sequences and cleaves the double-stranded DNA at those sequences, thereby producing free DNA ends (Urnov F. D. et al., Nat Rev Genet. 11:636-46, 2010; Carroll D., Genetics. 188:773-82, 2011).

Breaking DNA using site specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

In certain embodiments, the introduction of a DSB can be combined with the introduction of a polynucleotide modification template. A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10: 957-963.) The polynucleotide modification template may be present transiently in the cell or it can be introduced via a viral replicon.

The methods of the disclosure involve introducing a polynucleotide or polynucleotide construct into a plant. Methods for introducing polynucleotides or polynucleotide constructs into plants are known in the art including, but not limited to, stable transformation methods, transient transformation methods, and virus-mediated methods.

Depending on the desired outcome, the polynucleotides of the disclosure can be stably incorporated into the genome of the plant cell or not stably incorporated into genome of the plant cell. If, for example, the desired outcome is to produce a stably transformed plant with improved phosphorus use efficiency, then the polynucleotide can be, for example, fused into a plant transformation vector suitable for the stable incorporation of the polynucleotide into the genome of the plant cell. Typically, the stably transformed plant cell will be regenerated into a transformed plant that comprises in its genome the polynucleotide. Such a stably transformed plant is capable of transmitting the polynucleotide to progeny plants in subsequent generations via sexual and/or asexual reproduction. Plant transformation vectors, methods for stably transforming plants with an introduced polynucleotide and methods for plant regeneration from transformed plant cells and tissues are generally known in the art for both monocotyledonous and dicotyledonous plants or described elsewhere herein.

In other embodiments in which it is not desired to stably incorporate the polynucleotide in the genome of the plant, transient transformation methods can be utilized to introduce the polynucleotide into one or more plant cells of a plant. Such transient transformation methods include, for example, viral-based methods which involve the use of viral particles or at least viral nucleic acids. Generally, such viral-based methods involve constructing a modified viral nucleic acid comprising a heterologous polynucleotide of the disclosure operably linked to the viral nucleic acid and then contacting the plant either with a modified virus comprising the modified viral nucleic acid or with the viral nucleic acid or with the modified viral nucleic acid itself. The modified virus and/or modified viral nucleic acids can be applied to the plant or part thereof, for example, in accordance with conventional methods used in agriculture, for example, by spraying, irrigation, dusting, or the like. The modified virus and/or modified viral nucleic acids can be applied in the form of directly sprayable solutions, powders, suspensions or dispersions, emulsions, oil dispersions, pastes, dustable products, materials for spreading, or granules, by means of spraying, atomizing, dusting, spreading or pouring. It is recognized that it may be desirable to prepare formulations comprising the modified virus and/or modified viral nucleic acids before applying to the plant or part or parts thereof. Methods for making pesticidal formulations are generally known in the art or described elsewhere herein.

Numerous plant transformation vectors and methods for transforming plants are available. See, for example, An, G. et al. (1986) Plant Physiol., 81:301-305; Fry, J., et al. (1987) Plant Cell Rep. 6:321-325; Block. M. (1988) Theor. Appl Genet. 76:767-774; Hinchee, et al. (1990) Stadler. Genet. Symp. 203212.203-212; Cousins, et al. (1991) Aust. J. Plant Physiol. 18:481-494; Chee, P. P. and Slightom, J. L. (1992) Gene. 118:255-260; Christou, et al. (1992) Trends. Biotechnol. 10:239-246; D'Halluin, et al. (1992) Bio Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.-Plant; 29P:119-124; Davies, et al. (1993) Plant Cell Rep. 12:180-183; Dong, J. A. and Mchughen, A. (1993) Plant Sci. 91:139-148; Franklin. C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

For the transformation of plants and plant cells, the nucleotide sequences of the disclosure can be inserted using standard techniques into any vector known in the art that is suitable for expression of the nucleotide sequences in a plant or plant cell. The selection of the vector depends on the transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991)Mol. Gen. Genet., 228: 104-112; Guerche et al., (1987) Plant Science 52: 111-116; Neuhause et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Sci. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Led transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the disclosure may be introduced into plants by contacting plants with a virus or viral nucleic acids. Generally, such methods involve incorporating a polynucleotide or polynucleotide construct of the disclosure within a viral DNA or RNA molecule. Further, it is recognized that promoters of the disclosure also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

If desired, the modified viruses or modified viral nucleic acids can be prepared in formulations. Such formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48. Perry's Chemical Engineer's Handbook, 4th Ed., McGraw-Hill, New York, 1963, pages 8-57 and et seq. WO 91/13546, U.S. Pat. Nos. 4,172,714, 4,144,050, 3,920,442, 5,180,587, 5,232,701, 5,208,030, GB 2,095,558, U.S. Pat. No. 3,299,566, Klingman, Weed Control as a Science, John Wiley and Sons, Inc., New York, 1961, Hance et al. Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

In certain embodiments, the polynucleotides of the disclosure can be provided to a plant using a variety of transient transformation methods known in the art. Such methods include, for example, microinjection or particle bombardment. See, for example, Crossway et al. (1986)Mol Gen. Genet. 202:179-185; Nomura et al. (1986) Plant Sci. 44:53-58; Hepler et al. (1994) PNAS Sci. 91: 2176-2180 and Hush et al. (1994) J. Cell Science 107:775-784, all of which are herein incorporated by reference. Alternatively, the polynucleotide can be transiently transformed into the plant using techniques known in the art. Such techniques include viral vector system and Agrobacterium tumefaciens-mediated transient expression as described elsewhere herein.

The cells that have been transformed may be grown into plants in accordance with conventional ways. See, for example. McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide of the disclosure stably incorporated into their genome.

Unless expressly stated or apparent from the context of usage, the methods and compositions of the present disclosure can be used with any plant species including, for example, monocotyledonous plants and dicotyledonous plants. Examples of plant species of interest include, but are not limited to, corn (Zea mays), Brassica sp. (e.g., B. napus, B. rapa, B. juncea), alfalfa (Medicago sativa), rice (Oryza sativa), rye (Secale cereale), triticale (xTriticosecale or TriticumxSecale) sorghum (Sorghum bicolor, Sorghum vulgare), teff (Eragrostis tef), millet (e.g. pearl millet (Pennisetum glaucum), proso millet (Panicum miliaceum), foxtail millet (Setaria italica), finger millet (Eleusine coracana), switchgrass (Panicum virgatum), sunflower (Helianthus annuus), safflower (Carthamus tinctorius), wheat (Triticum aestivum), soybean (Glycine max), tobacco (Nicotiana tabacum), potato (Solanum tuberosum), peanut (Arachis hypogaea), cotton (Gossypium barbadense, Gossvpium hirsutum), strawberry (e.g. Fragariaxananassa, Fragaria vesca, Fragaria moschata, Fragaria virginiana, Fragaria chiloensis), sweet potato (Ipomoea batatus), yam (Dioscorea spp., D. rotundata, D. cayenensis, D. alata, D. polystachwya, D. bulbifera, D. esculenta, D. dumetorum, D. trifida), cassava (Manihot esculenta), coffee (Coffea spp.), coconut (Cocos nucifera), okra (Abelmoschus esculentus), oil palm (e.g. Elaeis guineensis, Elaeis oleifera), pineapple (Ananas comosus), citrus trees (Citrus spp.), cocoa (Theobroma cacao), tea (Camellia sinensis), banana (Musa spp.), avocado (Persea americana), fig (Ficus casica), grape (Vitis vinifera), guava (Psidium guajava), mango (Mangifera indica), olive (Olea europaea), papaya (Carica papaya), cashew (Anacardium occidentale), macadamia (Macadamia integrifolia), almond (Prunus amygdalus), date (Phoenix dactylifera), cultivated forms of Beta vulgaris (sugar beets, garden beets, chard or spinach beet, mangelwurzel or fodder beet), sugarcane (Saccharum spp.), oat (Avena sativa), barley (Hordeum vulgare), cannabis (Cannabis sativa, C. indica, C. ruderalis), poplar (Populus spp.), eucalyptus (Eucalyptus spp.), Arabidopsis thaliana, Arabidopsis rhizogenes, Nicotiana benthamiana, Brachypodium distachyon, tomato (Solanum lycopersicum), eggplant (Solanum melongena), pepper (Capsicum annuum), lettuce (e.g. Lactuca sativa), bean (Phaseolus vulgaris), lima bean (Phaseolus limensis), pea (Lathyrus spp.), chickpea (Cicer arietinum), and members of the genus Cucumis such as cucumber (C. sativus), cantaloupe (C. cantalupensis), and musk melon (C. melo), and ornamentals. Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and Chrysanthemum. In certain embodiments, the plant is a Brassica, wheat, maize, potato, soybean, or cotton plant.

Also provided are progeny plants and seeds thereof comprising a polynucleotide or modification of the present disclosure. The present disclosure also provides fruits, seeds, tubers, leaves, stems, roots, and other plant parts produced by the plants and/or progeny plants of the disclosure as well as biological samples comprising, or produced or derived from, the plants or any part or parts thereof including, but not limited to, fruits, tubers, leaves, stems, roots, and seed. In certain embodiments, the biological sample is a commodity plant product. As used herein, a “commodity plant product” refers to any composition or product that is comprised of material derived from a plant, seed, plant cell, or plant part of the present disclosure. Commodity plant products may be sold to consumers and can be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, flakes, bran, fiber, paper, tea, coffee, silage, crushed of whole grain, and any other food for human or animal consumption; and biomasses and fuel products; and raw material in industry. Industrial uses of oils derived from the agricultural plants described herein include ingredients for paints, plastics, fibers, detergents, cosmetics, lubricants, and biodiesel fuel. Commodity plant products also include industrial compounds, such as a wide variety of resins used in the formulation of adhesives, films, plastics, paints, coatings and foams. It is recognized that such commodity plant products can be consumed or used by humans and other animals including, but not limited to, pets (e.g. dogs and cats), livestock (e.g. pigs, cows, chickens, turkeys, and ducks), and animals produced in freshwater and marine aquaculture systems (e.g. fish, shrimp, prawns, crayfish, and lobsters).

The plants disclosed herein find use in methods for limiting use of phosphorus-based fertilizer in agricultural crop production. The methods of the disclosure comprise planting a seedling, cutting, tuber, or seed of the present disclosure, wherein the seedling, cutting, tuber, or seed comprises a polynucleotide or modification of the present disclosure. The methods further comprise growing the plant that is derived from the seedling, cutting, tuber, or seed under conditions favorable for the growth and development of the plant, and optionally harvesting harvesting at least part from the plant. In certain embodiments, the part is a fruit, tuber, root, leaf, or seed. The methods reduce phosphorus fertilizer application in the field while maintaining optimal crop growth and yield, thus producing more with less input.

The present disclosure additionally provides methods for identifying and/or selecting a plant with improved phosphorus use efficiency. The methods find use in breeding plants for improved phosphorus use efficiency. The methods comprise detecting in a plant, or in at least one part or cell thereof, the presence of a polynucleotide encoding a protein kinase polypeptide of the present disclosure or a modification in a genomic sequence encoding a protein kinase of the present disclosure. In certain embodiments, detecting the presence of the polynucleotide or modification comprises detecting the entire protein kinase nucleotide sequence in genomic DNA isolated from a plant. In certain embodiments, however, detecting the presence of the polynucleotide or modification comprises detecting the presence of at least one marker within the protein kinase nucleotide sequence. In other embodiments, detecting the presence of the polynucleotide or modification comprises detecting the presence of the polypeptide encoded by the polynucleotide using, for example, immunological detection methods involving antibodies specific to the polypeptide.

The methods can involve one or more of the following molecular biology techniques that are disclosed elsewhere herein or otherwise known in the art including, but not limited to, isolating genomic DNA and/or RNA from the plant, amplifying a nucleic acid molecule comprising the polynucleotide and/or marker therein by PCR amplification, sequencing a nucleic acid molecule comprising the polynucleotide and/or marker, identifying the polynucleotide, the marker, or a transcript of the polynucleotide by nucleic acid hybridization, and conducting an immunological assay for the detection of the polypeptide encoded by the polynucleotide. It is recognized that oligonucleotide probes and PCR primers can be designed to identity the polynucleotides of the present disclosure and that such probes and PCR primers can be utilized in methods disclosed elsewhere herein or otherwise known in the art to rapidly identify one or more plants comprising the presence of a polynucleotide of the present disclosure in a population of plants.

Additionally provided are methods for conferring improved phosphorus use efficiency to a plant. The methods comprise crossing (i.e. cross-pollinating) a first plant comprising in its genome a polynucleotide or modification of present disclosure with a second plant lacking in its genome the polynucleotide or modification. Either the first plant or the second plant can be the pollen donor plant. For example, if the first plant is the pollen donor plant, then the second plant is the pollen-recipient plant. Likewise, if the second plant is the pollen donor plant, then the first plant is the pollen-recipient plant. Following the crossing, the pollen-recipient plant is grown under conditions favorable for the growth and development of the plant and for a sufficient period of time for seed to mature or to achieve an otherwise desirable growth stage for use. The seed can then be harvested and those seed comprising the polynucleotide or modification identified by any method known in the art.

All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1

Target of rapamycin (TOR) is a widely conserved serine/threonine protein kinase that integrates nutritional, growth, and stress signals to adjust growth in eukaryotes. TOR operates in at least two multi-protein complexes (TORC1 and TORC2), though no evidence exists for TORC2 in plants. The TORC1 complex comprises the conserved Regulatory-Associated Protein of TOR (RAPTOR) and the Lethal with Sec 13 (LST8) proteins. RAPTOR scaffolds and recruits substrates to TOR for phosphorylation. TORC1 activity is modulated by exogenous and endogenous signals such as light, hormones, energy deprivation, and nutrients. In this example, a combination of gene regulatory network inference, genetics, and biochemistry was used to discover a functional module operating upstream of TORC1 to control plant root growth in response to phosphorus availability.

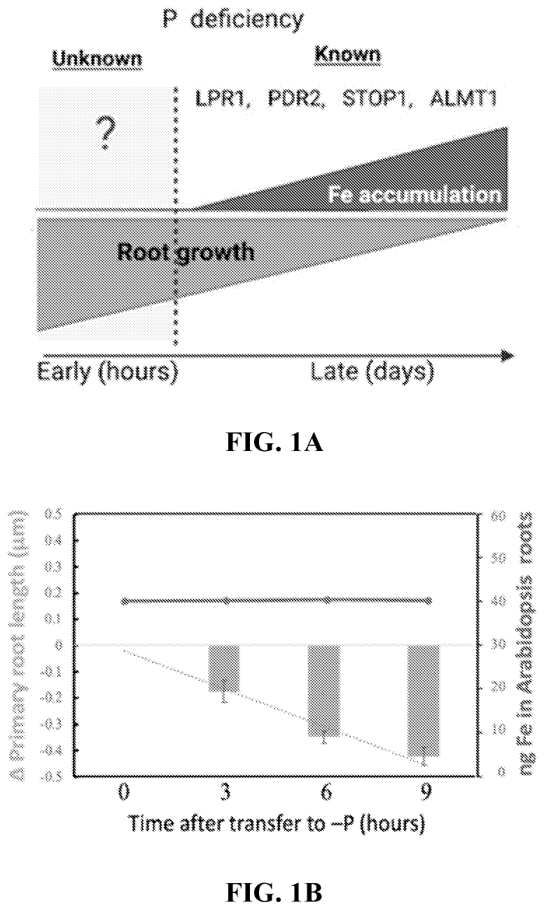

A typical feature of Arabidopsis thaliana response to phosphorus (P) deficiency is the inhibition of main root growth, which is proposed to be due to the accumulation of toxic levels of iron (Fe). This inhibition of main root growth is proposed to be due to the accumulation of toxic levels of iron (Fe), which involves LOW PHOSPHATE ROOT1 (LPR1)-PHOSPHATE DEFICIENCY RESPONSE2 (PDR2) and SENSITIVE TO PROTON RHIZOTOXICITY1 (STOP1), and ALUMINUM-ACTIVATED MALATE TRANSPORTERI (ALMT1) functional modules (FIG. 1A). However, the inhibition of root growth in P-limited environments occurs several hours before Fe overaccumulation (FIG. 1B), which concurs with the observed lack of change in induction of Fe-uptake transporters, that is consistent with the absence accumulation in the root tip 24 h after transfer to P deficiency. Therefore, the mechanism that triggers root growth inhibition in response to P deficiency is unknown.

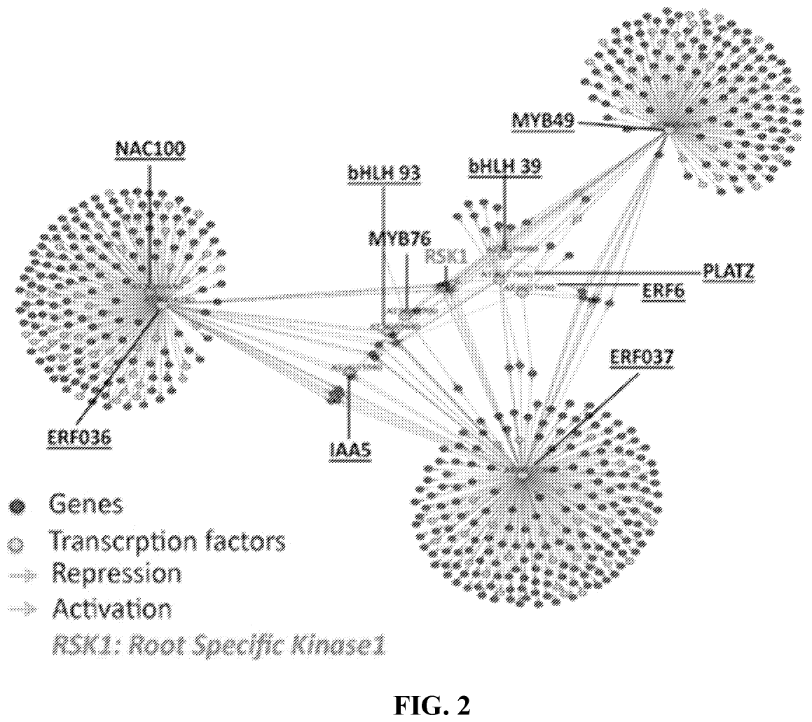

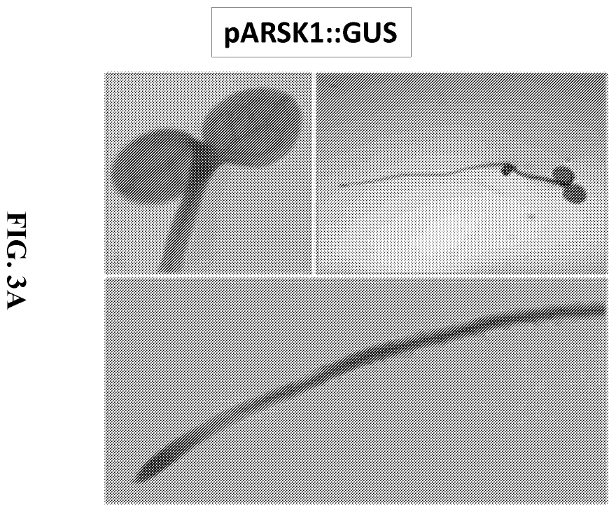

In order to study early signaling processes mediating plant response to P availability, global gene expression analysis in roots of A. thaliana (Col-0) plants exposed to different P regimes (+P and −P) for 3, 6, and 9 hours was performed. Given the established interaction between P and Fe homeostasis in plants, Fe availability was also altered to identify transcriptional responses specific to −P. Genes differentially expressed in at least one condition were used to generate gene regulatory networks (GRNs) to identify key genes mediating early transcriptional responses to P availability. GRN analysis for −P treatment identified the ten most-connected transcription factors (TFs) belonging to six families: AT1G77200 (ERF37), AT1G15580 (IAA5), AT3G16280 (ERF36), AT3G56980 (ORG3), AT5G07700 (MYB76), AT4G17900 (PLATZ11), AT5G61430 (NAC100), AT5G54230 (MYB49), AT4G17490 (ERF6), and AT5G65640 (CITF2) (FIG. 2). Notably, four of these TFs had a common transcriptional target: Arabidopsis Root Specific Kinase 1 (ARSK1, AT2G26290) (FIG. 2).

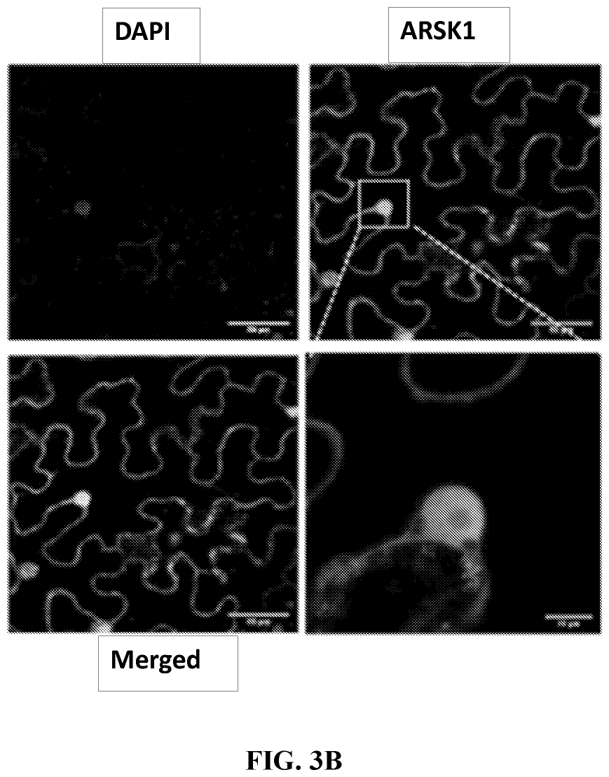

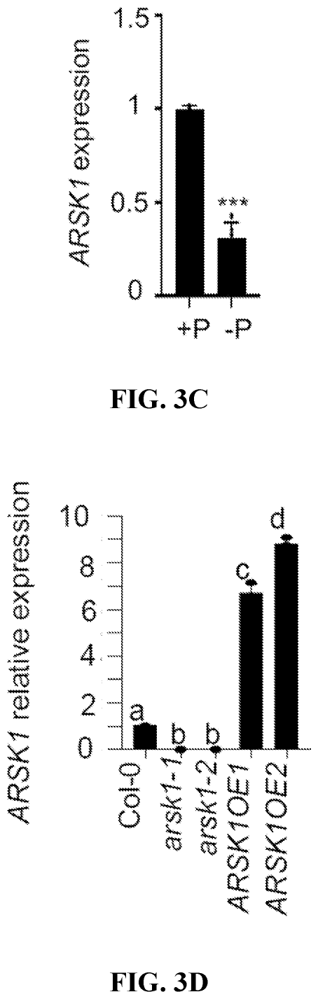

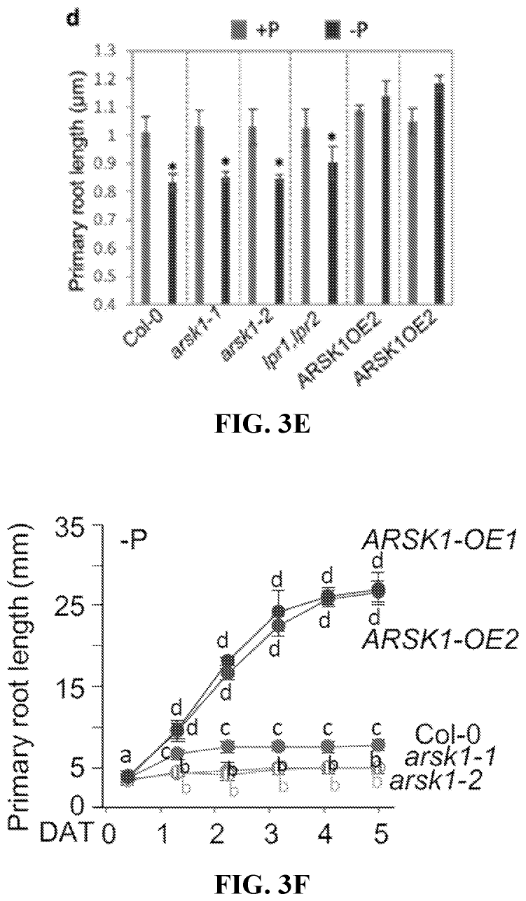

ARSK1 encodes a putative receptor-like cytoplasmic kinase (RLCK) that is mainly expressed in roots (FIG. 3A), nuclear localized (FIG. 3B), and its expression is repressed by −P (FIG. 3C). These observations suggest a role of ARSK1 in plant root responses to P limitation. ARSK1 was first described in 1995 as a root-specific and dehydration, ABA, NaCl induced gene and its function has remained elusive.

To assess the impact of ARSK1 on root growth, arsk1 mutant and ARSK1 overexpressing (ARSK1-OE) lines were characterized for root response to P limitation (FIG. 3D). Root growth assays revealed that arsk1 mutant plants display significantly stronger inhibition of root length by −P than wild-type Col-0 plants (FIG. 3E). In contrast, ARSK1 overexpression prevents root growth inhibition by −P (FIG. 3E). Since the phenotype of ARSK1 overexpressing lines mimics lpr1, lpr2, a mutant with maintained root growth in response to P deficiency, kinetic response between these lines were compared to identify the pathway controlling the initial root growth response to low P. After 3 to 4 h of transfer to low P, a significant decrease in root growth of wild-type Col-0 as well as lpr1, lpr2 mutant plants was detected; however, ARSK1-OE lines maintained root growth (FIG. 3E). These results revealed a role of ARSK1 in uncoupling root growth from the negative effect of Fe accumulation in −P conditions. The results indicate that ARSK1 is a key component of a new molecular pathway controlling root growth in a P-dependent manner.

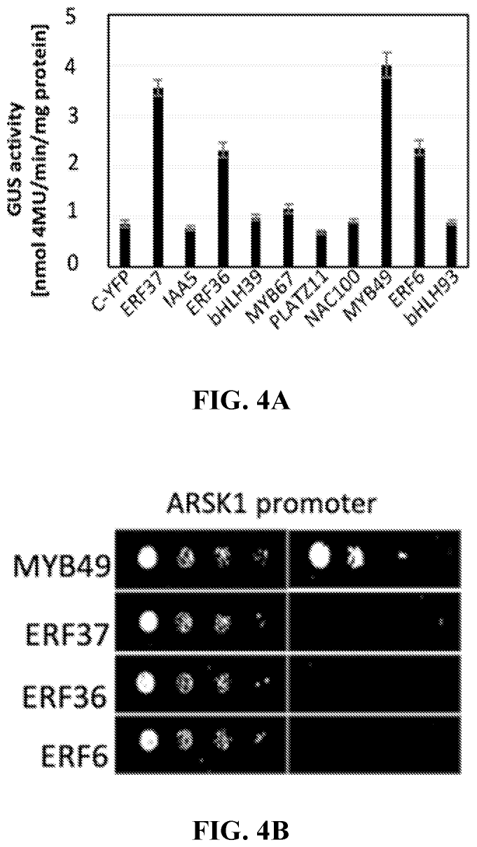

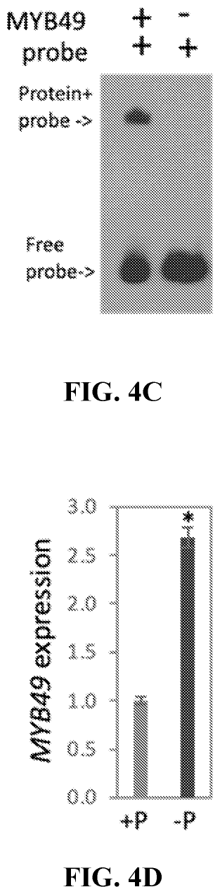

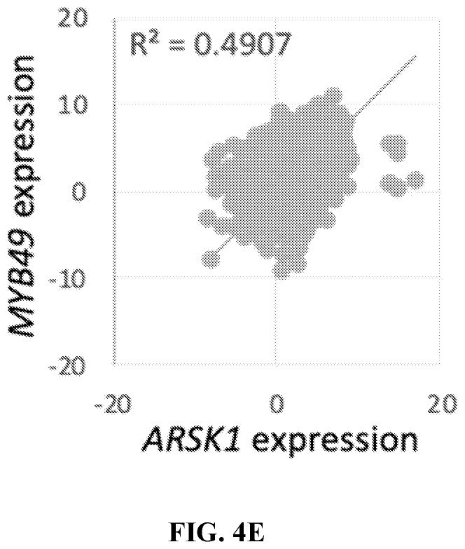

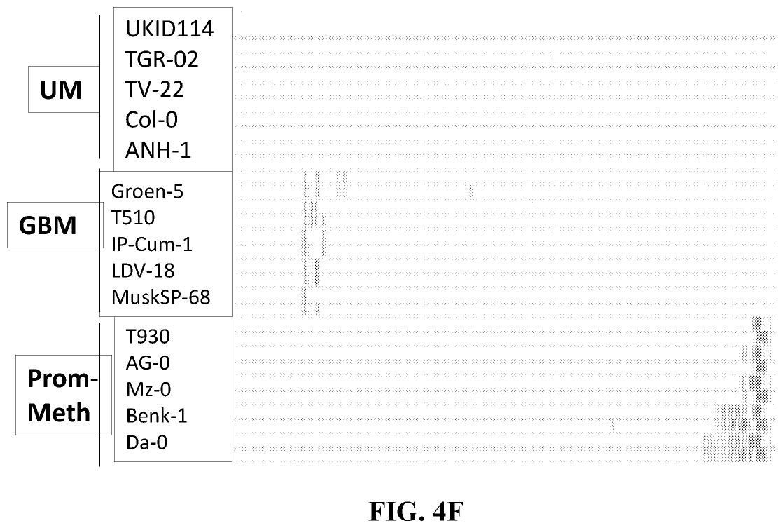

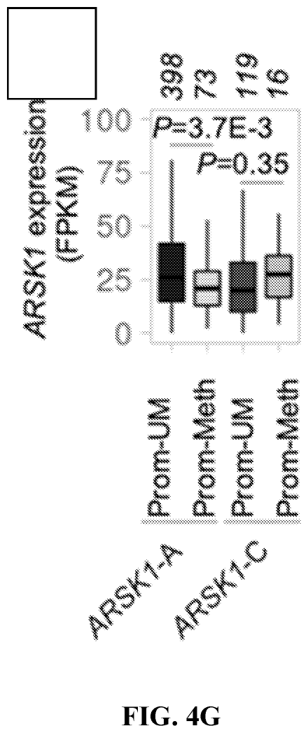

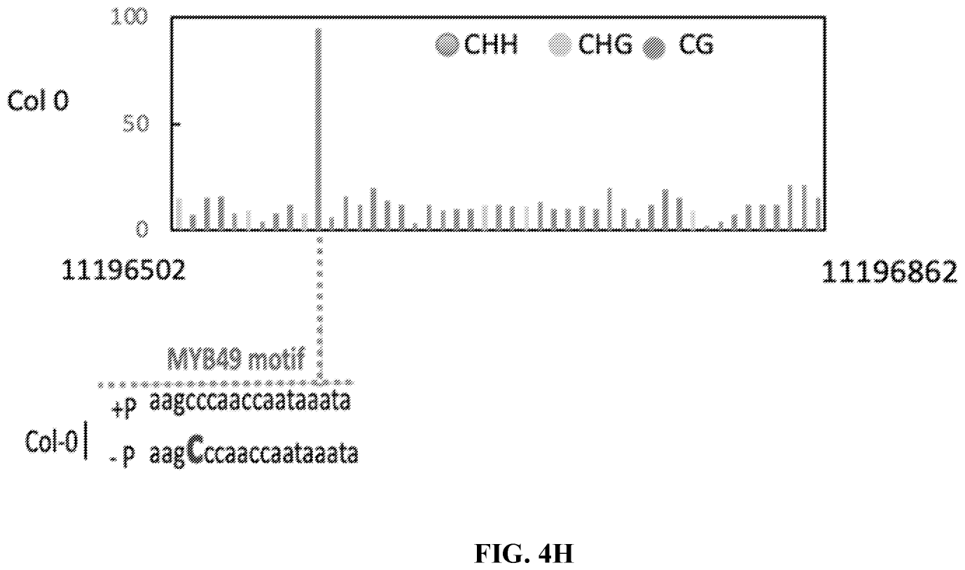

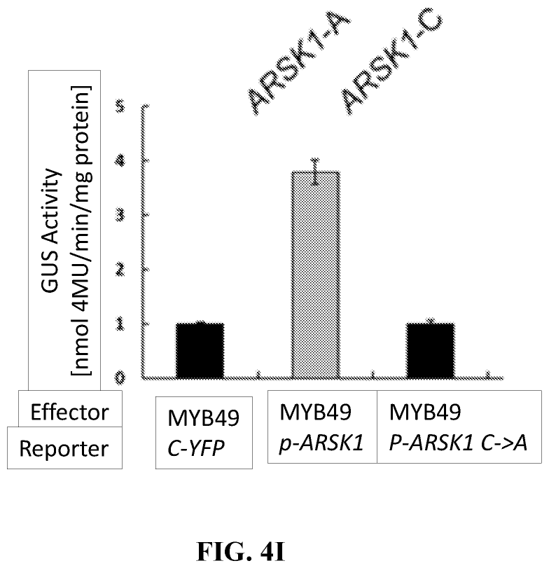

To gain insight on the transcriptional regulation of ARSK1 by P availability, whether TFs identified by GRN analysis influence directly or indirectly the expression of ARSK1 was tested. Transactivation assays using the TFs as effectors and ARSK1's promoter fused to GUS as reporter were performed. The results showed that ERF6 (AT4G17490), ERF36 (AT3G16280), ERF37 (AT1G77200) and MYB49 (AT5G54230) activate ARSK1 (FIG. 4A). Yeast-one hybrid (Y1H) confirmed a direct interaction only between MYB49 and the ARSK1 promoter (FIG. 4B). In silico analysis identified the presence of a putative binding motif for MYB49 in the ARSK1 promoter. Using an electromobility shift assay (EMSA), this binding site for MYB49 was validated (FIG. 4C). Taken together, the results identify MYB49-ARSK1 as a new module to mediate plant response to P deficiency. Although transactivation assays demonstrate that MYB49 activates ARSK1 expression, the observation that P deficiency regulates MYB49 and ARSK1 expression in an opposite manner is intriguing. In addition, in contrast to the negative relationship between MYB49 and ARSK1 under P-limitation, the expression of these two genes displays a strong positive correlation across different treatments, developmental stages, as well as natural Arabidopsis accessions (FIG. 4D). Interestingly, investigation of 1001 Arabidopsis methylomes pointed to an additional level of regulation of ARSK1 expression via MYB49. The MYB49 binding motif in ARSK1 promoter displays differential methylation across Arabidopsis accessions (FIG. 4E), and accessions harboring a methylated MYB49 binding motif display low ARSK1 expression (FIG. 4F). This motif is unmethylated in Col-0 under P-sufficient conditions and becomes methylated under −P (FIG. 4G-H). Therefore, it was hypothesized that MYB49 generally positively regulates ARSK1; however, P limitation-induced methylation of the ARSK1 promoter interferes with the binding of MYB49, which causes downregulation of ARSK1 during P deficiency. To assess whether P-dependent differential methylation in the promoter of ARSK1 affects the binding of MYB49, in planta transactivation assays in Arabidopsis protoplasts were performed using different versions of ARSK1 promoter that drive the expression of the firefly luciferase gene, methylated or mutated at the cytosine residues which influence MYB49 binding were used in these experiments. The results revealed luminescence detected only using the native promoter, but not the mutated version of the ARSK1 promoter (FIG. 4H). Collectively, the data provide evidence for P limitation-induced DNA methylation of the ASKR1 promoter, which led to downregulation of ARSK1 gene expression.

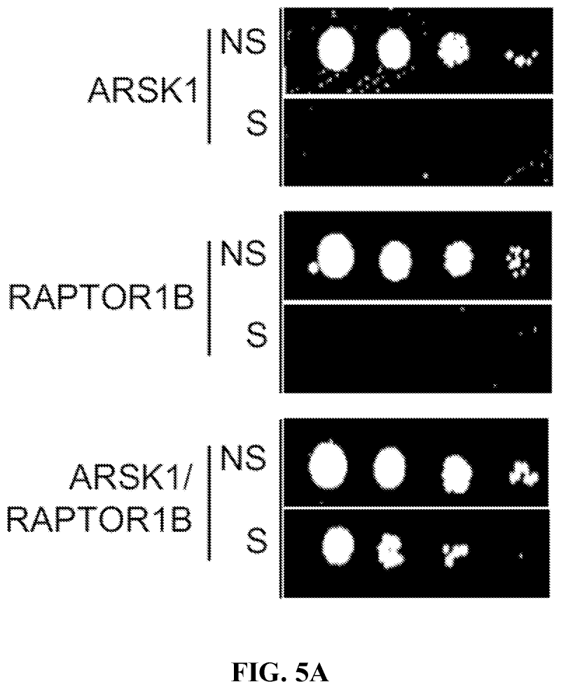

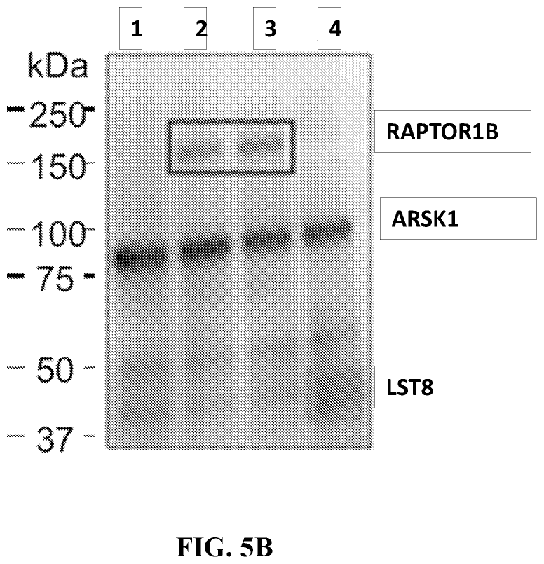

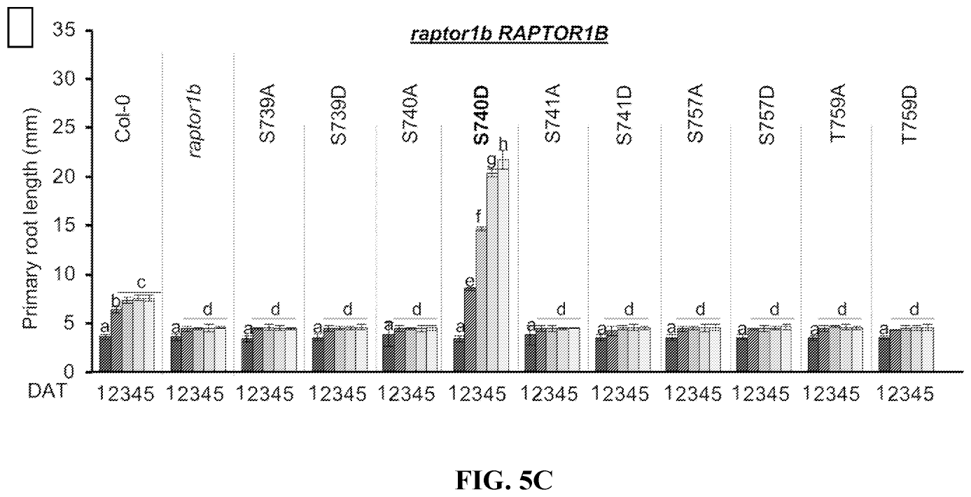

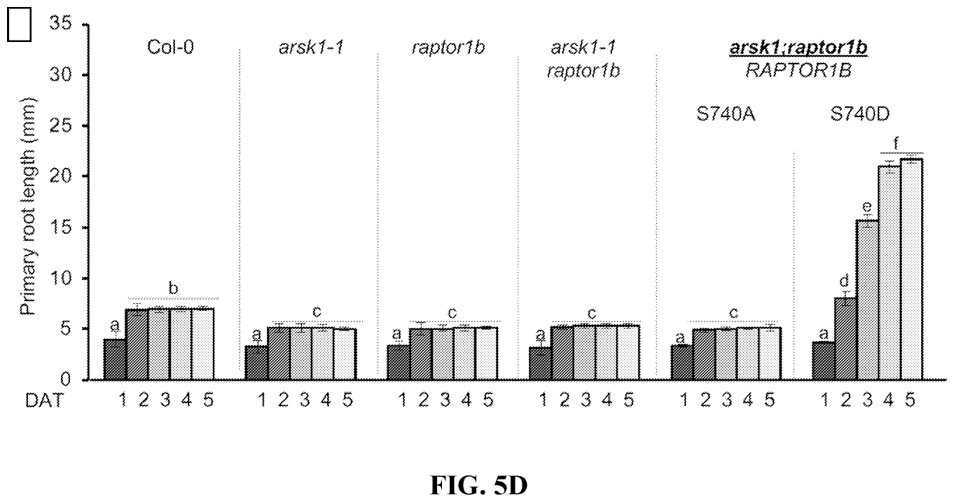

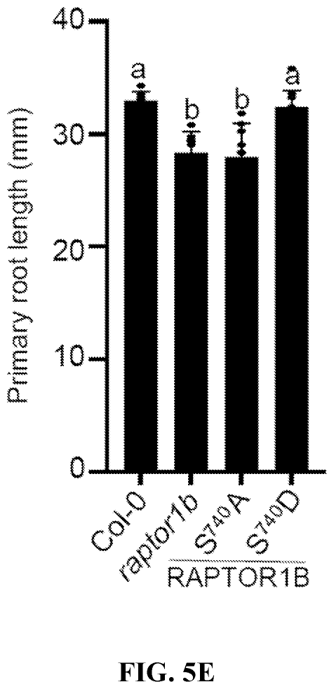

To identify putative partners of ARSK1, the published Arabidopsis interactome was investigated. In this high-throughput yeast-two hybrid (Y2H) screen, ARSK1 interacted with only RAPTOR1B, which is a scaffold protein of the TOR complex. ARSK1 was confirmed to interact with RAPTOR1B using the same assay (FIG. 5A). Since ARSK1 is a protein kinase, whether ARSK1 could phosphorylate RAPTOR1B was investigated. For this, in vitro kinase assays using recombinant ARSK1 with RAPTOR1B, followed by RAPTOR1B phosphopeptide identification using mass spectrometry was performed (FIG. 5B). These assays revealed that ARSK1 can phosphorylate two RAPTOR1B peptides. The first peptide contains three serine (S) residues (S⁷³⁹, S⁷⁴⁰ or S⁷⁴¹) as putative phosphorylation sites, and the second peptide harbors S757 or threonine (T⁷⁵⁹). Additionally, whether ARSK1 could phosphorylate TOR or LST8, which are other components of TORC1, was tested (FIG. 4C). ARSK1 could not phosphorylate these two proteins, indicating that ARSK1 specifically interacts and phosphorylates RAPTOR1B.

To determine whether RAPTOR1B affects root growth response to P limitation, raptor1b mutant plants grown in P-sufficient and P-deficient environments were characterized. The raptor1b-1 mutant plants displayed significantly shorter primary roots than WT plants under −P, which phenocopies the arsk1-1 mutant (FIG. 5C-D). Next, to test whether ARSK1 and RAPTOR1B mediate root growth response to P limitation through the same genetic pathway, root growth of the arsk1-1,raptor1b-1 double mutant under different P regimes was examined. The arsk1-1,raptor1b-1 root growth response to −P is very similar to that of raptor1b-1 or arsk1-1 single mutants (FIG. 5D), which demonstrates that ARSK1 and RAPTOR1B influence root growth in low P through the same genetic pathway. Next, the effects of RAPTOR1B phosphorylation on root growth was investigated using phospho-mimicking (replacing S or T with aspartate; D) and phospho-deficient (replacing S or T with alanine; A) forms of RAPTOR1B for each of the putative phosphosites (S⁷³⁹, S⁷⁴⁰ or S⁷⁴¹, S757, and T⁷⁵⁹) in raptor1b-1 mutant background (FIG. 5C). Expression of only the S⁷⁴⁰ phospho-mimicking RAPTOR1B form (RAPTOR1BS740D) alleviated root growth inhibition by −P (FIGS. 5C and 5E). Furthermore, the expression of the RAPTOR1BS740D phospho-mimicking form successfully complements arsk1-1,raptor1b-1 root growth under −P (FIG. 5D). These results suggest that ARSK1 mediates RAPTOR1B's S⁷⁴⁰ phosphorylation in a P-dependent manner that adjusts root growth to P availability.

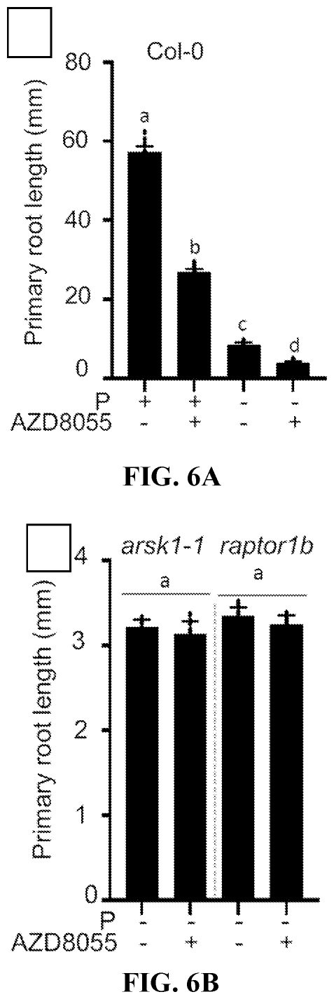

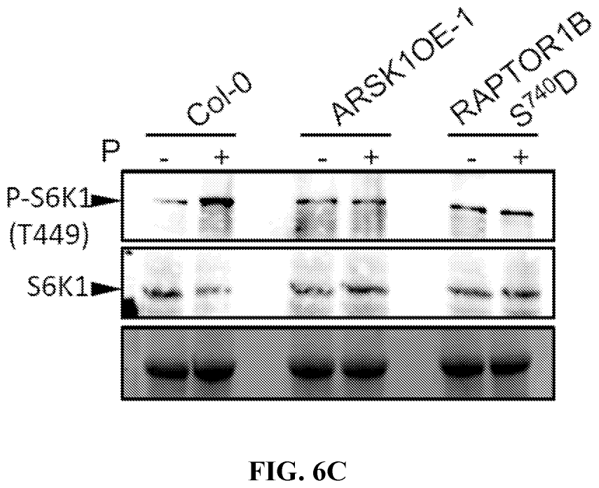

Since −P represses ARSK1 which phosphorylates a core component of TORC1, our biochemical and genetic experiments suggest that −P stress inhibits TORC1, thereby restricting root growth. To test this hypothesis, the effect of a specific TOR inhibitor, AZD-8055, on root growth response to P deficiency was examined. AZD-8055 treatment resulted in a strong reduction of root growth in WT plants in P-replete as well as P-depleted conditions (FIG. 6A). Therefore, AZD-8055 inhibits the root growth of arsk1-1 and raptor1b mutants and WT to a similar level under P sufficient conditions. However, neither raptor1b-1 nor arsk1-1 mutant plants responded to AZD-8055 treatment when grown in −P conditions (FIG. 6B). Furthermore, analysis of TOR activity using plant S6 kinase (S6-K) phosphorylation assays revealed that P deficiency remarkably decreased S6-K phosphorylation (S6K-p), indicating significantly reduced TOR activity in P-deprived roots (FIG. 6C). Interestingly, overexpression of ARSK1 (ARSK1-OE) or RAPTOR1BS⁷⁴⁰D prevented the decrease of S6K-p accumulation compared to Col-0 plants grown under P-deficient conditions (FIG. 6C). Overall, the results support a central role of TORC1 and ARSK1 in root growth response to P availability and validate the premises of our working model for an ARSK1-TORC1 axis to maintain root growth in −P conditions.

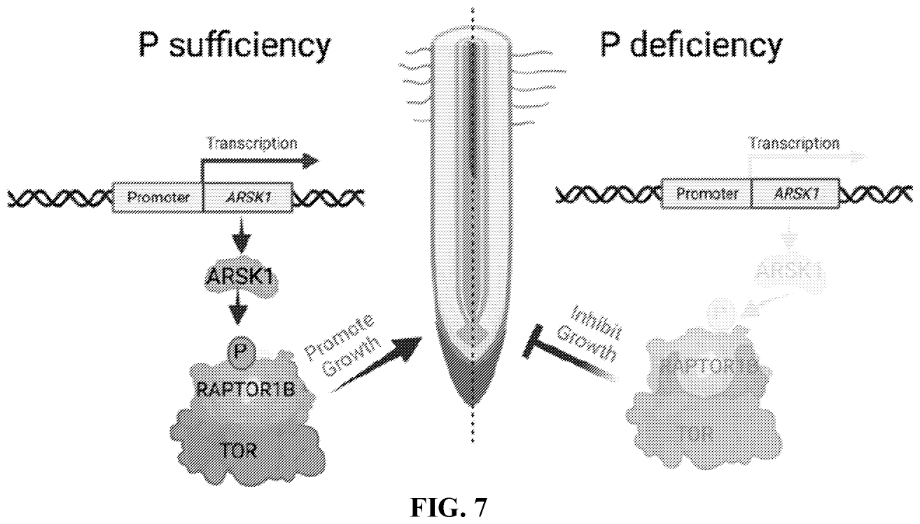

In conclusion, this work describes a molecular module that transmits nutritional (P) status to influence growth through TOR signaling (FIG. 7). Under P sufficiency environments, MYB49 promotes ARSK1 expression. In turn, ARSK1 protein phosphorylates RAPTOR1B, and the phosphorylated RAPTOR1-B promotes TOR activity to maintain root growth. P deficiency induces methylation of ARSK1 promoter to repress its expression, thus preventing accessibility to MYB49. In the absence of ARSK1, RAPTOR1B is not phosphorylated, and TOR activity is altered, leading to the inhibition of root growth. These findings uncover a novel pathway which integrates P availability and energy sensing TOR pathway. This discovery offers a new perspective on how to improve plant growth under P limitation through optimizing nutrient foraging by roots, which will become increasingly important for stewarding in sustainable agriculture. Of equal importance, and because of the evolutionary conservation of TORC1 and its role in nutrient signaling, the discovery paves the way to uncover how nutrient availability connects to growth machinery in metazoans in general, with great potential for biomedical applications to improve health- and life-span.

### Methods

### Plant Growth Conditions

Seeds of Arabidopsis thaliana wild type (ecotype Columbia, Col-0, CS60000) and knock-out mutant lines arsk1-1 SALK_050925, arsk1-2 SALK_092559, and raptor1b SALK_101990, were obtained from the Nottingham Arabidopsis Stock Centre (NASC). Homozygous mutant lines were confirmed by PCR. RSK1 overexpressed lines (ARSK1-OE) were generated by expressing ARSK1 CDS in the arsk1 mutant background. For p35S::ARSK1-GFP cloning, the full-length ARSK1-attB-flanked PCR product was obtained using specific primers. Using the gateway cloning vector set, the ARSK1 was fused with GFP cloned in pMDC83 under the control of CaMV 35S promoter (p35S::ARSK1-GFP). The construct was introduced into the A. tumefaciens strain GV3101. Arabidopsis plants were grown on control plates containing 1.249 mM KH2PO4; 0.25 mM Ca(NO3)2; 0.5 mM KNO3; 1 mM MgSO4; 100 μM FeSO4.7H2O; 30 μM H3BO3; 1 μM ZnCl2; 10 μM MnCl2; 1 μM CuCl2; 0.1 μM (NH4)6Mo7O24; and 50 μM KCl; 0.05% 2-(N-morpholino)ethanesulfonic acid (MES), 1% sucrose, and 0.8% washed agar. P-deficient media contained 12.49 μM KH2PO4 (+Fe−P). Fe-free media was obtained by omitting FeSO4.7H2O from the growth media (−Fe+P). P- and Fe-deficient media contained 12.49 μM KH2PO4 (+Fe−P), and no FeSO4.7H2O (−Fe−P). Seeds were stratified at 4° C. for 3 days and grown on vertical agar plates in a growth chamber with 22° C., 24 h of light at 100 μmol m-2s-1 fluorescent illumination. Plants were transformed by Agrobacterium-mediated transformation using the established floral dip method.

Agrobacterium-mediated transient expression was performed using Agrobacterium GV3101 strain. Briefly, overnight-grown Agrobacterium culture was resuspended in induction medium (10 mM MES-KOH, pH 5.7, 10 mM MgCl2, and 100 μM acetosyringone) to OD₆₀₀=0.2 and incubated for 2 h at room temperature, before infiltration into Nicotiana benthamiana leaves. Agrobacterium strain carrying the 35S:p19 construct was co-infiltrated to enhance the maximum levels of protein expression. Transiently expressed proteins were analyzed 4 days after infiltration with the Leica SP8 microscope. On the 4 DAI, the leaf disc was stained with 0.025% tween20 in 2 mg/L DAPI solution for 4 hours. For DAPI staining, On the 4 DAI(Day after infiltration), the leaf disc was stained in 2 mg/L DAPI solution containing 0.025% tween 20 for 4 hours.

### Genome Wide Expression Analysis

Total RNA was extracted from frozen and ground root tissues using TRIzol™ reagent (15596026, ThermoFisher Scientific) following the manufacturer's instructions. RNA integrity and concentration were determined using a 2100 Bioanalyzer Instrument (Agilent) and Agilent RNA 6000 Nano kit (5067-1511, Agilent). DNA contamination was removed by digestion with DNase I (AMPD1, SIGMA).

Genome-wide expression analysis in roots was based on 3 biological replicates obtained from independent experiments including four treatments (+P+Fe, +P−Fe, −P+Fe, −P−Fe) and 3 time points. Gene expression measurements were performed using Arabidopsis AFFYMETRIX® Gene1.1 ST array strips designed to measure whole transcript accumulation of 28,501 genes (or transcripts clusters), based on 600,941 probes defined on TAIR10 genome annotation. Biotin labeled and fragmented cRNAs were obtained using a GENECHIP® WT PLUS Reagent kit (902280, ThermoFisher Scientific) following manufacturer's instructions. Hybridization on array strips was performed for 16 h at 48° C. Arrays were washed, stained, and scanned using a GeneAtlas HWS Kit (901667, ThermoFisher Scientific) on the GENEATLAS® Fluidics and Imaging Station.

Microarray raw data were processed with GCRMA available on the Expression Console Software developed by Affymetrix. Data analysis was performed in the program R. Genes responding to the P and Fe treatment across time were identified using a three-way ANOVA that was modeled as follows: Y=μ+αP+βFe+γTime (αβ)P*Fe+(αγ)P*Time+(βγ)Fe*Time+(αβγ)P*Fe*Time+ε, where Y is the normalized expression signal of a gene, is the global mean, the α, β and γ-coefficients correspond to the effects of Phosphate, of Iron, of Time (3, 6, 9 hours) and of the interaction between the factors, and F represents unexplained variance. All the genes for which all the coefficients are significant (p-value<0.05), except for the γTime coefficient only, to explain variation of expression were selected. Given the three-way anova analysis, we retained differential expression events (p-value <0.05) between genes in each experimental treatment (+P/−Fe, −P/+Fe, −P/−Fe) per time point (3 h, 6 h, 9 h) and the corresponding control treatments (+P/+Fe), resulting in a total of 242 out of the 26320 genes being differentially expressed in at least one experiment and time point. In addition, we curated three short time course (3 h, 6 h, 9 h), differential expression datasets for each treatment (+P/−Fe, −P/+Fe, −P/−Fe) based on computing the log fold change between each treatment per time point and the corresponding control treatment (+P/+Fe).

### Large-Scale Root Specific Gene Expression Compendium

A recently published, comprehensive study on gene expression for A. thaliana was acquired. The collection had been subjected to consistent data processing and quality control. Given this dataset, we only retained conditions related to various stress and nutrient treatments in root for A. thaliana ecotype Columbia. Subsequently, we transformed this condition specific gene expression dataset into differential expression profiles computing the log fold change difference between individual treatments and corresponding control conditions. Our final differential expression dataset covered 82% (21678 out of 26320 genes) of the A. thaliana genome and consisted of 177 differential expression profiles.

### Transcription Factor Binding Information and Family Annotations

Most recent transcription factor family annotations were downloaded from iTAK. Further, the most comprehensive A. thaliana transcription factor binding dataset to date based on in-vitro-expressed transcription factors, consisting of˜2.8 million links for 387 transcription factors was acquired.

### Phosphate Iron Treatment Gene Expression Hierarchy

The hierarchy of the iron and phosphate specific differential expression experiments (+P/−Fe, −P/+Fe, −P/−Fe) was estimated based on computing the mean differential expression of each gene in each experiment in order to represent similarity in gene expression patterns of experiments.

### Gene Regulatory Network Inference Using an Unsupervised Ensemble Approach

Thirteen transcription factors (based on iTAK annotations) were observed to be differentially expressed in at least one of the iron and phosphate deficiency experiments. To derive the gene regulatory network based on these Nr=13 transcription factors, an ensemble model of transcriptional regulation was built, integrating several heterogeneous features to derive a score per regulatory link 1:r→g between a transcription factor r∈N_r and a putative target gene g.

Therefore, we integrate: (i) experimentally observed binding of r in the promoter of g, (ii) co-differential expression of r and g in the iron and phosphate experimental datasets, (iii) link strengths between r and g in a genome wide supportive gene regulatory network inferred from a large-scale root stress specific gene expression dataset.

For unsupervised data integration, we assigned binary weights w∈[0,1] to the transcription factor promoter binding as well as co-differential expression link evidences as either present or absent. To estimate the supportive gene regulatory network, we used the curated large-scale differential expression dataset of A. thaliana root stress treatments, applying a robust random forest regression based approach. Regression-based approaches to gene regulatory network inference are based on the assumption that the expression profiles of the transcription factors that directly regulate a target gene are the most informative, among all transcription factors, to predict the expression profile of the target gene. Tree-based regression approaches, such as random forests, have proven successful as they can handle complex interaction and apply resampling strategies for repeated subsampling of the data, providing an inherent cross-validation. To implement a genome wide background distribution, we ran random forest regression using Nr=13 differential expressed regulators for all genes covered in the expression dataset. Regression parameters were set for number of split variables k=√(N_r) and number of trees t=500. Subsequently, we computed the empirical cumulative distribution function over all predictions assigning probabilities as continuous weight between 0 and 1 per regulatory link.

Finally, we defined rank based on heterogeneous data integration as:

$${rank}_{i} = {\sum }_{i = 1}^{3} {w}_{i} = {w}_{random forest regression} + {w}_{co - differential expression} + {w}_{DNA binding}$$

We retain a link 1, if [symbol] rank [symbol] _1>t_v, otherwise it was removed. Here t_v denotes a threshold parameter. We set t_v=1.95 in order to prioritize experimentally observed transcription factor DNA binding and co-differential expression. Accordingly, if a link is supported by experimentally observed transcription factor DNA binding and co-differential expression, the supportive root stress specific regulatory network becomes less important. In contrast, if a link is supported by either co-differentially expressed or experimentally observed transcription factor DNA binding only, the root stress specific regulatory network is invoked to decide on whether to retain an individual regulatory link (if w_(random forest regression) (1)>0.95).

For the final gene regulatory network, we used the sign of the Pearson's correlation coefficient (pcc) based on the curated large-scale differential expression dataset of A. thaliana root stress treatments to estimate a putative mode of regulation between a transcription factor r and the target gene g, as either activation (pcc>0) or repression (pcc<0). However, since transcription factors typically act in sets onto a given target, this allows for changes in transcriptional modalities of individual transcription factors, such mode inference should be treated with caution.

### In Planta Transactivation Assay

The in planta transactivation assay was performed in N. benthamiana as previously described. AP2/ERF (3 TFs), AUX/IAA (1 TF), bHLH (2 TFs), MYB (2 TFs), NAC (1 TF), PLATZ (1 TF) CDS were cloned. The CDS was placed under the CaMV. 35S promoter. RSK1, ERF36, ERF37 promoters were cloned and then fused to the β-GUS-encoding reporter gene using the Gateway system. The 35S::C-YFP construct was used as negative control. Each construct was transformed into Agrobacterium. For each construct, positive clones were grown at 28° C. overnight before being washed four times in the infiltration buffer (10 mM MgCl2, 100 uM acetosyringone, and 10 mM MES (pH 5.6)). At OD600 of 0.8, the effector and reporter constructs were co-infiltrated at a 9:1 ratio in the fourth fully expanded leaves of six week-old tobacco plants. Three independent infiltrations per combination were performed, leading to siix biological repeats per construct. One leaf from different plants was used to infiltrate each plasmid combination. Three days later, the Infiltrated areas were excised and used for GUS extraction and GUS enzymatic activity measurements. Comparison of the effect of TF and C-YFP protein on each promoter determines the relative GUS enzymatic activity.

### Yeast Experiments

For Y2H experiments, ARSK1 and RAPTOR1B PCR products were obtained using high-fidelity Phusion DNA polymerase. The constructs were sequenced to ensure their integrity. ARSK1, and RAPTOR1B were recombined into pDEST32, allowing fusion with the GAL4 DNA binding domain. Each pDEST22 and pDEST32 vector containing either ARSK1 or RAPTOR1B was transformed alone or in combination into yeast (AH109 strain; Clontech). Subsequent steps were carried out according to the manufacturer's instructions using the ADE2 HIS3 reporter genes (Clontech). For Y1H experiments, bERF6, bERF36, bERF37 and MYB49 cDNAs was PCR-amplified from a pool of Col-0 cDNA using the cTF-B1 and cTF-B2 primers, introduced into the pDONR207 vector (Gateway) and then recombined into the pDEST22 vector (Gateway), allowing the expression of each TF fused to the GAL4 activation domain in yeast pDEST22. To assess if ERF6, ERF36, ERF37, and MYB49 could interact with the different MYB and bHLH putative binding sites present on the ARSK1 promoter, each one was separately cloned as hexamers into the pHis-LIC vector.

### Real-Time Quantitative Reverse-Transcription PCR

Seeds of Arabidopsis wild type (Col-0), and rsk1 mutant plants were germinated and grown for 7 days in control (+Fe+P) media, and then transferred to +Fe+P, Fe+P, or −Fe−P. Root tissues were collected, and then used for total RNA extraction. Each experiment was conducted with 16 plants and 4-6 plants were pooled for RNA extraction, resulting in 3-4 biological replicates. Two g of the total RNA was used for reverse transcription (Promega) to synthesize cDNA using oligo(dT) primer (Promega). Real-time quantitative reverse-transcription PCR (qRT-PCR) was performed using 384-well plates with a LightCycler 480 Real-Time PCR System (Roche diagnostics). The Ubiquitin 10 mRNA (UBQ10: At4g05320) was used as a control to calculate the relative mRNA level of each gene.

### In Vitro Phosphorylation Assay

Production of Recombinant RSK1 kinase in E. coli: For gateway cloning of RSK1, the RSK1 CDS was synthesized together with the flanking attL1 and attL2 gateway sites into the pUC57-Km cloning vector (Genscript) and the resulting entry vector was cloned into pDest-HisMBP through standard LR gateway reaction. The resulting His-MBP expression vector was transformed into E. coli BL21 for production of recombinant RSK1.

### In Vitro Kinase Assays

For in vitro kinase assays, substrates were TAP-purified from PSB-D cell cultures expressing Raptor1B fused N- or C-terminally to the GSrhino TAP tag or LST8-1 fused C-terminally to the GSrhino tag. As negative control, a mock TAP purification was performed on a wild-type PSB-D cell culture. TAP purifications were performed as described previously, with minor adjustments: phosphatase inhibitors (NaF, Na2VO4, β-glycerophosphate and p-NO2PhenylPO4) present in the TAP extraction buffer were added to all binding, wash and elution buffers, and protein complexes were not eluted from the Streptavidin beads. After standard washing of Streptavidin beads, the beads were washed with kinase wash buffer (25 mM HEPES, pH 7.4, 20 mM KCl). Washed beads were dissolved in kinase assay buffer (25 mM HEPES, pH 7.4, 50 mM KCl, 10 mM MgCl2, 10 μM cold ATP). For P32 ATP kinase assays, kinase reactions were performed for 1 h at 30° C. combining 20 μL TAP-purified substrates with 20 μL recombinant RSK1 kinase, in the presence of 5 μCi γ-32P ATP. As negative control, 20 μL MBP elution buffer was added to the TAP-purified substrates instead of the RSK1 kinase. Reactions were stopped by addition of SDS sample buffer and incubation for 10 min at 95° C. Proteins were separated by SDS-PAGE and stained with Coomassie brilliant blue R-250. Gels were dried and radioactivity was detected by autoradiography on a photographic film. For mass spectrometry-based identification of phosphopeptides, kinase assays were performed as described above, using 10 μM cold ATP instead of γ-32P ATP and reactions were incubated overnight at 30° C. Reactions were stopped by addition of NuPAGE sample buffer (ThermoFisher Scientific) and incubation at 70° C. for 10 min. Proteins were separated for 7 min at 200 V on a 4-12% NuPAGE gradient gel, stained with Coomassie G-250 and in-gel trypsin digested.

### LC-MS/MS Analysis

Peptides were re-dissolved in 20 μl loading solvent A (0.1% TFA in water/ACN (98:2, v/v)) of which 5 μl was injected for LC-MS/MS analysis on an Ultimate 3000 RSLC nano LC (Thermo Fisher Scientific, Bremen, Germany) in-line connected to a Q Exactive mass spectrometer (Thermo Fisher Scientific). The peptides were first loaded on a trapping column made in-house, 100 μm internal diameter (ID.)×20 mm, 5 μm beads C18 Reprosil-HD, Dr. Maisch, Ammerbuch-Entringen, Germany) and after flushing from the trapping column the peptides were separated on a 50 cm ρPAC™ column with C18-endcapped functionality (Pharmafluidics, Belgium) kept at a constant temperature of 35° C. Peptides were eluted by a linear gradient from 98% solvent A′ (0.1% formic acid in water) to 55% solvent B′ (0.1% formic acid in water/acetonitrile, 20/80 (v/v)) in 30 min at a flow rate of 300 nL/min, followed by a 5 min wash reaching 99% solvent B′. The mass spectrometer was operated in data-dependent, positive ionization mode, automatically switching between MS and MS/MS acquisition for the 5 most abundant peaks in a given MS spectrum. The source voltage was 3.2 kV, and the capillary temperature was 275° C. One MS1 scan (m/z 400-2,000, AGC target 3×106 ions, maximum ion injection time 80 ms), acquired at a resolution of 70,000 (at 200 m/z), was followed by up to 5 tandem MS scans (resolution 17,500 at 200 m/z) of the most intense ions fulfilling predefined selection criteria (AGC target 5×104 ions, maximum ion injection time 80 ms, isolation window 2 Da, fixed first mass 140 m/z, spectrum data type: centroid, intensity threshold 1.3×E4, exclusion of unassigned, 1, 5-8, >8 positively charged precursors, peptide match preferred, exclude isotopes on, dynamic exclusion time 12 s). The HCD collision energy was set to 25% Normalized Collision Energy and the polydimethylcyclosiloxane background ion at 445.120025 Da was used for internal calibration (lock mass). The raw files were processed with the MaxQuant software (version 1.6.10.43) (Cox and Mann, 2008), and searched with the built-in Andromeda search engine against the Araport11 plus database. This database consists of the Araport11 database with crap sequences, e.g. tags, keratins, trypsin etc. added. MaxQuant search parameters can be found in supplemental table x.

### Protein Analysis

Arabidopsis seedling were grown for 10 day, then transferred on medium in presence or absence of P for 9 hours. For the immunoblotting, 100 mg plant tissue was ground in liquid nitrogen and suspended in 1.5 volume of extraction buffer (50 mM Tris-HCl pH 7.5, 150 mM NaCl, 10% glycerol, 5 mM dithiothreitol, 2 mM Na2MoO4, 2.5 mM NaF, 1.5 mM activated Na3VO4, 1 mM phenylmethanesulfonyl fluoride (PMSF), 1% IGEPAL, and cOmplete™ Protease Inhibitor Cocktail (Roche)). To obtain soluble protein, cell debris was removed by centrifugation 3 times. After denaturing with 6×Laemmli buffer at 95° C. for 5 minutes, 25 μg of protein were loaded into each well on the 10% SDS PAGE gel and transferred to the PVDF membrane (Bio-Rad, 1620174). The membrane were incubated in the blocking solution (5% skim milk) for 1 h at room temperature (RT). For immunoblotting, RAPTOR1B primary antibody (PhytoAB, PHY2235S) was diluted to 1:750 and incubated overnight at 4° C. Anti-Rabbit IgG antibody (Sigma-Aldrich, A0545) diluted to 1:10000 was used as a secondary antibody for 2 hours at room temperature. TOR activity was assessed through the detection of S6K protein forms (phosphorylated and unphosphorylated forms), which were probed with Phospho-S6K antibody (Thr449, Abcam, ab207399) and S6K antibody (Cedarlane Labs, AS121855). Both antibodies were diluted to 1:2000 and incubated overnight at 4° C., The Goat anti-Rabbit IgG Antibody, HRP conjugate (Sigma-Aldrich, A0545, 1:4000 dilution) was used as secondary antibody. Immunoblotted bands were detected by Signalfire™ ECL Reagent (Cell Signaling Technology, 50-194-072 (6883S)) and visualized by Azure™ Biosystems Gel Doc.

### Statistical Analysis

Web based application “BoxPlotR” was used to generate Box plots. Statistical analyses of the data were performed using analysis of variance (ANOVA). One-way ANOVA with a Duncan post-hoc test, and two-way analysis of variance (ANOVA) and Tukey's honest significant difference (HSD) test were used to compare mean values. For all the statistical analyses, the difference was considered statistically significant when the test yielded a p-value <0.05.

### Example 2

In this example, overexpression of ARSK1 to promote root growth and PUE is validated in crops, specifically the oil crop Camelina sativa. The ARSK1 gene from Arabidopsis thaliana will be cloned and expressed under a root-specific promoter and constitutively overexpressing promoter in the biofuel crop Camelina sativa, which is a relative of canola. These transgenic plants will be characterized in terms of root growth, phosphorus uptake, and PUE.

To generate the ARSK1 overexpressing constructs, the full coding sequence of the ARSK1 gene (AT2G26290) from cDNA isolated from A. thaliana will be amplified. This sequence will then be inserted downstream of the Cauliflower mosaic virus (CaMV) 35S constitutive promoter or the root-specific promoter roID isolated from Agrobacterium rhizogenes; sequences will be introduced through InFusion cloning into the pSMAH621 construct, which contains a hygromycin resistance selectable marker. Successfully sequenced constructs will be used to transform the Agrobacterium tumefasciens strain GV3101.

C. sativa (cultivar Suneson) will be grown on soil until fully flowering prior to Agrobacterium-mediated transformation through floral dipping. Inflorescences will be dipped in a culture of Agrobacterium transformed with either the 35S::ARSK1 or roID::ARSK1 constructs, placed horizontally for 24 hours under high humidity, and returned to the growth chamber; this process will be repeated again one week after the initial floral dip to improve transformation efficiency. Plants will then be grown until seeds are collected (approximately 90 days from seed to seed) and successful transformants will be selected by growing plants on Murashige-Skoog plates containing hygromycin. Transgenic lines will continue to be selected until the T3 generation. Elevated ARSK1 expression in established T3 lines compared to wild-type Suneson control will be further confirmed using real-time quantitative PCR (RT-aPCR) with ARSK1-specific primers; for roID::ARSK1 lines, ARSK1 expression will be measured in root compared to shoot tissue to verify root-specific expression.

ARSK1 overexpressing lines and wild-type Suneson will be grown in pots of soil watered with Hoagland's No. 2 nutrient solution containing either 1 mM NH₄H2PO₄ (+P treatment) or 1 mM NH₄Cl (−P treatment) for 10 days. In addition to these controlled conditions, plants will also be grown on P-replete and P-deficient soils obtained from MSU Kellogg Biological Station to replicate field conditions. Roots and shoots of ten biological replicates per treatment will be collected; roots will be washed with water and scanned on an Epson Perfection V700 Photo Scanner. Root architecture parameters (primary root length, lateral root density, and lateral root lengths) will be determined from the scanned images using ImageJ.

After collection, the biomass of the root and shoot tissues will be measured and total phosphorus content within the tissue samples will be determined using the Microwave Plasma Atomic Emission Spectrometer (MP-AES) 200 (Agilent) in the Rouached lab. From this data, the following metrics describing phosphorus uptake and utilization will be calculated: phosphorus uptake efficiency (increase in phosphorus concentration in the plant per unit of phosphorus applied), phosphorus utilization efficiency (increase in plant biomass per unit increase of phosphorus concentration in the plant), and physiological phosphorus use efficiency (plant biomass per phosphorus concentration at a given level of phosphorus application.

Additional measurements of phosphorus uptake in ARSK1 overexpressing lines to validate any observed differences between the transgenic lines and the wild-type will be performed using radiolabeling experiments. ARSK1 overexpressing lines and Suneson will once again be grown for 10 days in soil supplemented with ³³P and watered under a controlled −P watering regimen. After harvesting, whole shoot tissue and whole root tissue will be isolated, frozen, and analyzed to determine ³³P amounts.

Due to the enhancement of root growth observed during ARSK1 overexpression in Arabidopsis (Example 1), a similar enhancement in primary root growth when this gene is overexpressed either constitutively or under root-specific expression in the closely related crop species, C. sativa, is expected. Potential deleterious effects on plant biomass due to the ectopic overexpression of 35S::ARSK1 may additionally be mitigated in the root-specific roID::ARSK1 line.

SEQUENCES
>OAP09119.1 ARSK1 [Arabidopsis thaliana]
(SEQ ID NO: 1)
MAVFKKKKTSLTSLFLGCYKAKNVSKYEGGEKAVMKIRTCPAFKRLSLSDISDPSSPMSVMDDLSHSFTSQKLRLFTLSE
LRVITHNFSRSNMLGEGGFGPVYKGFIDDKVKPGIEAQPVAVKALDLHGHQGHREWLAEILFLGQLSNKHLVKLIGFCCE
EEQRVLVYEYMPRGSLENQLFRRNSLAMAWGIRMKIALGAAKGLAFLHEAEKPVIYRDFKTSNILLDSDYNAKLSDFGLA
KDGPEGEHTHVTTRVMGTQGYAAPEYIMTGHLTTMNDVYSFGVVLLELITGKRSMDNTRTRREQSLVEWARPMLRDQRKL
ERIIDPRLANQHKTEAAQVAASLAYKCLSQHPKYRPTMCEVVKVLESIQEVDIRKHDGNNNKEGKKFVDINKFRHHRKGK
RRVNIAYSDSLVYKESKAKQNDGI
>NM_128186.3 Arabidopsis thaliana root-specific kinase 1 (ARSK1), mRNA
(SEQ ID NO: 2)
GTACGAATAAGACACAACAACTACTATAAATTAGAGGACTTTGAAGACAAGTAGGTTAACTAGAACATCCTTAATTTCTA
AACCTACGCACTCTACAAAAGATTCATCAAAAGGAGTAAAAGACTAACTTTCTCCATTTTTTCCCCAAATAACACAAAAG
AAGAAGTTGAAAATGGCAGTGTTCAAGAAGAAGAAAACATCATTGACATCTCTGTTCTTGGGGTGTTACAAGGCAAAGAA
CGCAAGCAAATACGAAGGAGGGGAGAAGGCTGTAATGAAAATAAGAACTTGTCCAGCGTTCAAGAGGCTGTCTTTATCGG
ACATAAGCGATCCAAGCTCGCCCATGTCGGTTATGGATGATCTCTCTCACTCCTTCACATCGCAGAAGCTTCGTTTGTTC
ACGTTGTCTGAGCTGAGAGTGATTACGCATAATTTCTCTAGAAGTAACATGTTGGGGGAAGGAGGGTTTGGGCCGGTTTA
CAAAGGGTTTATTGATGATAAGGTTAAACCGGGTATCGAAGCCCAACCGGTTGCTGTAAAGGCTCTTGATCTTCATGGCC
ATCAAGGCCATAGAGAATGGCTGGCGGAGATATTATTTTTGGGACAACTAAGTAACAAACATCTTGTGAAGCTGATAGGG
TTTTGTTGTGAAGAAGAGCAAAGAGTTCTTGTTTATGAGTACATGCCTCGAGGTAGCTTGGAGAATCAGCTCTTTCGAAG
AAATAGCTTAGCAATGGCTTGGGGAATAAGGATGAAGATAGCTCTCGGAGCCGCCAAAGGTTTAGCTTTTCTCCATGAAG
CAGAGAAACCTGTCATCTACAGAGACTTTAAAACCTCTAACATATTGTTAGACTCCGACTATAACGCCAAATTATCTGAC
TTCGGTCTAGCCAAAGATGGACCTGAAGGCGAACATACGCATGTAACTACCCGAGTGATGGGAACACAAGGTTATGCTGC
GCCCGAATATATCATGACTGGTCATTTGACGACGATGAATGATGTATACAGTTTTGGAGTAGTTTTGTTAGAGCTGATAA
CCGGAAAACGGTCAATGGACAATACCCGAACACGGAGGGAACAAAGCCTAGTGGAATGGGCGCGGCCTATGTTAAGGGAC
CAAAGAAAGTTAGAGAGGATAATTGACCCGAGGCTCGCGAACCAGCACAAAACAGAAGCGGCTCAGGTAGCTGCTTCCCT
AGCATACAAGTGTCTAAGCCAACATCCTAAGTATAGACCTACAATGTGTGAGGTGGTTAAGGTCTTAGAGTCTATCCAAG
AAGTTGATATTAGGAAGCATGATGGCAATAACAACAAAGAAGGAAAGAAGTTTGTTGATATAAATAAGTTTAGGCATCAC
CGTAAAGGCAAAAGACGTGTGAACATCGCTTATTCAGATTCTCTCGTTTACAAGGAATCCAAGGCAAAGCAAAACGATGG
CATTTGAAATACATCATTTTCTCATATGAATTATTAAGCATTCATATTCTATGAACGCAATTCTTATCTTAAGTTATGAC
CATTCTTCCAAAAATTTGAAGGAAAGAAAGGTGATGTTATAAGTATCCTAGTTCTCTGGTATTTTAGATTAATTTCCTTT
ACATAATTCAAAAATACTTTGTACAAAAGAGTCATGTACAAGAAATATAAAATAAATGTTTGTAAATCATATATATATAC
ACCAGTTTGTTGGGCGGACAACGTTG
>NP_001346584.1 putative protein kinase superfamily protein [Zea mays]
(SEQ ID NO: 3)
MGKPAESSSTSWSALFGFGCFSSSHADRSGDGPGKVASAGSRPPAPPAPLLSPDDLSLSLAGSDVLAFTVEELRVATRDF
SMSNFVGEGGFGPVYKGRVDERVRPGLRQPQAVAVKLLDLEGSQGHKEWLAEVIFLGQLRHPHLVKLIGYCYQDEHRLLV
YEFMARGSLEKHLFKRHSASLPWSTRMRIAIGAAKGLAFLHDAAKPVIYRDFKTSNILLDSDYTAKLSDFGLAKAGPGED
ETHVSTRVMGTQGYAAPEYIMTGHLTTKSDVYSFGVVLLELLTGRKALDKNRPPREQSLVEWARPCLRDARRLERVMDRR
LPRPTTPTRAAQKAAGIAHQCLSVSPKSRPQMSHVVQALESLLALDDAAVEPFVYTAPPESR
>XM_008653310.4 PREDICTED: Zea mays serine/threonine-protein kinase
(LOC103631789), transcript variant X1, mRNA
(SEQ ID NO: 4)
CACTCCTCCCCCCTGGAGGATTAAGCGTAATCAACCCACCCCACACAGCCACCGTATATGTATATATACCCCGATGCGTC
CAGCCAAAGCCGCTGGTGGGAGGCCGGCCGGGTAAAGGTAGTAAACCGTCGCAGCGCGCGGAGTCCCTCCATCTCCATCC
GGCGACCGACCAACCGACCTAGACTAGACGCGGCCGGCCGGGCATGGCGAGGCCGGGGTGGGGCTCGCTGTTCGGCTGCT
TCAGCAGCAGCAGCAGCTCCCGCGGGGACGGTAAGAAGAAGCAGGGCAAGGGCAAGGGAAAGGGCGCCGGGAATAAGATG
AAGAAGAAGCAGAAGAGCAAGCAGCAGCAGAAGGTAGCGGCGGGGAGCGGCGGCGGTAGCCGGCCGCGGTCGCTGCAGAG
CCGGATGTCGTTCACGGAGCTGAGCCTCAGCGGGATGGTGTCGCCCGAGGACCTGTCGCTCTCGCTCGTCGGCTCCAACC
TCCACGTCTTCACCATCGCCGAGCTGCGCGCCGTCACCCGCGACTTCTCCATGACCCACTTCATCGGCGAGGGCGGCTTC
GGCCCCGTCTACAGGGGCTACGTCGACGACAAGACCAAGCGGGGGCTCGCCGCGCAGCCCGTCGCCGTCAAGCTGCTCGA
CCTCGAGGGCGGCCAAGGACACACCGAATGGCTGACGGAGGTCTTCTTCCTGGGACAACTGAGGCACCCTCACCTCGTGA
AGCTGATCGGCTACTGCTACGAGGACGAGCACAGGCTGCTCGTCTACGAGTTCATGACCAGGGGCAGCCTGGAGAAGCAT
CTCTTCAAAAAGTACGCGGCGTCGTTGCCTTGGTCCACACGGCTGAAGATCGCCATTGGCGCTGCCAAAGGGTTGGCCTT
CCTCCATGAAGCCGAGAAGCCGGTCATCTACCGGGACTTCAAGACCTCCAACATCCTGCTGGACTCGGATTACAAGGCGA
AGCTGTCGGATTTCGGTCTAGCCAAAGACGGGCCGGAAGACGACGAGACGCACGTGTCGACCCGGGTGATGGGGACCCAA
GGATACGCCGCGCCGGAGTACATCATGACGGGCCACTTGACGGCGAAGAGCGACGTGTACGGCTTCGGCGTGGTGCTGCT
GGAGCTGCTGTCGGGGCGGAAGGCGGTGGACAAGAGCCGCCCCCCGCGGGAGCAGAACCTGGTGGAGTGGGCGCGGCCGT
ACCTGACGGACGCGCGCCGCCTGGACCGCGTCATGGACCCCAGCCTGGTGGGCCAGTACTCCAGCCGGGCCGCGCACAAG
GCCGCCGCCGTGGCGCACCAGTGCGTCGCCCTCAACCCCAAGTCCCGCCCACACATGTCCGCCGTCGTCGAAGCGCTCGA
GCCGCTCCTCGCGCTCGACGACTGCCTCGTCGGGCCCTTCGTCTACATCGCACCGCCGGAGACGACGACCGCCAACGGGG
ACGAAGCGGCGCCGGTGAGTAGTAATAAGGAAGGATCCGGCAGACGAGGAGGCCGGAGGAGGTCGTCAGGCGAAGGCGCG
GCCGTCGTGCGGCCAGAGGAGTGATGTTCCCTCTTCCGTCCGTGCTATGACGACGATGATAATATGGGTGGATTTACTAG
CTTCCACGTCTTGTACGTGACCGGACTGTTGTTGCCTGTCGCGTGCATGCAGCTTGGCTGAGATATATCTATCGATCGAG
CAGCTATACGATCGGTGGAAGTTAACGTCTTCGTTTGTGTGATTGAAAGCATCCAGCTGGTTGCGTGTTTTAGAGAGAAC
TCTATAGATTGAATTTGTTGCATATACAGTAATCTAACCAAACTTCTCTATTTTTATAGATCTAAAACGACTTGTA
>XP_003542362.1 serine/threonine-protein kinase RIPK [Glycine max]
(SEQ ID NO: 5)
MGSSFSSCYEGESVSPSPKPTKVVATKGGSSSNRVSITDLSFPGSTLSEDLSVSLVGSNLHVFSLSELKIITQSFSSSNF
LGEGGFGPVHKGFIDDKLRPGLEAQPVAVKLLDLDGSQGHKEWLTEVVELGQLRHPHLVKLIGYCCEEEHRLLVYEYLPR
GSLENQLFRRYTASLPWSTRMKIAAGAAKGLAFLHEAKKPVIYRDFKASNILLDSDYNAKLSDFGLAKDGPEGDDTHVST
RVMGTQGYAAPEYIMTGHLTAMSDVYSFGVVLLELLTGRRSVDKGRPQREQNLVEWARPALNDSRKLGRIMDPRLEGQYS
EVGARKAAALAYQCLSHRPRSRPLMSTVVNVLEPLQDFDDVPIGPFVYTVPAEQHNEVAKESETPKERKRENDHHHHNRH
HHHHRHNGHRHHPLKSPKTPMSSDQSQNDEHRNGRRSGSNSPDTSNASEAQ
>NM_001251652.1 Glycine max kinase-like protein (LOC100305397), mRNA
(SEQ ID NO: 6)
ATGGGATCAAGCTTCTCTAGTTGTTATGAGGGTGAATCTGTGTCTCCTTCTCCAAAGCCAACCAAAGTGGTGGCTACGAA
GGGTGGTTCTTCTTCCAACAGGGTCTCTATAACGGACTTGAGTTTTCCTGGCTCAACGCTTTCGGAGGATCTCTCCGTTT
CTCTAGTGGGGTCTAACCTGCATGTGTTTTCACTCGCTGAGCTCAAGATCATAACGCAGGGTTTCTCTTCGAGTAACTTT
CTCGGAGAAGGAGGTTTCGGACCCGTGCATAAGGGCTTCATTGATGACAAGCTTAGGCCTGGTCTCGAGGCTCAACCGGT
TGCCGTCAAGCTTTTGGACTTGGACGGTTCTCAGGGCCATAAAGAGTGGTTGACTGAAGTTGTGTTTCTGGGTCAACTAA
GGCATCCACATCTCGTGAAGCTGATTGGATATTGCTGTGAAGAAGAACACAGGCTTCTGGTATATGAGTATTTACCACGA
GGCAGCTTAGAGAATCAACTATTTAGAAGATATACGGCATCGTTACCATGGTCAACAAGAATGAAAATTGCAGCTGGAGC
TGCAAAGGGTCTAGCCTTTCTACATGAAGCAAAGAAACCAGTCATCTATAGAGATTTTAAAGCTTCAAACATCTTGTTAG
ACTCGGATTATAATGCAAAACTTTCCGATTTTGGCTTGGCCAAAGATGGTCCTGAAGGAGATGACACACACGTTTCAACT
AGAGTCATGGGCACACAAGGCTATGCGGCCCCAGAATACATCATGACAGGACACTTGACAGCAATGAGTGATGTGTACAG
TTTTGGAGTCGTGCTCTTGGAGCTTCTAACAGGACGAAGGTCAGTGGACAAAGGGCGCCCACAAAGAGAACAGAACCTCG
TGGAATGGGCCAGGTCTGCTTTGAATGATTCCCGGAAACTTAGCAGAATAATGGATCCAAGGCTTGAGGGTCAGTATTCT
GAGGTTGGGGCAAGAAAAGCAGCTGCATTGGCTTACCAATGCCTTAGCCACAGGCCAAGGAGTAGACCCTTGATGAGCAC
TGTGGTTAATGTTCTCGAGCCACTTCAGGACTTTGATGATGTTCCAATTGGACCCTTTGTTTACACTGTTCCAGCTGAAC
AACAACAATACAATGAAGTCGCAAAAGAGAGTGAAACACCTAAGGAGAGGAAGAGGGAAAATGGTCATCATCATAACCGA
CGTCATCATCATCATCGCCACAATGGTCATAGGCATCATCCACTTAAATCTCCTAAGACACCAATGCCATCAGATCAGTC
ACAGAATGATGAACACCGAAATGGTAGAAAAAGTGGGTCTAATTCACCTGATACATTCAATGCATCAGAAGCTCAACGAA
GCATGGGTTAG
>XP_013695593.1 probable serine/threonine-protein kinase PBL12 [Brassica napus]
(SEQ ID NO: 7)
MAVTKNKKTSLTSLFLGCYRTKNASRYEVEERGNAMKIRTCPVIKRLSLSDISDPSSPMSVMDDLSNSFTSQKLRMFTLS
ELRVITHNFSRSNMLGEGGFGPVYKGFIDDKVKPGLEAQPVAVKALDLHGHQGHREWLAEIIFLGQLSNKHLVKLIGFCC
EEEQRVLVYEYMPRGSLENQLFRRNSVAMAWGIRMKIALGAAKGLAFLHEAEKPVIYRDFKTSNILLDCNYNAKLSDEGL
AKDGPEGEHTHVTTRVMGTQGYAAPEYIMTGHLTTMNDVYSFGVVLLELITGKRSVDTTRARREQSLVEWARPMLRDQRK
LDRIIDPRLECQYKTEAAQAAAALAFKCLSQNPKYRPTMSDVVKVLESIQEVETKERNSNNKETKKFVDKRIIRHHRKGQ
KRVNIAYSDSLLYKESRAKQNNGV
>XM_013840139.2 PREDICTED: Brassica napus probable serine/threonine-protein
kinase PBL12 (LOC106399681), mRNA
(SEQ ID NO: 8)
TAGGTTAACTAAAACACCCTTAATTTCTAAACCTACGCAATCTACAAAAGATACATCAAAAAGAGTAAAACACTATCTAT
CTTCCATTTTCTCCAAAAAGTTGAAGAAGAAGAAGAAGAAGAAGAAGAAGAAGAAAATGGCAGTAACCAAGAACAAGAAG
ACATCATTGACATCTCTGTTTTTAGGATGTTACAGGACAAAGAACGCGAGTAGATACGAAGTAGAGGAGAGGGGCAACGC
AATGAAAATAAGAACTTGTCCGGTGATCAAGAGGCTGTCGTTATCGGACATAAGTGATCCAAGCTCGCCCATGTCGGTCA
TGGATGACCTCTCAAACTCCTTCACATCTCAAAAGCTTCGTATGTTCACCTTGTCTGAGCTGAGAGTTATTACGCATAAT
TTCTCAAGAAGTAACATGCTGGGAGAAGGAGGGTTTGGGCCAGTTTACAAAGGGTTCATTGATGACAAGGTTAAACCGGG
TTTAGAAGCCCAACCGGTTGCTGTTAAGGCACTTGATCTCCATGGTCACCAAGGCCATAGAGAATGGCTGGCGGAGATAA
TCTTTTTGGGACAGCTAAGTAACAAACATCTCGTGAAGCTGATAGGATTTTGTTGTGAAGAAGAGCAAAGAGTACTTGTT
TATGAGTACATGCCTCGAGGTAGCTTGGAGAATCAGCTCTTTAGACGAAACAGTGTAGCAATGGCATGGGGAATAAGGAT
GAAGATAGCCCTTGGAGCCGCTAAAGGCTTAGCTTTTCTCCATGAAGCAGAGAAACCTGTCATCTACCGAGACTTCAAAA
CCTCCAACATATTGTTAGACTGCAACTATAACGCAAAATTATCTGATTTCGGCCTAGCCAAAGATGGACCTGAAGGAGAA
CATACACATGTAACAACTCGAGTGATGGGAACGCAAGGTTATGCTGCACCCGAATATATCATGACTGGTCACTTGACGAC
AATGAATGACGTCTACAGTTTTGGAGTCGTTCTGTTGGAACTGATAACAGGGAAACGGTCAGTGGACACTACCCGGGCAC
GAAGGGAACAAAGCCTCGTGGAATGGGCGCGTCCAATGTTGAGGGACCAAAGAAAGCTAGATCGGATAATCGATCCAAGG
CTCGAATGCCAGTACAAAACTGAGGCAGCTCAGGCAGCAGCTGCCTTAGCCTTCAAGTGTCTAAGCCAAAATCCCAAGTA
TAGACCAACAATGTCTGACGTAGTAAAGGTTTTGGAGTCTATCCAAGAAGTTGAGACAAAGGAGCGTAATAGTAACAACA
AAGAAACAAAGAAGTTTGTTGATAAAAGAATCATTAGACATCACCGTAAAGGCCAAAAAAGAGTGAACATCGCTTATTCA
GATTCTCTCCTTTACAAGGAATCCAGGGCAAAGCAGAACAATGGGGTTTGAAATACATCAGTTTTCATATTAATTATTAA
GCATTGATATTTCTATATATGCACTTCTTATATTTAGTTCTAAGAGCCTTCTTCCATAAATTTTGGGATGGAAAGGTGGT
GTTATTTAATGTTAAAAAAAAAAGGTGGTATTATTTCTCCTAGTTATCTGGGGTTTCAGATTAG
>XP_040973069.1 serine/threonine-protein kinase RIPK-like [Gossypium hirsutum]
(SEQ ID NO: 9)
MTVKKKITWKSLMPSCYKSKDTSDSGENRLKLKPCQFQRLSLSDVSDPSSPICVNGLSTSLFGSNLYVFTLAELRLITHN
FARCNLLGEGGFGPVYKGFVDDKLRPGLKAQPVAVKTLDLDGLQGHREWLAEIIFLGQLRHPHLVKLIGYCYEDENRLLV
YEYMPRGSLENQLFRRYSATLPWSTRMKIALGAAKGLAFLHEADKPVIYRDFKSSNILLDSDYNCKLSDFGLAKDGPEGG
ETHVTTRVMGTQGYAAPEYIMTGHLTTRSDVYSFGVVLLELLTGRRSMDNTRPSREQSLVEWARPLLRDPKRLDRVIDPR
LEGQYSNKGAQKVAALAYKCLSHYPKPRPTMGDVVIVLESLQGFEDEFVGPFVYVVPNETENSNEFLAKKGENEHDSPLR
GWRNRIKLPPSSVANAESPCSI
>XM_016897685.2 PREDICTED: Gossypium hirsutum probable serine/threonine-protein
kinase PBL12 (LOC107961630), mRNA
(SEQ ID NO: 10)
GTCTTCTCACGCCCAAAGAAAAGTGAAAATGTGTTTGAAAAATTAGCAAATTATTTTAAAAAGAAAATGAAGAGGGCATT
GTATACATGAAGCATATAATTACTTTTTTTTCCATCTAAATTTTAAATACATTCGGATTTGAGAAGTGGTAGGTAAGTGA
ATGAATTACTATAAAAATCTCTAAAGCCTTCACAAAAATCCATAGACCCTGGCAGAAATTTTCCATTTTCTTTGTATTTT
CTTGCATATTGCAAAAGCAAACAATTCATTCAAACATGACAGTGAAGAAGATTACATGGAAATCTCTCATGTTAAGCTGT
TACAATAGCAAGGACATCATCTCTCCATCTGATTCAGGGGAGAAGGATTCGAAACCATGTCAATTCCAAAGACTATCAAT
GTCCGACGTAAGTGATCGCAGCTCGCCACTCTCTGTCGACGATCTCTCGACCTCTCTACTCGGCTCAAATCTTCATGTGT
TTACCTTTGCGGAGCTAAGACTAATAACCCATAATTTCGCACGCTGTAATCTGCTCGGTGAAGGAGGGTTCGGACCGGTT
TACAAAGGTTTCATTGATGACAAGCTTAGGCCTGGATTGAAGGCTCAGCCGGTTGCTGTCAAGGCACTGGACTTGTATGG
CTTGCAGGGTCATAGGGAGTGGCTGGCTGAAATAATCTTTCTTGGTCAACTACGGCATCCTCATCTGGTTAAGTTGATTG
GATATTGTTATGAAGAAGATAACAGAGTTTTAGTGTATGAATATATGCCAAGAGGAAGCTTAGAAAATCAACTCTTTAGA
AGGTATTCAGCTCCCTTACCATGGTCTGCTAGGATGAAAATTGCCTTGGCAGCAGCAAAGGGCCTGGCTTTCCTACATGA
AGCTGATAAACCAGTAATATTTCGGGACTTTAAATCTTCAAACATCTTGTTAGACTCTGATTACAATTGCAAACTATCGG
ATTTCGGGTTAGCAAAGGATGGTCCAGAAGGCGAACAGACTCATGTAACGACCCGAGTTATGGGCACACAAGGATATGCA
GCTCCAGAGTATATCATGACTGGTCATTTGACAACAATGAGTGATGTGTACAGCTTTGGAGTGGTTCTCTTAGAGTTACT
AACAGGGAAACGATCTGTGGACAATAGCCGGCCCGGTCGAGAGCAGAGCCTTGTGGAATGGGCAAGGCCATTATTAAGGG
ACCCTAAGAAGCTTGATAAGCTAATAGATGCTAGACTTGAGAGACAATTTTCCAACAAGGGGGCTCAAAAGGTGGCTGCG
TTGGCATACAAATGCTTGAGCCACCAACCAAAGCCAAGGCCTAGCATGGGCGATGTGGTCAAGATCCTGGACTCTGTTCA
GGGGTTTGAGGATGAATTTGTTGGACCTTTTGTTTATGTGGTGCCAAATGAAACCGATGGTGACAAGGAGTTGTTTACGA
CCAAGGATTGCGGTGTAGAAAAACACGAACTACATCGTTGTGGTTGGCGAAATGATCAAATTGCCTCTTTCTTTGGTAGC
TAATGCTGAATCTCCATGTGGCATAAGCATTTGAGAATTGCAGATATATGTGGTCGAATTTATGTGGTGCGAAATGAAAA
ACAAAGGTAGAAATTTCGACTGGAAA
>AGT16920.1 hypothetical protein SHCRBa 201 A23 R 220 [Saccharum hybrid cultivar
R570]
(SEQ ID NO: 11)
MTSPKRAAGREGCGCWAAVARGLRGACFRPAAPADGDGGGSAKGSHVHDAAAETRYLNASNRELGDHFQTKHDGENGVDA
SIEKRTPKLLQFTFQELKSATLNFRPDSILGEGGFGYVFKGWIEPNSTAPAKPGTGVTVAVKSLKPDALQGHREWVAEVD
FLGQLHHKHLVKLIGYCIEDDQRLLVYEFMARGSLENHLFRRALPLPWSNRMKIALGAAKGLAFLHGGPKPVIYRDEKTS
NVLLDAEYNAKLSDFGLAKAGPQGDKTHVSTRVVGTYGYAAPEYVMTGHLTSKSDVYSFGVVLLEMLTGRRSMDKKRPTG
EQNLVAWARPYLNDRRRLYQLIDPRLGLNYSVKGVQKVAQICHYCLTRDSKSRPSMDEVVKQLTPLQDLNDMASASPRPR
STQRGE
>AGT16920.1 hypothetical protein SHCRBa 201 A23 R 220, mRNA [Saccharum hybrid
cultivar R570]
(SEQ ID NO: 12)
CTACTCACCGCGCTGGGTTGAGCGAGGCCTTGGCGATGCAGATGCCATGTCATTTAGATCTTGTAATGGTGTGAGTTGCT
TGACAACTTCATCCATGGATGGACGAGACTTACTATCACGGGTAAGGCAATAGTGGCATATCTGAGCAACCTTTTGCACC
CCTTTGACAGAGTAGTTCAGCCCCAAGCGAGGATCTATGAGCTGATAGAGCCTTCGCCGATCATTCAGGTAAGGCCTCGC
CCATGCAACCAGGTTCTGCTCCCCTGTAGGTCGCTTCTTGTCCATTGATCTTCTCCCAGTCAACATCTCGAGTAACACAA
CTCCGAAGCTATAGACATCACTCTTAGATGTCAAGTGCCCTGTCATTACATACTCTGGTGCAGCATAACCGTAAGTGCCA
ACAACCCGAGTAGATACATGAGTTTTATCACCCTGAGGACCAGCTTTTGCTAAACCGAAATCAGATAGTTTTGCGTTGTA
TTCCGCATCAAGAAGAACATTCGATGTCTTAAAATCCCTGTAAATAACAGGTTTGGGACCTCCATGGAGGAAGGCTAGTC
CTTTCGCGGCACCTAGAGCTATCTTCATTCTGTTGGACCAAGGTAGGGGAAGGGCCCTTCTGAAAAGATGGTTTTCAAGA
CTTCCCCGTGCCATGAATTCATATACAAGTAACCTTTGATCATCCTCAATACAATATCCAATCAGCTTAACAAGGTGTTT
ATGATGCAACTGTCCCAGAAAGTCAACTTCTGCCACCCATTCTCTATGACCTTGAAGCGCATCTGGCTTCAAACTTTTGA
CTGCCACAGTAACCCCTGTGCCAGGTTTTGCAGGAGCAGTGCTGTTTGGCTCAATCCACCCCTTGAAGACATAACCAAAC
CCACCTTCCCCAAGAATACTGTCTGGCCTGAAGTTAAGGGTGGCAGATTTCAACTCTTGAAAGGTAAACTGAAGTAGCTT
GGGTGTTCTCTTTTCAATTGACGCATCAACACCATTTTCACCATCATGTTTTGTCTGAAAATGATCACCAAGCTCCCGAT
TGCTAGCATTCAGGTATCTTGTCTCTGCTG
>XP_015612825.1 serine/threonine-protein kinase RIPK [Oryza sativa Japonica
Group]
(SEQ ID NO: 13)
MGKKTTTSSSPSWSSLFGLGCFTSSHSDGGSAAAKNPGTPLPARPSSCNSNDGVAAAVMPSPEDLSQSLAGSGVEAFTVE
ELRRATRDFSVSNFVGEGGFGPVYKGYVDERLKPGVRAQAVAVKLLDLEGSQGHKEWLAEVIFLGQLRHHHLVKLIGYCY
EDEHRLLVYEFMARGSLEKHLFKKYSASLPWSTRLKIAIGAARGLAFLHEAAKPVIYRDFKTSNILLNSDYEAKLSDFGL
AKDGPQEDETHVSTRVMGTQGYAAPEYIMTGHLTTKSDVYSYGVVLLELLTGRKAVDKKRPPREQNLVEWARPCLHDSRR
LNRVIDKSLNGQYSTRAVQKAAAIAYQCLSVSPKSRPRMSAVVEALEPLLAMDDGIVEPFVYMAPPESK
>XM_015757339.2 PREDICTED: Oryza sativa Japonica Group serine/threonine-protein
kinase RIPK (LOC4348546), mRNA
(SEQ ID NO: 14)
CACCCCACCCACAAATTAAACACACCACCCTCAATATATATCATCATCATCATCCCCCAATTCCCAACACCGCGACGTGC
TTCCTATAAATTAACTCCCCCCGATCCTCCTCCCAACCATTCCACACCTTGGCAATCATGGGGAAGAAGACGACGACGTC
GTCGTCGCCGTCGTGGAGCTCGCTGTTCGGGCTCGGCTGCTTCACCTCCTCCCACAGCGACGGCGGCTCGGCGGCGGCCA
AGAACCCGGGGACGCCGCTGCCGGCGCGGCCGTCGTCGTGCAACAGCAACGACGGGGTGGCGGCGGCGGTGATGCCGTCG
CCGGAGGACCTGTCGCAGTCGCTGGCGGGGAGCGGGGTGGAGGCGTTCACGGTGGAGGAGCTGAGGAGGGCGACGCGGGA
CTTCTCGGTGAGCAACTTCGTGGGCGAGGGGGGGTTCGGGCCGGTGTACAAGGGGTACGTCGACGAGCGGCTCAAGCCCG
GCGTGCGCGCGCAGGCCGTCGCCGTCAAGCTGCTCGACCTCGAGGGCTCCCAGGGCCACAAGGAATGGCTGGCTGAAGTC
ATATTTCTTGGGCAATTGAGACATCATCACCTCGTCAAGCTGATCGGATACTGCTACGAGGATGAGCACAGACTTCTCGT
CTACGAGTTCATGGCTAGGGGCAGCTTGGAGAAGCACCTCTTCAAAAAGTACTCTGCGTCGCTGCCGTGGTCAACACGCT
TGAAGATCGCCATTGGAGCGGCCAGAGGTTTGGCCTTCCTCCATGAAGCTGCCAAGCCCGTCATCTATCGCGACTTCAAG
ACCTCCAACATCCTACTCAACTCGGACTACGAAGCAAAACTTTCGGATTTCGGACTGGCCAAAGATGGGCCTCAAGAAGA
CGAGACACATGTCTCCACTAGAGTGATGGGCACACAGGGCTATGCCGCACCAGAATACATCATGACTGGTCATCTAACGA
CAAAGAGCGATGTGTATAGCTATGGTGTGGTGCTGCTGGAGCTACTAACCGGACGGAAAGCCGTGGATAAGAAGAGACCG
CCGCGAGAGCAAAACCTAGTGGAATGGGCTCGGCCATGCCTACATGACTCTCGCCGGCTCAACCGTGTCATCGACAAAAG
CTTGAACGGGCAGTACTCGACTCGAGCCGTACAAAAGGCTGCGGCCATAGCGTATCAGTGCTTGAGTGTTAGCCCTAAGT
CTCGACCCCGGATGTCAGCGGTTGTGGAGGCCCTGGAACCACTTCTGGCCATGGATGATGGCATTGTAGAGCCATTTGTG
TACATGGCACCACCCGAGAGTAAATAAATGAATCAATAATTTGTGTGGAGCATATGTACAAACATGCCATTCTCCAATCC
TTGCAATCAATTTAAAATATAGAAAATTCTCCATTTGCATTTAGAAATTTCAGCATATCCCTGTATGCCTCTGAGTTTTT
CTTAAGAGTAAATTATATTGGTGATACA
>XP_006355769.1 PREDICTED: putative receptor-like prQtein kinase At1g72540
[Solanum tuberosum]
(SEQ ID NO: 15)
MKITWESLVPSCCISENPKPKPKQSKLVTKQSSFHRISVSDFSGSEDLSISLAGSNLHIFSVQELKVITQNESSSNELGE
GGFGPVHKGFIDDKLRPGLKAQPVAVKLLDLDGTQGHREWLTEVIFLGQLRHPHLVKLIGYCCEEEHRLLAYEYMPRGSL
ENQLFRRYSVSLPWSTRMKIALGAAKGLAFLHEAEKPVIYRDFKASNILLDSDYNAKLSDFGLAKDGPEGDDTHVSTRVM
GTQGYAAPEYLMTGHLTAASDVYSFGVVLLELLTGRRSVDKSRPNREQNLADWARPQLKDARKLGRIIDPRLEGLYSEEG
VQKAALVAYQCLSHRPKARPDMSTVVKTLETLKDYKDISTMTFVYIAPVDQHKQSPQKELVNKRNTNSHHHTKSPHKRLT
PNSPLHNDFHRA
>XM_006355707.2 PREDICTED: Solanum tuberosum putative receptor-like protein
kinase At1g72540 (LOC102599519), mRNA
(SEQ ID NO: 16)
GAGCAGTTGGGTACGACATATTTGAATATTATTATTACCCTCTATTCCTTTCTTGCTCATCAATCATTTTTCTATATATA
TAAACACACAATTCTCTTCAAGGCAATTAAGAGAATTTATTCATTTTATTTCAATTAAAGCTAGCGATGAAGATTACATG
GGAATCTCTAGTACCTAGCTGTTGTATATCCGAAAATCCAAAGCCAAAGCCAAAGCAATCGAAATTAGTAACAAAACAAA
GTTCATTTCATCGGATTTCAGTTTCAGATTTCAGTGGTTCAGAAGATTTATCAATATCATTAGCTGGTTCAAATCTTCAT
ATTTTCAGTGTTCAAGAATTGAAAGTGATTACTCAGAATTTTTCGTCGAGTAATTTTCTAGGGGAGGGAGGTTTTGGACC
AGTTCATAAAGGTTTTATTGATGATAAACTTAGACCTGGTCTAAAAGCTCAACCTGTTGCTGTAAAGCTTTTAGATTTAG
ATGGCACTCAAGGTCATCGAGAATGGCTGACAGAAGTGATATTTTTGGGGCAATTGAGACATCCACATCTGGTGAAGTTG
ATTGGATATTGTTGTGAAGAGGAACACAGGCTGTTGGCCTATGAATATATGCCCAGAGGCAGCTTGGAGAATCAACTTTT
TCGAAGATATTCTGTATCACTTCCATGGTCAACGCGGATGAAAATAGCACTTGGAGCTGCAAAAGGCTTAGCTTTCCTCC
ATGAAGCTGAAAAACCAGTCATATATCGTGATTTCAAGGCTTCAAACATCTTGTTAGACTCCGATTACAATGCTAAACTG
TCAGATTTTGGACTTGCAAAAGATGGGCCAGAAGGAGATGACACACACGTCTCCACCCGAGTCATGGGAACGCAAGGCTA
CGCTGCTCCTGAATACCTCATGACCGGTCATTTGACAGCTGCAAGTGATGTATACAGTTTCGGAGTAGTATTACTGGAGC
TTCTAACTGGGAGAAGATCGGTAGATAAAAGTCGTCCAAACAGAGAACAAAATTTAGCAGATTGGGCAAGACCTCAATTG
AAAGATGCGCGTAAACTAGGTAGAATAATTGATCCAAGATTAGAAGGTTTATATTCAGAAGAAGGGGTTCAAAAAGCAGC
ATTAGTAGCTTACCAATGCTTAAGCCATAGGCCAAAAGCTAGACCAGATATGAGTACAGTTGTCAAAACACTTGAAACAT
TGAAGGATTATAAGGATATTTCAACCATGACATTTGTGTATATAGCTCCAGTTGATCAACACAAACAAAGTCCTCAAAAG
GAATTAGTCAACAAAAGAAATACTAATTCTCATCATCATACAAAAAGTCCACATAAGAGATTAACTCCAAATTCACCATT
GCACAATGATTTCCATAGAGCTTAAACAATTGTATACATCCACATTTTTTTAATTTTTTTTACCTCATAATACTCAAAAT
TCTTTATTTTTTCATTGAGTGTATTTGCTAGATATCAGTGTTAGTGAAGACAAGGCAGATATCAACTTTGTATATGTTAT
ATTTCCTTTTGGAGTTTTTAAAGTAATATATACATACCGTTTAAATAA
>XP_044362147.1 serine/threonine-protein kinase RIPK-like A genome [Triticum
aestivum]
(SEQ ID NO: 17)
MARSAWRSLFGCESSHAEAAAAAGKGEKMSKASKKKKSKKKVAAAGKAPRPSRSLHGRMSFSDLSMSGGMVSPEDLSLSL
AGSDLHVFTIAELRAVTRDESMTNFIGEGGFGPVYKGYVDEKVKPGLRAQPVAVKLLDLEGNQGHNEWLTEVIFLGQLRH
PHLVKLIGYCYEDEHRLLVYEFMTRGSLEKLLFKKYAASLQWSTRLKIALGAAKGLAFLHEAENPVIYRDFKTSNILLDS
DYKAKLSDFGLAKDGPEEDETHVSTRVMGTQGYAAPEYIMTGHLTAKSDVYGFGVVLLELLTGRKSVDKSRPPREQNLVD
WARPYLNDSRRLDRVMDPNLAGQYAGGAVQKAAALAYKCVSLNPKSRPHMSAVVSALEPLLALEEDSLAGPFVYVAPAEN
GNSGGGREGRHRRRAGRRKSGDAAESVAVVVERE
>XM_044506212.1 LOC123084719 [organism = Triticum aestivum] [GeneID = 123084719]
(SEQ ID NO: 18)
GCAGCAGGTAGAGCAGAGCGACGCGCACACGCGGAAGGCACGCACGGATGGCGAGGTCGGCGTGGAGGTCCCTCTTCGGC
TGCTTCAGCTCGCACGCCGAGGCCGCGGCCGCCGCCGGCAAGGGCGAGAAGATGAGCAAGGCCTCCAAGAAGAAGAAGAG
CAAGAAGAAGGTGGCGGCGGCGGGGAAGGCGCCTCGGCCGAGCCGGTCGCTGCACGGCCGGATGTCCTTCTCGGACCTCA
GCATGAGCGGCGGCATGGTGTCGCCGGAGGACCTGTCGCTGTCTCTGGCGGGGTCGGACCTGCACGTCTTCACCATCGCC
GAGCTGCGCGCCGTCACCAGGGACTTCTCCATGACCAACTTCATCGGCGAGGGCGGCTTCGGCCCCGTCTACAAGGGCTA
CGTCGACGAGAAGGTCAAGCCGGGGCTCCGCGCGCAGCCCGTCGCCGTCAAGCTGCTCGACCTCGAGGGCAACCAGGGCC
ACAACGAGTGGCTGACCGAAGTCATATTCCTGGGGCAGTTGAGGCATCCTCACCTGGTCAAACTGATCGGTTACTGCTAC
GAGGACGAGCACAGGCTCCTCGTGTACGAGTTCATGACCAGGGGCAGCCTAGAGAAGCTTCTCTTCAAAAAATACGCGGC
GTCGTTGCAATGGTCAACACGGCTGAAGATCGCCCTCGGCGCCGCCAAAGGGTTGGCGTTCCTCCATGAGGCCGAGAACC
CGGTCATCTACCGGGACTTCAAGACCTCCAACATCCTGCTAGACTCGGATTACAAAGCGAAGCTATCGGATTTCGGGCTA
GCCAAAGATGGACCAGAGGAAGACGAGACGCACGTCTCCACCCGGGTCATGGGCACGCAGGGCTACGCCGCACCGGAGTA
CATCATGACAGGGCATCTGACGGCCAAGAGCGACGTGTACGGTTTCGGCGTGGTGCTGCTGGAGCTGCTCACCGGGCGCA
AGTCGGTGGACAAGAGCCGGCCGCCGCGGGAGCAGAACCTGGTGGACTGGGCGCGCCCCTACCTCAACGACTCCCGGCGC
CTGGACCGCGTCATGGACCCCAACCTCGCGGGGCAGTATGCCGGCGGGGCCGTGCAGAAGGCCGCGGCCCTGGCGTACAA
GTGCGTCAGCCTCAACCCCAAGTCCCGCCCGCACATGTCCGCCGTCGTCAGCGCGCTCGAGCCGCTCCTCGCGCTCGAGG
AGGATAGCCTCGCCGGGCCCTTCGTGTACGTTGCGCCGGCTGAGAACGGGAACAGCGGCGGCGGCAGGGAGGGGCGCCAC
AGGAGGCGAGCGGGCCGGAGGAAGTCCGGAGACGCGGCCGAGTCGGTGGCCGTTGTTGTTGAGCGCGAGTGATGGTCTTG
TGTAAGCGTGTTTCCGAACTTGGACGGGTCGAACTCGAACAGTGGGGAGGAGCGCGGGCGCATTCAGATTCGGATATTTG
TGGAACAACAAGGCTGCCTATGATACGAGAAGAGGGGAGGCTTGACGGATTGGAGTCCGTAAATAGAGCAATGTCATGAA
AGAAAAATGGACCATCTCTCTCATACTACTCCTCCTACTTTGCATTGGCATGCGCTTCAGTTTCTTAGTGTTTTAATTCA
GTTCCTTCAGAGTTTGTGGATACATGTTTCCCCAGGACTAGTAGTAGGGCAGAAAAAAGTACACGGATTTGCACCGAGTA
TAAATTAGGCAAGGTAGTGTTTCTGTATATGTACGATCACGGTCTAGTTCTCAAATTCGCTGTCATATTGGTAGACATCG
GACTGAACTTGTGGTCATGAAAGTAATCATAGGCAAGCGAAGATCTTGTGTGTTT
>XP_044371266.1 serine/threonine-protein kinase RIPK-like B genome [Triticum
aestivum]
(SEQ ID NO: 19)
MARSAWRSLFGCFSSHAEAAAAAAAAGKGEKKGKASKKKKSKKKVAAAGKAPRPSRSLHGRMSFSDLSMSGGMVSPEDLS
LSLAGSDLHIFTIAELRAVTRDFSMTNFIGEGGFGPVYKGYVDEKVKPGLRAQPVAVKLLDLEGNQGHNEWLTEVIFLGQ
LRHPHLVKLIGYCYEDEHRLLVYEFMTRGSLEKLLFKKYAASLQWSTRLKIALGAAKGLAFLHEAENPVIYRDFKTSNIL
LDSDYKAKLSDFGLAKDGPEEDETHVSTRVMGTQGYAAPEYIMTGHLTAKSDVYGFGVVLLELLTGRKSVDKSRPPREQN
LVDWARPYLNDSRRLDRVMDPNLAGQYAGGAAQRAAALAYKCVSLNPKSRPHMSAVVSALEPLLALEEDSLAGPFVYVAP
PDNGSSGGGSREGRHRRRAGRRESGDVAESVAVVVERE
>XM_044515331.1 LQC123093374 [organism = Triticum aestivum] [GeneID = 123093374]
(SEQ ID NO: 20)
GCACACGCGGAAGGCACGCACGGATGGCGAGGTCGGCGTGGAGGTCCCTGTTCGGGTGCTTCAGCTCGCACGCCGAGGCC
GCGGCCGCCGCAGCCGCCGCCGGCAAGGGCGAGAAAAAGGGCAAGGCCTCCAAGAAGAAGAAGAGCAAGAAGAAGGTGGC
GGCGGCGGGCAAGGCGCCGAGGCCGAGCCGGTCGCTGCACGGCCGGATGTCCTTCTCGGACCTCAGCATGAGCGGCGGCA
TGGTGTCGCCGGAGGACCTGTCGCTGTCGCTGGCGGGGTCCGACCTCCACATCTTCACCATCGCCGAGCTGCGCGCCGTC
ACCAGGGACTTCTCCATGACCAACTTCATCGGCGAGGGCGGGTTCGGCCCCGTCTACAAGGGCTACGTCGACGAGAAGGT
CAAGCCCGGCCTCCGCGCGCAGCCCGTCGCCGTCAAGCTGCTCGACCTCGAGGGGAACCAGGGGCACAACGAGTGGCTGA
CTGAAGTCATATTCCTGGGGCAGTTGAGGCATCCTCACCTGGTCAAACTGATTGGTTATTGCTACGAGGATGAGCACAGG
CTCCTCGTGTACGAGTTCATGACCAGGGGCAGCCTGGAGAAACTTCTCTTCAAAAAATACGCGGCGTCGTTGCAATGGTC
AACACGGCTGAAGATCGCCCTGGGCGCCGCCAAAGGGTTGGCGTTCCTCCATGAGGCCGAGAACCCGGTCATCTACCGGG
ACTTCAAGACCTCCAACATCCTGCTAGACTCGGATTACAAAGCGAAGCTGTCGGATTTCGGGCTAGCCAAAGATGGACCG
GAAGAAGACGAGACGCACGTCTCCACCCGGGTCATGGGCACGCAGGGCTACGCCGCGCCGGAGTACATTATGACAGGGCA
TCTGACGGCCAAGAGCGACGTGTACGGCTTCGGCGTGGTGCTGCTGGAGCTGCTGACGGGGCGCAAGTCGGTGGACAAGA
GCCGGCCGCCGCGGGAGCAGAACCTGGTGGACTGGGCGCGCCCCTACCTCAACGACTCCCGGCGCCTGGACCGCGTCATG
GACCCAAACCTCGCCGGGCAGTACGCCGGCGGGGCCGCGCAGAGGGCCGCCGCACTGGCCTACAAGTGCGTCAGCCTCAA
TCCCAAGTCCCGCCCGCACATGTCCGCCGTCGTCAGCGCGCTCGAGCCGCTCCTCGCGCTCGAAGAGGATAGCCTCGCCG
GGCCGTTCGTCTACGTTGCGCCGCCTGACAACGGCAGCAGCGGCGGTGGCAGCAGGGAGGGGCGCCACAGGAGGCGAGCG
GGCCGGAGGGAGTCCGGAGACGTGGCCGAGTCGGTGGCCGTTGTTGTTGAGCGCGAATGATGGTCTCAGCTTGACGGAGC
GTGTTTCTGAACTTGGACCGGTCCAACGAACAGTGGAGAGGAGCGCGGGCGCATTCAGCTTCGAATATTTGTGGAACAAC
AAGGGTTGCCTATGATACGAGAAGAGGAGAGGCTTGACGGACTGGAGTCCGTAAATAGAGCAATTTCATGAAAGAAAAAA
TGGACTATCTCTCTCGTGCTACTCCTCCTACTTTGCATTGGCATGCACTTCAGTTTGTTAGTGTTTTCTGAGTTTACGGA
CAAGTACTAGTAGCAGGGAAAAAAGTACACGGACTTGCACCGAGTATAAATTAGGCAAGGTAGTGTTTCTATATATGTAC
CTCACGGTCTAGTTCTCAAATTTGCACTCATCTCTACTCCTAATGTCTCAGTTAGTAGAATAGTCCATGGTTTTTTTCCG
TCTGATTAATTTTTATCGGGTTTATTTT
>XP_044376632.1 serine/threonine-protein kinase RIPK-like D genome [Triticum
aestivum]
(SEQ ID NO: 21)
MARSAWRSLFGCFSSHAEAAAAAAAAGKGERKPPGGKKKKKSKKKVAAAGKAPRPSRSLHGRMSFSDLSMSGGMVSPEDL
SLSLAGSDLHVFTIAELRAVTRDFSMTNFIGEGGFGPVYKGYVDEKVKPGLRAQPVAVKLLDLEGNQGHNEWLTEVIFLG
QLRHPHLVKLIGYCYEDEHRLLVYEFMTRGSLEKLLFRKYAASLQWSTRLKIALGAAKGLAFLHEAQNPVIYRDFKTSNI
LLDSDYKAKLSDFGLAKDGPEEDETHVSTRVMGTQGYAAPEYIMTGHLTAKSDVYGFGVVLLELLTGRKSVDKSRPPREQ
NLVDWARPYLNDSRRLDRVMDPNLAGQYAGGAAQRAAALAYKCVSLNPKSRPHMSAVVSALEPLLALEEDSLAGPFVYVA
PPDNGSSGGGGSREGRHRRRAGRRKSGDVAESVAVVVERE
>XM_044520697.1 LOC123098649 [organism = Triticum aestivum] [GeneID = 123098649]
(SEQ ID NO: 22)
CAGAGCGACGCGCACACGGGGAAGGCACGCACGCACGCGTACGGAATCGCCCGCGCGCGCCGATGGCGAGGTCGGCGTGG
AGGTCCCTCTTCGGGTGCTTCAGCTCGCACGCCGAGGCCGCGGCCGCCGCAGCCGCCGCCGGCAAGGGCGAGAGGAAGCC
CCCCGGCGGCAAGAAGAAGAAGAAGAGCAAGAAGAAGGTGGCGGCGGCGGGGAAGGCGCCGAGGCCGAGCCGGTCGCTGC
ACGGCCGGATGTCCTTCTCGGACCTCAGCATGAGCGGCGGCATGGTGTCGCCGGAGGACCTGTCGCTGTCGCTGGCGGGG
TCCGACCTGCACGTCTTCACCATCGCCGAGCTGCGCGCCGTCACCAGGGACTTCTCCATGACCAACTTCATCGGCGAGGG
CGGCTTCGGCCCCGTCTACAAGGGCTACGTCGACGAGAAGGTCAAGCCCGGGCTCCGCGCGCAGCCCGTCGCCGTCAAGC
TGCTCGACCTCGAGGGGAACCAGGGCCACAACGAGTGGCTGACTGAAGTCATATTCCTGGGGCAGTTGAGGCATCCTCAC
CTGGTCAAACTGATCGGTTACTGCTACGAGGATGAGCACAGGCTCCTCGTGTACGAGTTCATGACCAGGGGCAGCCTGGA
GAAGCTTCTCTTCAGAAAATACGCGGCGTCGTTGCAATGGTCAACACGGCTGAAGATCGCCCTGGGTGCCGCCAAAGGGT
TGGCGTTCCTCCATGAGGCCCAGAACCCGGTCATCTACCGGGACTTCAAGACCTCCAACATCTTGCTCGACTCGGATTAC
AAAGCGAAGCTATCGGATTTCGGGCTAGCCAAAGATGGGCCCGAAGAGGACGAGACGCACGTCTCCACCCGGGTCATGGG
CACGCAGGGCTACGCCGCACCCGAGTACATCATGACAGGGCATCTGACGGCAAAGAGCGACGTGTACGGCTTCGGCGTGG
TGCTGCTGGAGCTGCTGACGGGGCGCAAGTCGGTGGACAAGAGCCGGCCGCCGCGGGAGCAGAACCTGGTGGACTGGGCC
CGGCCCTACCTCAACGACTCCCGGCGCCTGGACCGCGTCATGGACCCCAACCTCGCCGGGCAGTACGCCGGCGGGGCCGC
GCAGAGGGCCGCCGCGCTGGCCTACAAGTGCGTCAGCCTCAACCCCAAGTCCCGCCCGCACATGTCCGCCGTCGTCAGTG
CGCTCGAGCCGCTCCTCGCGCTCGAAGAGGATAGCCTCGCCGGCCCGTTCGTGTATGTTGCGCCGCCTGACAATGGCAGC
AGCGGCGGCGGCGGGAGCAGGGAGGGGCGCCACAGGAGGCGAGCGGGCCGGAGGAAGTCCGGAGACGTGGCCGAGTCGGT
GGCCGTTGTTGTTGAGCGTGAATGATGGTCTCAGCTTGACGGAGCGTGTTTTCTGAACTTGGACGCGTCGAACAGTGGAG
AGGAGCGCGAGCGCATTCAGATTCGAATATTTGTGGAACAACAAGGGTTGCCTATGATACGAGAAGAGGGGAGGCTTGAC
GGATTGGAGTCCATAAATAGAGCAATTTCATGAAAGAAAAATGGACAATCTCTCTCGTACTACTCCTCCTACTTTGCATT
GGCATGCACTTCAGTTTCTTAGTGTTTTAATTCAGTTCATTTAGAGTTTGTACATACATGTTTCCCCAGGACTAAGTAGT
AGGGCAAAAAAAGTACACGGATTTGTACCGAGTATAAATTAGGCAAGGTCTGTCTAGGTCTAAGTCGACTGAGACTTCGC
GAAGTCTAAGTTGCTGACGTCATCAAATTTTCTTTTCCTTTTGAGAGTGTCATCCATGTTTGTACGCAGCCTATTGTTTG
TGCATTCTCACGGCTGTTACTTCTTCCTGTGTTGGGCTGGGCCCGCCTCTCTCTCTGTGTACCCTTTTCTTCTTTTGTAT
TTTGTATTATTAGGCTGGACTTGTCTCA
>(SEQ ID NO: 23)
SDFGLK[D/A]GP[E/Q/G][G/E][D/E/G][E/H/D/K]THV[S/T]TRV[M/V]GT[Q/Y]GYAAPEY
[I/V/L]MTGHLT[T/A/S][M/K/R/A][S/N]DVYS[F/Y]GVV
>(SEQ ID NO: 24)
QGH[R/K]EW[L/V][A/T]E[V/I][I/V/L/D]FLGQL[R/S/H][H/N][P/K/H]HLVKLIG[Y/F]C
[C/Y/I][E/Q][E/D][E/D][H/Q/N]R[L/V]L[V/A]YE[Y/F][M/L][P/A]RGSLE[N/K][Q/H]L
>(SEQ ID NO: 25)
[S/T/A][A/V/L][S/A/T/P][L/M][P/A]W[S/G][T/I/N]R[M/L][K/R]IA[L/I/A]GAA[K/R]
GLAFLH[E/G/D][A/G][E/A/P/K/D]KPVIYRDFK[T/A/S]SN[I/V]LL[D/N][S/C/A][D/N/E]Y
[N/T/E][A/C]KL
>AT3G08850 Arabidopsis RAPTOR1B with phospho-mimicking S740D mutation
(SEQ ID NO: 26)
MALGDLMVSRFSQSSVSLVSNHRYDEDCVSSHDDGDSRRKDSEAKSSSSYGNGTTEGAATATSMAYLPQTIVLCELRHDA
SEASAPLGTSEIVLVPKWRLKERMKTGCVALVLCLNITVDPPDVIKISPCARIEAWIDPFSMAPPKALETIGKNLSTQYE
RWQPRARYKVQLDPTVDEVRKLCLTCRKYAKTERVLFHYNGHGVPKPTANGEIWVENKSYTQYIPLPISELDSWLKTPSI
YVFDCSAARMILNAFAELHDWGSSGSSGSSRDCILLAACDVHETLPQSVEFPADVFTSCLTTPIKMALKWFCRRSLLKEI
IDESLIDRIPGRQNDRKTLLGELNWIFTAVTDTIAWNVLPHELFQRLFRQDLLVASLERNFLLAERIMRSANCNPISHPM
LPPTHQHHMWDAWDMAAEICLSQLPQLVLDPSTEFQPSPFFTEQLTAFEVWLDHGSEHKKPPEQLPIVLQVLLSQCHRER
ALVLLGRFLDMGSWAVDLALSVGIFPYVLKLLQTTTNELRQILVFIWTKILALDKSCQIDLVKDGGHTYFIRFLDSSGAF
PEQRAMAAFVLAVIVDGHRRGQEACLEANLIGVCLGHLEASRPSDPQPEPLFLQWLCLCLGKLWEDFMEAQIMGREANAF
EKLAPLLSEPQPEVRAAAVFALGTLLDIGFDSNKSVVEDEFDDDEKIRAEDAIIKSLLDVVSDGSPLVRAEVAVALARFA
FGHKQHLKLAAASYWKPQSDSLLTSLPSIAKFHDPGSATIVSLHMSPLTRASTDSQPVARESRISSSPLGSSGLMQGSPL
SDDSSLHSDSGMMHDSVSNGAVHQPRLLDNAVYSQCVRAMFALAKDPSPRIASLGRRVLSIIGIEQVVAKPSKPTGRPGE
AATTSHTPLAGLARSSSWFDMHAGNLPLSFRTPPVSPPRTNYLSGLRRVCSLEFRPHLLGSPDSGLADPLLGASGSERSL
LPLSTIYGWSCGHFSKPLLGGADASQEIAAKREEKEKFALEHIAKCQHSSISKLNNNPIANWDTRFETGTKTALLHPFSP
IVVAADENERIRVWNYEEATLLNGFDNHDFPDKGISKLCLINELDDSLLLVASCDGSVRIWKNYATKGKQKLVTGESSIQ
GHKPGARDLNAVVDWQQQSGYLYASGETSTVTLWDLEKEQLVRSVPSESECGVTALSASQVHGGQLAAGFADGSLRLYDV
RSPEPLVCATRPHQKVERVVGLSFQPGLDPAKVVSASQAGDIQFLDLRTTRDTYLTIDAHRGSLTALAVHRHAPIIASGS
AKQLIKVFSLQGEQLGIIRYYPSFMAQKIGSVSCLTFHPYQVLLAAGAADSFVSIYTHDNSQAR
>AT3G08850 Arabidopsis RAPTOR1B
(SEQ ID NO: 27)
MALGDLMVSRFSQSSVSLVSNHRYDEDCVSSHDDGDSRRKDSEAKSSSSYGNGTTEGAATATSMAYLPQTIVLCELRHDA
SEASAPLGTSEIVLVPKWRLKERMKTGCVALVLCLNITVDPPDVIKISPCARIEAWIDPFSMAPPKALETIGKNLSTQYE
RWQPRARYKVQLDPTVDEVRKLCLTCRKYAKTERVLFHYNGHGVPKPTANGEIWVENKSYTQYIPLPISELDSWLKTPSI
YVFDCSAARMILNAFAELHDWGSSGSSGSSRDCILLAACDVHETLPQSVEFPADVFTSCLTTPIKMALKWFCRRSLLKEI
IDESLIDRIPGRQNDRKTLLGELNWIFTAVTDTIAWNVLPHELFQRLFRQDLLVASLERNELLAERIMRSANCNPISHPM
LPPTHQHHMWDAWDMAAEICLSQLPQLVLDPSTEFQPSPFFTEQLTAFEVWLDHGSEHKKPPEQLPIVLQVLLSQCHRER
ALVLLGRFLDMGSWAVDLALSVGIFPYVLKLLQTTTNELRQILVFIWTKILALDKSCQIDLVKDGGHTYFIRFLDSSGAF
PEQRAMAAFVLAVIVDGHRRGQEACLEANLIGVCLGHLEASRPSDPQPEPLFLQWLCLCLGKLWEDFMEAQIMGREANAF
EKLAPLLSEPQPEVRAAAVFALGTLLDIGFDSNKSVVEDEFDDDEKIRAEDAIIKSLLDVVSDGSPLVRAEVAVALARFA
FGHKQHLKLAAASYWKPQSSSLLTSLPSIAKFHDPGSATIVSLHMSPLTRASTDSQPVARESRISSSPLGSSGLMQGSPL
SDDSSLHSDSGMMHDSVSNGAVHQPRLLDNAVYSQCVRAMFALAKDPSPRIASLGRRVLSIIGIEQVVAKPSKPTGRPGE
AATTSHTPLAGLARSSSWFDMHAGNLPLSFRTPPVSPPRTNYLSGLRRVCSLEFRPHLLGSPDSGLADPLLGASGSERSL
LPLSTIYGWSCGHFSKPLLGGADASQEIAAKREEKEKFALEHIAKCQHSSISKLNNNPIANWDTRFETGTKTALLHPFSP
IVVAADENERIRVWNYEEATLLNGFDNHDFPDKGISKLCLINELDDSLLLVASCDGSVRIWKNYATKGKQKLVTGESSIQ
GHKPGARDLNAVVDWQQQSGYLYASGETSTVTLWDLEKEQLVRSVPSESECGVTALSASQVHGGQLAAGFADGSLRLYDV
RSPEPLVCATRPHQKVERVVGLSFQPGLDPAKVVSASQAGDIQFLDLRTTRDTYLTIDAHRGSLTALAVHRHAPIIASGS
AKQLIKVFSLQGEQLGIIRYYPSFMAQKIGSVSCLTFHPYQVLLAAGAADSFVSIYTHDNSQAR
K
>NM_111719.3 RAPTOR1
[organism = Arabidopsis thaliana] [GeneID = 820032]
(SEQ ID NO: 28)
TTTGATCTGAAGAGGGTAAAACTAAAAACGGAAAGGAGAAAAAGAATGAAAGAGAAGTGGAAAGAAAACGCAAATGCATT
GAAGACTTTCCCTTTGTCTCTCTCTTTGACAAAATCCCAATTTTCGCATCCTCACTTTCTCCTCTGCCTTTTTTTTTTCT
CTTCTGCCGCCACCACCACCACCACTGTTAATTCGCCGCCGCTTCCACATCAATCGCCGTCGTAGCCAATCATCATCGTT
CTTCATCCTCCGCTTCTCCTGATTCTTAGGGTTTCCGCTTCCGCTTCTCTGTCGAGGGTTTTTCGAGCTGGATTCTCTGC
TCCTGCGCCGCTGGTGCAGCGGCTTTGTTGATTGATCTGGATTTCTCCGTCTTCCTTTCTATTTTATTTTCTGTATCTCG
CACAATCCAAACAAAAAAGCTAGGGCTGAGGATTTGATTTGCGTGATTTCTGGGGGTGTGTTAATTCGGGGGTGTGTTCT
CCGATTTGGATGGCATTAGGAGACTTAATGGTGTCTCGGTTCTCGCAATCTTCTGTTTCTTTGGTCTCCAATCACCGTTA
CGACGAAGACTGTGTCTCTAGTCACGATGACGGTGATTCCCGGAGGAAGGATTCTGAGGCTAAGAGCAGTAGTAGCTACG
GGAATGGTACAACGGAGGGAGCCGCCACCGCCACCAGCATGGCTTACTTGCCTCAGACTATTGTGCTCTGCGAGCTTAGA
CACGACGCATCCGAGGCTTCTGCTCCCTTGGGGACTTCTGAGATCGTATTGGTCCCCAAATGGCGGCTTAAAGAACGAAT
GAAAACAGGATGTGTAGCTTTAGTTCTGTGTTTAAACATTACTGTTGATCCGCCGGATGTTATAAAGATATCTCCTTGTG
CTAGAATCGAGGCATGGATAGATCCATTTTCTATGGCACCGCCTAAAGCTCTCGAGACAATTGGAAAAAATTTGAGCACT
CAGTATGAGAGATGGCAACCCAGGGCCCGCTATAAGGTTCAGCTTGATCCGACAGTAGATGAGGTGAGGAAGCTGTGCTT
GACTTGTCGGAAATATGCAAAGACCGAGAGAGTTCTATTCCATTATAATGGGCATGGTGTGCCAAAACCTACAGCTAATG
GTGAAATTTGGGTATTTAACAAGAGTTATACACAGTACATTCCCTTGCCAATCAGTGAACTTGATTCCTGGTTGAAGACA
CCCTCCATCTATGTTTTCGACTGCTCTGCTGCTAGGATGATTCTGAATGCCTTTGCTGAGCTTCATGATTGGGGTTCTTC
TGGTTCCTCTGGATCCTCAAGGGACTGCATTCTACTTGCTGCTTGTGATGTACATGAGACACTTCCTCAGAGCGTTGAGT
TTCCAGCTGATGTTTTTACGTCTTGCCTGACAACGCCCATTAAAATGGCGTTGAAATGGTTCTGCAGGCGTTCGCTTCTG
AAAGAAATTATCGATGAATCACTTATCGACAGGATTCCAGGCCGGCAAAACGATCGTAAGACATTGTTAGGGGAGTTGAA
CTGGATTTTCACAGCAGTGACGGATACAATTGCTTGGAATGTGCTTCCTCATGAACTTTTCCAGAGATTATTCAGACAGG
ACTTGTTGGTCGCTAGCCTTTTCCGGAATTTCTTACTTGCTGAGAGAATAATGCGGTCCGCAAACTGTAATCCAATATCT
CACCCTATGCTGCCTCCTACGCATCAACATCATATGTGGGATGCATGGGACATGGCTGCTGAAATCTGTCTTTCTCAGCT
TCCCCAACTTGTTCTGGACCCAAGCACAGAGTTCCAGCCAAGTCCGTTTTTTACTGAGCAACTGACAGCTTTTGAGGTGT
GGCTTGATCATGGATCTGAGCATAAGAAGCCACCGGAGCAGTTACCTATTGTTCTTCAGGTGTTACTTAGCCAGTGCCAT
CGGTTTCGTGCTCTTGTACTTCTTGGAAGATTTCTTGATATGGGTTCATGGGCTGTGGATCTGGCCTTGTCTGTTGGAAT
ATTCCCATATGTGCTGAAGCTTCTGCAAACAACAACGAATGAGCTAAGACAGATCCTGGTTTTCATATGGACAAAAATTC
TTGCACTTGACAAGTCATGTCAAATTGATCTTGTGAAGGATGGGGGGCATACATACTTCATACGATTTCTAGATAGCTCG
GGTGCATTTCCAGAACAACGAGCTATGGCTGCTTTTGTTCTGGCTGTCATTGTGGATGGACATCGACGAGGCCAGGAAGC
ATGTCTTGAAGCTAATTTAATTGGTGTTTGTCTCGGGCACCTTGAAGCATCCAGACCAAGTGATCCACAACCAGAACCAT
TGTTTCTACAATGGCTTTGTCTTTGTCTTGGAAAGTTATGGGAAGATTTTATGGAGGCCCAAATAATGGGCAGGGAGGCA
AATGCTTTTGAAAAGTTGGCACCTCTGCTTTCCGAGCCCCAACCTGAGGTAAGGGCTGCTGCAGTTTTTGCCCTGGGTAC
CTTGCTTGATATTGGGTTTGACTCTAATAAAAGTGTGGTGGAGGATGAATTTGATGATGATGAAAAGATTAGAGCCGAAG
ACGCTATCATTAAAAGTCTCTTAGATGTAGTTTCAGATGGGAGTCCACTTGTCCGAGCAGAGGTTGCCGTAGCTCTTGCA
CGTTTTGCCTTTGGCCACAAACAGCACCTTAAGTTAGCCGCAGCTTCATATTGGAAGCCTCAGTCAAGTTCTTTGCTTAC
TTCTCTACCTTCAATAGCTAAATTCCATGATCCTGGGAGTGCAACAATTGTTTCTTTACACATGAGTCCTCTGACTAGAG
CTAGCACGGATAGCCAACCAGTGGCTCGTGAGTCTAGGATCTCAAGCAGCCCTCTTGGCTCCTCTGGGCTGATGCAAGGA
TCTCCATTATCTGATGATTCTTCGTTACATTCTGATTCTGGAATGATGCATGACAGTGTCAGCAATGGAGCGGTCCATCA
GCCAAGACTGTTGGATAATGCTGTTTATTCGCAGTGCGTCCGAGCTATGTTTGCATTAGCTAAAGATCCATCTCCACGTA
TTGCAAGTCTCGGACGGCGTGTGCTTTCTATTATTGGAATCGAACAGGTTGTTGCGAAACCCTCGAAGCCCACTGGCCGA
CCAGGGGAAGCTGCAACGACATCTCACACTCCACTTGCTGGCCTAGCTCGTTCATCCTCATGGTTTGATATGCATGCAGG
TAATCTGCCTTTAAGTTTTAGGACTCCCCCGGTCAGCCCCCCTAGAACAAACTACCTGAGTGGATTGAGGAGAGTTTGTT
CATTAGAGTTCAGGCCTCATCTATTGGGTTCACCCGACTCAGGATTGGCTGATCCGCTTTTAGGCGCCAGTGGATCTGAA
CGGAGTTTGCTTCCACTATCAACTATCTACGGCTGGAGTTGTGGGCACTTTTCTAAGCCACTTCTTGGTGGTGCGGATGC
TAGTCAAGAGATTGCAGCCAAAAGAGAAGAGAAAGAAAAATTCGCACTTGAGCATATTGCAAAATGCCAGCACTCTTCTA
TTAGCAAGCTCAACAATAATCCTATTGCCAACTGGGATACAAGGTTTGAAACGGGAACAAAGACAGCCCTCCTTCACCCA
TTCTCTCCTATTGTAGTTGCTGCAGACGAGAATGAACGGATCAGAGTGTGGAACTATGAGGAAGCAACTCTTCTCAATGG
CTTTGACAATCATGATTTTCCTGACAAAGGAATTTCAAAGCTCTGCCTCATCAATGAACTTGACGACTCTCTGCTACTTG
TTGCATCATGCGATGGGTCGGTCCGGATATGGAAAAACTATGCAACTAAGGGTAAACAAAAGCTTGTTACTGGGTTTTCT
TCAATCCAGGGTCACAAGCCCGGTGCCCGTGACTTGAACGCTGTCGTGGACTGGCAACAACAGTCCGGTTACCTGTATGC
TTCTGGGGAGACGTCAACAGTCACACTTTGGGACCTGGAGAAAGAACAGCTTGTCAGATCTGTTCCCTCTGAATCAGAAT
GTGGAGTTACAGCACTTTCTGCTTCTCAAGTGCACGGAGGCCAACTCGCTGCTGGTTTTGCTGATGGATCTTTGAGACTC
TATGATGTTCGGTCACCTGAACCGCTTGTCTGCGCGACTCGGCCTCATCAGAAAGTTGAAAGGGTGGTTGGCCTCAGTTT
TCAACCTGGACTTGACCCCGCAAAGGTGGTGAGTGCATCACAGGCGGGTGACATACAGTTTCTTGACCTTAGAACAACAA
GGGACACATACCTGACGATTGATGCACACAGGGGTTCACTCACGGCCTTAGCTGTTCACAGACACGCTCCAATAATCGCG
AGTGGATCTGCAAAACAGCTCATTAAAGTATTCAGTCTTCAAGGGGAACAACTAGGGATAATCCGCTACTACCCATCCTT
CATGGCTCAAAAGATTGGATCAGTGAGTTGCCTCACATTTCATCCGTACCAGGTTCTGCTAGCAGCTGGAGCTGCTGACT
CATTTGTCTCCATATACACCCACGACAACTCGCAAGCAAGATGAGTCACAACCGTAAGTGAATATGTTGCATTTAGCATT
GCTCTTGTGATAAAAAGAAAAGCATAAATCAGTGCAGGTCGGGTCGGTTTTGGGTCGGGGTAAAGTGGGTGTGGTTTATA
GTATTCGTTAGCCTCCGTCCAAGATAAAACAAGATCTAAGTTAACACTAAAGAAGGAGGCTTCACCAAATAAGTTATATC
TCTACCTATGCAACAACGCACGTAAATGGTGTCTGTAAATAGTTTCCCTGTTTTGCTTTTCATTTTCTCATCTCTTGGGT
GTGTGATTTCCTTTTTATTAATCTTTTAAGTCATTCTGTTTCCTAATTTGTTTCATGTGTAAAAAAAATGGTAAAAGTAA
TGGTGGAAGAAAATTTGCATTTTCGTGACGATGCTTTGAAATGTTGTCTGTTGTTTGTTCAAGTTGGGATCCGTTGGAAA
TAGGCTTGCAATTTTTCATGCTACGCATCTCTATGATTGGTCAAATACCAGTTTATGTCAGAATCCAAGTAATTATCGAA
AAATC
>XP_035819171.1 regulatory-associated protein of TOR 1 isoform X1 [Zea mays]
(SEQ ID NO: 29)
MALGDLMASRLVHSSSSRGLSSSPTPPLPNQHHHQNHNHTGDGLLAASGPEPRNGLEADEVDKSAPVPYLPQLVVLCEQR
HEPEGIDEAAAAAAGPSTSGLVSKWRPKDRMKTGCVALVLCLNISVDPPDVIKISPCARMECWIDPFSMAPPKALESIGK
TLHSQYERWQPKARYKLQLDPTVEEVKKLCNTCRKYARSERVLFHYNGHGVPKPTSNGEIWVENKSYTQYIPLPITDLDS
WLKTPSIYVFDCSASGIIVKAFLERLDWSSSSSASSRKDCILLAACEAHQTLPQSAEFPADVFTACLTTPIKMALHWFCK
RSLLRGSLDLSLIDQIPGRQNDRKTLLGELNWIFTAITDTIAWNVLPHDLFQRLFRQDLLVASLFRNELLAERIMRSANC
SPVSYPLLPPTHQHHMWDAWDMAAEICLSKLPQLIADPNAEFQPSPFFTEQLTAFEVWLDHGSEDKKPPEQLPIVLQVLL
SQSHRFRALVLLGRFLDMGPWAVDLALSVGIFPYVLKLLQTSAMELRQILVFIWTKILSLDKSCQVDLVKDGGHTYFVRF
LDSLDAYPEQRAMAAFVLAVIVDGHRTGQEACMNAGLIDVCLRHLQPENPHDSQTEPLLLQWLCLCLGKLWEDFPDVQLL
TLQTNTPEIVLCLLSEPQPEVRAAAVFALGTLLDTTSSNGADDDSDYDEKVKAEINVVRSLLQISSDASPLVRSEVAIAL
TRFALGHNKHLKSVAAEYWKPQSNSLLKSLPSLANINNPNNVYSPNNILQGSIGPVLRVGSDSSAAGRDGRMSTSSPIAT
SSIMHGSPQSDDSSQLSDSGLLLRENASNGGPGYTRSRPVDNVIYSQFISTMCSFAKDPYPRIATIGRRALSLIGVEQVV
MKNTRFNSGGAHRGETSASNFGMARSSSWLDMNSGNNLIKFRTPPVSPPQHDFLPGLRRVCSMEFGQHPISSPDGLAVPL
LSSAAATSNAELSILPQSTIYNWSCGHFSRPLLIGSDDNEEANARREERERLALDYIAKCQRSSACKMTNQIASWDTRFE
LGTKTALLLPFSPIVVAADENEQIRVWNYDDALPVNSYQNHNLSDRGLSKLLLINELDESLLLAASSDGNVRVWKNFTQK
GGQKLVTAFSSVQGHRTAGRSIVIDWQQQSGYLYASGDMSSILLWDLDKEQQLSTIQSSGDSAISSLSASQVCSGHFAAG
FAAGSVRIYDIRSPDRVVYTARPHAPRTEKVVGIGFQPGFDPYKIVSASQAGDIQFLDVRRSAEPYLTIEAHRGSLTALA
VHRHAPVVASGSAKQMIKVFSLEGEQLTIIRYQPSFMGQRIGTVNCLSFHPYKSLLSAGAGDNALVSIYAEENYK
>XM_035963278.1 LOC103640651 [organism = Zea mays] [GeneID = 103640651]
[transcript = X1]
(SEQ ID NO: 30)
GGGCAGGCAATTTCCATTTCCAACCTTTATACTATAAATTTTATTTTGTTTTCCCTCAATCTCTCTCCTCGGCTGCTCGC
CTGGCGTCGCGTCGCCTGCTTCCTCGTTCCCGCATCGAGACGCCGCTTCCCGTCCCTTCCCCCTCCTCTCTCTCTCCTCC
CGGCCAACCAAACCCTAACCCTAAACCCCTCCCCCACCAACCTCGGCCCAGTCTCCCCACCCCCACTGACCCGGCTCCCC
ACCTCCGACTCGCCACGCTGGCCCGATTCGATCGATGTACGTGAGCGAGCTCCCGACCTCATGATGGTTTTGGCTGCGGG
GTGAAAAGAGGCACAGCAAGCGTCGATGGCATTGGGAGATCTCATGGCGTCCAGGCTCGTCCACTCCTCGTCCTCCAGGG
GACTGTCGTCCTCGCCGACGCCGCCGTTGCCCAATCAACACCACCACCAGAACCACAACCATACCGGGGATGGCCTCCTG
GCTGCCAGCGGGCCGGAGCCCAGGAACGGGCTGGAGGCCGACGAGGTGGATAAGTCGGCGCCCGTGCCGTACCTGCCCCA
GCTGGTCGTGCTGTGCGAGCAGCGCCACGAGCCTGAGGGGATCGATGAGGCTGCGGCCGCAGCCGCAGGGCCCTCCACCA
GTGGCCTCGTCTCCAAGTGGCGCCCCAAGGATCGGATGAAGACAGGATGTGTTGCACTTGTATTATGTTTAAACATAAGT
GTGGATCCACCTGATGTAATTAAAATTTCCCCTTGTGCAAGAATGGAGTGCTGGATAGATCCTTTTTCTATGGCACCTCC
TAAAGCCCTTGAGAGTATTGGAAAAACGTTGCATTCACAGTATGAGCGCTGGCAACCTAAGGCTCGATACAAGCTTCAGC
TGGACCCAACAGTGGAGGAAGTTAAGAAGCTTTGCAATACTTGCCGCAAATATGCCAGATCAGAGAGAGTTCTTTTCCAT
TATAATGGCCATGGCGTACCAAAGCCTACATCTAATGGTGAGATTTGGGTGTTTAACAAGAGTTACACACAGTATATTCC
ACTTCCAATTACTGATCTTGATTCATGGCTGAAGACACCTTCAATTTACGTTTTTGATTGCTCAGCGTCTGGAATTATTG
TGAAAGCTTTTCTAGAGCGCCTAGATTGGAGTTCTAGCTCCTCTGCATCTTCACGGAAGGACTGCATTCTTCTTGCTGCC
TGTGAGGCACATCAAACTCTTCCTCAGAGTGCAGAATTTCCTGCCGATGTGTTTACAGCTTGCCTCACAACACCCATCAA
AATGGCTTTGCACTGGTTTTGTAAGCGATCATTACTCCGTGGTTCTCTGGATCTTTCTCTTATTGACCAAATTCCTGGAA
GGCAAAATGACCGTAAAACTCTTCTTGGGGAGCTCAATTGGATTTTTACTGCTATAACAGATACAATTGCATGGAATGTT
CTTCCTCATGATCTGTTCCAAAGGCTTTTCAGGCAAGATCTTCTTGTTGCTAGCCTCTTTCGTAACTTCTTGCTTGCTGA
GAGAATCATGCGATCAGCAAATTGTTCTCCAGTTTCTTATCCGTTGTTACCTCCAACACATCAACACCATATGTGGGATG
CATGGGACATGGCTGCTGAGATTTGCCTTTCCAAGCTTCCTCAGTTAATTGCTGATCCAAATGCAGAGTTTCAGCCAAGT
CCGTTTTTTACGGAACAACTGACGGCTTTCGAAGTGTGGCTTGATCATGGCTCCGAGGACAAGAAACCTCCTGAACAGTT
ACCCATAGTTCTTCAGGTCCTGCTTAGTCAATCACACAGATTTAGAGCACTTGTTCTGCTCGGAAGATTTCTTGACATGG
GGCCGTGGGCAGTTGATTTGGCCCTGTCTGTCGGAATATTTCCTTATGTACTCAAACTGCTTCAAACAAGTGCAATGGAG
TTGCGTCAAATTCTTGTGTTTATATGGACAAAAATTCTCTCTCTTGATAAGTCGTGCCAGGTTGATTTGGTTAAAGATGG
AGGACACACATATTTTGTTAGGTTTCTTGACAGTTTGGATGCTTACCCAGAACAGCGTGCAATGGCTGCTTTTGTTTTAG
CAGTCATTGTGGATGGGCATAGGACAGGCCAAGAGGCTTGTATGAATGCAGGGCTTATAGATGTTTGCTTGAGACACCTG
CAACCTGAGAATCCTCATGATTCACAGACAGAACCTTTACTTCTACAGTGGCTTTGTTTATGCCTTGGGAAACTCTGGGA
AGATTTCCCTGATGTTCAGCTGCTTACTTTACAAACAAATACGCCAGAAATTGTACTATGCTTATTGTCGGAGCCTCAAC
CTGAGGTCAGAGCTGCTGCTGTTTTTGCACTTGGGACTCTCCTGGATACTACATCATCAAATGGCGCTGATGATGATTCT
GACTATGATGAAAAGGTGAAAGCTGAAATAAATGTAGTTCGAAGCCTTCTGCAGATATCTTCAGATGCCAGTCCCCTTGT
TCGATCTGAGGTTGCTATAGCGCTTACTCGCTTTGCATTGGGTCATAACAAACATCTCAAATCTGTTGCGGCTGAGTATT
GGAAACCTCAATCCAATTCATTGCTGAAGTCACTGCCATCATTGGCAAATATAAATAACCCAAACAATGTTTACAGTCCC
AACAATATTCTACAAGGCAGCATTGGTCCTGTGCTGAGGGTTGGCAGTGATAGCAGTGCTGCTGGTCGTGATGGAAGAAT
GTCTACAAGTAGCCCAATTGCAACAAGTAGCATCATGCATGGCTCTCCCCAGTCAGACGATTCTTCCCAACTCTCTGATT
CTGGCTTATTACTGAGAGAGAATGCTAGTAATGGTGGTCCCGGCTACACTAGGTCCAGGCCTGTTGATAATGTCATTTAT
TCTCAATTTATATCAACTATGTGTTCTTTTGCTAAAGATCCTTACCCAAGAATTGCAACTATTGGTCGGAGAGCACTGTC
CCTTATAGGTGTTGAACAAGTAGTCATGAAAAATACTAGATTTAATAGTGGAGGTGCACATCGGGGAGAGACATCTGCTT
CAAACTTTGGAATGGCACGCTCTTCATCCTGGCTTGATATGAATTCTGGAAACAACTTGATAAAATTTAGGACCCCTCCT
GTTAGCCCTCCTCAGCATGATTTTCTTCCAGGATTACGCCGAGTGTGTTCTATGGAGTTTGGACAACATCCTATAAGTTC
ACCTGATGGCTTAGCTGTTCCCCTTTTAAGCTCCGCTGCAGCTACCAGTAATGCTGAACTAAGTATACTCCCTCAATCAA
CAATTTATAACTGGAGTTGTGGTCACTTCTCTAGGCCTCTTTTAATTGGCTCTGATGATAACGAAGAGGCAAATGCTAGA
AGGGAAGAGAGAGAACGACTTGCACTAGATTATATCGCTAAGTGCCAGCGCTCCTCAGCTTGCAAGATGACAAACCAAAT
TGCTAGTTGGGATACGAGGTTTGAGTTGGGAACAAAAACAGCTTTATTGTTGCCATTTTCTCCGATCGTTGTTGCAGCTG
ATGAAAATGAGCAAATAAGAGTGTGGAACTACGACGATGCGCTGCCAGTGAACTCTTATCAAAACCATAATTTGTCAGAC
AGAGGGCTATCCAAACTTTTGCTGATTAATGAGCTTGATGAGAGCTTGCTTTTAGCTGCCTCAAGTGATGGAAATGTACG
TGTATGGAAAAATTTTACTCAAAAGGGAGGACAAAAACTTGTAACGGCTTTCTCATCCGTTCAGGGACATCGAACTGCTG
GTCGCAGTATTGTGATTGACTGGCAGCAGCAATCTGGTTATCTTTATGCATCTGGTGACATGTCTTCCATCTTGCTATGG
GATCTTGACAAGGAACAACAACTCAGCACTATTCAGTCATCTGGTGATAGTGCCATTTCTTCACTGTCTGCATCTCAGGT
CTGCTCTGGACATTTTGCTGCTGGTTTTGCTGCTGGTTCTGTCAGGATATATGACATACGCTCTCCTGATAGGGTTGTCT
ATACGGCCAGGCCACACGCACCAAGAACAGAAAAGGTTGTAGGCATAGGGTTTCAACCTGGGTTTGATCCTTACAAGATT
GTAAGCGCATCTCAAGCTGGAGACATTCAGTTTCTTGATGTTAGAAGGTCAGCAGAACCCTACCTGACTATTGAGGCACA
CAGGGGTTCACTTACAGCATTGGCGGTTCACCGGCATGCCCCAGTTGTTGCTAGTGGCTCAGCCAAGCAGATGATTAAAG
TGTTTAGTCTTGAAGGAGAGCAGTTGACAATAATTCGCTACCAGCCATCTTTTATGGGCCAAAGAATAGGCACTGTGAAC
TGCCTTTCTTTTCACCCATACAAATCACTCCTATCTGCTGGTGCTGGTGATAATGCTCTTGTCTCTATCTACGCCGAGGA
AAATTACAAGTAAGGTGAGGCCTACATCTCATGACAATGCAGCCACAAGGCAACCGGCAGGCCTGGTGTTTTTTGGCCAA
GTGATGCTCCCCCGCAGCATGGTCCAGCTACAAGATCCAAGATGGTGTGAAGAGGCTCCCGGTGCGTATTGCCTGTTGTT
GGACGACGTGTGCAATTTGTAAGATAGAGCCAAGATTCGTTAGCCGCCTCCTTGCAGAAGGTTTGTGGAACCCCTACCCT
GATCATGTCATTGAACTTGGAGGCTCCCCCCAAATAATTAATCATACCTAGGTTAACCAAAATGTAAATAGACACCGCGT
TTCGGGTTGTCGGTGGTCAGCATTCCTCCTTTTGAAGATGCTTGTGATGATATGAGAAATCAGGTTTGGTAGGGGCTGCT
GCCTGGGCAAGTGGGGTTTGTGCATATGGGCCCAGGTAGTATGGTTGCACATGTCTGCTCACAGATGCTGTTGAATGGAA
GGAATGTTGTCTAGTCGCTTGCATTGTAACCATTGGAGATTGTCCATTTGTATGTATGGATCTTGTCTGTACATCTGTAA
TCCGTGCGATGATGTGTTTTATGGAACAGTCCAAGGCTTCAGGTCTTGTGGA
>XP_025979669.1 regulatory-associated protein of TOR 1 isoform X1 [Glycine max]
(SEQ ID NO: 31)
MALGDLMASRFSQSTVLVVPTSHNHLDDSTTASSSSSAAAAVAALNNNSSSNDDADFAHRRDSEAAIAIISSGNYAGNAA
TSMAYLPHTVVLCELRHDAFEAAVPAGPSDSGLVSKWRPKDRMKTGCVALVLCLNISVDPPDVIKISPCARMECWIDPFS
MAPQKALESIGKTLSSQYERWQPKARYKCQLDPTVDEVKKLCTTCRKYAKSERVLFHYNGHGVPKPTANGEIWVENKSYT
QYIPLPINELDSWLKTPSIYVEDCSAAGMIVNSFIELHEWSASNSSVSQRDCILLAACEAHETLPQSAEFPADVFTSCLT
TPIKMALRWFCTRSLLRESLDYSLIDKIPGRPNDRKTLLGELNWIFTAVTDTIAWNVLPHDLFQRLFRQDLLVASLERNF
LLAERIMRSANCSPVSHPMLPPTHQHHMWDAWDMAAELCLSQLPSLVEDPNAEFQPSTFFTEQLTAFEVWLDHGSEHKKP
PEQLPIVLQVLLSQCHRFRALVLLGRFLDMGPWAVDLALSVGIFPYVLKLLQTTTPELRQILVFIWTKILALDKSCQVDL
VKDGGHIYFIKFLDSMEAYPEQRAMAAFVLAVIVDGHRRGQEACIEAGLIHVCLKHLQSSCPNDSQTEPLFLQWLCLCLG
KLWEDFSEAQTIGLQEDATTIFAPLLSEPQPEVRASAVFALGTLLDVGFDSCRSVGGDEECDDDDKFRAEVSIVKSMLDV
ASDGSPLVRAEVAVALARFAFGHNKHLKSIAAAYWKPQANSLINSLPSLTNIKGSVGGYAKQNQHMPHGSIVSPQIGPIR
VGNDNSPVVRDGRVSSSSPLAGSGIMHGSPLSDDSSHHSDSGILNDGFSNGVANHTGPKPFDNALYSQCVLAMCTLAKDP
SPRIANLGRRVLSIIGIEQVVAKPLKSSGVRTAESTASPLARSSSWFDMNGGHLPLTFRTPPVSPPRPSYITRMRRVCSL
EFRPHLMDSPDSGLADPLLGSGGASGTSDRSFLPQSTIYSWSCGHFSKPLLTAADDSEEVSARREEREKFALEHIAKCQH
SAVSRLTNPIAKWDIKGTQTALLQPFSPIVIAADENERIRIWNHEEATLLNSFDNHDFPDKGISKLCLVNELDESLLLAA
SCTSLYCLIFFILFSNYCQNINVRESFDAADGNIRIWKDYTLRGKQKLVTAFSSIHGHKPGVRNLNAVVDWQQQCGYLYA
SGEISSIMLWDVDKEQLVNSKSSSSDCSVSALAASQVHGGQFTAGFIDGSVRLYDVRTPDMLVCGLRPHTQRVEKVVGIG
FQPGLDQGKIVSASQAGDIQFLDIRNHSSAYLTIEAHRGSLTALAVHRHAPIIASGSAKQLIKVFSLEGDQLGTIRYYPT
LMAQKIGSVSCLNFHPYQVLLAAGAADACVCIYADDNTQAR
>XM_026123884.2 LOC100777178 [organism = Glycine max] [GeneID = 100777178]
[transcript = X1]
(SEQ ID NO: 32)
TAATTTTTCTCGTTTGGCTTTACAAACTCGCTATTTCGTATCGTATCACTCGTTTCGGTTTTCCCTTCAAGGAGAGACAG
TGCCTGCCGCCACTGTTCTATGCGCCCCCTTCATCATCATCATCATCACCATCCCCATCCCTACCATCATCAACGCCAAC
ACCATAACCATCACCATCGCCATCATCATCACCTTCACCTTCACCTTAATCTTCATCTTCATATCCCTCACACTGCGAAA
ACCTAGGGTTCCTTCTTTCCTCAAAACCTTTCCTATTATTATTCCATGTACGATCGCCACTGAGGAGGGTTTCCGATTTG
GATGGCATTGGGAGATTTGATGGCCTCTCGCTTTTCCCAATCCACAGTTCTCGTCGTTCCCACCTCTCACAACCACCTCG
ACGATTCCACCACCGCCTCCTCTTCCTCCTCTGCTGCCGCCGCCGTCGCCGCTCTTAACAACAACAGCAGCAGCAACGAC
GACGCCGATTTCGCGCACCGCCGCGACTCTGAAGCCGCCATCGCCATCATTAGCAGCGGCAACTACGCCGGGAATGCCGC
CACCAGCATGGCCTACCTGCCCCACACCGTCGTCCTGTGCGAGCTCCGCCACGACGCCTTCGAGGCCGCCGTCCCCGCCG
GCCCCTCCGACAGCGGCCTCGTCTCCAAGTGGCGACCTAAGGACAGAATGAAGACAGGATGTGTAGCTCTAGTATTATGT
TTAAACATTAGTGTCGATCCACCAGATGTAATAAAGATATCCCCTTGTGCCAGAATGGAGTGCTGGATAGATCCTTTTTC
TATGGCACCGCAAAAGGCATTGGAGTCAATTGGAAAAACTTTGAGCAGTCAGTATGAAAGATGGCAACCAAAGGCCCGCT
ATAAATGTCAACTTGATCCGACAGTGGATGAGGTGAAGAAGCTTTGTACTACTTGTCGTAAATATGCAAAGTCGGAAAGA
GTTTTATTCCATTACAATGGGCATGGTGTGCCAAAACCAACTGCTAATGGTGAAATTTGGGTCTTCAATAAGAGTTATAC
ACAGTATATTCCCTTACCTATCAATGAACTTGATTCCTGGCTGAAGACCCCATCAATTTATGTTTTTGATTGCTCTGCGG
CTGGGATGATTGTGAATTCCTTCATTGAGCTTCATGAGTGGAGTGCTTCCAACTCCTCTGTGTCCCAAAGGGATTGCATT
CTGCTTGCAGCATGTGAAGCACATGAGACTTTGCCTCAGAGTGCAGAATTCCCAGCTGATGTATTTACATCTTGCCTTAC
AACACCTATAAAGATGGCATTGCGATGGTTTTGTACTCGTTCATTACTTCGTGAATCACTTGATTATTCACTTATAGACA
AGATCCCTGGCCGTCCAAATGACCGAAAGACACTTCTGGGTGAATTGAATTGGATCTTTACAGCCGTAACTGATACGATT
GCCTGGAATGTTCTTCCTCATGATCTTTTTCAGAGACTGTTCAGACAGGATTTGCTTGTTGCAAGTCTGTTTCGAAATTT
TCTACTTGCTGAGCGAATCATGCGATCTGCAAATTGTTCTCCTGTCTCTCACCCAATGTTACCTCCGACCCATCAGCATC
ATATGTGGGATGCATGGGACATGGCTGCTGAGCTCTGCCTCTCTCAACTTCCGTCTTTAGTTGAGGATCCTAATGCTGAA
TTTCAGCCTAGTACATTTTTCACTGAGCAGTTGACAGCATTTGAGGTATGGCTTGACCATGGTTCTGAACATAAGAAACC
ACCAGAACAGTTGCCTATAGTTCTTCAGGTTTTACTTAGTCAATGCCATCGGTTTCGGGCTTTAGTACTCCTTGGAAGGT
TCCTTGATATGGGGCCATGGGCAGTAGATCTGGCATTATCTGTTGGAATATTTCCATATGTTCTAAAGCTGTTACAAACA
ACAACACCCGAACTACGCCAGATCCTTGTATTCATATGGACAAAGATTCTTGCACTTGACAAGTCATGTCAGGTTGATCT
AGTGAAGGATGGCGGTCATATCTATTTTATTAAGTTTCTTGATAGCATGGAAGCATATCCAGAGCAACGTGCAATGGCTG
CTTTTGTTTTGGCTGTGATTGTGGATGGTCATAGACGAGGCCAAGAAGCTTGTATTGAAGCTGGTTTAATTCATGTCTGC
TTAAAGCACCTTCAGAGTTCATGTCCTAATGATTCACAAACTGAACCCCTTTTCCTTCAGTGGCTTTGCCTGTGTCTGGG
GAAATTGTGGGAAGACTTTTCAGAGGCACAAACAATCGGTTTGCAGGAAGATGCCACTACTATATTTGCTCCTCTACTGT
CTGAACCCCAGCCAGAGGTTAGGGCATCTGCTGTTTTTGCACTAGGCACCCTTCTTGATGTGGGGTTTGATTCATGTAGA
AGTGTTGGTGGAGATGAAGAATGTGATGATGATGATAAGTTTAGGGCTGAAGTTAGTATAGTTAAGAGTATGTTAGATGT
TGCTTCAGATGGGAGTCCATTGGTGAGGGCAGAGGTAGCAGTGGCTCTAGCACGATTTGCTTTTGGCCACAACAAGCACC
TGAAATCTATTGCTGCTGCATATTGGAAGCCTCAAGCTAATTCTTTGATTAATTCCTTACCTTCGTTGACTAATATAAAA
GGTTCGGTTGGTGGATATGCTAAACAGAACCAACACATGCCTCATGGAAGTATTGTTTCTCCTCAAATTGGTCCTATCAG
GGTTGGAAATGACAACTCTCCAGTGGTTCGAGATGGCCGCGTCTCTTCTAGCAGTCCTCTCGCTGGTTCAGGAATCATGC
ATGGGTCCCCATTGTCTGATGATTCATCTCATCATTCTGATTCAGGAATTCTGAATGATGGTTTCAGCAATGGAGTGGCT
AACCACACTGGACCAAAGCCCTTTGACAATGCATTGTATTCACAATGTGTACTTGCTATGTGTACTTTGGCAAAGGATCC
ATCTCCACGCATTGCAAATCTTGGTCGCCGGGTACTGTCCATAATAGGTATTGAACAAGTGGTTGCAAAACCATTGAAGT
CTAGTGGTGTTCGGACTGCTGAATCTACAGCTTCTCCACTGGCTCGTTCATCTTCTTGGTTTGACATGAATGGAGGACAC
TTGCCTCTGACCTTCAGAACTCCTCCAGTCAGTCCTCCTCGCCCAAGTTATATAACTCGAATGCGTCGAGTTTGTTCATT
GGAGTTTAGGCCCCACCTGATGGATTCTCCAGACTCAGGATTAGCTGATCCACTCTTAGGTTCTGGTGGAGCTTCTGGGA
CTTCAGATCGAAGCTTTCTTCCACAATCAACTATATATAGTTGGAGTTGTGGGCACTTTTCCAAACCACTTTTAACTGCT
GCAGATGATAGCGAAGAGGTATCAGCCAGAAGAGAAGAGAGGGAAAAATTTGCATTGGAGCACATAGCAAAATGCCAGCA
CTCTGCTGTTAGCAGGCTAACAAATCCAATTGCTAAATGGGACATAAAGGGTACACAAACAGCATTGCTGCAACCTTTCT
CTCCTATAGTGATAGCTGCTGATGAGAATGAACGTATCAGGATATGGAACCATGAGGAGGCAACACTACTGAACAGTTTT
GATAATCATGATTTTCCTGACAAAGGAATTTCTAAGCTCTGCCTTGTAAATGAGCTTGATGAGAGCTTGCTTCTTGCTGC
TTCATGTACTTCTCTCTACTGTTTAATATTTTTCATTTTATTCTCAAATTATTGTCAGAATATTAATGTTAGGGAATCTT
TTGATGCAGCTGATGGAAACATTCGGATTTGGAAAGATTATACTCTGAGGGGTAAACAAAAACTTGTCACTGCATTCTCT
TCAATTCATGGTCATAAACCTGGTGTGAGGAATCTGAATGCAGTTGTTGATTGGCAACAACAATGTGGTTATCTGTATGC
ATCGGGTGAGATATCATCTATTATGCTATGGGATGTTGATAAAGAGCAGCTTGTCAATTCTAAATCTTCATCATCAGATT
GCAGTGTCTCAGCATTGGCTGCATCTCAAGTTCATGGGGGACAATTTACAGCTGGTTTCATAGATGGTTCTGTCCGACTT
TATGATGTCAGAACACCTGATATGCTCGTTTGTGGATTAAGGCCTCATACACAAAGAGTAGAAAAGGTTGTGGGGATTGG
CTTTCAGCCTGGATTAGACCAAGGAAAGATTGTTAGCGCTTCTCAGGCTGGGGATATCCAATTTCTTGATATAAGAAATC
ATAGCAGTGCCTATCTTACTATTGAAGCTCATAGGGGTTCACTCACAGCTTTAGCAGTACATAGACATGCCCCAATTATT
GCCAGTGGTTCAGCGAAACAACTCATTAAGGTCTTTAGCCTGGAGGGTGATCAACTAGGCACCATTCGATACTATCCTAC
CCTAATGGCCCAGAAAATTGGTTCTGTAAGCTGCCTCAATTTCCATCCATACCAAGTATTACTTGCTGCTGGTGCTGCAG
ATGCATGTGTTTGTATTTATGCTGATGACAACACTCAAGCTAGATGAATCCGCATTTTTTCCAATGATTTCATCGGTTGT
CTCCAGCTTAGAAGAGTGCATCAAACATCTCTCAGGATTACCATGCATATTTTCTTGTAGTTGGATAATCCCGTGATGAT
GCTTTAATCCCTCTCCGCTGTGAATACATCTGAAAATATTGGTAGGGGTACATTCTGCTGATAGTGTATGCAAGATATTC
ATTAGCCTCCATGCTTAGAGGGTTGCACTCGACAGGAAGAAAGTCATGCTTATTGATTGGAGGCTTCGGCAAATAATGTC
AATTCGTAAATGTGTAAATATCATTTTCTCCATAATTATTTTTCGTTCCTGTTTTGCGTGCTATTCCCATCCTTCCTTGA
TTTGTAAAATTTTTAGTCAGAATACATAAGCTAGTTTGCTGACAAATATGGGCGACTGGCTCCTGAAAATGAGCCATGTC
ATGATTGTAGTTTCTGTTGAATCGGGGGACCCTTGTTTTTAACCAACATGTAATTAAATTCAAGTTTGTCCAAGTGTGCA
A
>XP_013736091.1 regulatory-associated protein of TOR 1 [Brassica napus]
(SEQ ID NO: 33)
MALGDLMVSRFSQSSVSLASSHRYDDDCVSSSQGDSSRRKDSDATSSIYGNGTAERASATSMAYLPQTIVLRELRHDASE
ASALLGTSEGIVLAPKWRLKERMKTGCVALVLCLNITVDPPDVIKISPCARIEAWIDPFSMAPPKALETIGQNLSTQYER
WQPRARYKVQLDPTVDEVRKLCLTCRKYAKTERVLFHYNGHGVPKPTANGEIWVENKSYTQYIPLPISDLDSWLKTPSIY
VFDCSAARMILNAFAELHDWGSSGSSGSTRDCILLAACDVHETLPQSVEFPADVFTACLTTPIKMALKWFCRRSLLKEII
DESLIDRIPGRQNDRKTLLGELNWIFTAVTDTIAWNVLPHELFQRLFRQDLLVASLERNFLLAERIMRSANCNPISHPML
PPTHQHHMWDAWDMAAEICLSHLPQLVLDPNAEFQPSPFFTEQLTAFEVWLDHGSEHKKPPEQLPVVLQVLLSQCHRFRA
LVLLGRFLDMGSWAVDLALSVGIFPYVLKLLQTTTNELRQILVFIWTKILALDKSCQIDLVKDGGHTYFIRFLDSSGAFP
EQRAMAAFVLAVIVDGHRRGQEACLEANLIGVCLGHLEASIPSDPQPEPLFLQWLCLCLGKLWEDFMEAQIMGREANAFQ
KLAPLVSEPQPEVRAAAVFALGTLVDIGFDSSKSVVDDEFDDDEKIRAEEAIIKSLLDVVSDGSPLVRAEVAVALARFAF
GHKQHLKSAASSYCKPQSSSLLTSLPSIAKLHDAGNAKLVSLHMSPLTRASTDSQPVAREAKISSSPLGSAGLMNGSPLS
DDSSLHSDSGIMLDIVSNGAGHHQRLLDNAVYSQCIRAMFALARDPSPRIASLGRRVLSIIGIEQVVAKPSKPTGRLGEA
ATTSNTPLTGLARSSSWFDMHAGNLPLSFRTPPVSPPRTNYLLRRVCSLEFKPHLLGSPDSGLADPPLGVSGSERSLLPL
STIYSWSCGHFSKPLLGGTDASQEIVTKREEKEKFALEHIAKCQHSSISKLSNNPIANWDTRFETGTKTALLHPFSPIVV
AADENERIRVWNYEEATLLNGFDNHDFPDKGISKLCLINELDDSFLLVASCDGSVRIWKNYATKGKQKLVTGFSSIQGHK
PSARDLNAVVDWQQQSGYLYASGEVSTVALWDLEKEQLLRSIPSESECGVTALSASQVHGGQLAAGFADGSLRLYDIRSP
EPLVCATRPHQKVERVVGLSFQPGLDLAKVVSASQAGDIQFLDLRTTKDTYLTIDAHRGSLTALAVHRHAPIIASGSAKQ
LIKVFSLQGEQLGIIRYYPSFMAQKIGSVSCLAFHPYQVLLAAGAADSFVSVYTHDNSQAR
>XM_013880637.2 LOC106439232 [organism = Brassica napus] [GeneID = 106439232]
(SEQ ID NO: 34)
CGAACCTCGCCCCAATCGTGACAATAACTCTAGGGCCGAGCGATTCGATTTTGAGGGATTTGGGGTGTTTGATTCTTCTC
TGGTGTGTTCTCCGATTTGGATGGCATTGGGAGACTTAATGGTATCTCGGTTCTCGCAATCCTCAGTTTCTCTGGCTTCC
AGTCACCGCTACGACGACGACTGTGTCTCCTCCAGTCAAGGTGATTCCTCGCGGAGGAAAGATTCCGATGCTACGAGTAG
TATCTACGGAAATGGCACGGCGGAGAGAGCCTCCGCTACCAGCATGGCGTACTTGCCTCAGACTATCGTCCTCCGGGAGC
TTAGGCACGACGCGTCCGAGGCGTCTGCGCTTTTGGGGACTTCCGAGGGGATTGTGTTGGCTCCTAAATGGCGGCTTAAA
GAGCGAATGAAAACAGGATGTGTAGCTCTAGTTCTGTGTTTAAACATTACTGTTGATCCGCCGGATGTTATAAAGATATC
TCCTTGTGCAAGAATCGAGGCTTGGATAGACCCATTTTCCATGGCGCCGCCAAAAGCTCTCGAGACTATTGGACAAAACT
TGAGCACTCAGTATGAAAGATGGCAACCAAGGGCTCGATATAAGGTTCAGTTAGATCCGACGGTAGACGAAGTCAGGAAG
CTGTGCTTGACTTGTCGGAAATATGCAAAGACCGAGAGGGTTCTATTCCATTATAATGGGCATGGTGTGCCAAAGCCTAC
AGCTAATGGGGAAATTTGGGTATTCAACAAGAGTTATACGCAGTACATTCCATTGCCAATCAGTGATCTTGATTCCTGGT
TGAAGACACCGTCCATCTATGTTTTTGACTGCTCTGCTGCTAGGATGATTCTTAATGCGTTTGCTGAGCTTCATGATTGG
GGTTCGTCTGGTTCCTCTGGATCCACAAGGGATTGTATTCTACTTGCTGCTTGTGACGTACATGAGACACTTCCTCAGAG
CGTTGAATTTCCAGCTGATGTTTTTACTGCTTGCCTCACGACACCCATTAAAATGGCGTTAAAATGGTTCTGCAGGCGTT
CTCTTCTAAAAGAAATAATCGATGAATCACTTATTGACAGAATTCCCGGCCGCCAAAATGACCGTAAGACATTGTTAGGG
GAGTTGAATTGGATCTTCACAGCAGTGACGGATACAATTGCATGGAATGTGCTTCCTCATGAACTTTTCCAGAGATTATT
CAGACAGGATTTGTTGGTCGCTAGCCTTTTCCGAAATTTCTTACTTGCTGAGAGAATAATGCGGTCCGCAAATTGTAATC
CAATATCTCACCCTATGCTACCTCCTACGCATCAACATCATATGTGGGATGCATGGGATATGGCTGCTGAAATCTGCCTT
TCGCACCTTCCCCAACTTGTTCTGGACCCAAACGCAGAGTTCCAGCCAAGTCCATTTTTTACTGAGCAATTAACAGCTTT
TGAGGTGTGGCTTGATCATGGATCTGAGCATAAGAAGCCACCGGAGCAATTGCCTGTTGTTCTTCAGGTGTTACTTAGCC
AATGCCATCGGTTTCGTGCTCTTGTACTTCTTGGAAGATTTCTTGATATGGGTTCATGGGCCGTGGATCTGGCTTTGTCT
GTTGGAATATTCCCATATGTGCTGAAGCTTCTGCAAACAACAACAAATGAGCTAAGACAGATCCTTGTTTTCATATGGAC
AAAAATTCTGGCACTTGATAAGTCATGTCAAATTGACCTTGTGAAGGATGGGGGGCATACATATTTCATAAGATTTCTAG
ATAGCTCGGGTGCATTTCCAGAACAACGAGCTATGGCTGCTTTTGTTCTGGCTGTCATAGTGGACGGACACCGACGAGGC
CAGGAAGCATGTCTTGAAGCAAATTTGATTGGTGTTTGTCTGGGGCACCTTGAAGCATCGATACCAAGTGATCCACAGCC
AGAACCATTGTTTTTACAATGGCTTTGTCTTTGTCTTGGAAAGTTGTGGGAAGATTTTATGGAGGCCCAAATAATGGGCA
GGGAGGCAAATGCTTTTCAAAAATTGGCACCTCTGGTTTCCGAGCCCCAACCTGAGGTAAGGGCTGCTGCTGTTTTTGCC
CTGGGTACCTTAGTTGATATTGGGTTTGACTCTAGTAAAAGTGTTGTGGACGATGAATTTGATGATGATGAAAAGATTAG
AGCAGAAGAAGCTATCATTAAAAGTCTCTTAGATGTTGTCTCAGATGGGAGTCCACTTGTCCGGGCAGAGGTTGCTGTAG
CTCTTGCACGTTTTGCCTTCGGCCACAAACAGCACCTAAAGTCAGCCGCATCTTCATATTGCAAGCCTCAGTCAAGTTCT
TTGCTTACTTCGCTCCCTTCAATAGCTAAACTCCATGATGCTGGGAATGCAAAACTTGTTTCTTTACACATGAGTCCTCT
GACTAGAGCTAGCACTGATAGCCAACCAGTGGCTCGTGAGGCCAAGATCTCAAGCAGCCCTCTTGGCTCGGCTGGGCTGA
TGAATGGATCTCCATTATCTGATGATTCTTCGTTACATTCTGATTCTGGAATCATGCTTGACATTGTCAGCAATGGAGCT
GGCCATCACCAAAGACTATTGGACAATGCTGTTTATTCGCAGTGCATTCGAGCTATGTTTGCCTTAGCTAGAGACCCATC
TCCACGTATTGCAAGTCTCGGGCGGCGTGTGCTTTCTATCATTGGAATCGAACAAGTTGTTGCGAAACCCTCGAAGCCCA
CTGGCCGACTAGGGGAAGCCGCAACGACATCTAACACTCCTCTTACTGGCCTAGCTCGTTCATCCTCATGGTTTGATATG
CATGCAGGTAATCTGCCCTTAAGTTTTAGGACTCCTCCGGTCAGCCCTCCTAGAACAAACTATCTACTGAGGAGAGTTTG
TTCGCTAGAGTTCAAACCTCATCTGTTGGGTTCACCAGACTCAGGATTGGCTGATCCGCCTTTAGGCGTCAGTGGATCTG
AAAGGAGTTTGCTCCCATTATCAACTATCTACAGCTGGAGCTGTGGCCACTTTTCTAAACCGCTTCTTGGTGGGACGGAT
GCTAGTCAAGAGATAGTTACCAAAAGAGAAGAGAAAGAAAAATTCGCACTCGAGCATATTGCAAAATGCCAGCACTCCTC
TATTAGCAAGCTCAGCAATAATCCTATTGCCAACTGGGATACGAGGTTTGAAACGGGAACAAAGACAGCTCTCCTTCACC
CTTTCTCTCCTATTGTAGTTGCTGCGGATGAGAATGAACGGATCAGAGTCTGGAACTATGAGGAAGCAACTCTTCTCAAT
GGCTTTGACAATCATGATTTTCCTGACAAAGGAATTTCAAAGCTCTGCCTCATCAATGAACTTGACGACTCTTTTCTACT
TGTTGCATCATGTGATGGCTCGGTCCGAATATGGAAAAACTATGCAACGAAGGGTAAACAAAAGCTTGTTACGGGGTTTT
CTTCAATTCAGGGCCACAAGCCCAGTGCACGTGACTTGAACGCTGTTGTGGACTGGCAACAACAGTCTGGCTACCTGTAT
GCTTCTGGGGAGGTGTCAACAGTCGCACTTTGGGACCTGGAGAAAGAACAGCTTCTCAGATCTATTCCCTCTGAGTCAGA
GTGCGGAGTTACAGCACTTTCGGCTTCTCAAGTGCACGGGGGTCAACTTGCTGCTGGTTTCGCTGATGGATCTTTGAGAC
TCTATGATATTCGGTCACCTGAACCGCTTGTCTGTGCGACTCGGCCTCATCAGAAAGTTGAAAGAGTGGTTGGACTCAGT
TTTCAGCCTGGGCTTGATCTCGCAAAGGTGGTGAGTGCATCACAGGCCGGTGACATACAGTTCCTTGACCTCAGAACAAC
AAAGGACACATACCTGACGATTGATGCACACAGGGGTTCACTCACGGCCTTAGCTGTTCACAGACACGCTCCAATCATCG
CGAGCGGATCTGCAAAACAGCTGATTAAAGTGTTTAGCCTTCAGGGAGAACAACTAGGGATAATCCGCTACTACCCGTCC
TTCATGGCACAGAAGATCGGTTCAGTGAGCTGCCTCGCTTTTCATCCGTACCAGGTTCTGCTTGCAGCTGGAGCTGCGGA
CTCATTCGTCTCTGTATACACCCATGACAACTCACAAGCAAGGTGAGTCGCACACCGTAAGTCAAAATGTTGCATCGCTC
TTGTGATAAAGAAAGGTCCAGTCCTGGTCGGGGGTAAAGTGGTTTATAGTTATTCGTTAGCCTCCGTTGAAGTTTAGAAA
GGAGATCTAAGTTAACAACTGATGAAGGAGGAGGCTTCTCCAAATAAGTTCTCTATCTCTTCCTATGCCAACTCAAGTAA
ATGGTATCTGTAAATAGTTTCACTGTTTCGTAATTTGTTTTTTATTTAGCATGTTTCATTGGGTGTGTGCTTTCTTTCCT
TTCTATTAAAATCTTAAAAGTCTTTTCCGTTTCACAATTTGTTTCAAAATGTTAAAAAGTAATTGTGGAAAAAAAAAAGT
TTGCTTTCGTGATATTATTAGAAGGATTAGTATATTGAAAA
>XP_040958106.1 regulatory-associated protein of TOR 1 isoform X1 [Gossypium
hirsutum]
(SEQ ID NO: 35)
MALGDLMTSRFSQLSLAVSNHVMDGNGSNGDYLEDAASADMASQRRDEDTASTSSYANAVASSATVPTTMAYLPQTIVLC
ELRHAAFEASTPTGPSDSGLVSKWRPKDRMKTGCVALVLCLNISVDPPDVIKISPCARMECWIDPFSMAPQKALETIGKN
LRDQYERWQPKARCKVELDPTVDEVKKLCNTCRRYAKSERVLFHYNGHGVPKPTANGEIWLFNKSYTQYIPLPISDLDSW
LRTPSIYVFDCSAAGMIVNAFIELLDSGTSSYPGSARDCILLAACEAHETLPQSAEFPADVFTSCLTTPIKMALRWFCTR
SLLHESLDCSLIDKIPGRQNDRKTLLGELNWIFTAVTDTIAWNVLPHELFRRLFRQDLLVASLERNELLAERIMRSANCS
PVSYPMLPPTHQHHMWDAWDMAAEICLSQLPALVEDPNAEFQPSLFFTEQLTAFEVWLDHGSEHKKPPEQLPIVLQVLLS
QCHRFRALVLLGRFLDMGPWAVDLALSVGIFPYVLKLLQTTTPELRQILVFIWTKILALDKSCQVDLVKDGGHVYFIRFL
NSVEAYPEQRAMAAFVLAVIVDGHRRGQEACIEAGLIHVCLKHLHSSMQNDVQTEPLFLQWLCLCLGKLWEDFTEAQIIG
LQADAPAICAPLLSEPQPEVRASAVFALATLLDIGEDSFRDGVGGDEECDDDEKTRAEIIVIIKSLLNIVSDGSPLVRAE
VAVALARFAFGHKQHLKSIAAAYWKPQSNSLLSSLPSLANINGTGSGNIVSSQIGPLIRVGNDNSSVVRDGRVSTSSPLA
TAGVMHGSPLSDDSSQHSDSGILNDGISNGVIRHSRSKPLDNAMYSQCVPAMCTLAKDPSPRIATLGRRVLSIIGIEQVT
KSVKSAGSSSARPGEPTTSSTTPSFAGLARSSSWFDMNGGHLPLTFRTPPVSPPRQNFLTGMRRVCSLEFRPHLMNSPDS
GLADPLLGSSSGVSERSLLPQSTIYNFSCGHFSKPLLTASDDSEELLAIREERERFALERIAKCQHSSVSKLNNNSQIAS
WDTRFETGTRTALLQPFSPIVIAADESERIRVWNYEEATLLNGFDNHDFPEKGISKLCLLNELDESLLLVASGDGNIRIW
KDYTVRGKQKLVTAFSSIQGHKPGVRSLSTVVDWQQQSGYLYASGEISSVMIWDLDKEQLINSIPSSSDCSVSALASSQV
HAGQFAAGFVDGSVRLYDVRAPDMLVCATRPHTQQVERVVGIGFQPGLDQGKIVSASQAGDIQFLDIRNQRDAYLTIDVH
RGSLTALAVHRHAPIIASGSAKQLIKVFSLEGEQLGVIRYQHTFMAQKIGSVSCLTFHPYQVLLAAGAADACVSIYADDN
SQTR
>XM_041102172.1 LOC107914561 [organism = Gossypium hirsutum] [GeneID = 107914561]
[transcript = X1]
(SEQ ID NO: 36)
AGAAGGAAAGAGTACACAAAAATTAAATATACAGCAATGCACGCACAAAAACTGGAATGCATAGACAAAATCCTTATTTG
AGAGAGTTGAGTTGAAACCCCCCTTCCTTTCTTTCTCTCTCTCTTTCCATTTTTCTTTTAATTTATTTTTCTTGTGCCGC
CAGTGTTGTATTCGCCACCACCATCGTCGCTGCCGTAACCTAGGGTTTGTTCTAGGGTTTGAGATCTCTTTTCGCTTATC
TTTTCTTCGCACAACTTTCCGAGCTGGATTCTCTCTCTTTCCTCAGGCGAATCTCCATGCGCCGCTGGTTCAGCGGCTTT
GTTGATTAAATCACTTCCTCTTCTTCCTCCTCCTCCTCCTCCTCCTCTTCTTCTTCTTCTTCTTCTCTCGTTCTTTCTTG
CTTTCATTCCTCGATCAGTTTCGAGAAGAGAGGCTAGGGTTTTCTTGTTGTGGAATTTTTCTTTGTTTCTTATTAATTAA
ATTTTCATCCGAGGGCTTTGTTTGTTTCTTGTTTCATTTTGTTGGGGGATTTGGATGGCATTGGGCGATCTGATGACGTC
ACGGTTTTCGCAATTGTCGTTGGCAGTATCCAATCATGTCATGGACGGCAACGGCAGCAATGGCGACTACCTTGAGGATG
CTGCTTCTGCCGACATGGCCTCTCAAAGGAGGGATTTTGATACTGCATCCACCAGTAGTTATGCCAATGCCGTTGCATCC
TCCGCCACAGTGCCTACAACTATGGCTTACTTGCCTCAAACTATTGTCTTATGCGAGCTTCGGCATGCTGCGTTTGAAGC
TTCCACTCCTACTGGACCCTCTGATAGTGGCCTTGTCTCCAAATGGCGACCCAAGGATCGAATGAAGACAGGATGTGTAG
CACTAGTTTTATGTTTAAATATTAGTGTTGATCCACCTGATGTAATAAAGATATCTCCTTGTGCAAGAATGGAGTGTTGG
ATTGATCCTTTTTCTATGGCACCACAAAAAGCTCTCGAAACCATTGGGAAAAATTTGAGAGATCAATATGAGAGGTGGCA
GCCCAAGGCACGGTGCAAGGTTGAGCTTGATCCTACAGTGGATGAAGTGAAGAAACTTTGTAATACATGTCGTAGATATG
CCAAGTCAGAGAGAGTTCTATTTCATTATAATGGACATGGTGTTCCAAAGCCAACTGCAAATGGTGAAATCTGGCTTTTC
AATAAGAGTTATACACAGTATATTCCCTTGCCTATCAGTGACCTTGATTCATGGTTAAGAACACCTTCTATTTATGTTTT
TGATTGCTCTGCGGCTGGAATGATTGTTAATGCCTTCATTGAGCTTCTTGACTCTGGCACTTCTAGTTACCCTGGATCTG
CACGAGATTGCATTCTGCTGGCTGCATGTGAAGCACATGAGACACTTCCTCAAAGTGCTGAATTTCCTGCTGATGTGTTT
ACTTCTTGTCTTACTACACCTATCAAAATGGCATTGAGATGGTTTTGCACACGTTCGTTGCTTCATGAGTCTCTTGACTG
TTCTCTTATAGATAAAATTCCTGGCCGCCAAAATGATCGTAAAACACTTCTAGGGGAGTTGAATTGGATTTTTACTGCAG
TAACAGACACAATTGCTTGGAACGTTCTCCCTCATGAGCTTTTCCGGAGATTGTTTAGGCAGGATCTACTAGTTGCCAGT
TTGTTCAGAAATTTTTTGCTTGCTGAGAGGATTATGCGCTCTGCAAATTGTTCTCCAGTTTCTTACCCAATGTTGCCACC
AACTCATCAGCACCATATGTGGGATGCATGGGACATGGCTGCTGAAATTTGCCTTTCTCAGCTTCCAGCATTGGTCGAGG
ATCCTAATGCGGAGTTCCAGCCAAGTCTCTTTTTTACTGAACAGCTGACAGCTTTTGAGGTATGGCTTGACCATGGATCT
GAGCATAAAAAGCCACCGGAGCAATTGCCTATCGTTCTTCAGGTTTTACTTAGTCAATGCCATAGATTCCGTGCACTGGT
TCTTCTTGGAAGATTCCTTGATATGGGACCATGGGCTGTAGATCTGGCCCTTTCTGTTGGAATATTCCCCTATGTGCTGA
AGCTGTTGCAAACAACCACACCAGAACTGCGTCAAATCCTTGTCTTCATATGGACAAAAATTTTGGCCCTTGATAAGTCA
TGTCAGGTTGATCTGGTAAAAGATGGCGGTCATGTTTATTTCATTAGGTTTCTTAATAGCGTGGAAGCATATCCTGAACA
GCGAGCAATGGCTGCATTTGTTCTAGCTGTCATTGTGGATGGGCATAGACGGGGCCAGGAAGCCTGTATTGAGGCAGGGT
TGATCCACGTGTGCTTGAAGCACCTTCACAGTTCCATGCAAAATGATGTGCAAACCGAACCCTTGTTCCTTCAGTGGCTT
TGCTTGTGTCTTGGGAAGCTCTGGGAGGATTTTACTGAGGCTCAAATAATAGGTTTGCAGGCAGATGCACCTGCAATATG
CGCTCCTCTTCTTTCTGAGCCGCAGCCTGAAGTTAGGGCTTCTGCAGTTTTTGCATTGGCTACCTTGCTTGACATTGGGT
TTGATTCATTCAGAGATGGTGTTGGAGGGGATGAAGAATGTGATGATGATGAAAAGACTAGAGCCGAGATTATTGTTATT
ATTAAAAGCCTTCTGAATATTGTTTCTGATGGTAGCCCTCTTGTCAGAGCAGAAGTAGCTGTAGCTCTAGCAAGGTTTGC
CTTTGGACATAAGCAGCATCTCAAGTCAATTGCTGCTGCATATTGGAAACCTCAATCTAATTCCTTGTTGAGTTCATTGC
CTTCGCTGGCTAATATAAATGGCACAGGAAGTGGGAATATAGTTTCATCCCAGATAGGCCCTTTAATTCGTGTAGGAAAT
GACAACTCATCTGTAGTTCGGGATGGAAGGGTTTCCACCAGCAGTCCTCTTGCTACTGCTGGAGTCATGCATGGATCTCC
TTTATCTGATGATTCATCTCAACATTCTGATTCTGGGATTTTGAATGATGGTATTAGTAATGGAGTCATTCGTCATTCAA
GGTCAAAACCTTTGGACAATGCAATGTATTCACAGTGTGTACCGGCTATGTGTACTTTAGCTAAGGACCCTTCTCCACGC
ATAGCAACTCTAGGCAGGCGGGTTCTTTCAATTATTGGGATTGAACAAGTGACAAAATCTGTGAAGTCTGCTGGTAGCAG
CAGTGCTAGGCCTGGTGAGCCTACAACTTCCTCAACGACTCCTAGTTTTGCTGGGTTAGCTCGTTCTTCTTCATGGTTTG
ACATGAATGGAGGTCATCTGCCTTTAACGTTCAGAACTCCTCCTGTTAGCCCCCCTCGGCAAAATTTTTTGACAGGAATG
CGTAGAGTTTGCTCTTTAGAATTCAGGCCTCATCTGATGAATTCTCCGGACTCCGGATTGGCTGACCCACTTCTAGGGTC
TTCTTCTGGGGTTTCTGAGCGCAGTTTACTTCCACAATCAACTATCTATAATTTTAGTTGTGGCCACTTCTCAAAGCCAC
TTCTTACTGCATCAGATGATAGTGAAGAACTATTGGCTATAAGAGAAGAGCGGGAGAGATTTGCACTGGAGCGTATTGCT
AAATGTCAGCACTCCTCTGTTAGCAAACTTAACAATAATAGTCAAATTGCTAGTTGGGATACAAGATTCGAGACAGGTAC
AAGAACAGCATTGCTGCAACCTTTCTCTCCTATTGTTATCGCGGCAGATGAGAGTGAACGGATCAGGGTATGGAATTATG
AGGAAGCCACCCTTCTCAATGGTTTTGATAATCATGATTTTCCAGAGAAGGGAATTTCTAAACTTTGTCTTTTGAATGAG
CTTGATGAAAGCTTGTTGCTTGTTGCTTCAGGTGATGGAAATATACGGATTTGGAAAGATTATACAGTCAGGGGCAAACA
GAAACTCGTTACTGCATTTTCTTCTATTCAAGGTCACAAACCTGGTGTGCGGAGTTTGAGTACTGTTGTGGACTGGCAGC
AGCAGTCTGGATATCTTTATGCATCTGGAGAGATATCATCTGTCATGATCTGGGACCTGGATAAGGAGCAACTTATTAAT
TCAATACCATCCTCCTCAGATTGCAGTGTCTCAGCATTGGCTTCTTCTCAAGTTCATGCTGGTCAATTTGCAGCCGGTTT
TGTGGATGGTTCTGTCAGACTCTATGACGTCCGGGCACCTGACATGCTGGTTTGTGCAACTAGACCACATACCCAGCAAG
TTGAAAGAGTTGTGGGTATTGGCTTTCAGCCTGGACTTGATCAAGGAAAGATTGTTAGTGCTTCCCAGGCAGGTGATATT
CAGTTTCTTGATATTAGAAACCAAAGAGATGCTTACCTTACAATCGATGTGCACAGAGGCTCACTTACAGCTCTAGCTGT
TCATAGACATGCCCCGATAATTGCCAGTGGGTCAGCAAAACAGCTAATCAAAGTCTTCAGTCTGGAAGGCGAGCAACTAG
GCGTCATTCGGTACCAACATACCTTCATGGCCCAGAAGATTGGTTCTGTGAGCTGCCTTACCTTCCATCCCTACCAAGTA
TTGCTTGCTGCTGGTGCTGCAGATGCGTGTGTTTCTATCTACGCTGATGACAATTCTCAGACAAGATGAATATAAATTCT
TTCCCAATTTCATGAATTCGGGAGGTATTGCACGGTAGAACTGGCATCGGCACAAATTGCACGGAGTCAGGATCGAAGAT
AACCTGTAAATGTTGCCTGCCACATTTACCATTTCATTGTGGGTATTCCTGGAAATTGGGGGGGGGGATCTGCTGATTGT
AATGCTATATATATAGTGGGATATATATTCATTAGCCTCCGGTGTAGATGGTCTTACACATGTTCGGAAAAAGGAATCCT
ATCCTGCAGATATTTCATGGATGGGGGGGCTTTGGCAAATAATTTGTTCTTATAGGTGCATGTAAATAGTTTTCCCATTA
TTATTTCACCCTTTTCAGTGTGTTCATTGTT
>XP_006366877.1 PREDICTED: regulatory-associated protein of TOR 1-like [Solanum
tuberosum]
(SEQ ID NO: 37)
MALGDLMASRFSQSSAALDEFGNEDGERNNVRDLDTASSSYVGGGVADNAMTTTSMAYFPQTIVLCELRHDRFEDSVPSG
PSDTGLVSKWRPRDRMKTGCVALVLCLNISVDPPDVIKISPCARMECWVDPFSMAPQKALETIGRTLNQQYERWQPRAKY
KISLDPTVDEIKKLCTTCRKYAKSERVLFHYNGHGVPKPTANGEIWLFNKSYTQYIPLPISDLDSWLKTPSIYVEDCSAA
GMIVNAFIELQDWTASGSSATSTRDSILLAACEAHETLPQSAEFPADVFTSCLTTPIKMALRWFCTRSLLHESLDYSLID
RIPGRQTDRKTLLGELNWIFTAVTDTIAWNVLPHDLFQRLFRQDLLVASLERNELLAERIMRSANCSPISYPMLPPTHQH
HMWDAWDMAAEICLSQLPNLVEDPNAEFQPSPFFTEQLTAFEVWLDHGSKDKKPPEQLPIVLQVLLSQCHRFRALVLLGR
FLDMGPWAVDLALSVGIFPYVLKLLQTTTPDLRQILVFIWTKILALDKSCQVDLVKDGGHTYFIRFLDSVEAYPEQRAMA
AFVLAVIVDGHRRGQEACFEAALIHVCLKHLQGSTPNEAQTEPLFLQWLCLCLGKLWEDFTEAQVQGLQADAPAIFAPLL
SEPQPEVRAAATFALGTLLDVGFDSARDGVGGDEDCDDEEKVRTEVSIIKSLLSVASDGSPLVRVEVAVALARFAFGHNK
HLKSVAAAYWKPQANSLLTSLPSFAVKSSGSGYTTPTHSISHGSRVPSPIAPLLRVGGDSQSISRDGRVSTSSPLATPGV
IHGSPLSDDSSQLSDPGILNDAVTNGVVNHTRSRPLDNALYSQCVLAMCALAKDPSPRIAGLGRRVLSIIGIEQVVAKSV
KSTGESTTVPNTGYAGLARSSSWFDMNGGHLPLTFRTPPVSPPRPSYLTGMRRVCSLEFRPHLMHSQDSGLADPLLGSAG
SSGPSERSFLPQPTIYNWSCGHFSKPLLTAADDSEEMVARREEKEKLALDLIAKCQHSSVSKLHNQIASWDTKFEIGTKT
ALLQPFSPIVIAADESERIRVWNYEEATLLNSFDNHSYPDKGISKLCLVNELDESLLLVASSDGNIRIWKDYTLRGRQRL
VSAFSSIQGHRPGVRSVNAVVDWQQQSGYLFSSGEVSSIMAWDLDKEQLVNTIPTSSDCSISALSASQVHAGHFAAGFVD
GCVKLFDIRMPELLVCASRPHTQRVERVVGIGFQPGLEPAKIVSASQAGDIQFLDMRNLKEAYLTIDAHRGSLTALAVHR
HAPLIASGSAKQLIKVFNLEGEQLGTIRYLSTFMAQKIGSVRCLTFHPYQVLLAAGAADSCVSIYADEIAPTR
>XM_006366815.2 LOC102586700 [organism = Solanum tuberosum] [GeneID = 102586700]
(SEQ ID NO: 38)
TACAAAACAACAACTCTCTCCTTCTCTTCTTTTTCTCGTTTTGTTGCTTCAACAGCCGCACTGTCCTCCTTCTTCTTGTT
GTTCTTCTTCATCAACACCATCTTCTAGGGTTTCTTTTAGGGTTTTCCAGAACCTTCTTGAAGCTGGATTCTCCTTCCTT
CCTTCTCCCCATCTCTCACCGTCGCTGGTTCACTGGTTTCATTGATTAATCCTCAAGAAACAACCTTTTTCAATCTCCCT
TTACCATCACTACTGAAACCCTGATCTTTATTGCAAATTACCGGATAAAAACGGTATCTTTAGGGTTTTTGGTATCTGGG
TCGATCTGGGTTATGAGTTTCACTCCAAAATCTGTTCGATTAGGGCAAGGTTAGGAAAATTTTCAGCTTCAATTATTGCG
ATTTGAATGGCATTGGGGGATTTGATGGCGTCGAGGTTTTCACAATCATCAGCTGCTTTGGATGAGTTTGGAAATGAGGA
TGGGGAAAGGAATAATGTTAGAGATTTGGACACTGCTAGTAGTAGCTATGTTGGTGGTGGGGTTGCTGATAATGCGATGA
CGACAACAAGTATGGCTTATTTTCCGCAGACAATTGTGTTGTGTGAGCTTAGGCATGATCGTTTTGAGGATTCTGTTCCT
TCTGGTCCATCTGACACTGGGTTGGTCTCTAAATGGCGGCCTAGGGACCGAATGAAAACAGGATGTGTTGCCCTTGTTTT
GTGTTTAAACATCAGCGTTGATCCACCTGATGTAATAAAGATATCTCCTTGTGCTCGAATGGAGTGTTGGGTTGATCCAT
TTTCGATGGCACCTCAAAAAGCACTTGAGACAATTGGGAGAACTTTGAACCAACAATATGAGAGGTGGCAACCTAGGGCT
AAATATAAGATTTCCTTGGATCCTACTGTTGATGAAATCAAGAAGCTTTGCACAACTTGTCGAAAGTATGCTAAGTCAGA
GCGAGTTCTATTCCATTATAATGGACACGGTGTTCCTAAACCTACAGCCAATGGTGAAATTTGGCTATTTAATAAGAGTT
ATACGCAGTATATTCCTTTGCCAATCAGCGACCTGGATTCCTGGTTGAAGACACCATCGATCTATGTATTTGACTGCTCT
GCTGCTGGGATGATTGTTAATGCCTTCATAGAGCTTCAGGACTGGACTGCTTCTGGTTCTTCTGCAACTTCTACAAGGGA
TAGTATTCTGCTTGCTGCTTGTGAAGCACATGAGACCCTTCCACAGAGTGCTGAGTTTCCAGCCGATGTGTTCACTTCCT
GCCTCACAACACCCATCAAAATGGCATTGAGATGGTTTTGCACTCGTTCGTTGCTTCATGAGTCGCTTGATTACTCTCTG
ATTGATAGAATTCCTGGCCGGCAAACTGATCGGAAGACACTTCTTGGCGAGCTGAATTGGATTTTTACTGCAGTTACTGA
CACAATTGCCTGGAATGTTCTACCACATGATCTATTTCAGAGACTGTTCAGGCAAGATCTGTTAGTTGCTAGTTTGTTCA
GAAACTTCTTACTTGCTGAAAGAATCATGCGCTCTGCAAATTGTTCACCAATTTCATACCCGATGCTACCACCTACTCAT
CAGCACCATATGTGGGATGCATGGGACATGGCAGCAGAAATCTGTCTTTCACAGCTTCCAAATTTAGTTGAGGATCCCAA
TGCAGAATTTCAGCCAAGTCCATTTTTCACAGAGCAGTTGACAGCTTTTGAAGTATGGCTTGACCATGGATCAAAGGATA
AAAAGCCTCCAGAGCAGTTGCCCATAGTGTTACAGGTGTTACTTAGTCAATGCCACAGGTTTCGTGCACTTGTACTTCTT
GGAAGATTTCTCGATATGGGGCCTTGGGCTGTTGATCTGGCCTTGTCTGTTGGAATATTTCCTTATGTTCTAAAACTTTT
GCAAACCACCACTCCAGATCTTAGACAAATTCTTGTCTTTATCTGGACGAAGATCCTTGCACTCGATAAGTCTTGTCAGG
TTGACCTTGTAAAGGATGGAGGACATACATATTTCATTAGATTTCTTGACAGTGTGGAGGCTTATCCAGAGCAAAGGGCA
ATGGCTGCATTTGTTTTAGCAGTCATTGTGGATGGTCACAGACGGGGTCAGGAGGCCTGTTTTGAAGCGGCTCTTATACA
TGTCTGCTTGAAGCATCTTCAGGGATCGACGCCTAATGAGGCACAAACTGAACCACTTTTTCTTCAATGGCTGTGCCTTT
GTCTGGGAAAACTGTGGGAGGATTTCACAGAGGCTCAAGTACAAGGTTTGCAGGCAGATGCACCAGCAATATTTGCGCCT
CTGCTGTCTGAGCCCCAACCTGAGGTACGTGCTGCAGCTACTTTTGCTCTAGGGACTTTACTTGATGTTGGATTTGACTC
GGCTAGGGATGGTGTTGGAGGAGATGAAGACTGTGATGATGAGGAGAAGGTTAGGACTGAAGTCAGTATAATTAAGAGCC
TTTTAAGTGTTGCTTCTGATGGAAGCCCTTTGGTTCGAGTGGAGGTTGCTGTAGCTTTGGCACGTTTTGCTTTTGGGCAT
AACAAGCATCTGAAGTCAGTAGCCGCTGCATATTGGAAGCCTCAGGCTAATTCCTTGCTAACTTCCTTGCCTTCTTTCGC
GGTCAAGAGTTCTGGTAGTGGGTATACAACCCCGACTCACAGTATTTCACATGGAAGTAGAGTTCCGTCTCCTATTGCTC
CTTTATTGAGAGTGGGGGGTGACAGTCAGTCCATATCTCGGGATGGGAGAGTCTCGACCAGCAGCCCCCTTGCAACACCC
GGCGTTATACATGGATCACCTTTATCTGATGATTCCTCTCAACTTTCTGATCCCGGAATCTTGAATGATGCTGTTACCAA
TGGTGTTGTAAATCATACAAGATCAAGGCCTCTAGACAATGCACTCTATTCTCAGTGTGTACTGGCTATGTGTGCCTTGG
CAAAGGATCCGTCACCACGTATTGCAGGTCTTGGCCGGAGAGTCCTTTCCATTATCGGGATTGAACAGGTTGTTGCCAAA
TCTGTTAAGTCAACTGGTGAATCCACAACAGTTCCAAACACTGGTTATGCTGGATTGGCTCGATCTTCATCTTGGTTTGA
TATGAACGGAGGACATTTACCTCTGACTTTTAGAACTCCTCCTGTTAGTCCCCCACGGCCTAGTTACTTGACAGGAATGC
GAAGAGTTTGTTCTTTAGAGTTCAGGCCCCACCTAATGCACTCTCAAGACTCAGGATTAGCTGACCCATTGTTGGGGTCT
GCTGGATCTTCTGGACCTTCAGAACGCAGTTTTCTTCCCCAACCAACTATCTACAATTGGAGTTGTGGCCACTTCTCAAA
GCCACTTCTTACTGCAGCTGATGATAGTGAAGAGATGGTTGCTAGAAGGGAAGAGAAGGAAAAGTTGGCCCTTGACCTCA
TTGCGAAGTGCCAACACTCTTCTGTAAGCAAACTACACAATCAAATTGCTAGTTGGGATACTAAGTTTGAGATTGGCACC
AAAACTGCTTTGCTGCAGCCCTTCTCACCTATTGTTATTGCTGCAGATGAGAGTGAAAGAATCAGGGTATGGAATTACGA
GGAGGCTACCCTTCTTAATAGCTTTGATAACCACAGTTACCCTGATAAAGGAATTTCAAAACTGTGTCTTGTCAATGAGC
TAGATGAGAGTTTGCTCCTTGTTGCTTCAAGTGATGGGAACATCCGGATTTGGAAAGACTATACTCTAAGAGGCCGACAA
AGACTAGTTTCTGCATTTTCATCCATTCAAGGTCACAGACCTGGTGTCCGTAGTGTGAACGCTGTTGTGGATTGGCAGCA
GCAGTCTGGATATCTGTTTTCATCTGGTGAAGTTTCGTCAATCATGGCTTGGGATCTTGACAAAGAACAACTTGTTAATA
CAATTCCAACATCCTCAGATTGCAGCATCTCAGCATTGTCAGCTTCTCAAGTTCACGCAGGACACTTTGCTGCTGGCTTT
GTGGATGGATGTGTCAAACTATTTGATATTCGAATGCCTGAACTGCTTGTTTGTGCATCACGGCCGCACACCCAAAGAGT
AGAGAGAGTAGTGGGGATCGGCTTTCAACCTGGACTTGAACCAGCAAAGATCGTCAGTGCCTCTCAAGCTGGTGATATTC
AGTTCCTTGATATGAGAAATCTCAAGGAAGCTTACTTAACAATTGATGCTCATAGGGGATCGCTAACAGCCCTAGCTGTT
CATCGTCATGCACCCCTTATAGCAAGTGGTTCAGCTAAACAGCTAATCAAAGTCTTCAACCTGGAAGGTGAGCAATTAGG
CACCATAAGATATTTGTCCACCTTCATGGCTCAGAAGATAGGTTCAGTCAGATGTCTTACCTTCCATCCCTATCAAGTGC
TGCTTGCTGCTGGAGCAGCAGATTCTTGTGTTTCAATCTATGCTGATGAAATTGCCCCGACAAGATAATTTTCTGAAACA
TTCAAGATTGAGAAACCAAAAAAGATTACAGACATAAGTTGAGGTGGTCAGCTGCAAAAATGGACTGTTGACATCTCTGA
AACATATCAGCTCACTGAGGTCCTGTGCTACTTATTCATTGAGCCTCTGCAGCGACGAGTGTTGCCAAGTTGGGAAAAAA
TTCAACATATTGGAGGAGGCTTTAGCAAATAATTTGATAAATAGGTGGTAAAAGAAGGTGTAAATATCTGTTATTCATTG
TTTCTAGTTTGTAATTTTCTCCGTGTGCTTCCTTAGTTCAGCCCCCTCTACTTTCTTTTCTCCCCTGGTTCAATTTGTAC
AGCTGAATTCATAATTTATATATGTTGTTGGCAACCAGTGTTCTGTGTTC
>XP_044380624.1 regulatory-associated protein of TOR 1-like isoform X1 A genome
[Triticum aestivum]
(SEQ ID NO: 39)
MALGDLMASRLVHSSSSSSSSSLPTPSLAAVNHQTDRVDGELPVANGPELRREDAGEEDEEGRAVALVPCLPQVVVLCEQ
RHEGFNEAAVAAAAGPSTSGLVSKWRPKDRMKTGCVALVLCLNISVDPPDVIKISPCARMECWIDPFSMPPPKALENIGK
TLHSQYERWQPKARYKLQLDPTVEEVKKLCNTCRKYARSERVLFHYNGHGVPKPTANGEIWMENKSYTQYIPLPITDLDS
WLKTPSIYVFDCSAAGMIVKAFLERLDWSSSSSASSVKDCILLAACEAHQTLPQSAEYPADVFTACLTTPIKMALHWFCK
RSLLSGSLDHSLIDQIPGRQNDRKTLLGELNWIFTAITDTIAWNVLPHELFQRLFRQDLLVASLERNFLLAERIMRSANC
SPITYPLLPPTHQHHMWDAWDMAAEICLSKLPHLIADPNAEFQPSPFFTEQLTAFEVWLDHGSEDKKPPEQLPIVLQVLL
SQSHRFRALVLLGRFLDMGPWAVDLALSVGIFPYVLKLLQTSAMELRQILVFIWTKILSLDKSCQVDLVKDGGHAYFIRF
LDSLDAYPEQRAMAAFVLAVIVDGHRRGQEACMSAGLIDVCLRHLQPENPHDAQTEPLLLQWLCLCLGKLWEDFPEAQLL
GLQSNAPEIVTCLLSEPQPEVRASAVFALGNLLDIGSPSVNGGDDDSDDDEKARAEINVVRSLLQVTSDGSPLVRAEVSV
ALTRFALGHNKHLKSVAAEYWRPQTNSLLKSLPSLANINNPSNVYSPSNFLQGSSGLSSHIGPVLRVGSDTSATGRDGRI
STSSPIATNSIMHGSPQSDDSSQHSDSGILLRDNASNGGLSYTRSRPIIDSGIYSLFISTMCSIAKDPYPRIANIGRRAL
SLVGVEQVVMRNTRFGSGGAGESSAPSSNIGMARSSSWFDMNSGISMAFRTPPVSPPQHDYLTGLRRVCSMEFRPHLLNS
PDGLADPLLSSAAGPSTSELNILPQSTIYNWSCGHFSRPLLTGSDDNGEVSARREERERTALDCIAKCQRSSACKMTSQI
ASWDTKFELGTKSALLLPFSPIVVAADENEQIRVWNYDDALPVNTFENHKLSDRGLSKLLLINELDESLLLVGSSMPSLF
MAFSCICTYVFLVTSFFHTVGDGNVRIWRNYTQKGGQKLVTAFSSVQGHRAAGRSVVIDWQQQSGYLYASGDMSSILVWD
LDKEQLLNTIPSSADSGISALSASQVRSGQFAAGFIDASVRIFDVRTPDRLVYMARPHAPRTEKVVGIGFQPGFDPYKIV
SASQAGDIQFLDVRRAAEPYLTIEAHRGSLTALAVHRHAPVIASGSAKQMIKVFSLEGEQLTIIRYQPSEMGQRIGSVNC
LSFHPYKSLLAAGAGDNALVSIYAEDNYQVR
>XM_044524689.1 LOC123103186 [organism = Triticum aestivum] [GeneID = 123103186]
[transcript = X1]
(SEQ ID NO: 40)
GTTTCTCTCTCTCCCTCTTCCTCCCCACCCTCTCCGCCGCATCCCCGTCTCCCCCGACCCTCACCCCCACCCCGCTGCTC
CCGTCCAAACCCTAACCCTAAACCCCACCCTGCGCCTCCATCCAATCCCTGCCTGGCGTCCACCCGTCCCCGGCGGCACG
GATTCGGATTCGCCGCGCCGGACCGATTCGATCGATGTAGGCAGCGAGCATCGCTGCCCACTCCGTACACACGACTCGCG
CCCCCGCCTCGAGATGGCTGCCGCGGGGTGACGAGGAGGAGGGGAGAGTAGTAGAGGGAGCGCCCAAGCGTAGATGGCAT
TGGGAGATCTCATGGCGTCCAGGCTCGTGCACTCGTCCTCATCGTCGTCCTCGTCCTCGCTGCCCACCCCCTCCTTGGCG
GCGGTGAACCACCAGACGGACCGCGTGGATGGTGAGCTCCCTGTCGCCAACGGGCCGGAGCTTCGGAGGGAGGACGCCGG
CGAGGAGGATGAGGAGGGGAGGGCCGTGGCGCTCGTGCCGTGCCTGCCGCAGGTGGTTGTGCTGTGCGAGCAGCGGCACG
AGGGGTTCAATGAGGCTGCGGTGGCGGCGGCGGCCGGGCCATCGACGAGCGGGCTCGTCTCCAAGTGGCGCCCCAAGGAC
AGGATGAAGACTGGATGTGTTGCACTTGTATTATGTTTAAACATTAGTGTTGATCCGCCGGATGTAATTAAAATTTCCCC
TTGTGCAAGAATGGAGTGTTGGATAGATCCATTTTCTATGCCACCCCCCAAAGCCCTTGAAAATATTGGAAAAACATTAC
ACTCACAGTATGAGCGATGGCAGCCTAAGGCTCGTTACAAGCTTCAGCTGGATCCAACAGTCGAAGAAGTCAAGAAGCTT
TGTAATACTTGCCGCAAATATGCCAGATCAGAGAGGGTTCTTTTTCATTACAATGGTCATGGTGTACCAAAGCCTACGGC
TAATGGAGAGATTTGGATGTTTAACAAGAGTTATACACAGTATATCCCTCTTCCTATTACTGATCTCGATTCATGGCTGA
AAACACCATCGATTTATGTTTTTGACTGCTCAGCAGCTGGAATGATTGTGAAAGCTTTTCTAGAGCGCCTAGACTGGAGT
TCAAGCTCTTCTGCGTCTTCAGTGAAGGATTGCATTCTCCTTGCTGCCTGTGAGGCACATCAAACTCTTCCTCAGAGTGC
AGAATATCCCGCTGACGTGTTTACAGCTTGCCTGACCACTCCCATAAAAATGGCATTGCACTGGTTTTGCAAACGATCAT
TACTCAGTGGTTCTCTGGATCACTCTCTTATTGACCAAATTCCTGGAAGACAAAATGACCGTAAAACACTTCTGGGGGAG
TTGAACTGGATTTTCACTGCTATTACAGATACTATTGCATGGAATGTTCTTCCTCATGAATTATTCCAAAGGCTTTTCAG
GCAGGATCTTTTGGTCGCCAGTCTCTTTCGCAACTTCTTACTTGCTGAGAGAATCATGCGGTCTGCAAACTGCTCACCAA
TTACGTACCCACTATTGCCACCCACACATCAACATCATATGTGGGATGCATGGGACATGGCTGCGGAGATCTGCCTTTCC
AAGCTTCCTCATTTGATTGCTGATCCTAATGCAGAGTTTCAGCCGAGCCCATTTTTCACGGAACAATTGACAGCATTCGA
AGTGTGGCTTGATCATGGTTCTGAGGATAAGAAACCCCCTGAACAGTTACCCATTGTTCTTCAGGTCCTGCTTAGCCAAT
CACACAGATTTAGAGCACTTGTTCTGCTTGGAAGATTTCTTGATATGGGTCCATGGGCAGTTGATTTGGCCTTATCTGTT
GGTATCTTCCCTTACGTGCTCAAACTGCTCCAAACAAGTGCGATGGAGTTGCGTCAAATTCTTGTGTTCATATGGACAAA
AATTCTCTCTCTTGATAAGTCATGCCAGGTTGATTTGGTGAAAGATGGAGGGCATGCATATTTTATCAGGTTTCTTGATA
GTTTGGATGCTTACCCGGAGCAGCGTGCAATGGCTGCTTTCGTTTTAGCAGTTATTGTGGATGGGCATAGGAGGGGTCAA
GAGGCTTGCATGAGTGCAGGTCTTATAGATGTTTGCCTGAGACATCTGCAACCTGAGAATCCTCATGATGCACAGACAGA
GCCTTTGCTTTTGCAATGGCTTTGTTTATGCCTTGGCAAACTCTGGGAAGATTTCCCTGAGGCTCAGTTACTTGGTCTAC
AATCAAATGCACCCGAAATTGTTACATGTTTATTGTCTGAGCCTCAACCTGAGGTCAGAGCCTCTGCTGTTTTTGCACTT
GGAAATCTGTTGGACATTGGATCTCCATCAGTGAATGGAGGTGATGACGACTCTGATGATGATGAAAAGGCGAGAGCTGA
AATAAATGTTGTTCGAAGCCTTCTGCAAGTCACTTCAGATGGTAGCCCTCTTGTTAGAGCCGAGGTTTCTGTAGCTCTTA
CTCGCTTTGCGCTGGGCCACAACAAACATCTCAAATCTGTTGCTGCGGAGTACTGGAGACCTCAAACCAATTCACTGCTG
AAGTCACTACCATCATTGGCTAATATTAATAACCCAAGCAATGTTTACAGTCCCAGTAACTTTTTGCAAGGCAGCAGTGG
CCTTTCTTCTCATATTGGTCCTGTGTTAAGGGTTGGTAGTGATACCAGTGCCACAGGTCGTGATGGAAGAATTTCTACAA
GCAGCCCGATTGCGACAAATAGCATTATGCATGGGTCTCCCCAGTCAGATGATTCTTCTCAGCACTCTGATTCAGGCATA
TTGCTGAGAGATAATGCAAGTAACGGTGGTCTCAGCTACACCAGGTCAAGGCCTATTATTGACAGTGGAATTTATTCCCT
ATTTATTTCGACGATGTGCTCTATTGCTAAAGATCCTTACCCTAGAATTGCAAATATTGGCCGGAGAGCACTGTCCCTTG
TAGGGGTTGAGCAAGTGGTCATGAGAAATACTAGATTTGGCAGTGGGGGTGCAGGAGAGTCATCTGCTCCTTCATCAAAC
ATAGGAATGGCACGTTCTTCTTCCTGGTTTGACATGAATTCTGGAATCTCAATGGCATTTAGGACCCCTCCTGTTAGTCC
ACCTCAGCATGATTACCTTACAGGGTTGCGCCGCGTGTGTTCTATGGAGTTCAGACCACATCTCTTGAACTCACCTGATG
GCTTAGCTGATCCGCTCTTAAGCTCTGCTGCAGGCCCCAGTACCAGTGAGCTTAATATACTTCCCCAATCAACAATTTAC
AACTGGAGTTGTGGCCACTTCTCTAGGCCTCTTCTAACCGGGTCTGATGATAATGGGGAAGTAAGTGCTAGAAGAGAAGA
GAGGGAACGAACTGCACTGGATTGCATCGCTAAGTGCCAGCGTTCCTCAGCATGCAAAATGACTAGCCAAATTGCTAGCT
GGGATACAAAGTTTGAGTTGGGTACAAAATCAGCATTGCTGTTACCTTTTTCTCCAATCGTCGTTGCAGCTGATGAAAAC
GAGCAAATAAGAGTGTGGAATTATGACGATGCTCTACCAGTGAATACTTTCGAGAACCACAAGTTGTCTGACAGAGGACT
ATCCAAACTTTTACTTATCAATGAGCTTGATGAAAGCTTGCTTTTAGTTGGCTCAAGTATGCCTTCCCTCTTTATGGCTT
TCTCTTGTATCTGTACATATGTATTCCTTGTGACATCTTTTTTTCATACTGTAGGTGATGGAAATGTCCGTATATGGAGA
AACTATACTCAAAAGGGAGGACAGAAACTTGTAACTGCTTTCTCATCAGTTCAGGGCCATCGAGCTGCAGGCCGCAGCGT
TGTGATTGACTGGCAACAGCAATCTGGTTATCTGTATGCATCTGGTGACATGTCTTCCATCCTTGTATGGGATCTTGACA
AGGAGCAACTTCTCAACACCATTCCATCATCTGCTGATAGTGGCATTTCAGCTCTGTCTGCATCTCAGGTTAGATCTGGT
CAATTTGCTGCCGGTTTTATTGACGCATCCGTAAGGATATTTGATGTTCGTACCCCTGACAGGCTAGTTTATATGGCAAG
ACCACATGCCCCAAGAACTGAAAAGGTCGTCGGTATAGGATTTCAGCCTGGATTTGATCCATACAAGATTGTAAGCGCAT
CTCAAGCTGGAGACATTCAGTTTCTTGATGTTAGAAGGGCTGCTGAACCCTACCTCACTATTGAAGCTCACAGGGGTTCA
TTAACAGCATTGGCTGTTCACCGGCATGCCCCAGTCATTGCAAGTGGCTCAGCTAAGCAAATGATTAAAGTGTTCAGTCT
TGAAGGAGAACAGCTAACGATCATTCGGTACCAGCCATCTTTTATGGGCCAAAGGATAGGAAGTGTAAACTGCCTTTCGT
TCCATCCGTACAAATCTCTCCTGGCTGCTGGAGCTGGTGATAATGCTCTGGTCTCTATCTATGCTGAGGACAATTATCAA
GTAAGATGAGGTTTACATCCTGTGGCATGCAATGACGACACAATCAGTAGGCCATGATTGGCCGACTGCTGGATCCGCAG
CAGGTTCAGCTACAAGATACAAGATGATACAAAGCCTGGTCAGTGACATGGTGGTGATTGCAGTGCTGTGGCTTTTTGGT
CCATATTGCTTGTGTTGGGCATCTACATCTCTGGTCGGCGTGTGCAATTTGTAAGATACAAATCAAGATTCATTAGCCTC
CCTTGCAGAAGGTTGTGAAAGTGAAAGGCAGTTGTGGTCCAGTCAGTGAATTCGGAGGCTTCCCCAATAATCATACCTAG
GTTAACTGAAATGTAAATAGCCGTCCCCATTTGGATTGTTGTCCTGTCAGTCAGTTTTCTTCCTTTCCCAAAGTGTAAAA
AGGTTTGCCGATGCTTCATATCATAAGTCGGGGTCTGGCGGGGTCTGCCCGGGCAAGTGGTTGTTTATATGGGCCAGGAA
GGGAGAATGCATGCACATGTCTCCTCACAGATGTTCTTCAATAGATAGATGCTCCTTTCCAGCTAATATAGCATTAGAGG
TGAGTTGTTACTATGGCTTGCTTTAACCAGTGTTGTCCAGGTCATTCTTTCTAAGCATTGGACTATCATCATCCAGTGTT
GTCGTCATAGCAGGGCTTCATTGTCCATTTGTACATATGAACTGCATTTGTACAGTAGTTAATTCTCTTAACATAAGTTG
TAATGCATGTTTCTCCTTGGGTGTATCTGGCATATTCTGAATATTTAAGTGAAAAAGAAGCTTGTCTCTGTGTA
>XP_044388100.1 regulatory-associated protein of TQR 1 isoform X1 B genome
[Triticum aestivum]
(SEQ ID NO: 41)
MALGDLMASRLVHSSSSSSSSSLPTPSLAAVNHQTDRVDGELPVANGPELRREDAGEEDEEGKAVALVPCLPQVVVLCEQ
RHEGFDEAVAAAAGPSTSGLVSKWRPKDRMKTGCVALVLCLNISVDPPDVIKISPCARMECWIDPFSMPPPKALETIGKT
LHSQYERWQPKARYKLQLDPTVEEVKKLCNTCRKYARSERVLFHYNGHGVPKPTANGEIWVENKSYTQYIPLPITDLDSW
LKTPSIYVFDCSAAGMIVKAFLERLDWSSSSSASSVKDCILLAACEAHQTLPQSAEYPADVFTACLTTPIKMALHWFCKR
SLLSGALDHSLIDQIPGRQNDRKTLLGELNWIFTAITDTIAWNVLPHELFQRLFRQDLLVASLERNELLAERIMRSANCS
PITYPLLPPTHQHHMWDAWDMAAEICLSKLPHLIADPNAEFQPSPFFTEQLTAFEVWLDHGSEDKKPPEQLPIVLQVLLS
QSHRFRALVLLGRFLDMGPWAVDLALSVGIFPYVLKLLQTSAMELRQILVFIWTKILSLDKSCQVDLVKDGGHAYFIRFL
DSLDAYPEQRAMAAFVLAVIVDGHRRGQEACMSAGLIDVCLRHLQPENPHDAQTEPLLLQWLCLCLGKLWEDFPEAQLLG
LQSNAPEIVTCLLSEPQPEVRASAVFALGNLLDIGSPSVNGGDDDSDDDEKVRAEINVVRSLLQVTSDGSPLVRAEVSVA
LTRFALGHNKHLKSVAAEYWRPQTNSLLKSLPSLANINNPSNVYSPSNFLQSSSGLSSHIGPVLRVGSDTSATGRDGRIS
TSSPIATNSIMHGSPQSDDSSQHSDSGILLRENASNGGLSYTRSRPIIDSGIYSLFISTMCSVAKDPYPRIANIGRRALS
LVGVEQVVMRNTRFGSGGAGETSAPSSNIGMARSSSWFDMNSGISMAFRTPPVSPPQHDYLTGLRRVCSMEFRPHLLNSP
DGLADPLLSSAAAPSTSELNILPQSTIYNWSCGHFSRPLLTGSDDNGEVSARREERERTALDCIAKCQRSSACKMTSQIA
SWDTKFELGTKSALLLPFSPIVVAADENEQIRVWNYDDALPVNTFENHKLSDRGLSKLLLINELDESLLLVGSSDGNVRI
WRNYTQKGGQKLVTAFSSVQGHRAAGRSVVIDWQQQSGYLYASGDMSSILVWDLDKEQLLNTIPSSADSGISALSASQVR
PGQFAAGFIDASVRIFDVRTPDRLVYMARPHAPRTEKVVGIGFQPGFDPYKIVSASQAGDIQFLDVRRAAEPYLTIEAHR
GSLTALAVHRHAPVIASGSAKQMIKVFSLEGEQLTIIRYQPSFMGQRIGSVNCLSFHPYKSLLAAGAGDNALVSIYAEDN
YQVR
>XM_044532165.1 LOC123111378 [organism = Triticum aestivum] [GeneID = 123111378]
[transcript = X1]
(SEQ ID NO: 42)
CTCTCCGCCGCATCCCCGTCTCCCCCGACCCTCACCCCCACCCCGCTGCTCCCGTCCAAACCCTAACCCTAAACCCCACC
CGCGCCTCCATCCAATCCCTGCCTGGCGCCCACCCGTCCCCGGCGGCCCGGATTCGGATTCGCCGCGCCGGACCGATTCG
ATCGATGTAGGCAGCGAGCATCGCTGCCCACTCCGTACACAAGACACGCGCCCCCGCCTCGAGATGGCTGCTGCGGGGTG
ACAAGGAGGAGGGGAGAGTAGTAGAGGGAGCGCCCAAGCGTAGATGGCATTGGGAGATCTCATGGCGTCCAGGCTCGTGC
ACTCGTCCTCATCGTCGTCCTCGTCCTCGCTGCCCACTCCCTCCTTGGCGGCGGTGAACCACCAGACGGACCGCGTGGAT
GGTGAGCTCCCTGTCGCGAACGGGCCGGAGCTTCGGAGGGAGGACGCCGGCGAGGAGGATGAGGAGGGGAAGGCCGTTGC
GCTCGTGCCGTGCCTGCCGCAGGTGGTTGTGCTGTGCGAGCAGCGGCACGAGGGGTTCGATGAGGCCGTCGCTGCCGCGG
CGGGGCCATCGACCAGCGGCCTCGTCTCCAAGTGGCGCCCCAAGGACAGGATGAAGACTGGATGTGTTGCACTTGTATTA
TGTTTAAACATTAGTGTTGATCCGCCGGATGTAATTAAAATTTCCCCTTGTGCAAGAATGGAGTGTTGGATAGATCCATT
TTCTATGCCACCTCCCAAAGCCCTTGAAACTATTGGAAAAACATTACACTCACAGTATGAGCGGTGGCAGCCTAAGGCTC
GTTACAAGCTTCAGCTGGATCCAACAGTCGAAGAAGTCAAGAAGCTTTGTAATACTTGCCGCAAATATGCCAGATCAGAG
AGAGTTCTTTTTCATTACAATGGTCATGGTGTACCAAAGCCTACAGCTAATGGAGAGATTTGGGTGTTTAACAAGAGTTA
TACACAATATATCCCTCTTCCTATTACTGATCTCGATTCATGGCTGAAAACACCATCGATTTATGTTTTTGACTGCTCAG
CAGCTGGAATGATTGTGAAAGCTTTTCTAGAGCGCCTAGACTGGAGTTCAAGCTCTTCTGCGTCCTCAGTGAAGGATTGC
ATTCTCCTTGCTGCCTGTGAGGCACATCAAACTCTTCCTCAGAGTGCAGAATATCCCGCTGATGTGTTTACAGCTTGCCT
GACCACTCCCATAAAAATGGCATTGCACTGGTTTTGTAAACGATCATTACTCAGTGGTGCTCTGGATCACTCTCTTATTG
ACCAAATTCCTGGAAGGCAAAATGACCGTAAAACACTTCTGGGGGAGTTGAACTGGATTTTCACTGCTATTACAGATACT
ATTGCATGGAATGTTCTTCCTCATGAATTATTCCAAAGGCTTTTCAGGCAGGATCTTTTGGTCGCCAGTCTCTTTCGCAA
CTTCTTACTTGCTGAGAGAATCATGCGATCTGCAAACTGTTCTCCAATTACATACCCACTATTGCCACCCACACATCAAC
ACCATATGTGGGATGCATGGGACATGGCTGCGGAGATCTGCCTTTCCAAGCTTCCTCATTTGATTGCTGATCCTAATGCA
GAGTTTCAGCCGAGCCCATTTTTCACGGAGCAATTGACAGCATTTGAAGTTTGGCTTGATCATGGCTCTGAGGATAAGAA
ACCACCTGAACAGTTACCCATTGTTCTTCAGGTCCTGCTTAGCCAATCACACAGATTTAGAGCACTTGTTCTGCTTGGAA
GATTTCTTGATATGGGGCCATGGGCAGTTGATTTGGCCTTGTCTGTTGGTATCTTCCCTTACGTGCTCAAACTGCTCCAA
ACAAGTGCGATGGAGTTGCGTCAAATTCTTGTGTTCATATGGACAAAAATTCTCTCTCTTGATAAGTCATGCCAGGTTGA
TTTGGTGAAAGATGGAGGGCATGCATATTTTATCAGGTTTCTTGATAGTTTGGATGCTTACCCGGAGCAGCGTGCAATGG
CTGCTTTCGTTTTAGCAGTTATTGTGGATGGGCATAGGAGGGGTCAAGAGGCTTGCATGAGTGCAGGTCTTATAGATGTT
TGCCTGAGACATCTGCAACCTGAGAATCCTCATGATGCACAGACAGAGCCTTTGCTTTTGCAATGGCTTTGTTTATGCCT
TGGCAAACTCTGGGAAGATTTCCCTGAGGCTCAGTTACTTGGTCTACAATCAAATGCACCCGAAATTGTTACATGTTTAT
TGTCTGAGCCTCAACCTGAGGTCAGAGCCTCTGCTGTTTTTGCACTTGGAAATCTGTTGGACATTGGATCTCCATCAGTG
AATGGAGGTGATGACGACTCTGATGATGATGAAAAGGTGAGAGCTGAAATAAATGTTGTTCGAAGCCTTCTGCAAGTCAC
TTCAGATGGTAGCCCTCTTGTTAGAGCTGAGGTTTCTGTAGCTCTTACTCGCTTTGCGCTGGGCCACAACAAACATCTCA
AATCTGTTGCTGCGGAGTACTGGAGACCTCAAACCAATTCACTGCTGAAGTCACTACCATCATTGGCTAATATTAATAAC
CCAAGCAATGTTTACAGTCCCAGTAACTTTCTGCAAAGCAGCAGTGGCCTTTCTTCGCATATTGGTCCTGTGTTAAGGGT
TGGTAGTGATACCAGTGCCACAGGTCGTGATGGAAGAATTTCTACAAGCAGCCCGATTGCGACAAATAGCATCATGCATG
GGTCTCCCCAGTCAGATGATTCTTCTCAGCACTCTGATTCAGGCATATTGCTTAGAGAGAATGCAAGTAACGGTGGTCTC
AGCTACACCAGGTCAAGGCCTATTATTGACAGTGGAATTTATTCCCTATTTATTTCGACGATGTGCTCTGTTGCTAAAGA
TCCTTACCCTAGAATTGCAAATATTGGCCGGAGAGCACTGTCCCTTGTAGGGGTCGAGCAAGTGGTCATGAGAAATACTA
GATTTGGCAGTGGGGGTGCAGGAGAGACATCTGCTCCTTCATCAAACATAGGAATGGCACGTTCTTCTTCCTGGTTCGAC
ATGAATTCTGGAATCTCAATGGCATTTAGGACTCCTCCTGTTAGTCCACCTCAGCATGATTACCTTACAGGGTTGCGCCG
CGTGTGTTCTATGGAGTTCAGACCACATCTCTTGAATTCACCTGATGGCTTAGCTGATCCGCTCTTAAGCTCTGCTGCAG
CCCCCAGTACCAGTGAGCTTAATATACTTCCTCAATCAACAATATACAACTGGAGTTGTGGCCACTTCTCTAGGCCACTT
CTAACCGGTTCTGATGATAATGGGGAAGTAAGTGCTAGAAGAGAAGAGAGAGAACGAACTGCACTGGATTGCATCGCTAA
GTGCCAGCGTTCCTCAGCATGCAAGATGACTAGCCAAATTGCTAGCTGGGATACAAAGTTTGAGTTGGGTACAAAATCAG
CATTGCTGTTACCTTTTTCTCCTATCGTCGTTGCAGCTGATGAAAACGAGCAAATAAGAGTGTGGAATTATGACGATGCT
CTACCAGTGAATACTTTTGAAAACCACAAGTTATCTGACAGAGGACTATCCAAACTTCTACTTATCAATGAGCTTGATGA
AAGCTTGCTTTTAGTTGGCTCAAGTGATGGAAATGTCCGTATATGGAGAAACTATACTCAAAAGGGAGGACAGAAACTTG
TAACTGCCTTCTCATCAGTTCAGGGCCATCGAGCTGCAGGCCGCAGCGTTGTGATCGACTGGCAACAGCAATCTGGTTAT
CTGTATGCATCTGGTGACATGTCTTCCATCCTCGTATGGGATCTTGACAAGGAGCAACTTCTCAACACCATTCCATCATC
TGCTGATAGTGGCATTTCAGCTCTGTCTGCATCTCAGGTTAGACCTGGTCAATTTGCTGCCGGTTTTATTGATGCATCCG
TAAGGATATTTGATGTTCGTACCCCTGACAGGCTAGTTTACATGGCAAGACCACATGCCCCAAGAACTGAAAAGGTCGTC
GGTATAGGATTTCAGCCTGGATTTGATCCATACAAGATTGTAAGCGCATCTCAAGCTGGAGACATTCAGTTTCTTGATGT
TAGAAGGGCTGCTGAACCCTACCTCACTATTGAAGCTCACAGGGGTTCGTTAACAGCATTGGCTGTTCACCGGCATGCTC
CAGTCATTGCAAGTGGCTCAGCTAAGCAGATGATTAAAGTGTTCAGTCTTGAAGGAGAACAGCTAACAATCATTCGCTAC
CAGCCATCTTTTATGGGCCAAAGGATAGGAAGTGTAAACTGCCTTTCGTTCCATCCGTACAAATCTCTCCTGGCTGCTGG
AGCTGGTGATAATGCTCTGGTCTCTATCTATGCTGAGGACAATTATCAAGTAAGATGAGGTCTACATCCTGTGGCATGCA
ATGACGACACAATCAGTAGGCCATATGATTGGCTGACTGCTGGATCCGCAGCAGGTTCAGCTACAAGATACAAGATGATA
CAAAGCCTGGTCAGTGACATGGTGGTGATTGCAGTGCTGTGGCTTTTTGGTCCATATTGCTTGTGTTGGGCATCTACATC
TCTGGTCGGCGAGTGCAATTTGTAAGATACAAATCAAGATTCATTAGCCTCCCTTGCAGAAGGTTGTGAAAGTGAAAGGC
AGTTGTGGTCCTGTCAGTGAATTTGGAGGCTTCCCCAATAATCATACCTAGGTTAACTGAAATGTAAATAGCCGTCCCCA
TTTGGATTGTTGTCCTGTCAGTCAGTTTTCTTCCTTTCCCAAAGTGTAAAAAGGTTTGTCGATGCTTCATCATATCATAA
GTCGGGTCTGGCGGGGTCTGCCCGGGCAAGTGGTTGTTTATATGGGCCAGGAAGGGAGAATGCATGCACATGTCTCCTCA
CAGATGTTCTTCAATAGATAGATGCTCCTTTCCAGCTAATATAGCATTAGAGGTGAGTTGTTACTATGGCTTGCTTTAAC
CAGTGTTGTCCAGGTCATTCTTTCTAAGCATTGGACTATCATCATCCAGTGTTGTCGTCATAGCAGGGCTTCATTGTCCA
TTTGTACATATGAACTGCATTTGTACAGTAGTTAATTCTCTTAAGATAAGTTGTAATGCATGTTTGTCCTTGGGTGTATC
TGGCATATTTTGAATAATTAAGTGAAAAAGAAGCTTGTCTCTGTGTA
>XP_044396257.1 regulatory-associated protein of TOR 1-like isoform X1 D genome
[Triticum aestivum]
(SEQ ID NO: 43)
MALGDLMASRLVHSSSSSSSSSLPTPSSAAVNHQTDRVDGELPVANGPELRREDAGEEDEEGRAVVLVPCLPQVVVLCEQ
RHEGFDEAAAAVAGPSTSGLVSKWRPKDRMKTGCVALVLCLNISVDPPDVIKISPCARMECWIDPFSMPPPKALESIGKT
LHSQYERWQPKARYKLQLDPTVEEVKKLCNTCRKYARSERVLFHYNGHGVPKPTANGEIWVENKSYTQYIPLPITDLDSW
LKTPSIYVFDCSAAGMIVKAFLERLDWSSSSSASSVKDCILLAACEAHQTLPQSAEYPADVFTACLTTPIKMALHWFCKR
SLLSGSLDHSLIDQIPGRQNDRKTLLGELNWIFTAITDTIAWNVLPHELFQRLFRQDLLVASLERNFLLAERIMRSANCS
PITYPLLPPTHQHHMWDAWDMAAEICLSKLPHLIADPNAEFQPSPFFTEQLTAFEVWLDHGSEDKKPPEQLPIVLQVLLS
QSHRFRALVLLGRFLDMGPWAVDLALSVGIFPYVLKLLQTSAMELRQILVFIWTKILSLDKSCQVDLVKDGGHAYFIRFL
DSLDAYPEQRAMAAFVLAVIVDGHRRGQEACMSAGLIDVCLRHLQPENPHDAQTEPLLLQWLCLCLGKLWEDFPEAQLLG
LQSNAPEIVTCLLSEPQPEVRASAVFALGNLLDIGSPSVNGGDDDSDDDEKVRAEINVVRSLLQVTSDGSPLVRAEVSVA
LTRFALGHNKHLKSVAAEYWRPQTNSLLKSLPSLANINNPSNVYSPSNFLQGSSGLSSHIGPVLRVGSDTSAAGRDGRIS
TSSPIATNSIMHGSPQSDDSSQHSDSGILLRDNASNGGLSYTRSRPIIDSGIYSLFISTMCSIAKDPYPRIANIGRRALS
LVGVEQVVMRNTRFGSGGAGETSAPSSNIGMARSSSWFDMNSGISMAFRTPPVSPPQHDYLTGLRRVCSMEFRPHLLNSP
DGLADPLLSSAAAPSTSELNILPQSTIYNWSCGHFSRPLLTGSDDNGEVTARREERERTALDCIAKCQRSSACKMTSQIA
SWDTKFELGTKSALLLPFSPIVVAADENEQIRVWNYDDALPVNTFENHKLSDRGLSKLLLINELDESLLLVGSSTPCLEM
AVSCICTYVLLVISIFHTVGDGNVRIWRNYAQKGGQKLVTAFSSVQGHRAAGRSVVIDWQQQSGYLYASGDMSSILVWDL
DKEQLLNTIPSSADSGISALSASQVRPGQFAAGFIDASVRIFDVRTPDRLVYMARPHAPRTEKVVGIGFQPGFDPYKIVS
ASQAGDIQFLDVRRAAEPYLTIEAHRGSLTALAVHRHAPVIASGSAKQMIKVFSLEGEQLTIIRYQPSFMGQRIGSVNCL
SFHPYKSLLAAGAGDNALVSIYAEDNYQVR
>XM_044540322.1 LOC123120355 [organism = Triticum aestivum] [GeneID = 123120355]
[transcript = X1]
(SEQ ID NO: 44)
CTCCCCACCCTCTCCGCCGCATCCCCGTCTCCCCCGACCCTCACCCCCACCCCGCTGCTCCCGTCCAAACCCTAACCCTA
AACCCCACCCGCGCCTCCATCCAATCCCTGCCTGGCGTCCACCCGTCCCCGGCGGCCCGGATTCGGATTCGCCGCGCCGG
ACCGATTCGATCGATGTAGGCAGCGAGCATCGCTGCCCACTCCGTACACAGGACTCGCGCCCCCGCCTCGAGATGGCTGC
TGCGGGGTGACGAGGAGGAGGGGAGAGTAGTAGAGGGAGCGCCCAAGCGTAGATGGCATTGGGAGATCTCATGGCGTCCA
GGCTCGTGCACTCGTCCTCATCGTCGTCCTCGTCCTCGTTGCCCACCCCCTCCTCGGCGGCGGTGAACCACCAGACGGAC
CGCGTGGATGGTGAGCTCCCTGTCGCGAACGGGCCGGAGCTTCGGAGGGAGGACGCCGGCGAGGAGGATGAGGAGGGGAG
GGCCGTGGTGCTAGTGCCGTGCCTGCCGCAGGTGGTTGTGCTGTGCGAGCAGCGGCACGAGGGGTTCGATGAGGCCGCCG
CTGCCGTGGCCGGGCCATCGACCAGCGGGCTCGTCTCCAAGTGGCGCCCCAAGGACAGGATGAAGACCGGATGTGTTGCA
CTTGTATTATGTTTAAACATTAGTGTTGATCCGCCGGATGTAATTAAAATTTCGCCTTGTGCAAGAATGGAGTGTTGGAT
AGATCCATTTTCTATGCCACCTCCCAAAGCCCTTGAAAGTATTGGAAAAACATTACACTCACAGTATGAGCGATGGCAGC
CTAAGGCTCGTTACAAGCTTCAGCTGGATCCAACAGTCGAAGAAGTCAAGAAGCTTTGTAATACTTGCCGCAAATATGCC
AGATCAGAGAGGGTTCTTTTTCATTACAATGGTCATGGTGTACCAAAGCCTACAGCTAATGGAGAGATTTGGGTGTTTAA
CAAGAGTTATACACAGTATATCCCTCTTCCTATTACTGATCTCGATTCATGGCTGAAAACACCATCGATTTATGTTTTTG
ACTGCTCAGCAGCTGGAATGATCGTGAAAGCTTTTCTAGAGCGTCTAGACTGGAGTTCAAGCTCTTCTGCGTCTTCAGTG
AAGGATTGCATTCTCCTTGCTGCCTGCGAGGCACATCAAACTCTTCCTCAGAGTGCAGAATATCCTGCTGATGTGTTTAC
AGCTTGCCTGACCACTCCCATAAAAATGGCATTGCACTGGTTTTGCAAACGATCATTACTCAGTGGTTCTCTGGATCACT
CTCTTATTGACCAAATTCCTGGAAGACAAAATGACCGTAAAACACTTCTGGGGGAGTTGAACTGGATTTTCACTGCTATT
ACAGATACTATTGCATGGAATGTTCTTCCTCATGAATTATTCCAAAGGCTTTTCAGGCAGGATCTTTTGGTCGCCAGTCT
CTTTCGCAACTTCTTACTTGCTGAGAGAATCATGCGGTCTGCAAACTGCTCTCCAATTACGTACCCACTATTGCCACCCA
CACATCAACATCATATGTGGGATGCATGGGACATGGCTGCGGAGATCTGCCTTTCCAAGCTTCCTCATTTGATTGCTGAT
CCTAATGCAGAGTTTCAGCCGAGCCCATTTTTCACGGAACAATTGACAGCATTTGAAGTGTGGCTTGATCATGGCTCTGA
GGATAAGAAACCCCCTGAACAGTTACCCATTGTTCTTCAGGTCCTGCTTAGCCAATCACACAGATTTAGAGCACTTGTTC
TGCTTGGAAGATTTCTTGATATGGGGCCATGGGCAGTTGATTTGGCCTTATCTGTTGGTATCTTCCCTTACGTGCTCAAA
CTGCTCCAAACAAGTGCGATGGAGTTGCGTCAAATTCTTGTGTTCATATGGACAAAAATTCTCTCTCTTGATAAGTCATG
CCAGGTTGATTTGGTGAAAGATGGAGGGCATGCATATTTTATCAGGTTTCTTGATAGTTTGGATGCTTACCCGGAGCAGC
GTGCAATGGCTGCTTTCGTTTTAGCAGTTATTGTGGATGGGCATAGGAGGGGTCAAGAGGCTTGCATGAGTGCAGGTCTT
ATAGATGTTTGCCTGAGACATCTGCAACCTGAGAATCCTCATGATGCACAGACAGAGCCTTTGCTTTTGCAATGGCTTTG
TTTATGCCTTGGCAAACTCTGGGAAGATTTCCCTGAGGCTCAGTTACTTGGTCTACAATCAAATGCACCCGAAATTGTTA
CATGTTTATTGTCTGAGCCTCAACCTGAGGTCAGAGCCTCTGCTGTTTTTGCACTTGGAAATCTGTTGGACATTGGATCT
CCATCAGTGAATGGAGGTGATGACGACTCTGATGATGATGAAAAGGTGAGAGCTGAAATAAATGTTGTTCGAAGCCTTCT
GCAAGTCACTTCAGATGGTAGCCCTCTTGTTAGAGCTGAGGTTTCTGTAGCTCTTACTCGCTTTGCGCTGGGCCACAACA
AACATCTCAAATCTGTTGCTGCGGAGTACTGGAGACCTCAAACCAATTCACTGCTGAAGTCACTACCATCATTGGCTAAT
ATTAATAACCCAAGCAATGTTTACAGTCCCAGTAACTTTTTGCAAGGCAGCAGTGGCCTTTCTTCTCATATTGGTCCTGT
GTTAAGGGTTGGTAGTGATACCAGTGCCGCAGGTCGTGATGGAAGAATTTCTACAAGCAGCCCGATTGCGACAAATAGCA
TTATGCATGGGTCTCCCCAGTCAGATGATTCTTCTCAGCACTCTGATTCAGGCATATTGCTGAGAGATAATGCAAGTAAC
GGTGGTCTCAGCTACACAAGGTCAAGGCCTATTATTGACAGTGGAATTTATTCCCTATTTATTTCGACGATGTGCTCTAT
TGCTAAAGATCCTTACCCTAGAATTGCAAATATTGGCCGGAGAGCACTGTCCCTTGTAGGGGTCGAGCAAGTGGTCATGA
GAAATACTAGATTTGGCAGTGGGGGTGCAGGAGAGACATCTGCTCCTTCATCAAACATAGGAATGGCACGTTCTTCTTCC
TGGTTTGACATGAATTCTGGAATCTCAATGGCATTTAGGACCCCTCCTGTTAGTCCACCTCAGCATGATTACCTTACAGG
GTTGCGCCGCGTGTGTTCTATGGAGTTCAGACCACATCTCTTGAACTCACCTGATGGCTTAGCTGATCCGCTCTTAAGCT
CTGCTGCAGCCCCCAGTACCAGTGAGCTTAATATACTTCCCCAATCAACAATTTACAACTGGAGTTGTGGCCACTTCTCT
AGGCCTCTTCTAACCGGGTCTGATGATAATGGGGAAGTAACTGCTAGAAGAGAAGAGAGGGAACGAACTGCACTGGATTG
CATCGCTAAGTGCCAGCGTTCCTCAGCATGCAAGATGACTAGCCAAATTGCTAGCTGGGATACAAAGTTTGAGTTGGGTA
CAAAATCAGCATTGCTGTTACCTTTTTCTCCAATCGTCGTTGCAGCTGATGAAAACGAGCAAATAAGAGTGTGGAATTAT
GACGATGCTCTACCAGTGAATACTTTCGAAAACCACAAGTTATCTGACAGAGGACTATCCAAACTTTTACTTATCAATGA
GCTTGATGAAAGCTTGCTTTTAGTTGGCTCAAGTACGCCTTGCCTCTTTATGGCTGTCTCTTGTATCTGTACATATGTAC
TCCTTGTGATATCTATTTTTCATACTGTAGGTGATGGAAATGTCCGTATATGGAGAAACTATGCTCAAAAGGGAGGACAG
AAACTTGTAACTGCTTTCTCATCAGTTCAGGGCCATCGAGCTGCAGGCCGCAGCGTTGTGATTGACTGGCAACAGCAATC
TGGTTATCTGTATGCATCTGGTGACATGTCTTCCATCCTTGTATGGGATCTTGACAAGGAGCAACTTCTCAACACCATTC
CATCATCTGCTGATAGTGGCATTTCAGCTCTGTCTGCATCTCAGGTTAGACCTGGTCAATTTGCTGCCGGTTTTATTGAC
GCATCCGTAAGGATATTTGATGTTCGCACCCCTGACAGGCTAGTTTATATGGCAAGACCACATGCCCCAAGAACTGAAAA
GGTCGTCGGTATAGGGTTTCAGCCTGGATTTGATCCATACAAGATTGTAAGCGCATCTCAAGCTGGAGACATTCAGTTTC
TTGATGTTAGAAGGGCTGCTGAACCCTACCTCACTATTGAAGCTCACAGGGGTTCATTAACAGCACTGGCTGTTCACCGG
CATGCCCCAGTCATTGCAAGTGGCTCAGCTAAGCAAATGATTAAAGTGTTCAGTCTTGAAGGAGAACAGCTAACGATCAT
TCGGTACCAGCCATCTTTTATGGGCCAAAGGATAGGAAGTGTAAACTGCCTTTCGTTCCATCCGTACAAATCTCTCCTGG
CTGCTGGAGCTGGTGATAATGCTCTGGTCTCTATCTATGCTGAGGACAATTATCAAGTAAGATGAGGTTTACATCCTGTG
GCATGCAATGACGACACAATCAGTAGGCCATATGATTGGCCGACTGCTGGATCCGCAGCAGGTTCAGCTACAAGATACAA
GATGATACAAAGCCTGGTCAGTGACATGGTGGTGATTGCAGTGCTGTGGCTTTTTGGTCCATATTGCTTGTGTTGGGCAT
CTACATCTCTGGTCGGCATGTGCAATTTGTAAGATACAAATCAAGATTCATTAGCCTCCCTTGCAGAAGGTTGTGAAAGT
GAAAGGCAGTTGTGGTCCAGTCAGTGAATCCGGAGGCTTCCCCAATAATCATACCTAGGTTAACTGAAATGTAAATAGCC
GTCCCCATTTGGATTGTTGTCCTGTCAGTCAGTTTTCTTCCTTTCCCAAAGTGTAAAAAGGTTTGCCGATGCTTCATATC
ATAAGTCGGGTCTGGCGGGGTCTGCAAGTGGTTGTTTATATGGGCCAGGAAGGGAGAATGCATGCACATGTCTCCTCACA
GATGTTCTTCAATAGATAGATGCTCCTTTCCAGCTAATATAGCATTTAGAGGTGAGTTGTTACTATGGCTTGCTTTAACC
AGTGTTGTCCAGGTCATTCTTTGTAAGCATTGGACTATCATCATCCAGTGTTGTCGTCATAGCAGGGCTTCATTGTCCAT
TTGTACATATGAACTGCATTTGTACAGTAGTTAATTCTCTTAAGATAAGTTGTAATGCATGTTTGTCCTTGGGTGTATCT
GGCATATTCTGAATATTTAAGTGAAAAAGAAGCTTGTCTCTGTGTA

What is claimed is:

1. A plant, or a plant cell thereof, with increased phosphorus use efficiency or tolerance to phosphorus deficiency, wherein the plant comprises a heterologous polynucleotide encoding a protein kinase having at least 80%, at least 90%, at least 95%, at least 98%, or at least 99% sequence identity to at least one of the amino acid sequences set forth in SEQ ID NOs: 1, 3, 5, 7, 9, 11, 13, 15, 17, 19, or 21.

2. The plant of claim 1, wherein the polynucleotide encoding the protein kinase has at least 80%, at least 90%, at least 95%, at least 98%, or at least 99% sequence identity to at least one of the nucleotide sequences set forth in SEQ ID NO: 2, 4, 6, 8, 10, 12, 14, 16, 18, 20, or 22.

3. The plant of claim 1 or claim 2, wherein the polynucleotide is operably linked to a promoter functional in a plant cell.

4. The plant of claim 3, wherein the promoter is a constitutive promoter, a tissue-preferred promoter, or a chemical-regulated promoter.

5. The plant of claim 3 or claim 4, wherein the promoter is a root-preferred promoter.

6. The plant of any one of claims 1-5, wherein the protein kinase comprises one or more of the amino acid sequences set forth in SEQ ID NOs: 23, 24, or 25.

7. The plant of any one of claims 1-6, wherein the plant is a crop plant, optionally wherein the plant is a Brassica, wheat, maize, potato, soybean, or cotton plant.

8. The plant of any one of claims 1-7, wherein the plant has increased phosphorus use efficiency or tolerance to phosphorus deficiency as compared to a corresponding control plant without the heterologous polynucleotide.

9. The plant of any one of claims 1-7, wherein the plant has increased root biomass as compared to a corresponding control plant without the heterologous polynucleotide when grown in phosphorus deficient conditions.

10. A plant, or a plant cell thereof, with increased phosphorus use efficiency or tolerance to phosphorus deficiency, wherein the plant comprises a modification in at least one genomic sequence encoding a protein kinase having at least 80%, at least 90%, at least 95%, at least 98%, or at least 99% sequence identity to at least one of the amino acid sequences set forth in SEQ ID NOs: 1, 3, 5, 7, 9, 11, 13, 15, 17, 19, or 21, and wherein the modification results in increased expression or activity of the protein kinase.

11. The plant of claim 10, wherein the protein kinase comprises one or more of the amino acid sequences set forth in SEQ ID NOs: 23, 24, or 25.

12. The plant of claim 10 or claim 11, wherein the modification comprises a deletion, an insertion, or a substitution of one or more nucleotides in a transcription regulatory region of the genomic sequence.

13. The plant of any one of claims 10-12, wherein the modification disrupts a methylation site within the transcription regulatory region.

14. The plant of any one of claims 10-13, wherein the plant is a crop plant, optionally wherein the plant is a Brassica, wheat, maize, potato, soybean, or cotton plant.

15. The plant of any one of claims 10-14, wherein the plant has increased phosphorus use efficiency or tolerance to phosphorus deficiency as compared to a corresponding control plant without the modification.

16. The plant of any one of claims 10-15, wherein the plant has increased root biomass as compared to a corresponding control plant without the modification when grown in phosphorus deficient conditions.

17. A seed of the plant of any one of claims 1-16.

18. A method of increasing phosphorus use efficiency or tolerance to phosphorus deficiency in a plant, the method comprising:
introducing a heterologous polynucleotide encoding a protein kinase into at least one plant cell, wherein the protein kinase has at least 80%, at least 90%, at least 95%, at least 98%, or at least 99% sequence identity to at least one of the amino acid sequences set forth in SEQ ID NOs: 1, 3, 5, 7, 9, 11, 13, 15, 17, 19, or 21.

19. The method of claim 18, wherein the polynucleotide encoding the protein kinase has at least 80%, at least 90%, at least 95%, at least 98%, or at least 99% sequence identity to at least one of the nucleotide sequences set forth in SEQ ID NO: 2, 4, 6, 8, 10, 12, 14, 16, 18, 20, or 22.

20. The method of claim 18 or claim 19, wherein the polynucleotide is operably linked to a promoter functional in a plant cell.

21. The method of claim 20, wherein the promoter is a constitutive promoter or a tissue-preferred promoter, or a chemical-regulated promoter.

22. The method of claim 20 or claim 21, wherein the promoter is a root-preferred promoter.

23. The method of any one of claims 18-22, wherein the protein kinase comprises one or more of the amino acid sequences set forth in SEQ ID NOs: 23, 24, or 25.

24. The method of any one of claims 18-23, wherein the plant is a crop plant, optionally wherein the plant is a Brassica, wheat, maize, potato, soybean, or cotton plant.

25. The method of any one of claims 18-24, wherein the plant cell is regenerated into a plant comprising in its genome the polynucleotide.

26. The method of claim 25, further comprising selecting for a plant having increased phosphorus use efficiency or tolerance to phosphorus deficiency as compared to a corresponding control plant without the polynucleotide.

27. A method of increasing phosphorus use efficiency or tolerance to phosphorus deficiency in a plant, the method comprising:
introducing a modification in at least one genomic sequence encoding a protein kinase into at least one plant cell, wherein the protein kinase has at least 80%, at least 90%, at least 95%, at least 98%, or at least 99% sequence identity to at least one of the amino acid sequences set forth in SEQ ID NOs: 1, 3, 5, 7, 9, 11, 13, 15, 17, 19, or 21, and wherein the modification results in increased expression or activity of the protein kinase.

28. The method of claim 27, wherein the protein kinase comprises one or more of the amino acid sequences set forth in SEQ ID NOs: 23, 24, or 25.

29. The method of claim 27 or claim 28, wherein the modification comprises a deletion, an insertion, or a substitution of one or more nucleotides in a transcription regulatory region of the genomic sequence.

30. The method of any one of claims 27-29, wherein the modification disrupts a methylation site within the transcription regulatory region.

31. The method of any one of claims 27-30, wherein the modification is introduced using genome editing.

32. The method of claim 31, wherein the genome editing comprises use of a zinc-finger nuclease (ZFN), a TAL (transcription activator-like) effector nuclease (TALEN), or a Clustered Regularly Interspaced Short Palindromic Repeats/CRISPR-associated nuclease (CRISPR/Cas nuclease) system.

33. The method of any one of claims 27-32, wherein the plant is a crop plant, optionally wherein the plant is a Brassica, wheat, maize, potato, soybean, or cotton plant.

34. The method of any one of claims 27-33, wherein the plant cell is regenerated into a plant comprising in its genome the modification.

35. The method of claim 34, further comprising selecting for a plant having increased phosphorus use efficiency or tolerance to phosphorus deficiency as compared to a corresponding control plant without the modification.

36. A method for limiting use of phosphorus-based fertilizer in agricultural crop production, the method comprising:
planting a seedling, cutting, tuber, or seed of the plant of any one of claims 1-16; and growing the seedling, cutting, tuber, or seed under conditions favorable for the growth and development of a plant resulting therefrom.

37. The method of claim 36, further comprising harvesting at least part from the plant, optionally wherein the part is a fruit, tuber, root, leaf, or seed.

38. A method for conferring improved phosphorus use efficiency to a plant, the method comprising:
(a) crossing the plant of any one of claims 1-16 with a second plant lacking in its genome the heterologous polynucleotide or modification, whereby at least one progeny plant is produced; and
(b) selecting at least one progeny plant comprising in its genome the heterologous polynucleotide or modification.

39. The method of claim 38, further comprising (i) backcrossing at least one selected progeny plant of (b) to a plant lacking in its genome the heterologous polynucleotide or modification, whereby at least one progeny plant is produced from the backcrossing; and (ii) selecting at least one progeny plant comprising in its genome the heterologous polynucleotide or modification that is produced from the backcrossing of (i).

40. A plant, or a plant cell thereof, with increased phosphorus use efficiency or tolerance to phosphorus deficiency, wherein the plant comprises a polynucleotide encoding a Raptor polypeptide, and wherein the Raptor polypeptide comprises a serine to aspartic acid substitution at a position corresponding to position 740 of SEQ ID NO: 27.

41. The plant of claim 40, wherein the Raptor polypeptide has at least 80%, at least 90%, at least 95%, at least 98%, or at least 99% sequence identity to at least one of the amino acid sequences set forth in SEQ ID NO: 27, 29, 31, 33, 35, 37, 39, 41, or 43.

42. The plant of claim 40 or claim 41, wherein the polynucleotide encoding a Raptor polypeptide has at least 80%, at least 90%, at least 95%, at least 98%, or at least 99% sequence identity to at least one of the nucleotide sequences set forth in SEQ ID NO: 28, 30, 32, 34, 36, 38, 40, 42, or 44.

43. A method of increasing phosphorus use efficiency or tolerance to phosphorus deficiency in a plant, the method comprising:
expressing a polynucleotide encoding a Raptor polypeptide in at least one plant cell, wherein the Raptor polypeptide comprises a serine to aspartic acid substitution at a position corresponding to position 740 of SEQ ID NO: 27.

44. The method of claim 43, wherein the Raptor polypeptide has at least 80%, at least 90%, at least 95%, at least 98%, or at least 99% sequence identity to at least one of the amino acid sequences set forth in SEQ ID NO: 27, 29, 31, 33, 35, 37, 39, 41, or 43.

45. The method of claim 43 or claim 44, wherein the polynucleotide encoding a Raptor polypeptide has at least 80%, at least 90%, at least 95%, at least 98%, or at least 99% sequence identity to at least one of the nucleotide sequences set forth in SEQ ID NO: 28, 30, 32, 34, 36, 38, 40, 42, or 44.
